(19) United States
(12) Patent Application Publication
(10) Pub. No.: US 2012/0088746 A1
(43) Pub. Date: Apr. 12, 2012

(54) AMIDE DERIVATIVES AS ION-CHANNEL LIGANDS AND PHARMACEUTICAL COMPOSITIONS AND METHODS OF USING THE SAME

(75) Inventors: Yuji Shishido, Aichi-ken (JP); Kazunari Nakao, Aichi-ken (JP); Satoshi Nagayama, Aichi-ken (JP); Hirotaka Tanaka, Aichi-ken (JP); Matthew Alexander James Duncton, San Francisco, CA (US); Matthew Cox, San Francisco, CA (US); John Kincaid, San Francisco, CA (US); Kiran Sahasrabudhe, Newark, CA (US); Maria de Los Angeles Estiarte-Martinez, San Francisco, CA (US)

(73) Assignees: PFIZER INC., New York, NY (US); RENOVIS, INC., South San Francisco, CA (US)

(21) Appl. No.: 12/227,197

(22) PCT Filed: May 10, 2007
(86) PCT No.: PCT/US07/11310

§ 371 Date: Dec. 22, 2011

Related U.S. Application Data
(60) Provisional application No. 60/799,156, filed on May 10, 2006.
(60) Provisional application No. 60/845,997, filed on Sep. 20, 2006.

(51) Int. Cl. A61K 31/397; C07D 213/76; C07D 215/48; C07D 215/54; C07D 295/155; C07D 205/04; A61K 31/18; A61K 31/44; A61K 31/47; A61K 31/402; A61K 31/5375; A61K 31/451; A61P 19/02; A61P 19/08; A61P 1/00; A61P 25/16; A61P 29/00; A61P 25/28; A61P 25/18; A61P 25/22; A61P 25/08; A61P 13/00; A61P 13/12; A61P 13/10; A61P 9/12; A61P 35/00; A61P 3/04; A61P 3/00; A61P 17/06; A61P 17/14; A61P 17/04; A61P 9/10; A61P 11/00; A61P 11/06; A61P 25/00; C07C 311/16

(52) U.S. Cl. 514/210.01; 564/99; 546/312; 546/169; 548/577; 544/159; 548/950; 546/205; 514/605; 514/352; 514/311; 514/429; 514/239.5; 514/319

(57) ABSTRACT

Compounds are disclosed that have a formula represented by the following: Formula (I). The compounds may be prepared as pharmaceutical compositions, and may be used for the prevention and treatment of a variety of conditions in mammals including humans, including by way of non-limiting example, pain, inflammation, traumatic injury, and others.

### FIELD OF THE INVENTION

This invention relates to novel compounds and to pharmaceutical compositions containing such compounds. This invention also relates to methods for preventing and/or treating pain and inflammation-related conditions in mammals, such as (but not limited to) arthritis, Parkinson's disease, Alzheimer's disease, stroke, uveitis, asthma, myocardial infarction, the treatment and prophylaxis of pain syndromes (acute and chronic or neuropathic), traumatic brain injury, acute spinal cord injury, neurodegenerative disorders, alopecia (hair loss), inflammatory bowel disease, urinary incontinence, chronic obstructive pulmonary disease, irritable bowel disease, osteoarthritis, and autoimmune disorders, using the compounds and pharmaceutical compositions of the invention.

### BACKGROUND OF THE INVENTION

Studies of signaling pathways in the body have revealed the existence of ion channels and sought to explain their role. Ion channels are integral membrane proteins with two distinctive characteristics: they are gated (open and closed) by specific signals such as membrane voltage or the direct binding of chemical ligands and, once open, they conduct ions across the cell membrane at very high rates.

There are many types of ion channels. Based on their selectivity to ions, they can be divided into calcium channel, potassium channel, sodium channel, etc. The calcium channel is more permeable to calcium ions than other types of ions, the potassium channel selects potassium ions over other ions, and so forth. Ion channels may also be classified according to their gating mechanisms. In a voltage-gated ion channel, the opening probability depends on the membrane voltage, whereas in a ligand-gated ion channel, the opening probability is regulated by the binding of small molecules (the ligands). Since ligand-gated ion channels receive signals from the ligand, they may also be considered as “receptors” for ligands.

Examples of ligand-gated ion channels include nAChR (nicotinic acetylcholine receptor) channel, GluR (glutamate receptor) channel, ATP-sensitive potassium channel, G-protein activated channel, cyclic-nucleotide-gated channel, etc.

Transient receptor potential (TRP) channel proteins constitute a large and diverse family of proteins that are expressed in many tissues and cell types. This family of channels mediates responses to nerve growth factors, pheromones, olfaction, tone of blood vessels and metabolic stress et al., and the channels are found in a variety of organisms, tissues and cell types including nonexcitable, smooth muscle and neuronal cells. Furthermore, TRP-related channel proteins are implicated in several diseases, such as several tumors and neurodegenerative disorders and the like. See, for example, Minke, et al., APStracts 9:0006P (2002).

Nociceptors are specialized primary afferent neurons and the first cells in a series of neurons that lead to the sensation of pain. The receptors in these cells can be activated by different noxious chemical or physical stimuli. The essential functions of nociceptors include the transduction of noxious stimuli into depolarizations that trigger action potentials, conduction of action potentials from primary sensory sites to synapses in the central nervous system, and conversion of action potentials into neurotransmitter release at presynaptic terminals, all of which depend on ion channels.

One TRP channel protein of particular interest is the vanilloid receptor. Also known as VR1, the vanilloid receptor is a non-selective cation channel which is activated or sensitized by a series of different stimuli including capsaicin, heat and acid stimulation and products of lipid bilayer metabolism (anandamide), and lipoxygenase metabolites. See, for example Smith, et al., Nature, 418:186-190 (2002). VR1 does not discriminate among monovalent cations, however, it exhibits a notable preference for divalent cations with a permeability sequence of Ca²⁺>Mg²⁺>Na⁺=K⁺=Cs⁺. Ca²⁺ is especially important to VR1 function, as extracellular Ca²⁺ mediates desensitization, a process which enables a neuron to adapt to specific stimuli by diminishing its overall response to a particular chemical or physical signal. VR1 is highly expressed in primary sensory neurons in rats, mice and humans, and innervates many visceral organs including the dermis, bones, bladder, gastrointestinal tract and lungs. It is also expressed in other neuronal and non-neuronal tissues including the CNS, nuclei, kidney, stomach and T-cells. The VR1 channel is a member of the superfamily of ion channels with six membrane-spanning domains, with highest homology to the TRP family of ion channels.

VR1 gene knockout mice have been shown to have reduced sensory sensitivity to thermal and acid stimuli. See, for example, Caterina, et al. Science, 14:306-313 (2000). This supports the concept that VR1 contributes not only to generation of pain responses but also to the maintenance of basal activity of sensory nerves. VR1 agonists and antagonists have use as analgesics for the treatment of pain of various genesis or etiology, for example acute, inflammatory and neuropathic pain, dental pain and headache (such as migraine, cluster headache and tension headache). They are also useful as anti-inflammatory agents for the treatment of arthritis, Parkinson's Disease, Alzheimer's Disease, stroke, uveitis; asthma, myocardial infarction, the treatment and prophylaxis of pain syndromes (acute and chronic [neuropathic]), traumatic brain injury, spinal cord injury, neurodegenerative disorders, alopecia (hair loss), inflammatory bowel disease, irritable bowel disease and autoimmune disorders, renal disorders, obesity, eating disorders, cancer, schizophrenia, epilepsy, sleeping disorders, cognition, depression, anxiety, blood pressure, lipid disorders, osteoarthritis, and atherosclerosis.

Compounds, such as those of the present invention, which interact with the vanilloid receptor can thus play a role in treating or preventing or ameliorating these conditions.

A wide variety of Vanilloid compounds of different structures are known in the art, for example those disclosed in European Patent Application Numbers, EP 0 347 000 and EP 0 401 903, UK Patent Application Number GB 2226313 and International Patent Application, Publication Number WO 92/09285. Particularly notable examples of vanilloid compounds or vanilloid receptor modulators are capsaicin or trans 8-methyl-N-vanillyl-6-nonenamide which is isolated from the pepper plant, capsazepine (Tetrahedron, 53, 1997, 4791) and olvanil or —N-(4-hydroxy-3-methoxybenzyl)oleamide (J. Med. Chem., 36, 1993, 2595).

International Patent Application, Publication Number WO 02/08221 discloses diaryl piperazine and related compounds which bind with high selectivity and high affinity to vanilloid receptors, especially Type I Vanilloid receptors, also known as capsaicin or VR1 receptors. The compounds are said to be useful in the treatment of chronic and acute pain conditions, itch and urinary incontinence.

International Patent Application, Publication Numbers WO 02/16317, WO 02/16318 and WO 02/16319 suggest that compounds having a high affinity for the vanilloid receptor are useful for treating stomach-duodenal ulcers.

International Patent Application, Publication No. WO 2005/046683, published May 26, 2005, commonly owned, discloses a series of compounds that have demonstrated activity as VR-1 antagonists, and that are suggested as being useful for the treatment of conditions associated with VR-1 activity.

U.S. Pat. No. 3,424,760 and U.S. Pat. No. 3,424,761 both describe a series of 3-Ureidopyrrolidines that are said to exhibit analgesic, central nervous system, and pyschopharmacologic activities. These patents specifically disclose the compounds 1-(1-phenyl-3-pyrrolidinyl)-3-phenyl urea and 1-(1-phenyl-3-pyrrolidinyl)-3-(4-methoxyphenyl) urea respectively. International Patent Applications, Publication Numbers WO 01/62737 and WO 00/69849 disclose a series of pyrazole derivatives which are stated to be useful in the treatment of disorders and diseases associated with the NPY receptor subtype Y5, such as obesity. WO 01/62737 specifically discloses the compound 5-amino-N-isoquinolin-5-yl-1-[3-(trifluoromethyl)phenyl]-1H-pyrazole-3-carboxamide. WO 00/69849 specifically discloses the compounds 5-methyl-N-quinolin-8-yl-1-[3-(trifluoromethyl)phenyl]-1H-pyrazole-3-carboxamide, 5-methyl-N-quinolin-7-yl-1-[3-trifluoromethyl)phenyl]-1H-pyrazole-3-carboxamide, 5-methyl-N-quinolin-3-yl-1-[3-(trifluoromethyl)phenyl]-1H-pyrazole-3-carboxamide, N-isoquinolin-5-yl-5-methyl-1-[3-(trifluoromethyl)phenyl]-1H-pyrazole-3-carboxamide, 5-methyl-N-quinolin-5-yl-1-[3-(trifluoromethyl)phenyl]-1H-pyrazole-3-carboxamide, 1-(3-chlorophenyl)-N-isoquinolin-5-yl-5-methyl-1H-pyrazole-3-carboxamide, N-isoquinolin-5-yl-1-(3-methoxyphenyl)-5-methyl-1H-pyrazole-3-carboxamide, 1-(3-fluorophenyl)-N-isoquinolin-5-yl-5-methyl-1H-pyrazole-3-carboxamide, 1-(2-chloro-5-trifluoromethylphenyl)-N-isoquinolin-5-yl-5-methyl-1N-pyrazole-3-carboxamide, 5-methyl-N-(3-methylisoquinolin-5-yl)-1-[3-(trifluoromethyl)phenyl]-1N-pyrazole-3-carboxamide, 5-methyl-N-(1,2,3,4-tetrahydroisoquinolin-5-yl)-1-[3-(trifluoromethyl)phenyl]-1H-pyrazole-3-carboxamide.

German Patent Application Number 2502588 describes a series of piperazine derivatives. This application specifically discloses the compound N-[3-[2-(diethylamino) ethyl]-1,2-dihydro-4-methyl-2-oxo-7-quinolinyl]-4-phenyl-1-piperazinecarboxamide.

International Patent Application, Publication No. WO 05/003084 discloses 4-(methylsulfonylamino) phenyl analogs as vanilloid antagonists and their use as analgesics, and International Patent Application Publication No. WO02/16318 discloses thiourea derivatives as a modulator for vaniloid receptor and their use as analgesics.

We have now discovered that certain compounds have surprising potency and selectivity as VR-1 antagonists. The compounds of the present invention are considered to be particularly beneficial as VR-1 antagonists as certain compounds exhibit improved aqueous solubility and metabolic stability.

### SUMMARY OF THE INVENTION

It has now been found that compounds such as those set forth herein, are capable of modifying mammalian ion channels such as the VR1 cation channel. Accordingly, the present compounds are potent VR1 antagonists with analgesic activity by systemic administration. The compounds of the present invention may show less toxicity, good absorption, good half-life, good solubility, low protein binding affinity, less drug-drug interaction, a reduced inhibitory activity at the HERG channel, reduced QT prolongation and good metabolic stability. This finding leads to novel compounds having therapeutic value. It also leads to pharmaceutical compositions having the compounds of the present invention as active ingredients and to their use to treat, prevent or ameliorate a range of conditions in mammals such as but not limited to pain of various genesis or etiology, for example acute, chronic, inflammatory and neuropathic pain, dental pain and headache (such as migraine, cluster headache and tension headache).

Accordingly, in a first aspect of the invention, compounds are disclosed that are capable of modifying ion channels, in vivo, having a formula I:

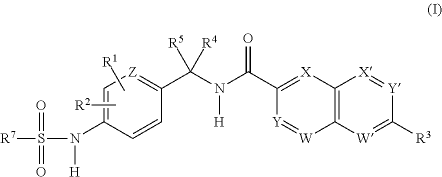

or a pharmaceutically acceptable salt thereof, and isotopic variants thereof, stereoisomers and tautomers thereof, wherein:
W, W′, X, X′, Y, Y′ and Z each independently represents CR⁸ or N;
R¹ and R² each independently represents hydrogen, halogen, hydroxy, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl;
R³ represents
hydrogen, halogen, hydroxy, (C₁-C₆)alkyl, halo(C₁-C₆)alkyl, hydroxy(C₁-C₆)alkyl, halo hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—, [(C₁-C₆)alkyl]₂N—, [hydroxy(C₁-C₆)alkyl]NH—, 3-6 membered cycloalkyl, [3-6 membered cycloalkyl]oxy, or [3-6 membered heterocycloalkyl]oxy
   or
   3-6 membered heterocycloalkyl, unsubstituted or substituted with
      halo, (C₁-C₆)alkyl, halo(C₁-C₆)alkyl, hydroxy(C₁-C₆)alkyl, (C₁-C₆)alkoxy, [(C₁-C₆)alkyl]₂N—, or hydroxy,
         or
   3-6 membered heteroaryl, 3-6 membered cycloalkyl (C₁-C₆)alkyl, or 3-6 membered cycloalkyl hydroxy(C₁-C₆)alkyl;
      R⁴ and R³ each independently represents hydrogen, (C₁-C₆)alkyl, halogen, halo(C₁-C₆) alkyl, or hydroxy(C₁-C₆)alkyl;
      each R⁸ independently represents
hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, halo hydroxy(C₁-C₆)alkyl, (C₁-C₆)alkylthio, (C₁-C₆)alkylsulfinyl, [(C₁-C₆)alkyl]NH—, [(C₁-C₆)cycloalkyl]NH—, [(C₁-C₆)alkyl]₂N—, [hydroxy(C₁-C₆)alkyl]NH—, [3-6 membered cycloalkyl]oxy, [3-6 membered heterocycloalkyl]oxy or
   3-6 membered heterocycloalkyl, unsubstituted or substituted with halo, (C₁-C₆)alkyl, (C₁-C₆)alkoxy, halo(C₁-C₆)alkyl, hydroxy(C₁-C₆)alkyl, aryl(C₁-C₆)alkyl, (C₁-C₆)carbalkoxy, hydroxy, aryl, (C₁-C₆)alkylaryl, halo(C₁-C₆)alkylaryl, haloaryl, (C₁-C₆)alkoxyaryl, or
3-10 membered heteroaryl, 3-6 membered cycloalkyl (C₁-C₆)alkyl, or 3-6 membered cycloalkyl hydroxy(C₁-C₆)alkyl or (C₁-C₆)alkylsulfonyl; and
   R⁷ represents (C₁-C₆)alkyl.

In a further embodiment of the invention, compounds of formula I above are disclosed, wherein:

W, W′, X, X′, Y, Y′ and Z each independently represents CR⁸. In a particular embodiment, one of W, W′, X, X′, Y, Y′ and Z represents N and the rest each independently represent CR⁸, and in a further particular embodiment, two of W, W′, X, X′, Y, Y′ and Z represents N and the rest each independently represent CR⁸.

In a further aspect of the invention compounds of a formula II are disclosed,

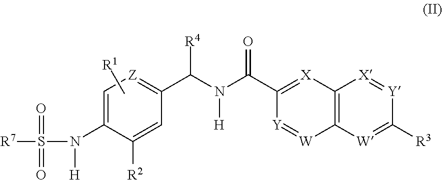

or a pharmaceutically acceptable salt, or thereof, and isotopic variants thereof, stereoisomers and tautomers thereof, wherein W, W, X, X′, Y, Y′, Z, R¹, R², R³, R⁴, R⁷, and R⁸, are as stated with respect to formula I.

In a particular embodiment of the invention, compounds of formula II above, are disclosed, wherein:

Z is independently selected from CR⁸ and N;
R¹ and R² each independently represents hydrogen, halogen, hydroxy, (C₁-C₆)alkoxy, hydroxy(C₁-C₆)alkoxy, (C₁-C₆)alkoxy(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl;
R³ represents hydrogen, halogen, (C₁-C₆)alkyl, halo(C₁-C₆)alkyl, hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—; [(C₁-C₆)alkyl]₂N—, 3-6 membered cycloalkyl, 3-6 membered heterocycloalkyl, 3-6 membered cycloalkyl (C₁-C₆)alkyl, or 3-6 membered cycloalkyl hydroxy (C₁-C₆)alkyl;
R⁴ and R⁵ each independently represents hydrogen, (C₁-C₆)alkyl, halogen, halo(C₁-C₆) alkyl, or hydroxy(C₁-C₆)alkyl;
R⁶ and R⁸ each independently represents hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl; and R⁷ represents (C₁-C₆)alkyl.

In a further embodiment in accordance with the compounds of formula II, R¹ represents hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl. Particularly, R¹ represents hydrogen, halogen or (C₁-C₆)alkyl, and more particularly, R¹ represents H or F.

In a further particular embodiment in accordance with the compounds of formula II, R² represents halogen, hydroxy, (C₁-C₆)alkyl, halo(C₁-C₆)alkyl, hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl. Particularly, R² represents halogen, (C₁-C₆)alkyl, halo(C₁-C₆)alkyl or hydroxy(C₁-C₆)alkyl, and more particularly, R² represents F or methyl. In a further particular embodiment, each of R¹ and R² represents F.

In another particular embodiment in accordance with the compounds of formula II, R⁴ is (C₁-C₆)alkyl, and a particular embodiment, R⁴ is methyl.

In a further particular embodiment in accordance with the compounds of formula II, R⁵ is hydrogen.

In a particular embodiment in accordance with the compounds of formula II, R⁷ is Me, Et, Pr, i-Pr, or t-butyl. More particularly, R⁷ is Me.

In a yet further embodiment in accordance with the compounds of formula II, Z represents CH. In an alternative embodiment, Z represents N.

In another embodiment in accordance with the compounds of formula I, R⁸ represents hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl. In a particular embodiment, R⁸ is H. In a still further alternate embodiment hereof, W, W′, X, and Y each independently represents CH and R³ represents halogen, (C₁-C₆)alkyl, halo(C₁-C₆)alkyl, hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—, ((C₁-C₆)alkyl)₂N—, 3-6 membered cycloalkyl, 3-6 membered heterocycloalkyl, 3-6 membered cycloalkyl (C₁-C₆)alkyl, or 3-6 membered cycloalkyl hydroxy (C₁-C₆)alkyl.

In a further alternate embodiment in accordance with compounds of formula I, W is N and each of W′, X, and Y are independently CR⁸, and in a particular embodiment hereof, R⁸ is H. In a yet further alternate embodiment, W is N, X is C—OH or C—OMe, and each of W′ and Y are independently CH. In another embodiment of the compounds of formula II, W is N; Y is C— Me; and each of W′ and X are independently CH. In a still further embodiment, X is N and each of W′, W and Y are independently CR⁸. More particularly, X is N and each of W′, W and Y are independently CH. Even further, W′ is N and each of W, X and Y are independently CR⁸, and in a variant of this embodiment, R⁸ is H.

In a still further alternate embodiment in accordance with compounds of formula I or II, R⁶ is H.

In yet further alternate embodiments of the compounds of formula II, R³ is halogen, (C₁-C₆)alkyl, halo(C₁-C₆)alkyl, hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—, [(C₁-C₆)alkyl]₂N—, 3-6 membered cycloalkyl, 3-6 membered heterocycloalkyl, 3-6 membered cycloalkyl (C₁-C₆)alkyl, or 3-6 membered cycloalkyl hydroxy (C₁-C₆)alkyl. More particularly, R³ may be halogen, (C₁-C₆)alkyl, halo(C₁-C₆)alkyl, (C₁-C₆)alkoxy, 3-6 membered cycloalkyl, or 3-6 membered heterocycloalkyl, and yet further, R³ is F, Br, or Cl. Further variants of R³ include Me, i-Pr, t-Bu, COMe, or CF₃; a 3-6 membered cycloalkyl, including cyclopropyl, cyclobutyl and cyclopentyl; a 3-6 membered heterocycloalkyl, including

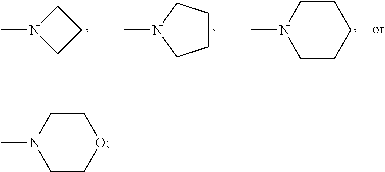

and yet further, R³ may be —C(Me)₂OH or —C(Me)(OH)-cyclopropyl.

In yet further particular embodiments, the compounds of the invention are set forth and may be selected from a comprehensive listing of such compounds, set forth later on herein in Table 1. The Table contains in excess of 118 compounds that have been or can be synthesized and have as a group, demonstrated activity in their capacity of modifying ion channels, in vivo, and thereby functioning in the therapeutic applications set forth herein in relation to capsaicin and the vanilloid receptor.

The compounds of the present invention are useful for the treatment of inflammatory pain and associated hyperalgesia and allodynia. They are also useful for the treatment of neuropathic pain and associated hyperalgesia and allodynia (e.g. trigeminal or herpetic neuralgia, diabetic neuropathy, causalgia, sympathetically maintained pain and deafferentation syndromes such as brachial plexus avulsion). The compounds of the present invention are also useful as anti-inflammatory agents for the treatment of arthritis, and as agents to treat Parkinson's Disease, Alzheimer's Disease, stroke, uveitis, asthma, myocardial infarction, traumatic brain injury, spinal cord injury, neurodegenerative disorders, alopecia (hair loss), inflammatory bowel disease and autoimmune disorders, renal disorders, obesity, eating disorders, cancer, schizophrenia, epilepsy, sleeping disorders, cognition, depression, anxiety, blood pressure, lipid disorders, and atherosclerosis.

In one aspect, this invention provides compounds which are capable of modifying ion channels, in vivo. Representative ion channels so modified include voltage-gated channels and ligand-gated channels, including cation channels such as vanilloid channels.

In a further aspect, the present invention provides pharmaceutical compositions comprising a compound of the invention, and a pharmaceutical carrier, excipient or diluent. In this aspect of the invention, the pharmaceutical composition can comprise one or more of the compounds described herein.

In a further aspect of the invention, a method is disclosed for treating mammals, including humans, as well as lower mammalian species, susceptible to or afflicted with a condition from among those listed herein, and particularly, such condition as may be associated with e.g. arthritis, uveitis, asthma, myocardial infarction, traumatic brain injury, acute spinal cord injury, alopecia (hair loss), inflammatory bowel disease and autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. Compounds have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

In additional method of treatment aspects, this invention provides methods of treating a mammal susceptible to or afflicted with neurodegenerative diseases and disorders such as, for example Parkinson's disease, Alzheimer's disease and multiple sclerosis; diseases and disorders which are mediated by or result in neuroinflammation such as, for example traumatic brain injury, stroke, and encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; irritable bowel syndrome, over active bladder, respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; alopecia (hair loss); obesity; lipid disorders; cancer; blood pressure; spinal cord injury; and renal disorders method comprises administering an effective condition-treating or condition-preventing amount of one or more of the pharmaceutical compositions just described.

In additional aspects, this invention provides methods for synthesizing the compounds of the invention, with representative synthetic protocols and pathways disclosed later on herein.

Other objects and advantages will become apparent to those skilled in the art from a consideration of the ensuing detailed description.

### DETAILED DESCRIPTION OF THE INVENTION

### Definitions

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope. By way of non-limiting example, such substituents may include e.g. halo (such as fluoro, chloro, bromo), —CN, —CF₃, —OH, —OCF₃, C₂-C₆ alkenyl, C₃-C₆ alkynyl, C₁-C₆alkoxy, aryl and di-C₁-C₆ alkylamino.

As used herein, the term “halogen” means fluoro, chloro, bromo or iodo, preferably fluoro or chloro.

As used herein, the terms “(C₁-C₆)alkyl”, “(C₁-C₄)alkyl” and “(C₁-C₃)alkyl” mean straight or branched chain saturated radicals having the required number of carbon atoms, including, but not limited to methyl, ethyl, n-propyl, iso-propyl, n-butyl, iso-butyl, secondary-butyl, tert-butyl and 2-methylbutyl groups. Preferred groups are methyl, ethyl, n-propyl, n-butyl, tert-butyl and 2-methylbutyl groups.

As used herein, the term “(C₁-C₆)alkoxy” means (C₁-C₆)alkyl-O— wherein (C₁-C₆)alkyl radical is as defined above, including, but not limited to methoxy, ethoxy, n-propoxy, iso-propoxy, n-butoxy, iso-butoxy, sec-butoxy and tert-butoxy. Preferred groups are methoxy, ethoxy, n-propoxy, n-butoxy and tert-butoxy.

As used herein, the term “hydroxy(C₁-C₆)alkyl” means (C₁-C₆)alkyl radical as defined above which is substituted by at least one hydroxy group including, but not limited to, hydroxymethyl, hydroxyethyl, hydroxy n-propyl, hydroxy iso-propyl (e.g. 2-hydroxy-1,1-dimethylethyl), hydroxy n-butyl, hydroxy iso-butyl, hydroxy secondary-butyl and hydroxy tert-butyl. Preferred groups are hydroxymethyl, hydroxyethyl, hydroxy n-propyl, hydroxy iso-propyl (e.g. 2-hydroxy-1,1-dimethylethyl) and hydroxy n-butyl.

As used herein, the term “(C₁-C₆)alkoxy-(C₁-C₆)alkyl” means (C₁-C₆)alkyl radical as defined above which is substituted by (C₁-C₆)alkoxy group as defined above.

As used herein, the term “(C₁-C₆)alkoxy-(C₁-C₆)alkoxy” means (C₁-C₆)alkoxy radical as defined above which is substituted by (C₁-C₆)alkoxy as defined above. Preferred groups are methoxy methoxy, methoxy ethoxy or ethoxy ethoxy groups.

As used herein the term “halo(C₁-C₆)alkyl” and “halo(C₁-C₄)alkyl” mean (C₁-C₆)alkyl or (C₁-C₃)alkyl radical which is substituted by one or more halogen atoms as defined above including, but not limited to, fluoromethyl, difluoromethyl, trifluoromethyl, 2-fluoroethyl, 2,2-difluoroethyl, 2,2,2-trifluoroethyl, 2,2,2-trifluoro-1,1-dimethylethyl, 2,2,2-trichloroethyl, 3-fluoropropyl, 4-fluorobutyl, chloromethyl, trichloromethyl, iodomethyl, bromomethyl and 4,4,4-trifluoro-3-methylbutyl groups. Preferred groups are fluoromethyl, difluoromethyl, trifluoromethyl, 2-fluoroethyl, 2,2-difluoroethyl, 2,2,2-trifluoroethyl and 2,2,2-trifluoro-1,1-dimethylethyl groups.

As used herein, the terms “(C₁-C₆)alkylthio” means (C₁-C₆)alkyl-S— wherein (C₁-C₆)alkyl radical is as defined above, including, but not limited to methylthio, ethylthio, propylthio and butylthio. Preferred groups are methylthio and methylthio groups.

As used herein, the terms “(C₁-C₆)alkylsulfinyl” means (C₁-C₆)alkyl-SO— wherein (C₁-C₆)alkyl radical is as defined above, including, but not limited to methylsulfinyl, ethylsulfinyl, propylsulfinyl and butylsulfinyl. Preferred groups are methylsulfinyl and methylsulfinyl groups.

As used herein, the terms “(C₁-C₆)alkylsulfonyl” means (C₁-C₆)alkyl-SO₂— wherein (C₁-C₆)alkyl radical is as defined above, including, but not limited to methylsulfonyl, ethylsulfonyl, propylsulfonyl and butylsulfonyl. Preferred groups are methylsulfonyl and methylsulfonyl groups.

As used herein, the term “[(C₁-C₆)alkyl]NH-” means alkyl-NH— wherein alkyl is defined above, including, but not limited to methylamino, ethylamino, n-propylamino, iso-propylamino, n-butylamino, iso-butylamino, secondary-butylamino, tert-butylamino. Preferred alkylamino groups are methylamino, ethylamino, n-propylamino, and n-butylamino.

As used herein, the term “[(C₁-C₆)alkyl]₂N—” means dialkyl-N— wherein alkyl is defined above, including, but not limited to dimethylamino, diethylamino, methylethylamino, di n-propylamino, methyl n-propylamino, ethyl n-propylamino di iso-propylamino, di n-butylamino, methyl n-butylamino di iso-butylamino, di secondary-butylamino, di tert-butylamino. Preferred dialkylamino groups are dimethylamino, diethylamino, di n-propylamino, di n-butylamino.

As used herein, the term “3- to 6-membered cycloalkyl” means non-aromatic saturated or unsaturated hydrocarbon ring, having from 3 to 6 carbon atoms. Typically, carbocyclyl is saturated, for example (C₃-C₆)cycloalkyl. Examples include cyclopropyl, cyclobutyl, cyclopentyl and cyclohexyl.

As used herein, the term “3- to 6-membered heterocycloalkyl” means non aromatic, saturated or unsaturated (C₃-C₆)carbocyclic ring in which, one or more, for example 1, 2 or 3, of the carbon atoms are replaced by a heteroatom selected from N, O or S. Examples include pyrrolidinyl, imidazolidinyl, tetrahydrofuranyl, tetrahydrothiophenyl, dioxolanyl, dithiolanyl, oxazolidinyl, thiazolidinyl, piperidinyl, piperazinyl, tetrahydropyranyl, tetrahydrothiopyranyl, dioxanyl, dithianyl, morpholinyl and thiomorpholinyl.

“Cycloalkoxy” refers to the group —OR where R is cycloalkyl. Such cycloalkoxy groups include, by way of example, cyclopentoxy, cyclohexoxy and the like.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Cyanato” refers to the radical —OCN.

“Cyano” refers to the radical —CN.

“Hydroxy” refers to the radical —OH.

“Nitro” refers to the radical —NO₂.

Examples of representative substituted aryls include the following

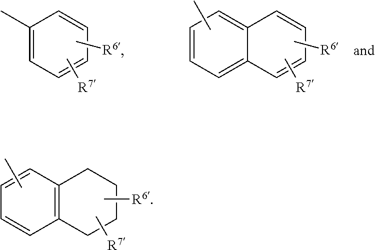

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. cycloheteroalkyl, aryl, e.g. heteroaryl, cycloalkenyl, cycloheteroalkenyl, and the like having from 1 to 5, and especially from 1 to 3 heteroatoms.

Examples of representative cycloheteroalkyls include the following

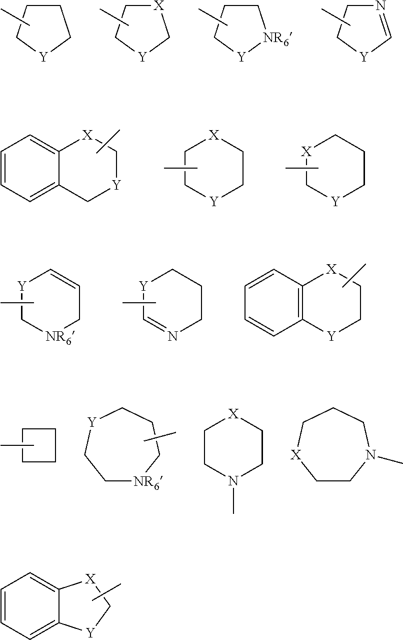

wherein each X is selected from CR⁴₂, NR⁴, O and S; and each Y is selected from NR⁴, O and S, and where R^{6′} is R².

Examples of representative cycloheteroalkenyls include the following:

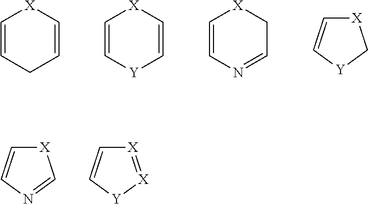

wherein each X is selected from CR⁴₂, NR⁴, O and S; and each Y is selected from carbonyl, N, NR⁴, O and S.

Examples of representative aryl having hetero atoms containing substitution include the following:

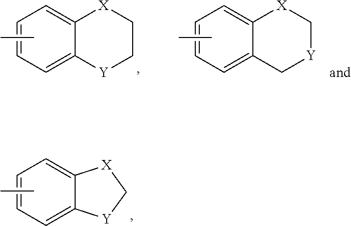

wherein each X is selected from C—R⁴, CR⁴₂.NR⁴, O and S; and each Y is selected from carbonyl, NR⁴, O and S.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl; and are shown in the following illustrative examples:

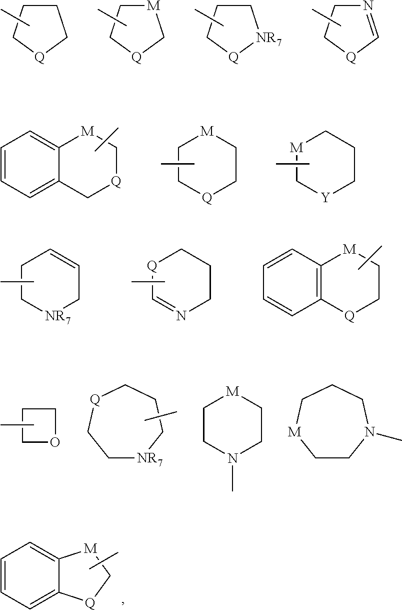

optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives. In the examples, M is CR⁷, NR²; O, or S; Q is O, NR² or S. R⁷ and R⁸ are independently selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Dihydroxyphosphoryl” refers to the radical —PO(OH)₂.

“Aminohydroxyphosphoryl” refers to the radical —PO(OH)NH₂.

“Thioalkoxy” refers to the group —SR where R is alkyl.

“Sulfanyl” refers to the radical HS—. “Substituted sulfanyl” refers to a radical such as RS— wherein R is any substituent described herein.

“Sulfonyl” refers to the divalent radical —S(O₂)—. “Substituted sulfonyl” refers to a radical such as R—(O₂)S— wherein R is any substituent described herein. “Aminosulfonyl” or “Sulfonamide” refers to the radical H₂N(O₂)S—, and “substituted aminosulfonyl” “substituted sulfonamide” refers to a radical such as R₂N(O₂)S— wherein each R is independently any substituent described herein.

“Sulfone” refers to the group —SO₂R. In particular embodiments, R is selected from H, lower alkyl, alkyl, aryl and heteroaryl.

“Thioaryloxy” refers to the group —SR where R is aryl.

“Thioketo” refers to the group ═S.

“Thiol” refers to the group —SH.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

“Solvate” refers to forms of the compound that are associated with a solvent, usually by a solvolysis reaction. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates.

“Subject” includes humans. The terms “human,” “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂arylalkyl esters of the compounds of the invention.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that ace substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### Compounds

As set forth earlier herein, the compounds of the present invention are useful for preventing and/or treating a broad range of conditions, among them, arthritis, Parkinson's disease, Alzheimer's disease, stroke, uveitis, asthma, myocardial infarction, the treatment and prophylaxis of pain syndromes (acute and chronic or neuropathic), traumatic brain injury, acute spinal cord injury, neurodegenerative disorders, alopecia (hair loss), inflammatory bowel disease and autoimmune disorders or conditions in mammals.

In order that the invention described herein may be more fully understood, the following structures representing compounds typical of the invention are set forth. It should be understood that these examples are for illustrative purposes only and are not to be construed as limiting this invention in any manner.

Accordingly, in a first aspect of the invention, compounds are disclosed that are capable of modifying ion channels, in vivo, having a formula I:

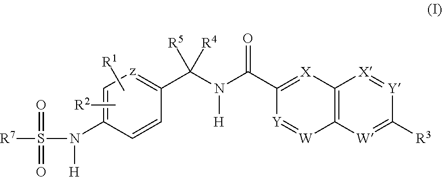

or a pharmaceutically acceptable salt thereof, and isotopic variants thereof, stereoisomers and tautomers thereof, wherein:
W, W′, X, X′, Y, Y′ and Z each independently represents CR⁸ or N;
R¹ and R² each independently represents hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl;
R³ represents
hydrogen, halogen, hydroxy, (C₁-C₆)alkyl, halo(C₁-C₆)alkyl, hydroxy(C₁-C₆)alkyl, halo hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—, [(C₁-C₆)alkyl]₂N—, [hydroxy(C₁-C₆)alkyl]NH—, 3-6 membered cycloalkyl, [3-6 membered cycloalkyl]oxy, or [3-6 membered heterocycloalkyl]oxy
   or
   3-6 membered heterocycloalkyl, unsubstituted or substituted with
      halo, (C₁-C₆)alkyl, halo(C₁-C₆)alkyl, hydroxy(C₁-C₆)alkyl, (C₁-C₆)alkoxy, [(C₁-C₆)alkyl]₂N—, or hydroxy,
         or
   3-6 membered heteroaryl, 3-6 membered cycloalkyl (C₁-C₆)alkyl, or 3-6 membered cycloalkyl hydroxy (C₁-C₆)alkyl;
      R⁴ and R⁵ each independently represents hydrogen, (C₁-C₆)alkyl, halogen, halo(C₁-C₆) alkyl, or hydroxy(C₁-C₆)alkyl;
      each R⁸ independently represents
hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, halo hydroxy(C₁-C₆)alkyl, (C₁-C₆)alkylthio, (C₁-C₆)alkylsulfinyl, [(C₁-C₆)alkyl]NH—, [(C₁-C₆)cycloalkyl]NH—, [(C₁-C₆)alkyl]₂N—, [hydroxy(C₁-C₆)alkyl]NH—, [3-6 membered cycloalkyl]oxy, [3-6 membered heterocycloalkyl]oxy or
   3-6 membered heterocycloalkyl, unsubstituted or substituted with halo, (C₁-C₆)alkyl, (C₁-C₆)alkoxy, halo(C₁-C₆)alkyl, hydroxy(C₁-C₆)alkyl, aryl(C₁-C₆)alkyl, [(C₁-C₆)alkyl]₂N—, (C₁-C₆)carbalkoxy, hydroxy, aryl, (C₁-C₆)alkylaryl, halo(C₁-C₆)alkylaryl, haloaryl, (C₁-C₆)alkoxyaryl, or
3-10 membered heteroaryl, 3-6 membered cycloalkyl (C₁-C₆)alkyl, or 3-6 membered cycloalkyl hydroxy (C₁-C₆)alkyl or (C₁-C₆)alkylsulfonyl; and
   R⁷ represents (C₁-C₆)alkyl.

In a further embodiment of the invention, compounds of formula I above are disclosed, wherein W, W′, X, X′, Y, Y′ and Z each independently represents CR⁸. In a particular embodiment, one of W, W′, X, X′, Y, Y′ and Z represents N and the rest each independently represent CR⁸, and in a further particular embodiment, two of W, W′, X, X′, Y, Y′ and Z represents N and the rest each independently represent CR⁸.

In a further aspect of the invention compounds of a formula II are disclosed,

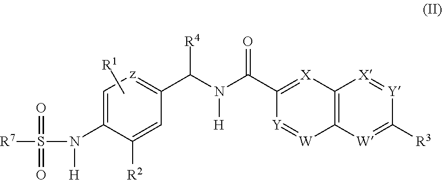

or a pharmaceutically acceptable salt, or thereof, and isotopic variants thereof, stereoisomers and tautomers thereof, wherein W, W′, X, X′, Y, Y′, Z, R¹, R², R³, R⁴, R⁷, and R⁸, are as stated with respect to formula I.

In a particular embodiment of compounds of formula II, R⁴ is (C₁-C₆)alkyl, and in a particular embodiment, R⁴ is methyl.

In a particular embodiment of compounds of formula II, R⁷ is Me, Et, Pr, i-Pr, or t-butyl, and in a particular embodiment, R⁷ is Me.

In a particular embodiment of compounds of formula II, R¹ represents hydrogen, halogen or (C₁-C₆)alkyl, and in a particular embodiment thereof, R¹ represents H or F.

In a further particular embodiment of compounds of formula II, R² represents halogen, halo(C₁-C₆)alkyl, or hydroxy(C₁-C₆)alkyl, and in a particular embodiment thereof, R² represents F or methyl.

In a still further particular embodiment of compounds of formula II, each of R¹ and R² represents F.

In a further particular embodiment of compounds of formula II, Z represents C—CH, CF or CCl, and in a further particular embodiment, Z represents N.

In a particular embodiment of compounds of formula II, R¹ represents H; R² represents Me and Z represents CF.

In a particular embodiment of compounds of formula II, W, W′, X, X′, Y and Y′ each independently represent CR⁸. In a further particular embodiment of compounds of formula II, W, W′, X, X′, Y and Y′ each independently represent CH. In a particular embodiment of compounds of formula II, W, W′, X, X′, Y and Y′ represent N and the rest each independently represent CR⁸.

In a particular embodiment of compounds of formula II, W is N and each of W′, X, X′, Y and Y′ is independently CR⁸. In a further particular embodiment of compounds of formula II, W is N and each of W′, X, X′, Y and Y′ is independently CH.

In a particular embodiment of compounds of formula II, X is N and each of W, W′, X′, Y and Y′ is independently CR⁸. In a further particular embodiment of compounds of formula II, X is N and each of W, W′, X′, Y and Y′ is independently CH.

In a particular embodiment of compounds of formula II, W′ is N and each of W, X, X′, Y and Y′ is independently CR⁸. In a further particular embodiment of compounds of formula II, W′ is N and each of W, X, X′, Y and Y′ is independently CH. In a further particular embodiment of compounds of formula II, W′ is N, each of W, X, Y and Y′ is independently CH, and X′ is CR⁸.

In a particular embodiment of compounds of formula II, W′ is N; each of W, X, Y and Y′ is independently CH; X′ is CR⁸ and R⁸ is 3-6 membered heterocycloalkyl. In a further particular embodiment of compounds of formula II, W′ is N; each of W, X, Y and Y′ is independently CH; X′ is CR⁸ and R⁸ is piperidinyl, morpholinyl, pyrrolidinyl, piperazinyl, and azetidinyl.

In a further particular embodiment of compounds of formula II, W′ is N; each of W, X, Y and Y′ is independently CH; X′ is CR⁸ and R⁸ is 3-6 membered heterocycloalkyl substituted with halo, (C₁-C₆)alkyl, (C₁-C₆)alkoxy, halo(C₁-C₆)alkyl, hydroxy(C₁-C₆)alkyl, aryl(C₁-C₆)alkyl, [(C₁-C₆)alkyl]₂N—, (C₁-C₆)carbalkoxy, hydroxy, aryl, (C₁-C₆)alkylaryl, halo(C₁-C₆)alkylaryl, haloaryl, (C₁-C₆)alkoxyaryl.

In a particular embodiment of compounds of formula II, W′ is N; each of W, X, Y and Y′ is independently CH; X′ is CR⁸ and R⁸ is piperidinyl, morpholinyl, pyrrolidinyl, piperazinyl, and azetidinyl, substituted with halo, (C₁-C₆)alkyl, (C₁-C₆)alkoxy, halo(C₁-C₆)alkyl, hydroxy(C₁-C₆)alkyl, aryl(C₁-C₆)alkyl, [(C₁-C₆)alkyl]₂N—, (C₁-C₆)carbalkoxy, hydroxy, aryl, (C₁-C₆)alkylaryl, halo(C₁-C₆)alkylaryl, haloaryl, (C₁-C₆)alkoxyaryl. In a further particular embodiment of compounds of formula II, W′ is N; each of W, X, Y and Y′ is independently CH; X′ is CR⁸ and R⁸ is piperidinyl, morpholinyl, pyrrolidinyl, piperazinyl, and azetidinyl, substituted with fluoro, methyl, difluoro, trifluoromethyl, dimethyl, hydroxyl, hydroxymethyl, carbethoxy, benzyl, phenyl, methoxyphenyl, chlorophenyl, and fluorophenyl.

In a particular embodiment of compounds of formula II, W′ and Y′ are each N; each of W, X, and Y is independently CH; X′ is CR⁸. In a further particular embodiment of compounds of formula II, W′ and Y′ are each N; each of W, X, and Y is independently CH; X′ is CR⁸ and R⁸ is 3-6 membered heterocycloalkyl. In a further particular embodiment of compounds of formula II, W′ and Y′ are each N; each of W, X, and Y is independently CH; X′ is CR⁸ and R⁸ is piperidinyl, morpholinyl, pyrrolidinyl, piperazinyl, and azetidinyl.

In a particular embodiment of compounds of formula II, W′ and Y′ are each N; each of W, X, and Y is independently CH; X′ is CR⁸ and R⁸ is 3-6 membered heterocycloalkyl substituted with halo, (C₁-C₆)alkyl, (C₁-C₆)alkoxy, halo(C₁-C₆)alkyl, hydroxy(C₁-C₆)alkyl, aryl(C₁-C₆)alkyl, [(C₁-C₆)alkyl]₂N—, (C₁-C₆)carbalkoxy, hydroxy, aryl, (C₁-C₆)alkylaryl, halo(C₁-C₆)alkylaryl, haloaryl, (C₁-C₆)alkoxyaryl. In a further particular embodiment of compounds of formula II, W′ and Y′ are each N; each of W, X, and Y is independently CH; X′ is CR⁸ and R⁸ is piperidinyl, morpholinyl, pyrrolidinyl, piperazinyl, and azetidinyl, substituted with halo, (C₁-C₆)alkyl, (C₁-C₆)alkoxy, halo(C₁-C₆)alkyl, hydroxy(C₁-C₆)alkyl, aryl(C₁-C₆)alkyl, [(C₁-C₆)alkyl]₂N—, (C₁-C₆)carbalkoxy, hydroxy; aryl, (C₁-C₆)alkylaryl, halo(C₁-C₆)alkylaryl, haloaryl, (C₁-C₆)alkoxyaryl.

In a particular embodiment of compounds of formula II, X′ is CR⁸ and R⁸ is Me, OH, OMe, Cl or CF₃.

In a particular embodiment of compounds of formula II, W, W′, X, X′, Y and Y′ each independently represent CH and R³ represents halogen, (C₁-C₆)alkyl, halo(C₁-C₆)alkyl, hydroxy(C₁-C₆)alkyl, (C₁-C₆)alkoxy, hydroxy(C₁-C₆)alkoxy, (C₁-C₆)alkoxy-(C₁-C₄alkyl, (C₁-C₆)acyl, (C₁-C₆)alkoxy-(C₁-C₆)alkoxy, [(C₁-C₆)alkyl]NH—, [(C₁-C₆)alkyl]₂N—, 3-6 membered cycloalkyl, 36 membered heterocycloalkyl, 3-6 membered cycloalkyl (C₁-C₆)alkyl, or 3-6 membered cycloalkyl hydroxy (C₁-C₆)alkyl.

In a particular embodiment of compounds of formula II, R³ is halogen, (C₁-C₆)alkyl, halo(C₁-C₆)alkyl, (C₁-C₆)alkoxy, 3-6 membered cycloalkyl, or 3-6 membered heterocycloalkyl. In a further particular embodiment of compounds of formula II, R³ is F, Br, or Cl.

In a particular embodiment of compounds of formula U, W, W′, X, X′, Y and Y′ each independently represent CH and R³ represents OMe, OEt, COMe, NMe₂, or NEt₂.

In a particular embodiment of compounds of formula II, R³ is Me, i-Pr, t-Bu, 1-methyl-1-trifluoromethylethyl, or 1-methyl-1-hydroxyethyl. In a further particular embodiment of compounds of formula II, R³ is CF₃.

In a particular embodiment of compounds of formula II, R³ is 3-6 membered cycloalkyl. In a further particular embodiment of compounds of formula II, R³ is cyclopropyl, 1-methylcyclopropyl, 1-hydroxycyclopropyl, 1-trifluoromethylcyclopropyl, cyclobutyl or cyclopentyl.

In a particular embodiment of compounds of formula II, R³ is 3-6 membered heterocycloalkyl. In a further particular embodiment of compounds of formula II, R³ is

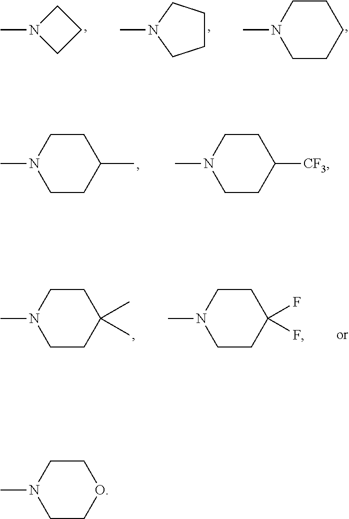

In a particular embodiment of compounds of formula II, R³ is —C(OMe)(Me)CF₃, —C(OH)(Me)CF₃, —C(Me)₂OH or —C(Me)(OH)-cyclopropyl.

In a particular embodiment of compounds of formula II, R³ is heteroaryl.

In a specific embodiment of compounds of formula I, the compounds may be selected from:
6-tert-Butyl-naphthalene-2-carboxylic acid [(R)-1-(4-methanesulfonylamino-3-methyl-phenyl)-ethyl]-amide;
6-tert-Butyl-naphthalene-2-carboxylic acid [(R)-1-(5-methanesulfonylamino-6-methyl-pyridin-2-yl)-ethyl]-amide;
6-Trifluoromethyl-quinoline-2-carboxylic acid [(R)-1-(3,5-difluoro-4-methanesulfonylamino-phenyl)-ethyl]-amide;
7-Trifluoromethyl-quinoline-3-carboxylic acid [(R)-1-(4-methanesulfonylamino-3-methyl-phenyl)-ethyl]-amide;
6-Trifluoromethyl-quinoline-2-carboxylic acid [(R)-1-(4-methanesulfonylamino-3-methyl-phenyl)-ethyl]-amide;
6-tert-Butyl-quinoline-2-carboxylic acid [(R)-1-(4-methanesulfonylamino-3-methyl-phenyl)-ethyl]-amide;
6-tert-Butyl-quinoline-2-carboxylic acid [(R)-1-(3,5-difluoro-4-methanesulfonylamino-phenyl)-ethyl]-amide;
2-tert-Butyl-quinoline-6-carboxylic acid [(R)-1-(4-methanesulfonylamino-3-methyl-phenyl)-ethyl]amide;
6-Isopropyl-quinoline-2-carboxylic acid [(R)-1-(4-methanesulfonylamino-3-methyl-phenyl)-ethyl]amide;
2-Trifluoromethyl-quinoline-6-carboxylic acid [(R)-1-(4-methanesulfonylamino-3-methyl-phenyl)-ethyl]-amide;
4-Methyl-7-trifluoromethyl-quinoline-3-carboxylic acid [(R)-1-(4-methanesulfonylamino-3-methyl-phenyl)-ethyl]-amide;
6-Bromo-quinoline-2-carboxylic acid [(R)-1-(4-methanesulfonylamino-3-methyl-phenyl)-ethyl]-amide;
6-tert-Butyl-naphthalene-2-carboxylic acid [(R)-1-(2-fluoro-4-methanesulfonylamino-5-methyl-phenyl)-ethyl]-amide;
6-tert-Butyl-quinoline-2-carboxylic acid [(R)-1-(2-fluoro-4-methanesulfonylamino-5-methyl-phenyl)-ethyl]-amide;
2-tert-Butyl-quinoline-6-carboxylic acid [(R)-1-(3,5-difluoro-4-methanesulfonylamino-phenyl)-ethyl]-amide;
6-(1-Hydroxy-1-methyl-ethyl)-quinoline-2-carboxylic acid [(R)-1-(4-methanesulfonylamino-3-methyl-phenyl)-ethyl]-amide;
6-Bromo-naphthalene-2-carboxylic acid [(R)-1-(4-methanesulfonylamino-3-methyl-phenyl)-ethyl]amide;
6-Fluoro-naphthalene-2-carboxylic acid [(R)-1-(4-methanesulfonylamino-3-methyl-phenyl)-ethyl]amide;
Naphthalene-2-carboxylic acid [(R)-1-(4-methanesulfonylamino-3-methyl-phenyl)-ethyl]amide;
6-Methoxy-naphthalene-2-carboxylic acid [(R)-1-(4-methanesulfonylamino-3-methyl-phenyl)-ethyl]-amide;
6-Pyrrolidin-1-yl-naphthalene-2-carboxylic acid [(R)-1-(4-methanesulfonylamino-3-methyl-phenyl)-ethyl]-amide;
6-Cyclopropyl-naphthalene-2-carboxylic acid [(R)-1-(4-methanesulfonylamino-3-methyl-phenyl)-ethyl]-amide;
7-Chloro-2-methyl-quinoline-3-carboxylic acid [(R)-1-(4-methanesulfonylamino-3-methyl-phenyl)-ethyl]-amide;
6-(1-Methyl-cyclopropyl)-naphthalene-2-carboxylic acid [(R)-1-(5-methanesulfonylamino-4-methyl-pyridin-2-yl)-ethyl]-amide;
7-Trifluoromethyl-quinoline-3-carboxylic acid [(R)-1-(3,5-difluoro-4-methanesulfonylamino-phenyl)-ethyl]-amide;
6-Acetyl-quinoline-2-carboxylic acid [(R)-1-(4-methanesulfonylamino-3-methyl-phenyl)-ethyl]-amide;
6-tert-Butyl-quinoline-3-carboxylic acid [(R)-1-(4-methanesulfonylamino-3-methyl-phenyl)-ethyl]-amide;
6-(1-Cyclopropyl-1-hydroxy-ethyl)-quinoline-2-carboxylic acid [(R)-1-(4-methanesulfonylamino-3-methyl-phenyl)-ethyl]-amide;
2-Isopropyl-quinoline-6-carboxylic acid [(R)-1-(3,5-difluoro-4-methanesulfonylamino-phenyl)-ethyl]-amide;
2-Trifluoromethyl-quinoline-6-carboxylic acid [(R)-1-(2-fluoro-4-methanesulfonylamino-5-methyl-phenyl)-ethyl]-amide;
7-tert-Butyl-quinoline-3-carboxylic acid [(R)-1-(4-methanesulfonylamino-3-methyl-phenyl)-ethyl]-amide;
7-Trifluoromethyl-quinoline-3-carboxylic acid [(R)-1-(3-fluoro-4-methanesulfonylamino-phenyl)-ethyl]-amide;
2-Isopropyl-quinoline-6-carboxylic acid [(R)-1-(4-methanesulfonylamino-3-methyl-phenyl)-ethyl]-amide;
7-tert-Butyl-quinoline-3-carboxylic acid [(R)-1-(3,5-difluoro-4-methanesulfonylamino-phenyl)-ethyl]-amide;
2-Isopropyl-quinoline-6-carboxylic acid [(R)-1-(2-fluoro-4-methanesulfonylamino-5-methyl-phenyl)-ethyl]-amide;
7-Isopropyl-quinoline-3-carboxylic acid [(R)-1-(4-methanesulfonylamino-3-methyl-phenyl)-ethyl]amide;
7-Isopropyl-quinoline-3-carboxylic acid [(R)-1-(3,5-difluoro-4-methanesulfonylamino-phenyl)-ethyl]-amide;
6-tert-Butyl-naphthalene-2-carboxylic acid [(R)-1-(5-methanesulfonylamino-4-methyl-pyridin-2-yl)-ethyl]-amide;
7-tert-Butyl-quinoline-3-carboxylic acid [(R)-1-(4-methanesulfonylamino-phenyl)-ethyl]amide;
7-tert-Butyl-4-methyl-quinoline-3-carboxylic acid [(R)-1-(4-methanesulfonylamino-3-methyl-phenyl)-ethyl]-amide;
7-Trifluoromethyl-quinoline-3-carboxylic acid [(R)-1-(2-fluoro-4-methanesulfonylamino-5-methyl-phenyl)-ethyl]-amide;
2-Ethoxy-quinoline-6-carboxylic acid [(R)-1-(4-methanesulfonylamino-3-methyl-phenyl)-ethyl]amide;
6-Cyclopropyl-naphthalene-2-carboxylic acid [(R)-1-(2-fluoro-4-methanesulfonylamino-5-methyl-phenyl)-ethyl]-amide;
2-Cyclopropyl-quinoline-6-carboxylic acid [(R)-1-(3,5-difluoro-4-methanesulfonylamino-phenyl)-ethyl]-amide;
6-Trifluoromethyl-naphthalene-2-carboxylic acid [(R)-1-(2-fluoro-4-methanesulfonylamino-5-methyl-phenyl)-ethyl]-amide;
2-(1-Methyl-cyclopropyl)-quinoline-6-carboxylic acid [(R)-1-(3,5-difluoro-4-methanesulfonylamino-phenyl)-ethyl]-amide;
2-(2,2,2-Trifluoro-1,1-dimethyl-ethyl)-quinoline-6-carboxylic acid [(R)-1-(4-methanesulfonylamino-3-methyl-phenyl)-ethyl]-amide;
6-(1-Methyl-cyclopropyl)-naphthalene-2-carboxylic acid [(R)-1-(2-fluoro-4-methanesulfonylamino-5-methyl-phenyl)-ethyl]-amide;
6-Trifluoromethyl-naphthalene-2-carboxylic acid [(R)-1-(4-methanesulfonylamino-3-methyl-phenyl)-ethyl]-amide;
6-Cyclopropyl-naphthalene-2-carboxylic acid [(R)-1-(2-chloro-4-methanesulfonylamino-5-methyl-phenyl)-ethyl]-amide;
7-Trifluoromethyl-quinoline-3-carboxylic acid [(R)-1-(2-chloro-4-methanesulfonylamino-5-methyl-phenyl)-ethyl]-amide;
6-Trifluoromethyl-naphthalene-2-carboxylic acid [(R)-1-(5-methanesulfonylamino-4-methyl-pyridin-2-yl)-ethyl]-amide;
2-(2,2,2-Trifluoro-1,1-dimethyl-ethyl)-quinoline-6-carboxylic acid [(R)-1-(2-fluoro-4-methanesulfonylamino-5-methyl-phenyl)-ethyl]-amide;
6-Trifluoromethyl-quinoline-2-carboxylic acid [(R)-1-(2-fluoro-4-methanesulfonylamino-5-methyl-phenyl)-ethyl]-amide;
6-Trifluoromethyl-quinoline-2-carboxylic acid [(R)-1-(2-chloro-4-methanesulfonylamino-5-methyl-phenyl)-ethyl]-amide;
4-Hydroxy-7-trifluoromethyl-quinoline-3-carboxylic acid [(R)-1-(4-methanesulfonylamino-3-methyl-phenyl)-ethyl]-amide;
7-Trifluoromethyl-quinoline-2-carboxylic acid [(R)-1-(4-methanesulfonylamino-3-methyl-phenyl)-ethyl]-amide;
Quinoline-3-carboxylic acid [(R)-1-(4-methanesulfonylamino-3-methyl-phenyl)-ethyl]amide;
4-Morpholin-4-yl-2-trifluoromethyl-quinoline-6-carboxylic acid [(R)-1-(2-fluoro-4-methanesulfonylamino-5-methyl-phenyl)-ethyl]-amide;
6-Fluoro-7-trifluoromethyl-quinoline-3-carboxylic acid [(R)-1-(2-fluoro-4-methanesulfonylamino-5-methyl-phenyl)-ethyl]-amide;
6-Chloro-7-trifluoromethyl-quinoline-3-carboxylic acid [(R)-1-(2-fluoro-4-methanesulfonylamino-5-methyl-phenyl)-ethyl]-amide;
2-Pyrrolidin-1-yl-quinoline-6-carboxylic acid [(R)-1-(2-fluoro-4-methanesulfonylamino-5-methyl-phenyl)-ethyl]-amide;
2-Dimethylamino-quinoline-6-carboxylic acid [(R)-1-(2-fluoro-4-methanesulfonylamino-5-methyl-phenyl)-ethyl]-amide;
4-Piperidin-1-yl-2-trifluoromethyl-quinoline-6-carboxylic acid [(R)-1-(2-fluoro-4-methanesulfonylamino-5-methyl-phenyl)-ethyl]-amide;
4-Pyrrolidin-1-yl-2-trifluoromethyl-quinoline-6-carboxylic acid [(R)-1-(2-fluoro-4-methanesulfonylamino-5-methyl-phenyl)-ethyl]-amide;
4-(4,4-Difluoro-piperidin-1-yl)-2-trifluoromethyl-quinoline-6-carboxylic acid [(R)-1-(2-fluoro-4-methanesulfonylamino-5-methyl-phenyl)-ethyl]-amide;
6-Trifluoromethyl-naphthalene-2-carboxylic acid [(R)-1-(3-hydroxymethyl-4-methanesulfonylamino-phenyl)-ethyl]-amide;
2-Piperidin-1-yl-quinoline-6-carboxylic acid [(R)-1-(2-fluoro-4-methanesulfonylamino-5-methyl-phenyl)-ethyl]-amide;
2-Trifluoromethyl-4-(4-trifluoromethyl-piperidin-1-yl)-quinoline-6-carboxylic acid [(R)-1-(2-fluoro-4-methanesulfonylamino-5-methyl-phenyl)-ethyl]-amide;
7-Trifluoromethyl-naphthalene-2-carboxylic acid [(R)-1-(4-methanesulfonylamino-3-methyl-phenyl)-ethyl]-amide;
4-[4-(2-Hydroxy-ethyl)-piperazin-1-yl]-2-trifluoromethyl-quinoline-6-carboxylic acid [(R)-1-(2-fluoro-4-methanesulfonylamino-5-methyl-phenyl)-ethyl]-amide;
2-Morpholin-4-yl-quinoline-6-carboxylic acid [(R)-1-(2-fluoro-4-methanesulfonylamino-5-methyl-phenyl)-ethyl]-amide;
4-Piperidin-1-yl-2-trifluoromethyl-quinazoline-6-carboxylic acid [(R)-1-(2-fluoro-4-methanesulfonylamino-5-methyl-phenyl)-ethyl]-amide;
4-[4-(3-Chloro-phenyl)-piperazin-1-yl]-2-trifluoromethyl-quinazoline-6-carboxylic acid [(R)-1-(2-fluoro-4-methanesulfonylamino-5-methyl-phenyl)-ethyl]-amide;
4-(2,6-Dimethyl-morpholin-4-yl)-2-trifluoromethyl-quinazoline-6-carboxylic acid [(R)-1-(2-fluoro-4-methanesulfonylamino-5-methyl-phenyl)-ethyl]-amide;
7-Cyclopropyl-[1,5]naphthyridine-3-carboxylic acid [(R)-1-(2-fluoro-4-methanesulfonylamino-5-methyl-phenyl)-ethyl]-amide;
4-[4-(2-Methoxy-phenyl)-piperazin-1-yl]-2-trifluoromethyl-quinazoline-6-carboxylic acid [(R)-1-(2-fluoro-4-methanesulfonylamino-5-methyl-phenyl)-ethyl]-amide;
4-((R)-3-Hydroxy-pyrrolidin-1-yl)-2-trifluoromethyl-quinoline-6-carboxylic acid [(R)-1-(2-fluoro-4-methanesulfonylamino-5-methyl-phenyl)-ethyl]-amide;
4-((S)-3-Hydroxy-pyrrolidin-1-yl)-2-trifluoromethyl-quinoline-6-carboxylic acid [(R)-1-(2-fluoro-4-methanesulfonylamino-5-methyl-phenyl)ethyl]-amide;
4-(3,3-Difluoro-azetidin-1-yl)-2-trifluoromethyl-quinoline-6-carboxylic acid [(R)-1-(2-fluoro-4-methanesulfonylamino-5-methyl-phenyl)-ethyl]-amide;
4-((R)-2-Hydroxymethyl-pyrrolidin-1-yl)-2-trifluoromethyl-quinoline-6-carboxylic acid [(R)-1-(2-fluoro-4-methanesulfonylamino-5-methyl-phenyl)-ethyl]-amide;
4-(Tetrahydro-pyran-4-yloxy)-2-trifluoromethyl-quinoline-6-carboxylic acid [(R)-1-(2-fluoro-4-methanesulfonylamino-5-methyl-phenyl)-ethyl]-amide;
4-(4-Hydroxy-piperidin-1-yl)-2-trifluoromethyl-quinoline-6-carboxylic acid [(R)-1-(2-fluoro-4-methanesulfonylamino-5-methyl-phenyl)-ethyl]-amide;
4-{6-[(R)-1-(2-Fluoro-4-methanesulfonylamino-5-methyl-phenyl)-ethylcarbamoyl]-2-trifluoromethyl-quinazolin-4-yl}-piperazine-1-carboxylic acid ethyl ester
4-Cyclohexylamino-2-trifluoromethyl-quinoline-6-carboxylic acid [(R)-1-(2-fluoro-4-methanesulfonylamino-5-methyl-phenyl)-ethyl]-amide;
7-Pyrrolidin-1-yl-[1,5]naphthyridine-3-carboxylic acid [(R)-1-(2-fluoro-4-methanesulfonylamino-5-methyl-phenyl)-ethyl]-amide;
4-(4-Hydroxymethyl-piperidin-1-yl)-2-trifluoromethyl-quinoline-6-carboxylic acid [(R)-1-(2-fluoro-4-methanesulfonylamino-5-methyl-phenyl)-ethyl]-amide;
6-(2,2,2-Trifluoro-1-hydroxy-1-methyl-ethyl)-naphthalene-2-carboxylic acid [(R)-1-(2-fluoro-4-methanesulfonylamino-5-methyl-phenyl)-ethyl]-amide;
6-Pyrazol-1-yl-naphthalene-2-carboxylic acid [(R)-1-(2-fluoro-4-methanesulfonylamino-5-methyl-phenyl)-ethyl]-amide;
4-(4-Benzyl-piperidin-1-yl)-2-trifluoromethyl-quinazoline-6-carboxylic acid [(R)-1-(2-fluoro-4-methanesulfonylamino-5-methyl-phenyl)ethyl]-amide;
2-(4-Methoxy-piperidin-1-yl)-quinoline-6-carboxylic acid [(R)-1-(2-fluoro-4-methanesulfonylamino-5-methyl-phenyl)-ethyl]-amide;
2-(1-Methyl-cyclopropyl)-quinoline-6-carboxylic acid [(R)-1-(2-fluoro-4-methanesulfonylamino-5-methyl-phenyl)-ethyl]-amide;
2-(4,4-Difluoro-piperidin-1-yl)-quinoline-6-carboxylic acid KR)-1-(2-fluoro-4-methanesulfonylamino-5-methyl-phenyl)-ethyl]-amide;
4-Morpholin-4-yl-2-trifluoromethyl-quinazoline-6-carboxylic acid [(R)-1-(2-fluoro-4-methanesulfonylamino-5-methyl-phenyl)-ethyl]-amide;
2-(4,4-Dimethyl-piperidin-1-yl)-quinoline-6-carboxylic acid [(R)-1-(2-fluoro-4-methanesulfonylamino-5-methyl-phenyl)-ethyl]-amide;
2-Diethylamino-quinoline-6-carboxylic acid [(R)-1-(2-fluoro-4-methanesulfonylamino-5-methyl-phenyl)-ethyl]-amide;
2-(4-Trifluoromethyl-piperidin-1-yl)-quinoline-6-carboxylic acid [(R)-1-(2-fluoro-4-methanesulfonylamino-5-methyl-phenyl)-ethyl]-amide;
6-(2,2,2-Trifluoro-1-methoxy-1-methyl-ethyl)-naphthalene-2-carboxylic acid [(R)-1-(2-fluoro-4-methanesulfonylamino-5-methyl-phenyl)-ethyl]amide;
   or a pharmaceutically acceptable salt thereof, and isotopic variants thereof, stereoisomers and tautomers thereof.

Accordingly, additional groups of particular compounds are provided. Thus, and as discussed earlier herein, suitable compounds capable of modifying ion channels in vivo, may be selected from those listed in Table 1, below, and may be prepared either as shown or in the form of a pharmaceutically acceptable salt, solvate or prodrug thereof; and stereoisomers and tautomers thereof. All such variants are contemplated herein and are within the scope of the present invention.

In certain aspects, the present invention provides prodrugs and derivatives of the compounds according to the formulae above. Prodrugs are derivatives of the compounds of the invention, which have cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

### Assay Methods

### Human VR1 Antagonist Assay

VR1 antagonistic activity can be determined by the Ca²⁺ imaging assay using human VR1 highly expressing cells. The cells that highly express human VR1 receptors are obtainable from several different conventional methods. The one standard method is cloning from human Dorsal Root Ganglion (DRG) or kidney according to the methods such as described in the journal article; Nature, 389, pp 816-824, 1997. Alternatively VR1 receptors highly expressing human keratinocytes are also known and published in the journal article (Biochemical and Biophysical Research Communications, 291, pp 124-129, 2002). In this article, human keratinocytes demonstrated VR1 mediated intracellular Ca²⁺ increase by addition of capsaicin. Furthermore, the method to up regulate human VR1 gene, which is usually a silent gene or don't produce detectable level of VR1 receptors, is also available to obtain propriety cells. Such genetic modification method was described in detail; Nat. Biotechnol., 19, pp 440-445, 2001.

The cells that express human VR1 receptors were maintained in culture flask at 37° C. in an environment containing 5% CO₂ until use in the assay. The intracellular Ca²⁺ imaging assay to determine VR1 antagonistic activities were done by following procedures.

The culture medium was removed from the flask and fura-2/AM fluorescent calcium indicator was added to the flask at a concentration of 5 μM in the medium. The flask was placed in CO₂ incubator and incubated for 1 hour. Then the cells expressing the human VR1 receptors were detached from the flask follow by washing with phosphate buffer saline, PBS(−) and re-suspended in assay buffer. The 80 tit of aliquot of cell suspension (3.75×10⁵ cells/ml) was added to the assay plate and the cells were spun down by centrifuge (950 rpm, 20° C., 3 minutes).

### Capsaicin Stimulation Assay

The capsaicin-induced changes in the intracellular calcium concentration were monitored using FDSS 6000 (Hamamatsu Photonics, Japan), a fluorometric imaging system. The cell suspension in Krebs-Ringer HEPES (KRH) buffer (115 mM NaCl, 5.4 mM KCl, 1 mM MgSO₄, 1.8 mM CaCl₂, 11 mM D-Glucose, 25 mM HEPES, 0.96 mM Na₂HPO₄, pH 7.3) were pre-incubated with varying concentrations of the test compounds or KRH buffer (buffer control) for 15 minutes at room temperature under the dark condition. Then capsaicin solution, which gives 300 nM in assay mixture, was automatically added to the assay plate by the FDSS 6000.

### Acid Stimulation Assay

The Acid-induced changes in the intracellular calcium concentration were monitored using FDSS 6000 (Hamamatsu Photonics, Japan), a fluorometric imaging system. The cell suspension in resting buffer (HBSS supplemented with 10 mM HEPES, pH 7.4) were pre-incubated with varying concentrations of the test compounds or resting buffer (buffer control) for 15 minutes at room temperature under the dark condition. The cells were automatically added the stimulating solution (HBSS supplemented with MES, final assay buffer pH5.8) by the FDSS 6000. The IC₅₀ values of VR1 antagonists were determined from the half of the increase demonstrated by buffer control samples after acidic stimulation.

### Determination of Antagonist Activity

The monitoring of the changes in the fluorescence signals (λₑₓ=340 nm/380 nm, λₑₘ=510-520 nm) was initiated at 1 minute prior to the addition of capsaicin solution or acidic buffer and continued for 5 minutes. The IC₅₀ values of VR1 antagonists were determined from the half of the increase demonstrated by buffer control samples after agonist stimulation.

### Chronic Constriction Injury Model (CCI Model):

Male Sprague-Dawley rats (270-300 g; B. W., Charles River, Tsukuba, Japan) are used. The chronic constriction injury (CCI) operation is performed according to the method described by Bennett and Xie (Bennett, G. J. and Xie, Y. K. Pain, 33:87-107, 1988). Briefly, animals are anesthetized with sodium pentobarbital (64.8 mg/kg, i.p.) and the left common sciatic nerve is exposed at the level of the middle of the thigh by blunt dissection through the biceps femoris. A portion of the sciatic nerve proximal to its trifurcation is freed of adhering tissue and 4 ligatures (4-0 silk) are tied loosely around it with about 1 mm space. A sham operation is performed as same as CCI surgery except for sciatic nerve ligation. Two weeks after surgery, mechanical allodynia is evaluated by application of von Frey hairs (VFHs) to the plantar surface of the hind paw. The lowest amount of force of VFH required to elicit a response is recorded as the paw withdrawal threshold (PWT). VFH testing is performed at 0.5, 1 and 2 hr post-dosing. Experimental data are analyzed using Kruskal-Wallis test followed by Dunn's test for multiple comparisons or Mann-Whitney U-test for paired comparison.

### Caco-2 Permeability

Caco-2 permeability is measured according to the method described in Shiyin Yee, Pharmaceutical Research, 763 (1997).

Caco-2 cells are grown on filter supports (Falcon HTS multiwell insert system) for 14 days. Culture medium is removed from both the apical and basolateral compartments and the monolayers are preincubated with pre-warmed 0.3 ml apical buffer and 1.0 ml basolateral buffer for 0.75 hour at 37° C. in a shaker water bath at 50 cycles/min. The apical buffer consists of Hanks Balanced Salt Solution, 25 mM D-glucose monohydrate, 20 mM MES Biological Buffer, 1.25 mM CaCl₂ and 0.5 mM MgCl₂ (pH 6.5). The basolateral buffer consists of Hanks Balanced Salt Solution, 25 mM D-glucose monohydrate, 20 mM HEPES Biological Buffer, 1.25 mM CaCl₂ and 0.5 mM MgCl2 (pH 7.4). At the end of the preincubation, the media is removed and test compound solution (10 μM) in buffer is added to the apical compartment. The inserts are moved to wells containing fresh basolateral buffer and incubated for 1 hr. Drug concentration in the buffer is measured by LC/MS analysis.

Flux rate (F, mass/time) is calculated from the slope of the cumulative appearance of substrate on the receiver side and apparent permeability coefficient (Papp) is calculated from the following equation:

Papp(cm/sec)=(F*VD)/(SA*MD)

where SA is surface area for transport (0.3 cm²), VD is the donor volume (0.3 ml), MD is the total amount of drug on the donor side at t=0. All data represent the mean of 2 inserts. Monolayer integrity is determined by Lucifer Yellow transport.

### Human Dofetilide Binding

A cell paste of HEK-293 cells expressing the HERG product can be suspended in 10-fold volume of 50 mM Tris buffer adjusted at pH 7.5 at 25° C. with 2 M HCl containing 1 mM MgCl₂, 10 mM KCl. The cells are homogenized using a Polytron homogenizer (at the maximum power for 20 seconds) and centrifuged at 48,000 g for 20 minutes at 4° C. The pellet is resuspended, homogenized and centrifuged once more in the same manner. The resultant supernatant is discarded and the final pellet is resuspended (10-fold volume of 50 mM Tris buffer) and homogenized at the maximum power for 20 seconds. The membrane homogenate is aliquoted and stored at −80° C. until use. An aliquot is used for protein concentration determination using a Protein Assay Rapid Kit and ARVO SX plate reader (Wallac). All the manipulation, stock solution and equipment are kept on ice at all times. For saturation assays, experiments are conducted in a total volume of 200 μl. Saturation is determined by incubating 20 μl of [3H]-dofetilide and 160 μl of membrane homogenates (20-30 μg protein per well) for 60 min at room temperature in the absence or presence of 10 μM dofetilide at final concentrations (20 μl) for total or nonspecific binding, respectively. All incubations are terminated by rapid vacuum filtration over polyetherimide (PEI) soaked glass fiber filter papers using Skatron cell harvester followed by two washes with 50 mM Tris buffer (pH 7.5 at 25° C.). Receptor-bound radioactivity is quantified by liquid scintillation counting using a Packard LS counter.

For the competition assay, compounds are diluted in 96 well polypropylene plates as 4-point dilutions in semi-log format. All dilutions are performed in DMSO first and then transferred into 50 mM Tris buffer (pH 7.5 at 25° C.) containing 1 mM MgCl₂, 10 mM KCl so that the final DMSO concentration becomes equal to 1%. Compounds are dispensed in triplicate in assay plates (4 μl). Total binding and nonspecific binding wells are set up in 6 wells as vehicle and 10 μM dofetilide at final concentration, respectively. The radioligand is prepared at 5.6× final concentration and this solution is added to each well (36 μl). The assay is initiated by addition of YSi poly-L-lysine Scintillation Proximity Assay (SPA) beads (50 μl, 1 mg/well) and membranes (110 μl, 20 μg/well). Incubation is continued for 60 min at room temperature. Plates are incubated for a further 3 hours at room temperature for beads to settle. Receptor-bound radioactivity is quantified by counting Wallac MicroBeta plate counter.

### HERG Assay

HEK 293 cells which stably express the HERG potassium channel are used for electrophysiological study. The methodology for stable transfection of this channel in HEK cells can be found elsewhere (Z. Zhou et al., 1998, Biophysical Journal, 74, pp 230-241). Before the day of experimentation, the cells are harvested from culture flasks and plated onto glass coverslips in a standard Minimum Essential Medium (MEM) medium with 10% Fetal Calf Serum (FCS). The plated cells are stored in an incubator at 37° C. maintained in an atmosphere of 95% O₂/5% CO₂. Cells are studied between 15-28 hrs after harvest.

HERG currents are studied using standard patch clamp techniques in the whole-cell mode. During the experiment the cells are superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings are made using a patch clamp amplifier and patch pipettes which have a resistance of 1-3 MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15MΩ and seal resistances >1 GΩ are accepted for further experimentation. Series resistance compensation is applied up to a maximum of 80%. No leak subtraction is done. However, acceptable access resistance depends on the size of the recorded currents and the level of series resistance compensation that can safely be used. Following the achievement of whole cell configuration and sufficient time for cell dialysis with pipette solution (>5 min), a standard voltage protocol is applied to the cell to evoke membrane currents. The voltage protocol is as follows. The membrane is depolarized from a holding potential of −80 mV to +40 mV for 1000 ms. This is followed by a descending voltage ramp (rate 0.5 mV msec-1) back to the holding potential. The voltage protocol is applied to a cell continuously throughout the experiment every 4 seconds (0.25 Hz). The amplitude of the peak current elicited around −40 mV during the ramp is measured. Once stable evoked current responses are obtained in the external solution, vehicle (0.5% DMSO in the standard external solution) is applied for 10-20 min by a peristalic pump. Provided there are minimal changes in the amplitude of the evoked current response in the vehicle control condition, the test compound of either 0.3, 1, 3, 10 mM is applied for a 10 min period. The 10 min period includes the time during which supplying solution is passing through the tube from solution reservoir to the recording chamber via the pump. Exposure time of cells to the compound solution is more than 5 min after the drug concentration in the chamber well reaches the intended concentration. There is a subsequent wash period of a 10-20 min to assess reversibility. Finally, the cells are exposed to high dose of dofetilide (5 mM), a specific IKr blocker, to evaluate the insensitive endogenous current.

All experiments are performed at room temperature (23±1° C.). Evoked membrane currents are recorded on-line on a computer, filtered at 500-1 KHz (Bessel −3 dB) and sampled at 1-2 KHz using the patch clamp amplifier and a specific data analyzing software. Peak current amplitude, which generally occurs at around −40 mV, is measured off line on the computer.

The arithmetic mean of the ten values of amplitude is calculated under vehicle control conditions and in the presence of drug. Percent decrease of IN in each experiment is obtained by the normalized current value using the following formula: IN=(1−ID/IC)×100, where ID is the mean current value in the presence of drug and IC is the mean current value under control conditions. Separate experiments are performed for each drug concentration or time-matched control, and arithmetic mean in each experiment is defined as the result of the study.

### Half-Life in Human Liver Microsomes (HLM)

Test compounds (1 μg) are incubated with 3.3 mM MgCl₂ and 0.78 mg/mL HLM (HL101) in 100 mM potassium phosphate buffer (pH 7.4) at 37° C. on the 96-deep well plate. The reaction mixture is split into two groups, a non-P450 and a P450 group. NADPH is only added to the reaction mixture of the P450 group. An aliquot of samples of the P450 group is collected at 0, 10, 30, and 60 min time point, where 0 min time point indicates the time when NADPH is added into the reaction mixture of the P450 group. An aliquot of samples of non-P450 group is collected at −10 and 65 min time point. Collected aliquots are extracted with acetonitrile solution containing an internal standard. The precipitated protein is spun down in a centrifuge (2000 rpm, 15 min). The compound concentration in the supernatant is measured by LC/MS/MS system.

The half-life value is obtained by plotting the natural logarithm of the peak area ratio of compounds/internal standard versus time. The slope of the line of best fit through the points yields the rate of metabolism (k). This is converted to a half-life value using following equations:

Half-life=ln 2/k

### Mono-Iodoacetate (MIA)-Induced OA Model

Male 6-weeks-old Sprague-Dawley (SD, Japan SLC or Charles River Japan) rats are anesthetized with pentobarbital. The injection site (knee) of MIA is shaved and cleaned with 70% ethanol. Twenty-five ml of MIA solution or saline is injected in the right knee joint using a 29 G needle. The effect of joint damage on the weight distribution through the right (damaged) and left (untreated) knee is assessed using an incapacitance tester (Linton Instrumentation, Norfolk, UK). The force exerted by each hind limb is measured in grams. The weight-bearing (WB) deficit is determined by a difference of weight loaded on each paw. Rats are trained to measure the WB once a week until 20 days post MIA-injection. Analgesic effects of compounds are measured at 21 days after the MIA injection. Before the compound administration, the “pre value” of WB deficit is measured. After the administration of compounds, attenuation of WB deficits is determined as analgesic effects.

### Complete Freund's Adjuvant (CFA) Induced Thermal and Mechanical Hyperalgesia in Rats Thermal Hyperalgesia

Male 6-week-old SD rats are used. Complete Freund's adjuvant (CFA, 300 mg of Mycobacterium Tuberculosis H37RA (Difco, MI) in 100 μL of liquid paraffin (Wako, Osaka, Japan)) is injected into the plantar surface of a hind paw of the rats. Two days after CFA-injection, thermal hyperalgesia is determined by the method described previously (Hargreaves et al., 1988) using the plantar test apparatus (Ugo-Basil, Varese, Italy). Rats are adapted to the testing environment for at least 15 minutes prior to any stimulation. Radiant heat is applied to the plantar surface of a hind paw and paw withdrawal latencies (PWL, seconds) are determined. The intensity of radiant heat is adjusted to produce the stable PWL of 10 to 15 seconds. The test compound is administered in a volume of 0.5 mL per 100 g body weight. PWL are measured after 1, 3 or 5 hours after drug administration.

### Mechanical Hyperalgesia

Male 4-week-old SD rats are used. CFA (300 mg of Mycobacterium Tuberculosis H37RA (Difco, MI) in 100 μL of liquid paraffin (Wako, Osaka, Japan)) is injected into the plantar surface of a hind paw of the rats. Two days after CFA-injection, mechanical hyperalgesia is tested by measuring paw withdrawal threshold (PWT, grams) to pressure using the analgesy-Meter (Ugo-Basile, Varese, Italy). The animals are gently restrained, and steadily increasing pressure is applied to the dorsal surface of a hind paw via a plastic tip. The pressure required to elicit paw withdrawal is determined. The test compound is administered in a volume of 0.5 mL per 100 g body weight. PWT are measured after 1, 3 or 5 hours after drug administration.

### Pharmaceutical Compositions

When employed as pharmaceuticals, the amide compounds of this invention are typically administered in the form of a pharmaceutical composition. Such compositions can be prepared in a manner well known in the pharmaceutical art and comprise at least one active compound.

Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of this invention can be administered by a variety of routes including by way of non limiting example, oral, rectal, transdermal, subcutaneous, intravenous, intramuscular and intranasal. Depending upon the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions of this invention. The present invention, however, is not limited to the following pharmaceutical compositions.

### Formulation 1

### Tablets

A compound of formula I is admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2

### Capsules

A compound of formula I is admixed as a dry powder with a starch diluent in an approximate 1:1 weight ratio. The mixture is filled into 250 mg capsules (125 mg of active compound per capsule).

### Formulation 3

### Liquid

A compound of formula I (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4

### Tablets

The compound of formula I is admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5

### Injection

The compound of formula I is dissolved or suspended in a buffered sterile saline injectable aqueous medium to, a concentration of approximately 5 mg/ml.

### Formulation 6

### Topical

Stearyl alcohol (250 g) and a white petrolatum (250 g) are melted at about 75° C. and then a mixture of a compound of formula I (50 g) methylparaben (0.25 g), propylparaben (0.15 g), sodium lauryl sulfate (10 g), and propylene glycol (120 g) dissolved in water (about 370 g) is added and the resulting-mixture is stirred until it congeals.

### Methods of Treatment

The present compounds are used as therapeutic agents for the treatment of conditions in mammals. Accordingly, the compounds and pharmaceutical compositions of this invention find use as therapeutics for preventing and/or treating neurodegenerative, autoimmune and inflammatory conditions in mammals including humans.

In a method of treatment aspect, this invention provides a method of treating a mammal susceptible to or afflicted with a condition associated with arthritis, uveitis, asthma, myocardial infarction, traumatic brain injury, acute spinal cord injury, alopecia (hair loss), inflammatory bowel disease and autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. Compounds have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

In additional method of treatment aspects, this invention provides methods of treating a mammal susceptible to or afflicted with neurodegenerative diseases and disorders such as, for example Parkinson's disease, Alzheimer's disease and multiple sclerosis; diseases and disorders which are mediated by or result in neuroinflammation such as, for example traumatic brain injury, stroke, and encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders, uveitis and atherosclerosis; itch/pruritus such as, example psoriasis; alopecia (hair loss); obesity; lipid disorders; cancer; blood pressure; spinal cord injury; and renal disorders method comprises administering an effective condition-treating or condition-preventing amount of one or more of the pharmaceutical compositions just described.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as neurodegenerative and autoimmune conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound or its derivative, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of a neurodegenerative, autoimmune or inflammatory condition, the compounds or their derivatives of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other active derivatives. A VR1 antagonist may be usefully combined with another pharmacologically active compound, or with two or more other pharmacologically active compounds, particularly in the treatment of pain. For example, a VR1 antagonist, particularly a compound of formula (I), or a pharmaceutically acceptable salt or solvate thereof, as defined above, may be administered simultaneously, sequentially or separately in combination with one or more agents selected from:

an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;

a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;

a barbiturate sedative, e.g. amobarbital, aprobarbital, butabarbital, butabital, mephobarbital, metharbital, methohexital, pentobarbital, phenobartital, secobarbital, talbutal, theamylal or thiopental;

a benzodiazepine having a sedative action, e.g. chlordiazepoxide, clorazepate, diazepam, flurazepam, lorazepam, oxazepam, temazepam or triazolam;

an H1 antagonist having a sedative action, e.g. diphenhydramine, pyrilamine, promethazine, chlorpheniramine or chlorcyclizine;

a sedative such as glutethimide, meprobamate, methaqualone or dichloralphenazone;

a skeletal muscle relaxant; e.g. baclofen, carisoprodol, chlorzoxazone, cyclobenzaprine, methocarbamol or orphrenadine;

an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;

an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;

a tricyclic antidepressant, e.g. desipramine, imipramine, amitriptyline or nortriptyline;

an anticonvulsant, e.g. carbamazepine, lamotrigine, topiratmate or valproate;

a tachykinin (NK) antagonist, particularly an NK-3, NK-2 or NK-1 antagonist, e.g. (aR,9R)-7-[3,5-bis(trifluoromethyl)benzyl]-8,9,10,11-tetrahydro-9-methyl-5-(4-methyl phenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);

a muscarinic antagonist, e.g. oxybutynin, tolterodine, propiverine, tropsium chloride, darifenacin, solifenacin, temiverine and ipratropium;

a COX-2 selective inhibitor, e.g. celecoxib, rofecoxib, parecoxib, valdecoxib, deracoxib, etoricoxib, or lumiracoxib;

a coal-tar analgesic, in particular paracetamol;

a neuroleptic such as droperidol; chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;

a beta-adrenergic such as propranolol;

a local anaesthetic such as mexiletine;

a corticosteroid such as dexamethasone;

a 5-HT receptor agonist or antagonist, particularly a 5-HT1B/1D agonist such as eletriptan, sumatriptan, naratriptan, zolmitriptan or rizatriptan;

a 5-HT2A receptor antagonist such as R(+)-alpha-(2,3-dimethoxy-phenyl)-1-[2-(4-fluorophenylethyl)]-4-piperidinemethanol (MDL-100907);

a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;

Tramadol®;

a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-c]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;

an alpha-2-delta ligand such as gabapentin, pregabalin, 3-methylgabapentin, (1a,3a,5a)(3-amino-methyl-bicyclo[3.2.0]hept-3-yl)-acetic acid, (3S,5R)-3_aminomethyl-5_methyl-heptanoic acid, (3S,5R)-3_amino-5_methyl-heptanoic acid, (3S,5R)-3_amino-5_methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3_aminomethyl-5_methyl-octanoic acid, (3S,5R)-3_amino-5_methyl-nonanoic acid, (3S,5R)-3_amino-5_methyl-octanoic acid, (3R,4R,5R)-3-amino-4,5-dimethyl-heptanoic acid and (3R,4R,5R)-3-amino-4,5-dimethyl-octanoic acid;

a cannabinoid;

a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;

a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;

a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;

an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol,

2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;

an acetylcholinesterase inhibitor such as donepezil;

a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;

a leukotriene B4 antagonist; such as 1-(3-biphenyl-4-ylmethyl-4-hydroxy-chroman-7-yl)-cyclopentanecarboxylic acid (CP-105096), 5-[2-(2-Carboxyethyl)-3-[6-(4-methoxyphenyl)-5E-hexenyl]oxyphenoxy]-valeric acid (ONO-4057) or DPC-11870,

a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);

a sodium channel blocker, such as lidocaine;

a 5-HT3 antagonist, such as ondansetron;

and the pharmaceutically acceptable salts and solvates thereof.

In as much as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

### Preparation of the Compounds

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The target compounds are synthesized by known reactions outlined in the following schemes. The products are isolated and purified by known standard procedures. Such procedures include (but are not limited to) recrystallization, column chromatography or HPLC.

In this specification, especially in “General Synthesis” and “Examples”, the following abbreviations can and may be used:

BEP 2-bromo-1-ethylpyridinium tetrafluoroborate
BOP benzotriazol-1-yloxy-tris(dimethylamino)phosphonium hexafluorophosphate
CDI 2-chloro-1,3-dimethylimidazolinium chloride
Co(TPP) 5, 10, 15, 20 tetraphenyl-21H, 23H porphine Co(II)
DCC dicyclohexylcarbodiimide
DCM dichloromethane
DME 1,2-dimethoxyethane dimethoxyethane
DMF N,N-dimethyl formamide
DMSO dimethyl sulfoxide
EDC 1-ethyl-3-(3′-dimethylaminopropyl)carbodiimide hydrogen chloride)
EtOAc ethyl acetate
EtOH ethanol
HBTU O-Benzotriazole-N,N,N′,N′-tetramethyl-uronium-hexafluoro-phosphate
HOBt 1-hydroxybenzotriazole
MeOH methanol
NMP N-methyl-2-pyrroliidone
PdCl₂ (pddf).CH₂Cl₂ palladiumdichloro-1,1′-bis(diphenylphosphino)ferrocene-dichloromethane complex
THF tetrahydrofuran
TFA trifluoroacetic acid

### General Synthesis

The compounds of the present invention may be prepared by a variety of processes well known for the preparation of compounds of this type, for example as shown in the following reaction Schemes. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1999). In the following general methods, X, Y, W, W′, R¹, R², R³, R⁴, R⁵, R⁶, R⁷, and R⁸ are as previously defined for a compound of the formula (I) unless otherwise stated.

The following reaction scheme illustrates the preparation of compounds of formula (I).

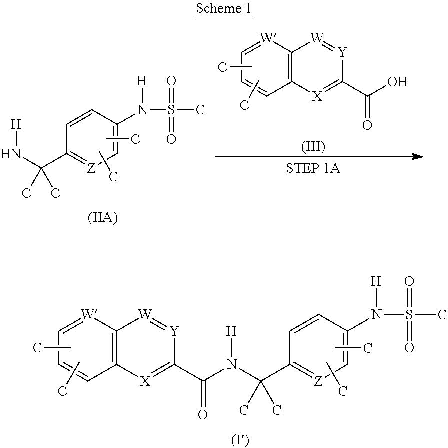

For the purposes of Scheme 1 above, and Scheme 2 (below) the symbols “C” are taken as the corresponding R¹-R⁷ groups, defined for formula I.

In this Step, an amide compound of formula (I′) can be prepared by the coupling reaction of an amine compound of formula (IIA) with the acid compound of formula (III) in the presence or absence of a coupling reagent in an inert solvent. This reaction can be also carried out via activated carboxylic derivatives. Suitable coupling reagents are those typically used in peptide synthesis including, for example, diimides (e.g., DCC, EDC), 2-ethoxy-N-ethoxycarbonyl-1,2-dihydroquinoline, BEP, CDI, BOP, diethyl azodicarboxylate-triphenylphosphine, diethylcyanophosphate, diethylphosphorylazide, 2-chloro-1-methylpyridinium iodide, N,N′-carbonyldiimidazole, benzotriazole-1-yl diethyl phosphate, ethyl chloroformate and isobutyl chloroformate.

The reaction can be carried out in the presence of a base such as, HOBt, N,N-diisopropylethylamine, N-methylmorpholine or triethylamine.

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: acetone; nitromethane; DMF; NMP; sulfolane; DMSO; 2-butanone; acetonitrile; halogenated hydrocarbons, such as DCM, dichloroethane, chloroform; and ethers, such as THF and 1,4-dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −20′C to 100° C., more preferably from about 0° C. to 60° C. The time required for the reaction can also vary widely, depending on many factors, notably the reaction temperature and the nature of the reagents and solvent employed. However, provided that the reaction is effected under, the preferred conditions outlined above, a period of from 5 minutes to 1 week, more preferably from 30 minutes to 24 hours, will usually suffice.

Alternatively, the compound of formula (III) can first be converted to an acylhalide derivative by reaction with halogenating agents such as oxalylchloride, phosphorus oxychloride and thionyl chloride. The resulting acylhalide derivative can then be reacted with a compound of formula (IIA) as described above to provide a compound of formula (I′).

### Scheme 2

This illustrates preparation of compounds of formula (IIA).

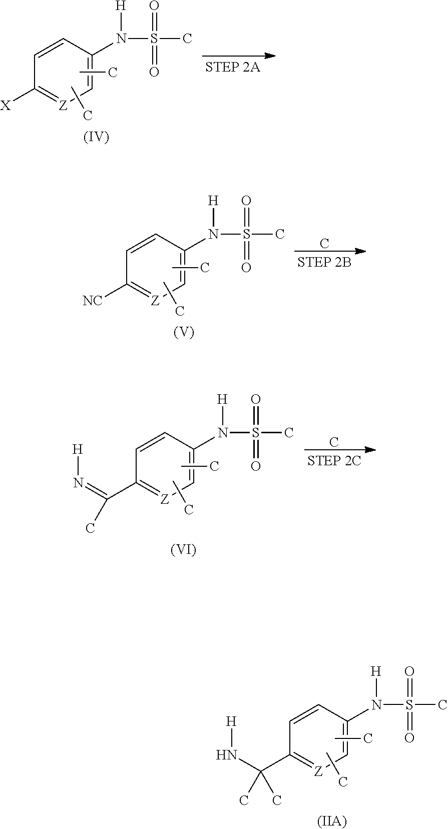

wherein X is a suitable leaving group such as sulfoxy or halogen, for example chloro; Z is independently selected from CR⁸ and N;
M1 is a metal, such as lithium, or MgY, wherein Y represents hydrogen or halogen such as fluorine, chlorine, bromine or iodine; and
M² is a metal, such as lithium, or MgY, wherein Y represents hydrogen or halogen, such as, fluorine, chlorine, bromine or iodine.

### Step 2A

In this step, the compound of formula (V) can be prepared by cyanating the compound of formula (IV) with a metal cyanide reagent in the presence of a transition metal catalyst in an inert solvent.

Examples of suitable solvents include: THF; 1,4-dioxane; DMF; acetonitrile; alcohols, such as MeOH or ethanol; halogenated hydrocarbons, such as DCM, 1,2-dichloroethane, chloroform or carbon tetrachloride; and DME. Example of suitable metal cyanide reagents include alkalimetal cyanide such as lithium cyanide, sodium cyanide or potassium cyanide; transition metal cyanide such as ferric(II) cyanide, cobalt(II) cyanide, Copper(I) cyanide, copper(II) cyanide or zinc(II) cyanide; sodium cyanide borohydride cyanide; and trimethylsilyl cyanide.

This reaction can be carried out in the presence of a suitable transition metal catalyst. There is likewise no particular restriction on the nature of the catalysts used, and any catalysts commonly used in reactions of this type can equally be used here. Examples of such catalysts include: tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride. Preferred catalysts are tetrakis(triphenylphosphine)-palladium, bis(triphenylphosphine)palladium(II) chloride, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride.

This reaction can be carried out in the presence of a suitable additive agent. Examples of such additive agents include: triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl and triphenylarsine.

The reaction can be carried out at a temperature of from 0° C. to 200° C., more preferably from 20° C. to 120° C. Reaction times are, in general, from 5 minutes to 48 hours, more preferably from 30 minutes to 24 hours.

### Step 2B

In this Step, an imine compound of formula (VI) can be prepared by the nucleophilic addition of a cyano compound of formula (V) with the organometallic compound of formula R³M¹. The reaction may be carried out in the presence of a solvent. Examples of suitable solvents include hydrocarbons, such as hexane; ethers, such as diethyl ether, diisopropyl ether, DME THF and 1,4-dioxane; or mixtures thereof. Reaction temperatures are generally in the range of from −100 to 50° C., preferably in the range of from −100° C. to room temperature. Reaction times are, in general, from 1 minute to a day, preferably from 1 hour to 10 hours.

The organometallic compound of formula R³M¹ can be prepared by reaction of a halide compound of R³. This reaction may be carried out in the presence of an organometallic reagent or a metal. Examples of suitable organometallic reagents include; alkyllithiums such as n-butyllithium, sec-butyllithium and tert-butyllithium; and aryllithiums such as phenyllithium and lithium naphthylide. Examples of suitable metals include magnesium. Examples of preferred inert solvents include hydrocarbons, such as hexane; ethers, such as diethyl ether, diisopropyl ether, DME, THF and 1,4-dioxane; or mixtures thereof. Reaction temperatures are generally in the range of from −100° C. to 50° C., preferably in the range of from −100° C. to room temperature. Reaction times are, in general, from 1 minute to a day, preferably from 1 hour to 10 hours.

### Step 2C

In this step, an amine of compound of formula (IIA) can be prepared by the nucleophilic addition of an imine compound of formula (VI) with the organometallic compound of formula R⁴M². The reaction may be carried out in the presence of a solvent. Examples of suitable solvents include hydrocarbons, such as hexane; ethers, such as diethyl ether, diisopropyl ether, DME, THF and 1,4-dioxane; or mixtures thereof. Reaction temperatures are generally in the range of from −100 to 50° C., preferably in the range of from −100° C. to room temperature. Reaction times are, in general, from 1 minute to a day, preferably from 1 hour to 10 hours.

The organometallic compound of formula R⁴M² can be prepared by reaction of a halide compound of R⁴. This reaction may be carried out in the presence of an organometallic reagent or a metal. Examples of suitable organometallic reagents include; alkyllithiums such as n-butyllithium, sec-butyllithium and tert-butyllithium; and aryllithiums such as phenyllithium and lithium naphtilide. Examples of suitable metals include magnesium. Examples of preferred inert solvents include hydrocarbons, such as hexane; ethers, such as diethyl ether, diisopropyl ether, DME, THF and 1,4-dioxane; or mixtures thereof. Reaction temperatures are generally in the range of from −100 to 50° C., preferably in the range of from −100° C. to room temperature. Reaction times are, in general, from 1 minute to a day, preferably from 1 hour to 10 hours.

When R³ and R⁴ are both hydrogen, a compound of formula (IIA) may be prepared from a compound of formula (V) as illustrated in Scheme 3.

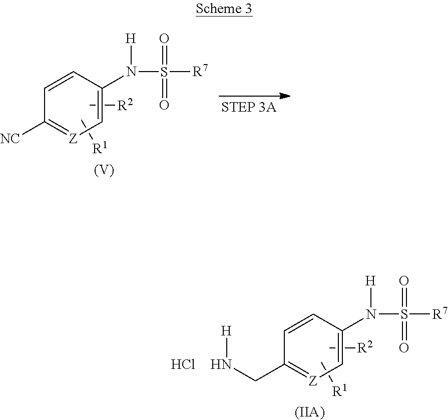

### Step 3A

In this step, the compounds of formula (IIA) can be prepared by hydrogenation of a compound of formula (V) under, for example, known hydrogenolysis conditions in the presence of a metal catalyst under a hydrogen atmosphere, or in the presence of hydrogen sources such as formic acid or ammonium formate, in an inert solvent. If desired, the reaction may be carried out under acidic conditions, for example, in the presence of hydrochloric acid or acetic acid. Examples of preferred metal catalysts include nickel catalysts such as Raney nickel; Pd—C; palladiumhydroxide-carbon; platinumoxide; platinum-carbon; ruthenium-carbon; rhodium-aluminumoxide; and tris[triphenyphosphine]rhodiumchloride. Examples of suitable inert aqueous or non-aqueous organic solvents include alcohols, such as methanol and ethanol; ethers, such as THF or 1,4-dioxane; acetone; dimethylformamide; halogenated hydrocarbons, such as DCM, dichloroethane or chloroform; and acetic acid; or mixtures thereof. The reaction can be carried out at a temperature in the range of from 20° C. to 100° C., preferably in the range of from 20° C. to 60° C. Reaction times are, in general, from 10 minutes to 4 days, preferably from 30 minutes to 24 hours. This reaction can be carried out under a hydrogen atmosphere at a pressure ranging from 1 to 100 atm, preferably from 1 to 10 atm.

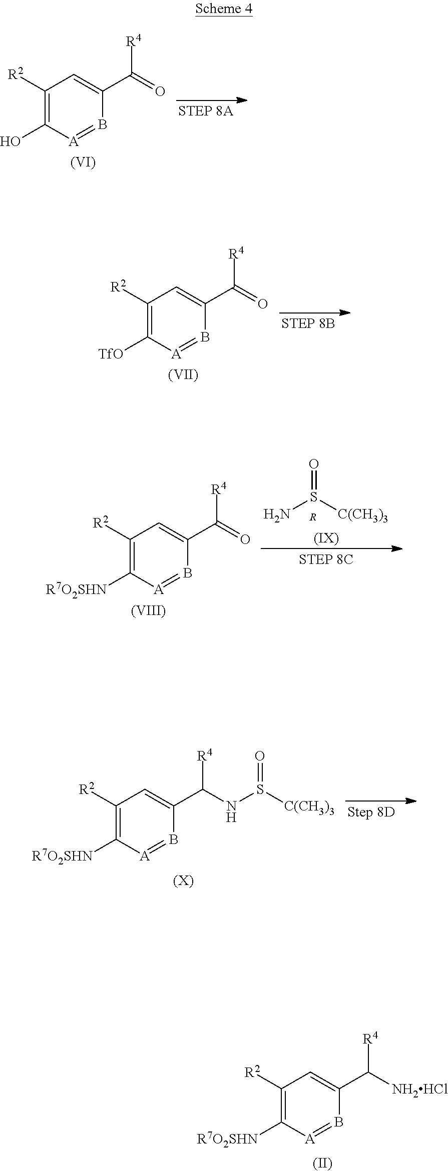

### Step 4A

For the purposes of Scheme 4 the symbols “A” and “B” are taken as the corresponding Z and CR⁸ groups.

In this Step, the compound of formula (VII) can be prepared by triflic reaction of the compound of formula (VI) using trifilic anhydrate under basic conditions in an inert solvent.

Examples of preferred bases include an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride; or an amine such as triethylamine, tributylamine, diisopropylethylamine, 2,6-lutidine, pyridine or dimethylaminopyridine. Examples of suitable solvents include THF; 1,4-dioxane; DMF; acetonitrile; alcohols, such as methanol or ethanol; halogenated hydrocarbons, such as DCM, 1,2-dichloroethane, chloroform or carbon tetrachloride; and acetic acid. Reaction temperatures are generally in the range of from −78° C. to 200° C., preferably in the range of from 0° C. to room temperature. Reaction times are, in general, from 1 minute to a day, preferably from 1 hour to 20 hours.

### Step 4B

In this Step, the compound of formula (VIII) can be prepared by coupling the compound of formula (VII) with alkyl sulfonamide in the presence of a catalyst and 4,5-bis(diphenylphosphino)-9,9-demethylxanthene (Xantphos) under basic conditions in an inert solvent, as described in Buchwald, S. L. Journal of American chemical society, 2002, 124, 6043-6048. Examples of suitable catalysts include tris(dibenzylidenacetone)dipalladium (0) and palladium reagents, such as palladium acetate and palladium dibenzylacetone. Examples of preferred base include an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, cesium carbonate, potassium fluoride, sodium hydride or potassium hydride; or an amine such as triethylamine, tributylamine, diisopropylethylamine, 2,6-lutidine, pyridine or dimethylaminopyridine. Examples of suitable solvents include THF; 1,4-dioxane; DMF; acetonitrile; alcohols, such as methanol or ethanol; halogenated hydrocarbons, such as DCM, 1,2-dichloroethane, chloroform or carbon tetrachloride; and acetic acid. Reaction temperatures are generally in the range of from 0 to 200° C., preferably in the range of from 100° C. to 140° C. Reaction times are, in general, from 1 minute to a day, preferably from 5 minutes to 1 hour.

### Step 4C

In this Step, the compound of formula (X) can be prepared by dehydration and reduction of the compound of formula (VIII) and sulfinamide of formula (IX) in the presence of a catalyst and reducing agent in an inert solvent. Dehydration is conducted in the presence of a dehydrating agent. Examples of suitable dehydrating agents include hydrogen halides such as hydrogen chloride and hydrogen bromide; sulfonic acids such as p-toluenesulfonic acid and benzenesulfonic acid; sulfonylchlorides such as methansulfonylchloride and p-toluenesulfonylchloride; methoxycarbonylsulfamoyltriethylammonium hydroxide; p-toluenesulfonylisocyanate; and titanium(IV) ethoxide. Reaction temperatures are generally in the range of from 0 to 200° C., preferably in the range of from 50° C. to 100° C. Reaction times are, in general, from 1 minute to 48 hours, preferably from 12 hours to 24 hours. The reduction may be carried out in the presence of a suitable reducing agent in an inert solvent or without solvent. Examples of preferred reducing agents include NaBH₄, LiAlH₄, LiBa₄, Fe, Sn or Zn. Reaction temperatures are generally in the range of from −78° C. to room temperature, preferably in the range of from −70° C. to 0° C. Reaction times are, in general, from 1 minute to a day, preferably from 3 hours to 6 hours. Examples of suitable solvents include THF; 1,4-dioxane; DMF; acetonitrile; alcohols, such as methanol or ethanol; halogenated hydrocarbons, such as DCM, 1,2-dichloroethane, chloroform or carbon tetrachloride; and acetic acid.

### Step 4D

In this Step, the compound of formula (IIA) can be prepared by deprotection and salt formation of the compound of formula (X) under acidic conditions in an inert solvent, using the method of D. Cogan et. al., Journal of American Chemical Society, 1999, 121, 268-269. Reaction temperatures are generally in the range of from 0 to 200° C., preferably room temperature. Reaction times are, in general, from 1 minute to 24 hours, preferably from 5 minutes to 1 hour. Examples of suitable solvents include THF; 1,4-dioxane; DMF; acetonitrile; alcohols, such as methanol or ethanol; halogenated hydrocarbons, such as DCM, 1,2-dichloroethane, chloroform or carbon tetrachloride; and acetic acid.

### Scheme 5

This illustrates an alternative preparation of compounds of formula (VIII).

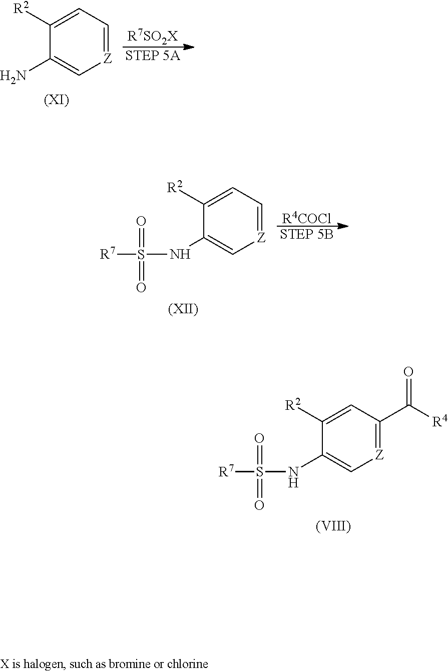

### Step 5A

In this Step, the compounds of formula (XII) can be prepared by sulfonylation of the compound of formula (XI) with R⁷SO₂X under, for example, known sulfonylation conditions in the presence of a base in an inert solvent. Examples of preferred base include an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride; or an amine such as triethylamine, tributylamine, diisopropylethylamine, 2,6-lutidine, pyridine or dimethylaminopyridine. Examples of suitable inert aqueous or non-aqueous organic solvents include alcohols, such as methanol or ethanol; ethers, such as THF or 1,4-dioxane; acetone; dimethylformamide; halogenated hydrocarbons, such as DCM, dichloroethane or chloroform; and acetic acid; or mixtures thereof. The reaction can be carried out at a temperature in the range of from 20° C. to 100° C., preferably in the range of from 20° C. to 60° C. Reaction times are, in general, from 10 minutes to 4 days, preferably from 30 minutes to 24 hours.

### Step 5B

In this step, the compounds of formula (VIII) can be prepared by Friedel-Crafts acylation of the compound of formula (XII) with R⁴COCl under, for example, known Friedel-Crafts acylation conditions in the presence of a metal and acylhalide. This reaction may be carried out in an inert solvent. Examples of suitable solvents include halogenated hydrocarbons, such as DCM, dichloroethane or chloroform; and aromatic hydrocarbons, such as nitrobenzene and chlorobenzene. Examples of suitable catalysts include aluminum halides, such as aluminum chloride and aluminum bromide. This reaction can be carried out at temperature of from −50° C. to 200° C., preferably from about −10° C. to 150° C. for from 5 minutes to 48 hours, preferably from 30 minutes to 24 hours.

When R¹ is hydrogen, compounds of formula (IIA) may be prepared from compounds of formula (VIII) as illustrated in Scheme 6.

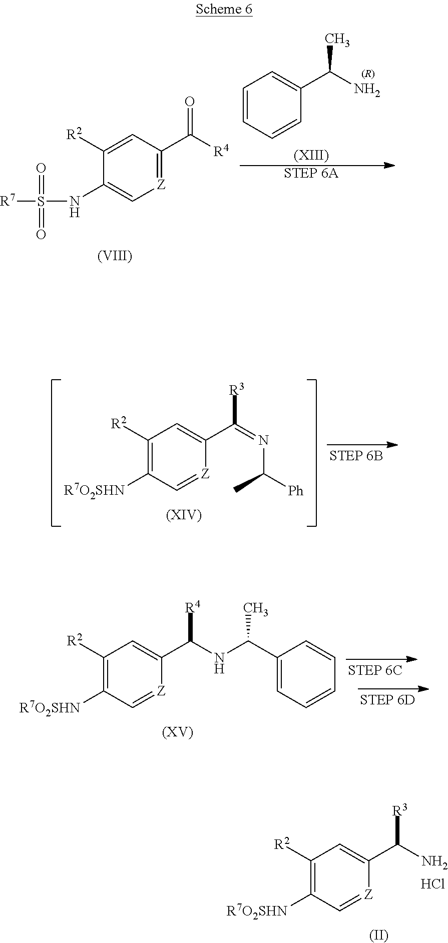

### Step 6A

In this step, the compound of formula (XIV) can be prepared by dehydration of the compound of formula (VIII) using a Lewis acid under basic conditions in an inert solvent. Examples of preferred Lewis acids include titanium tetrachloride, aluminium tetrachloride or zirconium tetrachloride. Examples of preferred bases include an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride; or an amine such as methylamine, tributylamine, diisopropylethylamine, 2,6-lutidine, pyridine or dimethylaminopyridine. Examples of suitable solvents include THF; 1,4-dioxane; DMF; acetonitrile; alcohols, such as methanol or ethanol; halogenated hydrocarbons, such as DCM, 1,2-dichloroethane, chloroform or carbon tetrachloride; and acetic acid. Reaction temperatures are generally in the range of from −78 to 200° C., preferably in the range of from 0° C. to room temperature. Reaction times are, in general, from 1 minute to a day, preferably from 1 hour to 20 hours.

### Step 6B

In this Step, the compound of formula (XV) can be prepared by the reduction of the compound of formula (XIV) in the presence of a suitable reducing agent in an inert solvent or without solvent. Examples of preferred reducing agents include NaBH₄, LiAlH₄, LiBH₄, Fe, Sn or Zn. Reaction temperatures are generally in the range of from −78° C. to room temperature, preferably in the range of from −70° C. to 0° C. Reaction times are, in general, from 1 minute to a day, preferably from 3 hours to 6 hours. Examples of suitable solvents include THF; 1,4-dioxane; DMF; acetonitrile; alcohols, such as methanol or ethanol; halogenated hydrocarbons; such as DCM, 1,2-dichloroethane, chloroform or carbon tetrachloride; and acetic acid.

The reduction may also be carried out in the presence of a suitable metal catalyst under a hydrogen atmosphere in an inert solvent. Example of preferred metal catalysts include nickel catalysts such as Raney nickel; Pd—C; palladiumhydroxide-carbon; platinumoxide; platinum-carbon; ruthenium-carbon; rhodium-aluminumoxide; and tris[triphenyphosphine]rhodiumchloride. Examples of suitable inert aqueous or non-aqueous organic solvents include: alcohols, such as methanol or ethanol; ethers, such as THF or 1,4-dioxane; acetone; dimethylformamide; halogenated hydrocarbons, such as DCM, dichloroethane or chloroform; and acetic acid; or mixtures thereof. The reaction can be carried out at a temperature in the range of from 20° C. to 100° C., preferably in the range of from 20° C. to 60° C. Reaction times are, in general, from 10 minutes to 4 days, preferably from 30 minutes to 24 hours. This reaction can be carried out under a hydrogen atmosphere at a pressure ranging from 1 to 100 atoms, preferably from 1 to 10 atom.

### Step 6C

In this step, the compounds of formula (IIA) can be prepared by hydrogenation of the compound of formula (XV) under, for example, known hydrogenolysis conditions in the presence of a metal catalyst under hydrogen atmosphere, or in the presence of hydrogen sources such as formic acid or ammonium formate, in an inert solvent. If desired, the reaction is carried out under acidic conditions, for example, in the presence of hydrochloric acid or acetic acid. Examples of preferred metal catalysts include nickel catalysts such as Raney nickel; Pd—C; palladiumhydroxide-carbon; platinumoxide; platinum-carbon; ruthenium-carbon; rhodium-aluminumoxide; and tris[triphenyphosphine]rhodiumchloride. Examples of suitable inert aqueous or non-aqueous organic solvents include alcohols, such as methanol or ethanol; ethers, such as THF or 1,4-dioxane; acetone; dimethylformamide; halogenated hydrocarbons, such as DCM, dichloroethane or chloroform; and acetic acid; or mixtures thereof. The reaction can be carried out at a temperature in the range of from 20° C. to 100° C., preferably in the range of from 20° C. to 60° C. Reaction times are, in general, from 10 minutes to 4 days, preferably from 30 minutes to 24 hours. This reaction can be carried out under a hydrogen atmosphere at a pressure ranging from 1 to 100 atm, preferably from 1 to 10 atm.

### Step 6D

In this step, the compounds of formula (IIA) can be prepared from the compound of formula (XV) by salt formation with, for example, hydrogen-chloride methanol solution, 1,4-dioxane solution and aqueous solution. The reaction can be carried out at a temperature in the range from of from 20° C. to 100° C., preferably in the range of from 20° C. to 60° C. Reaction times are, in general, from 10 minutes to 4 days, preferably from 30 minutes to 24 hours.

### Scheme 7

When Z is N, compounds of formula (VIII) can be prepared from compounds of formula (XVI) as illustrated by Scheme 7.

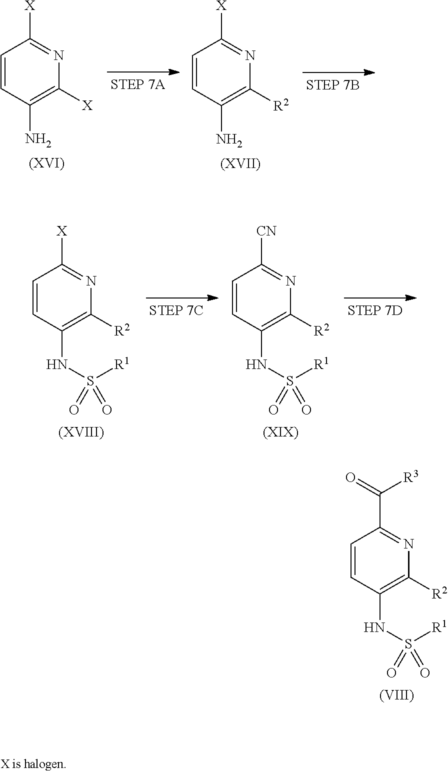

### Step 7A

In this Step, a compound of formula (XVII) can be prepared by alkylation of a compound of formula (XVI) with an alkylating agent in the presence of a suitable metal catalyst in an inert solvent. A preferred alkylating agent is selected from, but not limited to: trialkylmetals such as trimethylaluminum or triethylaluminum; and alkylmagnesium halides such as methylmagnesium bromide. The reaction can be carried out in the presence of an additive compound such as lithium bromide or a dialkylzinc halide such as dimethylzinc dichloride prepared by dimethylzinc and titanium chloride, preferably trimethylaluminum. Examples of suitable metal catalysts include tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride. Preferred catalysts are tetrakis(triphenylphosphine)-palladium, bis(triphenylphosphine)palladium(II) chloride, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride. Examples of preferred reaction inert solvents include halogenated hydrocarbons such as DCM, 1,2-dichloroethane, chloroform or carbon tetrachloride; acetic acid; 1,4-dioxane; THF; DMF; dimethylsulfoxide; and dyglime.

This reaction can be carried out in the presence of a suitable additive agent. Examples of such additive agents include triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, tetrabutylammonium chloride, tetrabutylammonium fluoride, lithium acetate, lithium chloride, triethylamine, potassium sodium methoxide, sodium hydroxide, sodium carbonate, sodium bicarbonate and/or sodium iodide.

Reaction temperatures are generally in the range of from −100° C. to 200° C., preferably in the range of from −40° C. to 100° C. Reaction times are, in general, from 1 minute to a day, preferably from 1 hour to 10 hours.

### Step 7B

In this Step, a compound of formula (XVIII) can be prepared from a compound of formula (XVII) by the method described in Step 5A above.

### Step 7C

In this Step, a compound of formula (XIX) can be prepared from a compound of formula (XVIII) by the method described in Step 2A above.

### Step 7D

In this Step, a compound of formula (VIII) can be prepared by alkylation of the compound of formula (XIX) with an alkylating agent in an inert solvent. Preferred alkylating agents and inert solvents are the same as those of Step 14A. The reaction can be carried out at a temperature of from 0° C. to 200° C., more preferably from 20° C. to 120° C. Reaction times are, in general, from 5 minutes to 96 hours, more preferably from 30 minutes to 24 hours.

### Scheme 8

Compounds of formula (VIII) may be prepared from compounds of formula (XX) as illustrated in Scheme 8, below.

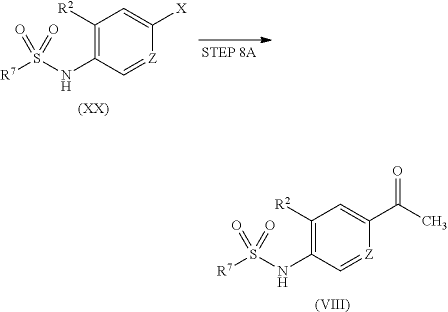

X represents halogen such as iodide, bromide, chloride or fluoride.

### Step 8A

In this step, a compound of formula (VIII) can be prepared by acylation of a compound of formula (XX) under acylating conditions using n-buthyl vinyl ether as a reagent in water-organic co-solvent mixture in the presence of a suitable transition metal catalyst and in the presence or absence of a base, followed by hydrolysis under acidic conditions.

Examples of suitable organic solvents include THF; 1,4-dioxane; DMF; acetonitrile; alcohols, such as methanol or ethanol; halogenated hydrocarbons, such as DCM, 1,2-dichloroethane, chloroform or carbon tetrachloride; and diethylether in the presence or absence of an aqueous base such as aqueous KOH, NaOH, LiOH or K₂CO₃. Examples of suitable catalysts include tetrakis(triphenylphosphine)-palladium, bis(triphenylphosphine)palladium(II) chloride, copper(0), copper(I) acetate, copper(I) bromide; copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride. Preferred catalysts are tetrakis(triphenylphosphine)palladium, bis(triphenylphosphine)palladium(II) chloride, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride.

This reaction can be carried out in the presence of a suitable additive agent. Examples of such additive agents include triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, tetrabutylammonium chloride, tetrabutylammonium fluoride, lithium acetate, lithium chloride, triethylamine, potassium sodium methoxide, sodium hydroxide, sodium carbonate, sodium bicarbonate, and/or sodium iodide.

This reaction can be acidified with a suitable acid. Examples of such acid agents include concentrated hydrogen chloride aqueous solution, sulfonic acid in the presence of water.

The reaction can be carried out at a temperature of from 0° C. to 200° C., more preferably from 20° C. to 120° C. Reaction times are, in general, from 5 minutes to 96 hours, more preferably from 30 minutes to 24 hours.

### Scheme 9

When Z is CR⁸; R² is hydrogen, halogen, (C₁-C₆)alkyl, (C₁-C₆)alkoxy or (C₁-C₆)alkoxy-(C₁-C₆)alkyl; and R⁸ is hydrogen or (C₁-C₆)alkyl, a compound of formula (VIII) may be prepared from a compound of formula (XI) as illustrated in Scheme 9.

This illustrates an improved method of Scheme 5 to prepare compounds of formula (VIII) from compounds of formula (XI).

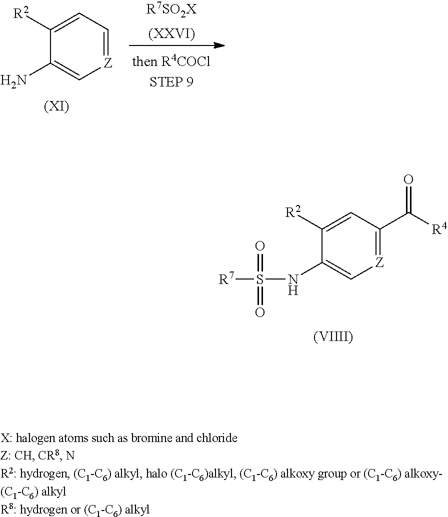

### Step 9

In this step, the compounds of formula (VIII) can be prepared by one-pot process of sulfonylation reaction of the compound of formula (XI) with R⁷SO₂X and subsequent Friedel-Crafts acylation reaction with R⁴COCl. The formation of undesirable N-acylated products is substantially suppressed by the one-pot procedure. The sulfonylation reaction is carried out under, for example, known sulfonylation conditions in the presence of a base in an inert solvent. The reaction may be carried out without the use of a solvent. Examples of preferred bases and suitable inert organic solvents are the same as Step 5A. The reaction can be carried out at a temperature in the range from of 20° C. to 100° C., preferably in the range of −20° C. to 40° C. Reaction time is, in general, from 5 minutes to 4 days, preferably 10 minutes to 3 hours. After the completion of the sulfonylation, Friedel-Crafts acylation reaction with R³Cl should follow without any work-up procedure for the preceding reaction. Friedel-Crafts acylation reaction with R³Cl is carried out under, for example, known Friedel-Crafts acylation in the presence of a metal and acylhalide. This reaction may be carried out in an inert solvent. Examples of suitable solvents and suitable catalysts are the same as Step 5B. This reaction can be carried out at temperature of −50° C. to 200° C., preferably from about −10° C. to 150° C. for 5 minutes to 48 hours, preferably 10 minutes to 24 hours.

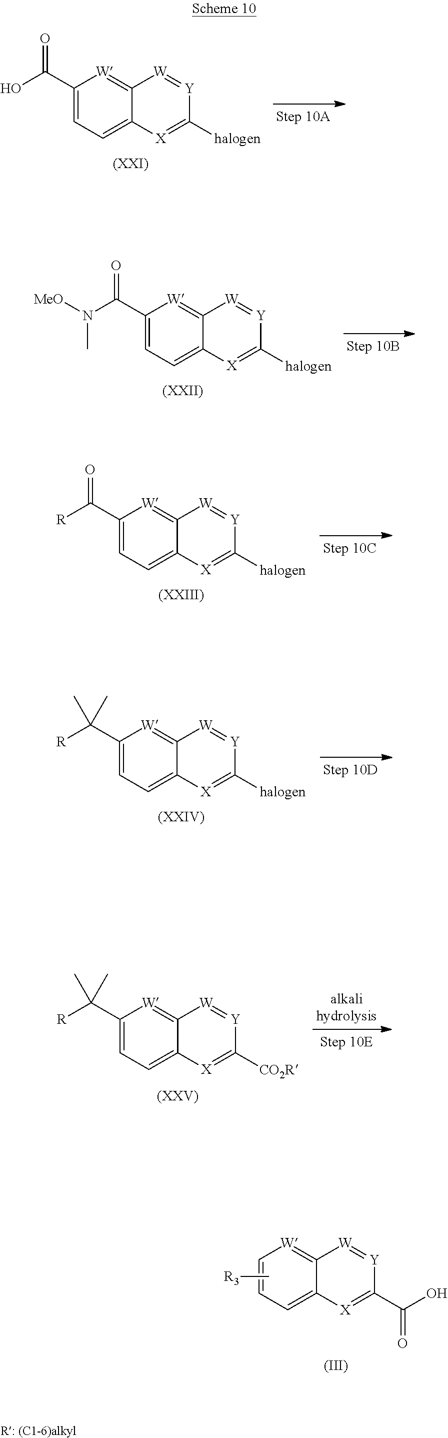

In this Step, an amide compound of formula (XXII) can be prepared by the coupling reaction of an N,O-dimethylhydroxylamine with the acid compound of formula (XXI) in the presence of a coupling reagent in an inert solvent. This reaction can be also carried out via activated carboxylic derivatives. Suitable coupling reagents are those typically used in peptide synthesis including, for example, diimides (e.g., DCC, EDC), 2-ethoxy-N-ethoxycarbonyl-1,2-dihydroquinoline, BEP, CDI, BOP, HBTU, diethyl azodicarboxylate-triphenylphosphine, diethylcyanophosphate, diethylphosphorylazide, 2-chloro-1-methylpyridinium iodide, N,N′-carbonyldiimidazole, benzotriazole-1-yl diethyl phosphate, ethyl chloroformate and isobutyl chloroformate.

The reaction can be carried out in the presence of a base such as, HOBt, N,N-diisopropylethylamine, N-methylmorpholine or triethylamine. The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include acetone; nitromethane; DMF; NMP; sulfolane; DMSO; 2-butanone; acetonitrile; halogenated hydrocarbons, such as DCM, dichloroethane, chloroform; and ethers, such as THF and 1,4-dioxane.

The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −20° C. to 100° C., more preferably from about 0° C. to 60° C. The time required for the reaction can also vary widely, depending on many factors, notably the reaction temperature and the nature of the reagents and solvent employed. However, provided that the reaction is effected under the preferred conditions outlined above, a period of from 5 minutes to 1 week, more preferably from 30 minutes to 24 hours, will usually suffice.

### Step 10 B

In this Step, the ketone compound of formula (XXIII) can also be prepared by acylation of the compound of formula (XXII) with organometallic reagents in an inert solvent. The organometallic compound of formula R-MX can be prepared by reaction of a halide compound of R, wherein R is alkyl. M represents metal such as lithium, or MgX, wherein X represents a hydrogen atom, a halogen atom such as, fluorine, chlorine, bromine or iodine. Examples of suitable organometallic reagents include alkyllithiums such as methyllithium, n-butyllithium, sec-butyllithium and tert-butyllithium; aryllithiums such as phenyllithium and lithium naphtilide; alkylmagnesium halide such as methylmagnesium halide, isopropylmagnesium halide, and t-butylmagnesium halide; arylmagnesium halide such as phenylmagnesium halide. The reaction is normally and preferably effected in the presence of a solvent. Examples of suitable solvents include ethers such as tetrahydrofuran (THF), 1,2-dimethoxyethane (DME), and 1,4-dioxane. This reaction can be carried out at a temperature in the range from −78 to 100° C., usually from 0° C. to ambient temperature for 30 minutes to 24 hours, usually 60 minutes to 6 hours.

### Step 10 C

In this Step, a compound of formula (XXIV) can be prepared by an alkylation reaction of the compound of formula (XXIII) with geminal-alkylating reagent in an inert solvent. Examples of preferred alkylating agents include trialkylmetals such as trimethylaluminum, triethylaluminum; alkylmagnesium halides such as methylmagnesium bromide in the presence of additive compound such as lithium bromide; dialkyltitanium halides such as dimethyltitanium dichloride prepared by dimethylzinc and titanium chloride; and is most preferably dimethyltitanium dichloride. Examples of preferred inert solvents for the reaction include halogenated hydrocarbons, such as DCM, 1,2-dichloroethane, chloroform or carbon tetrachloride; ethers, such as diethyl ether, diisopropyl ether, DME, THF and 1,4-dioxane; hydrocarbons, such as n-hexane, cyclohexane, benzene and toluene; or mixtures thereof. Reaction temperatures are generally in the range of from −100 to 200° C., preferably in the range of from −40° C. to 100° C. Reaction times are, in general, from 1 minute to a day, preferably from 1 hour to 10 hours.

### Step 10 D

In this Step, the compound of formula (XXV) can also be prepared by alkoxycarbonyl insertion of the compound of formula (XXIV) with carbon monoxide and alcohol (e.g. methanol or ethanol) in the presence of a catalyst and/or base in an inert solvent. Examples of suitable catalysts include palladium reagents, such as palladium acetate and palladium dibenzylacetone. Example of suitable bases include N,N-diisopropylethylamine, N-methylmorpholine and triethylamine. If desired, this reaction may be carried out in the presence or absence of an additive such as 1,1′-bis(diphenylphosphino)ferrocene, triphenylphosphine or 1,3-bis-(diphenylphosphino)propane (DPPP).

The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include acetone, nitromethane, DMF, sulfolane, DMSO, NMP, 2-butanone, acetonitrile; halogenated hydrocarbons, such as dichloromethane, dichloroethane, chloroform; and ethers, such as tetrahydrofuran and dioxane.

The reaction can take place over a wide range of temperatures, and the precise reaction temperature is not critical to the invention. The preferred reaction temperature will depend upon such factors as the nature of the solvent, and the starting material or reagent used. However, in general, it is found to convenient to carry out the reaction at a temperature of from −20° C. to 150° C., more preferably from about 50° C. to 80° C. The time required for the reaction may also vary widely, depending on many factors, notably the reaction temperature and the nature of the reagents and solvent employed. However, provided that the reaction is effected under the preferred conditions outlined above, a period of 30 minutes to 24 hours, more preferably 1 hour to 10 hours, will usually suffice.

### Step 10E

In this Step, an acid compound of formula (III) can be prepared by hydrolysis of the ester compound of formula (XXV) in a solvent.

The hydrolysis can be carried out by conventional procedures. In a typical procedure, the hydrolysis carried out under the basic condition, e.g. in the presence of sodium hydroxide, potassium hydroxide or lithium hydroxide. Examples of suitable solvents include alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). Preferable solvents are methanol, ethanol, propanol, tetrahydrofuran (THF), dimethoxyethane (DME), 1,4-dioxane, N,N-dimethylformamide (DMF), and dimethyl sulfoxide (DMSO). This reaction can be carried out at a temperature in the range from −20 to 100° C., usually from 20° C. to 65° C. for 30 minutes to 24 hours, usually 60 minutes to 10 hour.

The hydrolysis can also be carried out under the acidic condition, e.g. in the presence of e.g. in the presence of hydrogen halides, such as hydrogen chloride and hydrogen bromide; sulfonic acids, such as p-toluenesulfonic acid and benzenesulfonic acid; pyridium p-toluenesulfonate; and carboxylic acid, such as acetic acid and trifluoroacetic acid. Examples of suitable solvents include alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). Preferable solvents are methanol, ethanol, propanol, tetrahydrofuran (THF), dimethoxyethane (DME), 1,4-dioxane, N,N-dimethylformamide (DMF), and dimethyl sulfoxide (DMSO). This reaction can be carried out at a temperature in the range from −20 to 100° C., usually from 20° C. to 65° C. for 30 minutes to 24 hours, usually 60 minutes to 10 hour.

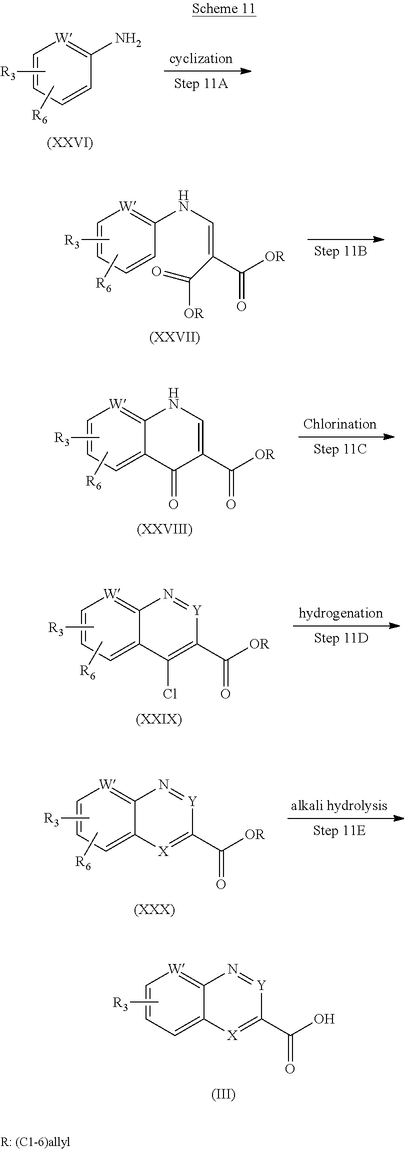

In this Step; a compound of formula (XXVII) can be prepared by N-substituted acrylation of the compound of formula (XXVI) with dialkyl alkoxy methylenemalonate in a reaction inert solvent or without solvent. Examples of suitable solvents include alcohols such as methanol, ethanol, propanol, butanol, 2-methoxyethanol, and ethylene glycol; ethers such as tetrahydrofuran (THF), 1,2-dimethoxyethane (DME), and 1,4-dioxane. As stated, this reaction may be performed without a solvent as well. The reaction can be carried out at a temperature in the range from 50° C. to 150° C. for 30 minutes to 24 hours, usually 60 minutes to 3 hours.

### Step 11B

In this Step, a compound of formula (XXVIII) can be prepared by thermal cyclization of the compound of formula (XXVII) in a reaction inert solvent. Examples of suitable solvents include ethers such as phenyl ether. This reaction can be carried out at a temperature in the range from 200 to 300° C. for 30 minutes to 24 hours, usually 250° C. for 30 minutes to 5 hours. (reference: Journal of Medicinal chemistry, 1998, Vol 41, No 25.)

### Step 11C

In this Step, a compound of formula (XXIX) can be prepared by halogenation of the compound of formula (XXVIII). The reaction is carried out under halogenation conditions with a halogenating reagent in a reaction inert solvent or without solvent.

Examples of suitable solvents include tetrahydrofuran, 1,4-dioxane, N,N-dimethylformamide, acetonitrile; halogenated hydrocarbons, such as dichloromethane, 1,2-dichloroethane, chloroform or carbon tetrachloride and acetic acid. Examples of suitable halogenating reagents include phosphorus oxyhalide such as phosphorus oxychloride and phosphorus oxybromide. The reaction can be carried out at a temperature of from 0° C. to 200° C., more preferably from ambient temperature to 150° C. Reaction times are, in general, from 5 minutes to 48 hours, more preferably 30 minutes to 6 hours, will usually suffice.

### Step 11D

In this Step, a dehalogenated compound of formula (XXX) can be prepared by hydrogenation of the compound of formula (XXIX) in a solvent. Hydrogenation reaction is carried out under, for example, known hydrogenolysis conditions in the presence of a metal catalyst under hydrogen atmosphere or in the presence of hydrogen sources such as formic acid or ammonium formate in a reaction inert solvent. If desired, the reaction is carried out under basic conditions, for example, in the presence of triethylamine. Preferable reagents are selected from, for example, nickel catalysts such as Raney nickel, palladium-carbon, palladiumhydroxide-carbon, platinumoxide, platinum-carbon, ruthenium-carbon; rhodium-aluminumoxide, and tris[triphenyphosphine]rhodiumchloride. Examples of suitable reaction inert aqueous or non-aqueous organic solvents include alcohols, such as methanol, ethanol; ethers, such as tetrahydrofuran or dioxane; acetone; dimethylformamide; halogenated hydrocarbons, such as dichloromethane, dichloroethane or chloroform; and acetic acid or mixtures thereof. The reaction can be carried out at a temperature in the range from of 20° C. to 100° C., preferably in the range of 20° C. to 60° C. Reaction times are, in general, from 10 minutes to 48 hours, preferably 30 minutes to 24 hours. This reaction can be carried out under hydrogen atmosphere at a pressure ranging from 1 to 100 atom, preferably from 1 to 10 atm. The preferable condition is the use of 5 or 10% palladium-carbon at ambient temperature for 1 to 24 hours under hydrogen atmosphere using a balloon.

### Step 11E

In this Step, an acid compound of formula (III) can be prepared by hydrolysis of the compound of formula (XXX) in a solvent by the method as described in Step 10E.

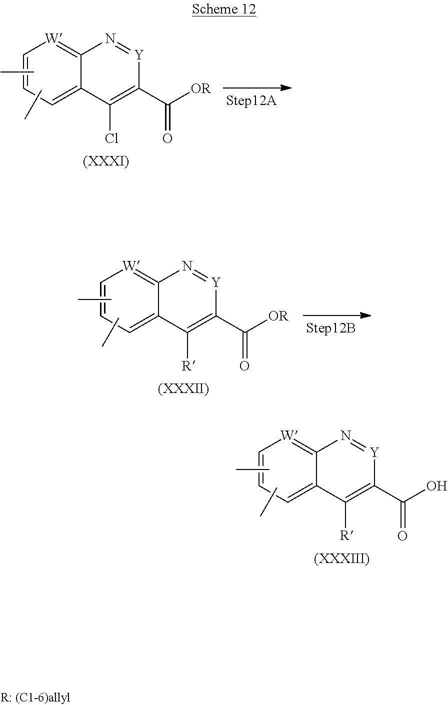

In this Step, a compound of formula (XXXII) can be prepared by coupling reaction of the compound of formula (XXXI) with R′—B(OH)₂ in a solvent. The coupling reaction may be carried out in the absence or presence of a base in a reaction inert solvent or without solvent. Examples of preferred base include an alkali or alkaline earth metal hydroxide, alkoxide, carbonate, or hydride, such as sodium hydroxide, potassium hydroxide, sodium methoxide, sodium ethoxide, potassium tert-butoxide, sodium carbonate, cesium carbonate or potassium carbonate, 2-tert-butylimino-2-diethylamino-1,3-dimethyl-perhydro-1,3,2-diazaphosphorine (HEMP), tert-butylimino-tri(pyrrolidino)phosphorane (BTPP), cesium fluoride (CsF), potassium fluoride, sodium hydride or potassium hydride, or an amine such as triethylamine, tributylamine, diisopropylethylamine, 2,6-lutidine, pyridine or dimethylaminopyridine. Examples of preferred reaction inert solvents include aromatic hydrocarbons, such as benzene, toluene, xylene, nitrobenzene and pyridine; halogenated hydrocarbons, such as methylene chloride, chloroform, carbon tetrachloride and dichloroethane; ethers, such as diethyl ether, diisopropyl ether, 1,2-dimethoxyethane (DME) tetrahydrofuran and dioxane; ethyl acetate, acetonitrile, N,N-dimethylformamide, dimethylsulfoxide and water or mixtures thereof. Reaction temperatures are generally in the range of −100° C. to 250° C., more preferably in the range of 0° C. to reflux temperature. Reaction times are, in general, from 1 Minute to a 10 day, more preferably from 20 minutes to 24 hours. This reaction may be carried out in the presence of a suitable catalyst. There is likewise no particular restriction on the nature of the catalyst used, and any catalyst commonly used in reactions of this type may equally be used here. Examples of such catalysts include tetrakis(triphenylphosphine)palladium, bis(triphenylphosphine)palladium(0)chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate palladium(II) acetate, palladium(II) chloride, bisacetonitriledichloropalladium(0), bis(dibenzylidenacetone)palladium(0), tris(dibenzylidenacetone)dipalladium(0) or [1,1′-bis(diphenylphosphino)ferrocene]palladium(II)dichloride.

This reaction may be carried out in the presence of a suitable additive agent. Example of such additive agents include triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl or triphenylarsine.

### Step 12B

In this Step, an acid compound of formula (XXXIII) which is a part of formula (III) can be prepared by hydrolysis of the compound of formula (XXXII) in a solvent by the method described in Step 10E.

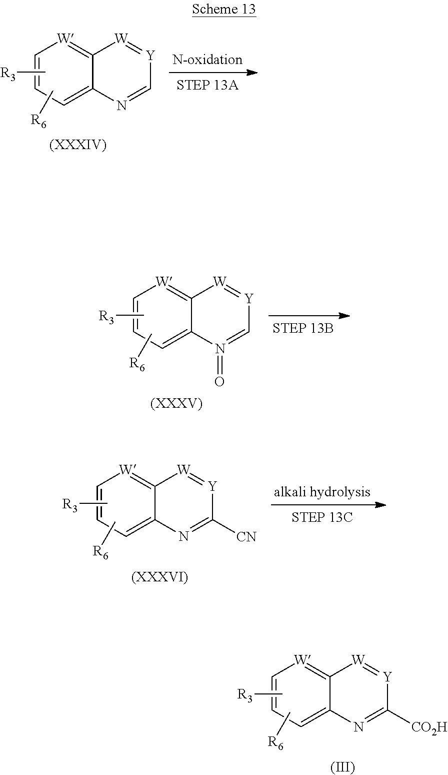

### Step 13A

In this Step, a N-oxide compound of formula (XXXV) can be prepared by oxidation of the compound of formula (XXXIV) in a reaction inert solvent. The oxidation reaction may be carried out in the absence or presence of an additive agent in a reaction inert solvent. Examples of preferred oxidation reagents meta-chloroperbenzoic acid (mCPBA), hydrogen peroxide, peracetic acid. Examples of preferred reaction inert solvents include halogenated hydrocarbons, such as methylene chloride, chloroform, carbon tetrachloride and dichloroethane; ethers, such as diethyl ether, diisopropyl ether, 1,2-dimethoxyethane (DME) tetrahydrofuran and dioxane; acetonitrile, acetic acid and water or mixtures thereof. Reaction temperatures are generally in the range of 0° C. to 250° C., more preferably in the range of 0° C. to 100° C. Reaction times are, in general, from 1 minute to 10 days, more preferably from 20 minutes to 6 hours. This reaction may be carried out in the presence of a suitable catalyst. There is likewise no particular restriction on the nature of the catalyst used, and any catalyst commonly used in reactions of this type may equally be used here. Examples of such catalysts include methyltrioxorhenium(VII), tungstic acid and sodium tungstate dehydrate.

### Step 13B

In this Step, a cyano compound of formula (XXXVI) can be prepared by cyanation of the compound of formula (XXXV) in an inert solvent. Examples of preferred cyanation reagents include trimethylsilanecarbonitrile (TMSCN), the combination of trimethylchlorosilane and sodium cyanide, the combination of acylating agents such as N,N-dimethylcarbamoyl chloride with trimethylsilanecarbonitrile (TMSCN). A preferred cyanation reagent is trimethylsilanecarbonitrile (TMSCN) in the presence of a base such triethylamine in a reaction inert solvent. Examples of preferred reaction inert solvents include halogenated hydrocarbons, such as methylene chloride, chloroform, carbon tetrachloride and dichloroethane; ethers, such as diethyl ether, 1,2-dimethoxyethane (DME), tetrahydrofuran (THF) and dioxane; acetonitrile, N,N-dimethylformamide, dimethylsulfoxide or mixtures thereof. Reaction temperatures are generally in the range of 0° C. to 250° C., more preferably in the range of 0° C. to 100° C. Reaction times are, in general, from 1 minute to 10 days, more preferably from 20 minutes to 24 hours.

### Step 13C

In this Step, an acid compound of formula (III) can be prepared by hydrolysis of the cyano compound of formula (XXXVI) in a solvent.

The hydrolysis can be carried out by conventional procedures. In a typical procedure, the hydrolysis may be carried out under basic conditions, e.g. in the presence of sodium hydroxide, potassium hydroxide or lithium hydroxide. Examples of suitable solvents include alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). Preferable solvents are methanol, ethanol, propanol, tetrahydrofuran (THE), dimethoxyethane (DME), 1,4-dioxane, N,N-dimethylformamide (DMF), and dimethyl sulfoxide (DMSO). This reaction can be carried out at a temperature in the range from −20 to 150° C., usually from 20° C. to 100° C. for 30 minutes to 24 hours, usually 60 minutes to 10 hours.

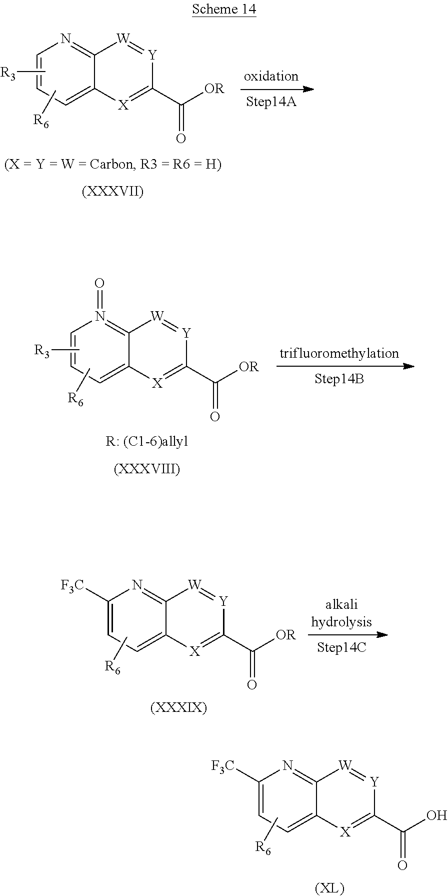

In this Step, a N-oxide compound of formula (XXXVIII) can be prepared by oxidation of the compound of formula (XXXVII) in a solvent by the method as described in Step 13A.

### Step 14B

In this Step, a compound of formula (XXXIX) can be prepared by trifluoromethylation of the compound of formula (XXXVIII) in a reaction inert solvent. Examples of preferred trifluoromethylation reagents include the combination of trifluoromethyltrimethylsilane (TMSCF₃) and initiator reagents. Examples of preferred catalytic initiator reagents include tetrabutylammonium fluoride cesium fluoride, lithium acetate, sodium acetate, potassium acetate, tetrabutylammonium acetate, lithium pivalate, lithium benzoate, potassium t-butoxide, sodium t-butoxide. Examples of preferred reaction inert solvents include hydrocarbons, such as hexane, benzene, toluene; halogenated hydrocarbons, such as methylene chloride, chloroform, carbon tetrachloride and dichloroethane; ethers, such as diethyl ether, diisopropyl ether, 1,2-dimethoxyethane (DME), tetrahydrofuran and dioxane; acetonitrile, ethyl acetate, N,N-dimethylformamide (DMF), dimethylsulfoxide (DMSO) or mixtures thereof. Reaction temperatures are generally in the range of −78′C to 200° C., more preferably in the range of −78° C. to 100° C. Reaction times are, in general, from 1 minute to 10 days, more preferably from 20 minutes to 24 hours.

### Step 14C

In this Step, an acid compound of formula (XL) which is apart of formula (III) can be prepared by hydrolysis of the compound of formula (XXXIX) in a solvent by the method as described in Step 10E.

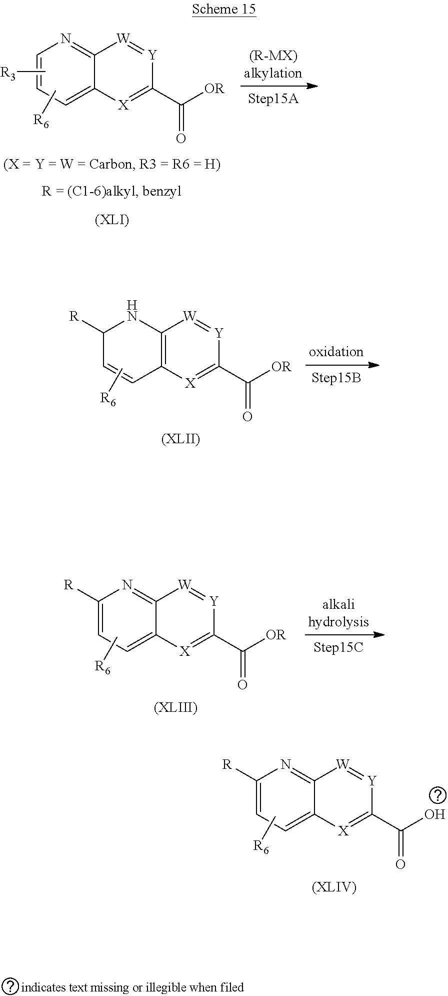

### Step 15A

In this Step, a 1,2-dihydroquinoline compound of formula (XLII) can be prepared by alkylation of the compound of formula (XLI) in a reaction inert solvent. The organometallic compound of formula R-MX can be prepared by reaction of a halide compound of R, wherein R is alkyl. M represents metal such as lithium, or MgX, wherein X represents a hydrogen atom, a halogen atom such as, fluorine, chlorine, bromine or iodine. Examples of suitable organometallic reagents include alkyllithiums such as methyllithium, n-butyllithium, sec-butyllithium and tert-butyllithium; aryllithiums such as phenyllithium and lithium naphtilide; alkylmagnesium halide such as methylmagnesium halide, isopropylmagnesium halide, and t-butylmagnesium halide; arylmagnesium halide such as phenylmagnesium halide. Examples of preferred reaction inert solvents include hydrocarbons, such as hexane; ethers, such as diethyl ether, diisopropyl ether, dimethoxyethane (DME), tetrahydrofuran (THF) and dioxane; or mixtures thereof. Reaction temperatures are generally in the range of −100 to 100° C., preferably in the range of from 100° C. to room temperature. Reaction times are, in general, from 1 minute to a day, preferably from 1 hour to 24 hours.

### Step 15B

In this Step, a compound of formula (XLIII) can be prepared by oxidation of the compound of formula (XLII) in a solvent. Examples of suitable oxidative agents include Cr-reagents, such as chromium trioxide (CrO₃), potassium chromate (K₂CrO₄), potassium dichromate (C₂Cr₂O₇); Mn-reagents, such as manganese dioxide (MnO₂), potassium permanganate (KMnO₄), quinine reagents, such as 2,3,5,6,-tetrachloro-1,4-benzoquinone (p-chloranil), 2,3-dichloro-5,6-dicyano-1,4-benzoquinone (DDQ), and air oxidation. Examples of suitable solvents include tetrahydrofuran, dioxane, acetone, N,N-dimethylformamide, acetonitrile, halogenated hydrocarbons (e.g., dichloromethane, dichloroethane, chloroform), water; or mixtures thereof. The reaction can take place over a wide range of temperatures, and the precise reaction temperature is not critical to the invention. The preferred reaction temperature will depend upon such factors as the nature of solvent, and the starting material or reagent used. However, in general, it is convenient to carry out the reaction at a temperature of from −78° C. to 100° C., more preferably from about −60° C. to 60° C. The time required for the reaction may also vary widely, depending on many factors, notably the reaction temperature and the nature of the reagents and solvent employed. However, provided that the reaction is effected under the preferred conditions outlined above, a period of 1 minute to 24 hours, more preferably 30 minutes to 12 hours, will usually suffice.

### Step 15C

In this Step, an acid compound of formula (XLIV) can be prepared by hydrolysis of the compound of formula (XLIV) in a solvent by the method as described in Step 10E.

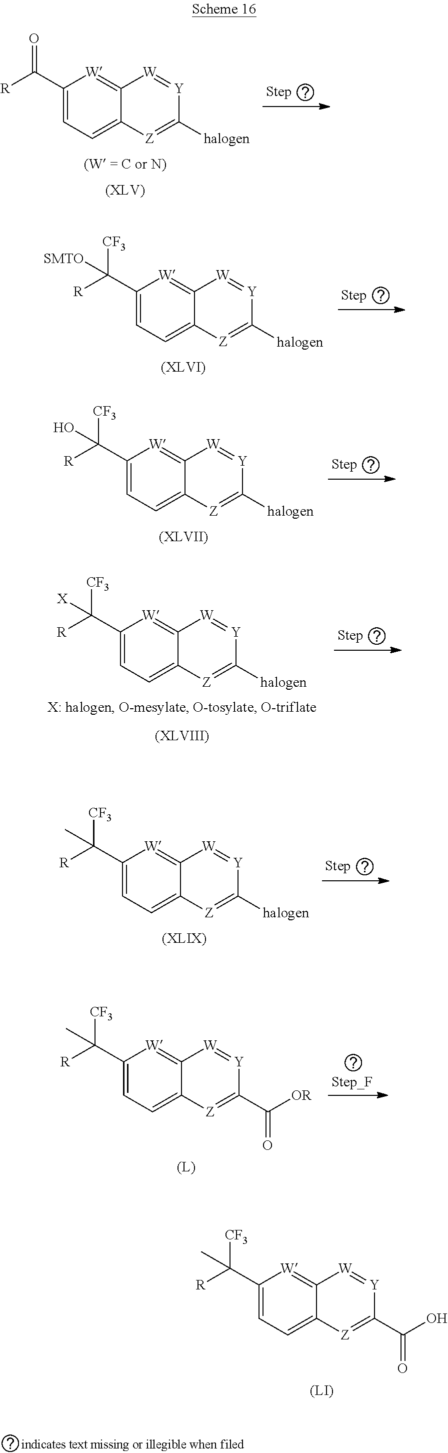

### Step 16A

In this Step, a compound of formula (XLVI) can be prepared by nucleophilic trifluoromethylation of formula (XLV) in a reaction inert solvent. Examples of preferred trifluoromethylation reagents include the combination of trifluoromethyltrimethylsilane (TMSCF3) and initiator reagents. Examples of preferred catalytic initiator reagents include tetrabutylammonium fluoride (TBAF), cesium fluoride (CsF), lithium acetate (AcOLi), sodium acetate (AcONa), potassium acetate (AcOK), tetrabutylammonium acetate (AcO-nNBu4), lithium pivalate (t-BuCO2Li), lithium benzoate (PhCO2Li), potassium t-butoxide (KO-tBu), and sodium t-butoxide (NaO-tBu). Examples of preferred reaction inert solvents include hydrocarbons, such as hexane, benzene, toluene; halogenated hydrocarbons, such as methylene chloride, chloroform, carbon tetrachloride and dichloroethane; ethers; such as diethyl ether, diisopropyl ether, 1,2-dimethoxyethane (DME), tetrahydrofuran and dioxane; acetonitrile, ethyl acetate, N,N-dimethylformamide (DMF), dimethylsulfoxide (DMSO) or mixtures thereof. Reaction temperatures are generally in the range of −78° C. to 200° C., more preferably in the range of −78° C. to 100° C. Reaction times are, in general, from 1 minute to 10 days, more preferably from 10 minutes to 24 hours.

### Step 16B

In this Step, a hydroxyl compound of formula (XLVII) can be prepared by hydrolysis under acid condition of the O-trimethylsilyl compound of formula (XLVI) in a solvent by the method as described in Step 10E.

### Step 16C

In this Step, a compound of formula (XLVIII) can be prepared by halogenation, O-mesylation, O-tosylation and O-triflate of the compound of formula (XLVII) in a reaction inert solvent or without solvent.

The halogenation reaction can be carried out under halogenating reagent in an inert solvent or without solvent. Examples of suitable solvents'include tetrahydrofuran, 1,4-dioxane, N,N-dimethylformamide, acetonitrile; halogenated hydrocarbons, such as dichloromethane, 1,2-dichloroethane, chloroform or carbon tetrachloride and acetic acid. Example of suitable halogenating reagents include thionyl chloride, oxalyl chloride, phosphorus pentachloride, phosphorus tribromide; phosphorus oxyhalide such as phosphorus oxychloride and phosphorus oxybromide; lewis acids such as titanium chloride, tin chloride and aluminium chloride

The reaction can be carried out at a temperature of from −78° C. to 200° C., more preferably from −20° C. to 150° C. Reaction times are, in general, from 5 minute to 10 days, more preferably from 30 minutes to 24 hours.

The O-mesylation, O-tosylation and O-triflate reactions can be carried out by the reaction of O-activating reagents with the compound of formula (XLVII) in the presence of a base in an inert solvent or without solvent. Examples of suitable O-activation reagents include methanesulfonyl chloride, p-toluenesulfonyl chloride, trifluoromethanesulfonyl chloride and trifluoromethanesulfonic acid anhydride. Examples of suitable base include alkyl lithium such as n-butyl lithium, sec-butyl lithium and tert-butyl lithium; potassium t-butoxide and sodium t-butoxide (NaO-tBu); triethylamine, diisopropylethylamine, 4-dimethylaminopyridine and pyridine. Examples of preferred reaction inert solvents include hydrocarbons, such as hexane, benzene, toluene; halogenated hydrocarbons, such as methylene chloride, chloroform, carbon tetrachloride and dichloroethane; ethers; such as diethyl ether, diisopropyl ether, 1,2-dimethoxyethane (DME), tetrahydrofuran and dioxane; acetonitrile, N,N-dimethylformamide (DMF), dimethylsulfoxide (DMSO) or mixtures thereof. The reaction can be carried out at a temperature of from −78° C. to 150° C., more preferably from −78° C. to 100° C. Reaction times are, in general, from 5 minute to 48 days, more preferably from 30 minutes to 24 hours.

### Step 16D

In this Step, a compound of formula (XLIX) can be prepared by an alkylation reaction of the compound of formula (XLVIII) with alkylating reagent in an inert solvent. Examples of preferred alkylating agents include trialkylmetals such as trimethylaluminum, triethylaluminum; alkylmagnesium halides such as methylmagnesium bromide in the presence of additive compound such as lithium bromide; dialkyltitanium halides such as dimethyltitanium dichloride prepared by dimethylzinc and titanium chloride; and is most preferably trimethylaluminium. Examples of preferred inert solvents for the reaction include halogenated hydrocarbons, such as dichloromethane (DCM), 1,2-dichloroethane, chloroform or carbon tetrachloride; ethers, such as diethyl ether, diisopropyl ether, 1,2-dimethoxyethane (DME), tetrahydrofuran (THF) and 1,4-dioxane; hydrocarbons, such as n-hexane, cyclohexane, benzene and toluene; or mixtures thereof. Reaction temperatures are generally in the range of from −100° C. to 200° C., preferably in the range of from −40° C. to 100° C. Reaction times are, in general, from 1 minute to 10 days, preferably from 1 hour to 24 hours.

### Step 16E

In this Step, a compound of formula (L) can be prepared by alkoxycarbonyl insertion reaction of the compound of formula (XLIX) in a solvent by the method as described in Step 10D.

### Step 16F

In this Step, an acid compound of formula (LI) can be prepared by hydrolysis of the compound of formula (L) in a solvent by the method as described in Step 10E

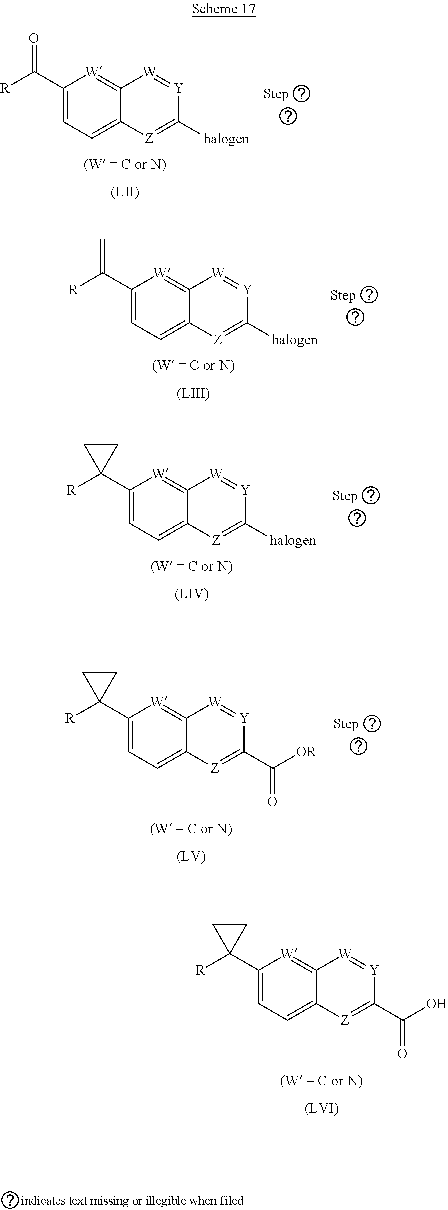

### Step 17A

In this step, the compound of formula (LIU) can be prepared by olefinating of the compound of formula (LII) using titanium-aluminum methylidene complex (Tebbe reagent) or a phosphinilide (Wittig reagent) prepared in situ from a suitable phosphine reagent and a methylene halide reagent or phosphorane under olefination conditions or basic conditions in an inert solvent.

Examples of suitable solvents include: toluene; benzene; xylene; diglyme; dimethylsulfoxide (DMSO); 1,2-dimethoxyethane (DME); tetrahydrofuran (THF); diethylether; 1,4-dioxane; N,N-dimethylformamide (DMF) acetonitrile; alcohols such as methanol or ethanol; halogenated hydrocarbons such as dichloromethane (DCM), 1,2-dichloroethane, chloroform or carbon tetrachloride; and acetic acid. Suitable phosphine reagents include, for example, triphenylphosphine and tributylphosphine. Suitable methylene halide reagents include, for example, methyl bromide, ethyl bromide, methyl iodide, ethyl idolide, methyl chloride, ethyl chloride, methyl bromoacetate, bromoacetonitrile, 1-bromoacetone, ethylidene(triphenyl)phosphorane, (triphenylphosphoranylidene)acetonitrile and methyl (triphenylphosphoranylidene)acetate.

A preferred base is selected from, for example, but not limited to: an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride; or an amine such as triethylamine, tributylamine, diisopropylethylamine, 2,6-lutidine, pyridine or dimethylaminopyridine.

The reaction can be carried out at a temperature of from 0° C. to 300° C., more preferably from 20° C. to 200° C. Reaction times are, in general, from 5 minutes to 96 hours, more preferably from 30 minutes to 24 hours.

### Step 17B

In this Step, the compound of formula (LIV) can be prepared by cyclopropanating reaction of the compound of formula (LIII) using a carbene or methylid prepared in situ under cyclopropanation conditions in an inert solvent. Examples of suitable solvents include: diglyme; dimethylsulfoxide (DMSO); 1,2-dimethoxyethane (DME); THF; diethylether; 1,4-dioxane; N,N-dimethylformamide (DMF); acetonitrile; alcohols, such as methanol or ethanol; halogenated hydrocarbons, such as dichloromethane (DCM), 1,2-dichloroethane, chloroform or carbon tetrachloride; and acetic acid. Suitable reagents include, for example, CH₂I₂-Zinc/Copper complex or dialkyl zinc such as dimethyl zinc and diethyl zinc (Simmons-Smith reagent), trimethylsulfoxonium iodide and diazomethane.

This reaction can be carried out in the presence or absence of a suitable catalyst. There is likewise no particular restriction on the nature of the catalysts used, and any catalysts commonly used in reactions of this type can equally be used here. Examples of such catalysts include: Zirconium(0), Copper(0), Copper(acetylacetone)₂, Co(TPP) and Pd(OAc)₂.

This reaction can be carried out in the presence of a suitable additive agent. Examples of such additive agents include: diphenyl phosphate, acetylchloride, methylbenzoate, sodium fluoride, triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, sodium hydride, potassium hydride, sodium methoxide, potassium t-butoxide and lithium diisopropyl amide. The reaction can be, carried out at a temperature of from −78° C. to 250° C., more preferably from −40° C. to 150° C. Reaction times are, in general, from 5 minutes to 96 hours, more preferably from 30 minutes to 24 hours.

### Step 17C

In this Step, a compound of formula (LVI) can be prepared by alkoxycarbonyl insertion reaction of the compound of formula (LV) in a solvent by the method as described in Step 10D.

### Step 17D

In this Step, an acid compound of formula (LVI) can be prepared by hydrolysis of the compound of formula (LV) in a solvent by the method as described in Step 10E.

### Example 1

### 6-TERT-BUTYL-N-((1R)-1-{3-METHYL-4-[(METHYLSULFONYL)AMINO]PHENYL}ETHYL)-2-NAPHTHAMIDE

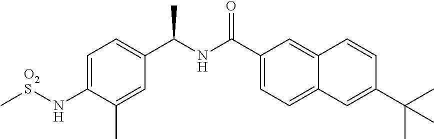

### 1A) 4-ACETYL-2-METHYLPHENYL TRIFLUOROMETHANESULFONATE

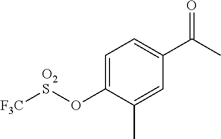

To a stirred solution of 1-(4-hydroxy-3-methylphenyl)ethanone (6.0 g, 40 mmol) in DCM (100 ml) was added triflic anhydride (8.7 ml, 52 mmol) and triethylamine (10 ml) successively. The mixture was stirred at room temperature for 16 hours, quenched with water and extracted with DCM. The organic layer was dried over sodium sulfate and concentrated in vacuo. The crude material was purified by silica gel column chromatography, eluting with DCM/EtOAc (5:1), to furnish 9.6 g (85% yield) of the title compound as yellow oil.

¹H NMR (270 MHz, CDCl₃) [symbol] ppm 2.45 (3H, s), 2.62 (3H, s), 7.35 (1H, d, J=8.6 Hz), 7.86 (1H, dd, J=8.6, 2.5 Hz), 7.92 (1H, s).

### 1B) N-(4-ACETYL-2-METHYLPHENYL)METHANESULFONAMIDE

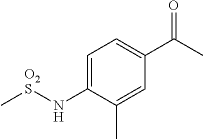

A test tube suitable for microwave use was charged with tris(dibenzylidenacetone)dipalladium (0) chloroform adduct (205 mg, 0.20 mmol), the compound of Example 1A (1.41 g, 5.0 mmol), methanesulfonamide (570 mg, 6.0 mmol), and cesium carbonate (1.63 g, 7.0 mmol). The mixture was subjected to microwave irradiation at 120° C. with stirring for 10 minutes. The reaction mixture was filtered and the filtrate was concentrated in vacuo. The crude material was purified by silica gel column chromatograph, eluting with hexane/ethylacetate (2:1), to furnish 390 mg (34% yield) of the title compound as a yellow solid.

¹H NMR (270 MHz, CDCl₃) §2.34 (3H, s), 2.59 (3H, s), 3.11 (3H, s), 6.47 (1H, br.s), 7.58 (1H, d, J=8.1 Hz), 7.84 (2H, m).

MS (ESI): m/z 228 (M+H)⁺, 226 (M−H)⁻.

### 1C) N-[4-((1R)-1-{[(R)-TERT-BUTYLSULFINYL]AMINO}ETHYL)-2-METHYLPHENYL]METHANESULFONAMIDE

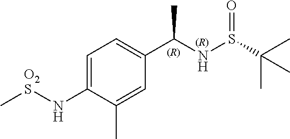

To a solution of titanium(IV) ethoxide (1.32 g, 5.8 mol) and the compound of Example 1B (800 mg, 3.5 mmol) in THF (20 ml), (R)-(+)-tert-butanesulfinamide was added under nitrogen atmosphere and the mixture was heated at 70° C. for 16 hours. The reaction was quenched with water and the resulting white precipitate was filtered off. The filtrate was partitioned between EtOAc and water and the organic layer was dried over sodium sulfate and concentrated in vacuo. The crude material was purified by silica gel column chromatography, eluting with hexane/EtOAc (4:1). The resulting yellow oil was dissolved in THF (10 ml) and the solution was added to sodium borohydride (242 mg, 6.4 mmol) in THF (10 ml) at −70° C. The mixture was stirred at −70° C. for 5 hours and then quenched with MeOH, stirred at room temperature for 1 hour, and concentrated in vacuo to furnish 530 mg (45% yield) of the title compound as a slightly yellow solid.

MS (ESI): m/z 333 (M+H)⁺, 331 (M−H)⁻.

### 1D) N-{4-[(1R)-1-AMINOETHYL]-2-METHYLPHENYL}METHANESULFONAMIDE HYDROCHLORIDE

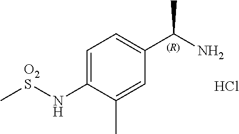

To the compound of Example 1C (530 mg, 1.60 mmol) was added hydrogenchloride-MeOH (2.0 M, 5.0 ml) and 1,4-dioxane (5.0 ml). The solution was stirred at room temperature for 30 minutes and then concentrated in vacuo. Diethyl ether was added to precipitate the amine hydrochloride. The precipitate was then filtered and washed with diethyl ether to furnish 450 mg (quant.) of the title compound as a white solid. The enantiomeric purity was determined by Daicel Chiralcel OD-H (4.6×250 mm), eluting with 0.1% diethylamine in hexane/ethylalcohol (80:20 by volume) in the condition of column temperature (40° C.), Retension time: 10.2 min (R-form), 12.8 min (S-form).

¹H NMR (270 MHz, DMSO-d₆) [symbol] 1.45 (3H, m), 2.31 (3H, s), 2.98 (3H, s), 4.27 (1H, m), 7.31-7.38 (3H, m). MS (ESI): m/z 227 (M−H)⁻.

### 1E) METHYL 6-TERT-BUTYL-2-NAPHTHOATE

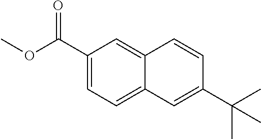

A mixture of 2-bromo-6-tert-butylnaphthalene (980 mg, 3.72 mmol), palladium acetate (84 mg, 0.37 mmol), 1,3-bis(diphenylphosphino)propane (153 mg, 0.37 mmol) and triethylamine (1.56 ml, 11.2 mmol) in methanol (6 ml) and DMF (10 ml) was heated at 80° C. under carbon monooxide gas (balloon pressure) for 15 hours. After cooling to ambient temperature, the mixture was diluted with ethyl acetate-toluene (8:1) (160 ml) and filtered through a pad of celite. The filtrate and washings were washed with water, brine, dried over sodium sulfate and evaporated in vacuo to give the crude product which was purified through silica gel column chromatography eluting with hexane/EtOAc (10:1) to furnish the title compound as colorless oil (843 mg, 94%).

¹H NMR (CDCl₃) δ 1.43 (9H, s), 3.97 (3H, s), 7.61-7.67 (1H, m), 7.79-7.93 (3H, m), 8.01-8.07 (1H, m), 8.57 (1H, br, s).

### 1F) 6-TERT-BUTYL-2-NAPHTHOIC ACID

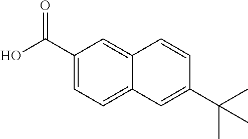

A mixture of methyl 6-tert-butyl-2-naphthoate (843 mg, 3.48 mmol) and 2M sodium hydroxide solution (6.96 mmol, 3.48 mmol) in methanol (30 ml) was heated at 60° C. for 3 hours. After cooling to ambient temperature, the solvent was evaporated in vacuo and the residue was acidified to pH 2 with 2M hydrochloric aqueous solution. The aqueous layer was extracted with ethyl acetate and the combined solution was washed with brine, dried over sodium sulfate and evaporated in vacuo to give the crude product which was recrystallized from ethyl acetate and hexane to furnish the title compound as a white solid (614 mg, 77%).

¹H NMR (DMSO-d₆) [symbol] 1.39 (9H, s), 7.70-7.76 (1H, m), 7.90-8.08 (4H, m), 8.55 (1H, br, s), 13.00 (1H, br, s).

### 1G) 6-TERT-BUTYL-N-((1R)-1-{3-METHYL-4-[(METHYLSULFONYL)AMINO]PHENYL}ETHYL)-2-NAPHTHAMIDE

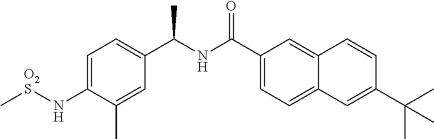

To a DMF (15 ml) solution of the amine compound of Example 1D (174 mg, 0.657 mmol), the acid of Example 1F (150 mg, 0.657 mmol) and HBTU (300 mg, 0.788 mmol) was added triethylamine (0.275 ml, 1.97 mmol) and the mixture was stirred for 2 hours at room temperature. Then the reaction was diluted with ethyl acetate-toluene (8:1) (150 ml) and washed 1M hydrochloric aqueous solution, water, dried over sodium sulfate and concentrated in vacuo to give crude product. The crude product was purified by column chromatography on amino-bounded silica gel with dichloromethane-methanol (100:1) to give a white solid, which was recrystallized from ethyl acetate-hexane to furnish the title compound as a white solid (235 mg, 82%).

¹H NMR (DMSO-d₆) δ 1.36 (9H, s), 1.51 (3H, d, J=6.5 Hz), 2.30 (3H, s), 2.96 (3H, s), 5.12-5.26 (1H, m), 7.20-7.33 (3H, m), 7.68-7.74 (1H, m), 7.87-8.00 (4H, m), 8.45 (1H, br, s), 8.89-8.95 (1H, m), 9.01 (1H, br, s). MS (ESI) m/z 437 (M−H)⁻, 439 (M+H)⁺.

### Example 2

### 6-TERT-BUTYL-N-((1R)-1-{6-METHYL-5-[(METHYLSULFONYL)AMINO]PYRIDIN-2-YL}ETHYL)-2-NAPHTHAMIDE

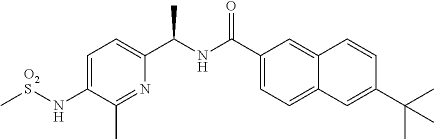

### 2A) N-(6-CHLORO-2-METHYLPYRIDIN-3-YL)METHANESULFONAMIDE

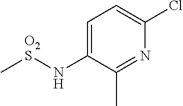

A mixture of 3-amino-6-chloro-2-picoline (2.0 g, 14.0 mmol) and methanesulfonyl chloride (1.92 g, 16.8 mmol) in pyridine (40 ml) was stirred for 1 hour at room temperature. After removal of the solvent, the resulting crude product was purified by silica gel column chromatography, eluting with hexane/EtOAc (3:2), to furnish 1.70 g (55% yield) of the title compound as a pale yellow solid.

¹H NMR (DMSO-d₆) δ 2.47 (3H, s), 3.05 (3H, s), 7.37 (1H, d, J=8.6 Hz), 7.71 (1H, d, J=8.6 Hz), 9.47 (1H, s). MS (ESI): m/z 221 (M+H)⁺.

### 2B) N-(6-CYANO-2-METHYLPYRIDIN-3-YL)METHANESULFONAMIDE

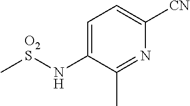

A test tube suitable for microwave use was charged with the compound of Example 2A (1.66 g, 7.52 mmol), zinc cyanide (1.11 g, 9.45 mmol) and tetrakis(triphenylphosphine)palladium(0) (872 mg, 0.754 mmol) in DMF (14.1 ml). The mixture was subjected to microwave irradiation at 100° C. with stirring for 30 minutes. Then, the mixture was diluted with toluene/EtOAc (1:10) and the precipitate was filtered off. The organic layer was washed with water, then brine, and dried over magnesium sulfate. After the filtration, the organic layer was evaporated in vacuo to give the crude product which was purified by silica gel column chromatography, eluting with hexane/EtOAc (3:2), to give the title compound (835 mg, 53%) as a pale yellow solid.

¹H NMR (DMSO-d₆) [symbol] 2.50 (3H, s), 3.15 (3H, s), 7.85 (2H, s), 9.81 (1H, s).

MS (ESI): m/z 212 (M+H)⁺.

### 2C) N-(6-ACETYL-2-METHYLPYRIDIN-3-YL)METHANESULFONAMIDE

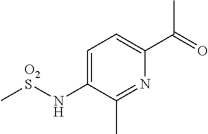

To a solution of the compound of Example 2B (423 mg, 2.0 mmol) in THF (9.9 ml) was added dropwise a diethyl ether solution of methyl magnesium bromide (6.7 ml, 6.0 mmol) at 0° C. with stirring. After being stirred for 2 hours at the same temperature, the reaction mixture was poured into ice cold water (10 ml) and extracted with EtOAc. The organic layer was dried over magnesium sulfate and concentrated to give a dark red solid, which was isolated from EtOAc-hexane to furnish 246 mg (54% yield) of the title compound as a red solid.

¹H NMR (300 MHz, DMSO-d₆) [symbol] 2.56 (3H, s), 2.59 (3H, s), 3.13 (3H, s), 7.80-7.89 (2H, m), 9.68 (1H, s). MS (ESI): m/z 229 (M+H)⁺.

### 2D) N-[2-METHYL-6-((1R)-1-{[(1R)-1-PHENYLETHYL]AMINO}ETHYL)PYRIDIN-3-YL]-METHANESULFONAMIDE

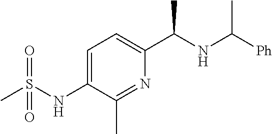

To a solution of the compound of Example 2C (959 mg, 4.20 mmol), (1R)-1-phenylethanamine (611 mg, 5.04 mmol) and triethylamine (2.34 ml, 16.8 mmol) in DCM (30 ml) was added a solution of titanium (IV) chloride (495 mg, 2.61 mmol) in DCM (5 ml) at room temperature under N₂. After being stirred for 17 hours at the same temperature, the reaction volume was reduced to the extent of half by evaporation (ca. 20 m). The mixture was diluted with EtOH (40 ml) and then it was hydrogenated over Raney-Ni under H₂ pressure (4.3 kg/cm₂) at room temperature. After being stirred for 5 hours, the reaction mixture was filtered through a celite pad with DCM. The filtrate was concentrated and the residue was purified by silica gel column chromatography, eluting with acetone/hexane (1:1), to furnish 0.67 g (48% yield) of the title compound as yellow viscous oil.

¹H NMR (300 MHz, DMSO-d₆) [symbol] 1.09-1.25 (6H, m), 2.45 (3H, s), 3.02 (3H, s), 3.26-3.48 (2H, m), 7.13-7.37 (6H, m), 7.61 (1H, d, J=8.1 Hz).

MS (ESI): m/z 334 (M+H)⁺.

### 2E) N-{6-[(1R)-1-AMINOETHYL]-2-METHYLPYRIDIN-3-YL}METHANESULFONAMIDE HYDROCHLORIDE SALT

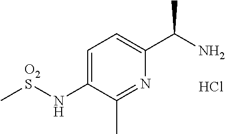

To a solution of the compound of Example 2D (0.82 g, 2.46 mmol) in EtOH (25 ml) was added 10% Pd—C (0.32 g) and ammonium formate (6.20 g, 98 mmol) at room temperature under N₂. The resulting mixture was stirred for 2 hours at 65° C. The reaction mixture was cooled to room temperature and filtered through a celite pad. The filtrate was treated with 10% HCl-MeOH, then concentrated and the product isolated from MeOH-ether to furnish 0.54 g (83% yield) of the title compound as a white solid.

¹H NMR (300 MHz, DMSO-d₆) δ 1.48 (3H, d, J=6.6 Hz), 2.56 (3H, s), 3.06 (3H, s), 4.38-4.54 (1H, m), 7.40 (2H, d, J=9.0 Hz), 7.76 (1H, d, J=9.0 Hz), 8.40 (2H, br.s.), 9.50 (1H, s).

MS (ESI): m/z 230 (M+H)⁺.

### 2F) 6-TERT-BUTYL-N-((1R)-1-{6-METHYL-5-[(METHYLSULFONYL)AMINO]PYRIDIN-2-YL}ETHYL)-2-NAPHTHAMIDE

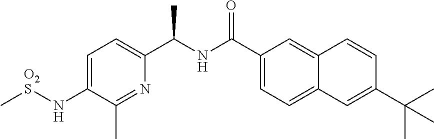

To a DMF (4.3 ml) solution of the compound of Example 2E (100 mg, 0.434 mmol), Example 1F (99 mg, 0.434 mmol) and HBTU (198 mg, 0.521 mmol) was added triethylamine (0.183 ml, 1.30 mmol) and the mixture was stirred for 3 hours at room temperature. The same procedure as described in Example 1G was performed to give the title compound (138 mg, 72% yield) as a white solid.

¹H NMR (300 MHz, CDCl₃) δ 1.44 (9H, s), 1.61 (3H, d, J=6.6 Hz), 2.62 (3H, s), 3.06 (3H, s), 5.31-5.44 (1H, m), 6.17-6.45 (1H, m), 7.23 (1H, d, J=8.8 Hz), 7.65 (1H, dd, J=8.8, 2.2 Hz), 7.74 (1H, d, J=7.3 Hz), 7.76-7.94 (5H, m), 8.34 (1H, s).

MS (ESI) m/z 440 (M−H)⁻, 438 (M+H)⁺.

### Example 3

### ((1R)-1-{3,5-DIFLUORO-4-[(METHYLSULFONYL)AMINO]PHENYL}ETHYL)-6-(TRIFLUORO-METHYL)QUINOLINE-2-CARBOXAMIDE

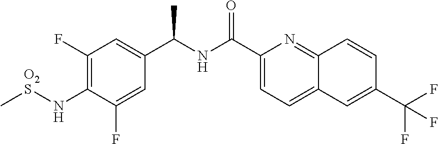

### 3A) N-(4-BROMO-2,6-DIFLUOROPHENYL)METHANESULFONAMIDE

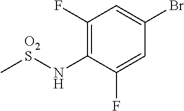

To a solution of 4-bromo-2,6-difluoroaniline (3.0 g, 14.4 mmol) in pyridine (20 ml) was added methanesulfonyl chloride (2.23 ml, 28.8 mmol) at room temperature. Then the mixture was stirred at 50° C. for 6 hours. After cooing to room temperature, the mixture was concentrated in vacuo. The resulting residue was dissolved in THF (40 ml). To this solution was added 2M sodium hydroxide aqueous solution (40 ml) and the reaction was stirred at room temperature for 4 hours. The mixture was acidified with 2M HCl aqueous solution and extracted with EtOAc. The organic layer was washed with 2M HCl aqueous solution and brine, dried over sodium sulfate and concentrated in vacuo, to give the title compound (4.05 g, 98%) as an orange solid.

¹H NMR (270 MHz, CDCl₃) δ 3.22 (3H, s), 6.08 (1H, br s), 7.17-7.24 (2H, m).

MS (ESI) m/z 286 (M+H)⁺, 284 (M−H)⁻.

### 3B) N-(4-ACETYL-2,6-DIFLUOROPHENYL)METHANESULFONAMIDE

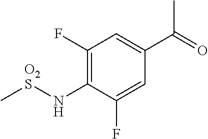

A test tube suitable for microwave use was charged with palladium (II) acetate (12 mg, 0.05 mmol), 1,3-bis(diphenylphosphino)propane (43 mg, 0.11 mmol), the compound of Example 3A (500 mg, 1.75 mmol), n-butyl vinyl ether (1.1 ml, 8.75 mmol), and potassium carbonate (290 mg, 2.10 mmol) in DMF (4.8 ml)-water (1.2 ml). The mixture was subjected to microwave irradiation at 100° C. with stirring for 30 minutes. The mixture was diluted with THF, acidified with concentrated HCl and stirred at room temperature for 14 hours. The mixture was partitioned between EtOAc and water. The organic layer was dried over sodium sulfate and concentrated in vacuo. The crude material was purified by silica gel column chromatography, eluting with gradually from hexane/EtOAc (2:1) to hexane/EtOAc (1:1), to give the title compound (214 mg, 49%) as a white solid.

¹H NMR (270 MHz, CDCl₃) δ 2.59 (3H, s), 3.32 (3H, s), 7.55-7.63 (2H, m). A signal due to NH was not observed. MS (ESI) m/z 248 (M−H)⁻.

### 3C) N-[4-((1R)-1-{[(R)-TERT-BUTYLSULFINYL]AMINO}ETHYL)-2,6-DIFLUOROPHENYL]METHANESULFONAMIDE

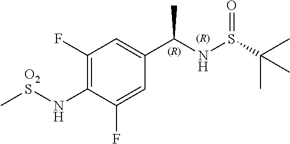

To a solution of the compound of Example 3B (270 mg, 1.1 mmol) and titanium(IV) ethoxide (2 ml) in THF (2 ml) was added (R)-(+)-2-methyl-2-propanesulfininamide (131 mg, 1.1 mmol) under a nitrogen atmosphere and the mixture was stirred for 18 hours at 70° C. After cooling to −20° C., sodium borohydrate (123 mg, 3.2 mmol) was added to the mixture. The mixture was warmed to room temperature and stirred for 16 hours, then quenched with MeOH and water, and the resulting white precipitate was filtered off. The filtrate was concentrated in vacuo to furnish the title compound (423 mg, 100%) as a yellow solid.

¹H NMR (270 MHz, CDCl₃) δ 1.18 (9H, s), 1.40 (3H, d, J=6.6 Hz), 2.92 (3H, s), 3.84-3.85 (1H, m), 4.30-4.38 (1H, m), 6.87 (2H, d, J=8.6 Hz). A signal due to NH was not observed.

### 3D) N-{4-[(1R)-1-AMINOETHYL]-2,6-DIFLUOROPHENYL}METHANESULFONAMIDE HYDROCHLORIDE

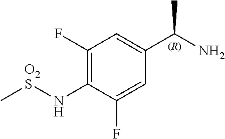

A mixture of the compound of Example 3C (423 mg, 1.1 mmol) and HCl-MeOH (10%, 10 ml) was stirred at room temperature for 24 hours and then concentrated in vacuo. Diethyl ether and MeOH were added to precipitate the amine hydrochloride. The precipitate was then filtered and washed with diethyl ether to furnish the title compound (290 mg, 94%) as a yellow solid.

¹H NMR (270 MHz, DMSO-d₆) δ 1.51 (3H, d, J=6.6 Hz), 3.08 (3H, s), 4.44 (1H, br s), 7.44-7.47 (2H, m), 8.67 (2H, br s), 9.67 (1H, s). MS (ESI) m/z 249 (M−H)⁻.

### 3E) ((1R)-1-{3,5-DIFLUORO-4-[(METHYLSULFONYL)AMINO]PHENYL}ETHYL)-6-(TRIFLUOROMETHYL)QUINOLINE-2-CARBOXAMIDE

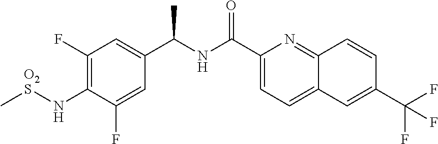

To a DMF (10 ml) solution of the compound of Example 3D (178 mg, 0.622 mmol); 6-(trifluoromethyl)quinoline-2-carboxylic acid (150 mg, 0.622 mmol) and HBTU (283 mg, 0.746 mmol) was added triethylamine (0.26 ml, 1.86 mmol) and the mixture was stirred for 3 hours at room temperature. The same procedure as described in Example 1G was performed to give the title compound (196 mg, 67% yield) as a white solid.

¹H NMR (DMSO-d₆) δ 1.59 (3H, d, J=6.5 Hz), 3.05 (3H, s), 5.18-5.32 (1H, m), 7.29-7.38 (2H, m), 8.11-8.18 (1H, m), 8.23-8.28 (1H, m), 8.38-8.44 (1H, m), 8.66 (1H, br, s), 8.77-8.82 (m, 1H), 9.42-9.52 (2H, m).

MS (ESI) m/z 472.11 (M−H)⁻, 474.14 (M+H)⁺.

### Example 4

### (1R)-1-{3-METHYL-4-[(METHYLSULFONYL)AMINO]PHENYL}ETHYL)-7-(TRIFLUOROMETHYL)QUINOLINE-3-CARBOXAMIDE

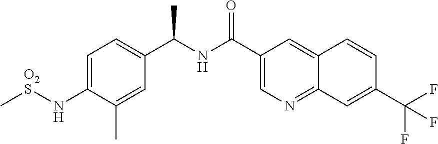

To a DMF (10 ml) solution of the compound of Example 1D (265 mg, 1.0 mmol), 7-(trifluoromethyl)quinoline-3-carboxylic acid (241 mg, 1.0 mmol) and HBTU (455 mg, 1.2 mmol) was added triethylamine (0.7 ml, 5.0 mmol) and the mixture was stirred for 3 hours at room temperature. The same procedure as described in Example 1G was performed to give the title compound (374 mg, 83% yield) as a white solid.

¹H NMR (DMSO-d₆) δ 1.53 (3H, d, J=7.3 Hz), 2.32 (3H, s), 2.97 (3H, s), 5.13-5.28 (1H, m), 7.22-7.35 (3H, m), 7.96-8.01 (1H, m), 8.35-8.47 (2H, m), 8.99-9.05 (2H, m), 9.25-8.31 (1H, m), 9.41-9.44 (1H, m).

MS (ESI) m/z 450.03 (M−H)⁻, 452.10 (M+H)⁺.

### Example 5

### N-((1R)-1-{3-METHYL-4-[(METHYLSULFONYL)AMINO]PHENYL}ETHYL)-6-(TRIFLUORO-METHYL)QUINOLINE-2-CARBOXAMIDE

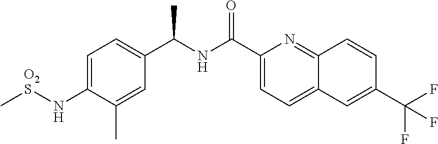

To a DMF (10 ml) solution of the compound of Example 1D (165 mg, 0.622 mmol), 6-(trifluoromethyl)quinoline-2-carboxylic acid (150 mg, 0.622 mmol) and HBTU (283 mg, 0.746 mmol) was added triethylamine (0.26 ml, 1.86 mmol) and the mixture was stirred for 3 hours at room temperature. The same procedure as described in Example 1G was performed to give the title compound (258 mg, 92% yield) as a white solid.

¹H NMR (DMSO-d₆) δ 1.58 (3H, d, J=6.6 Hz), 2.31 (3H, s), 2.97 (3H, s), 5.15-5.28 (1H, m), 7.21-7.38 (3H, m), 8.10-8.16 (1H, m), 8.24-8.28 (1H, m), 8.37-8.42 (1H, m), 8.65 (1H, br, s), 8.76-8.81 (m, 1H), 9.03 (1H, s), 9.25-9.30 (1H, m).

MS (ESI) m/z 450.14 (M−H)⁻, 452.20 (M+H)⁺.

### Example 6

### 6-TERT-BUTYL-N-((1R)-1-{3-METHYL-4-[(METHYLSULFONYL)AMINO]PHENYL}ETHYL)-QUINOLINE-2-CARBOXAMIDE

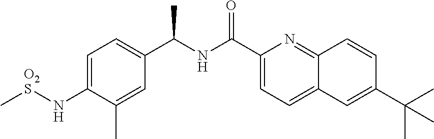

### 6A) 6-TERT-BUTYLQUINOLINE 1-OXIDE

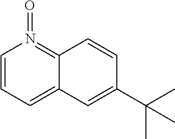

A mixture of 6-tert-butylquinoline (400 mg, 2.16 mmol, Journal of the Indian Chemical Society 1998, 823) and mCPBA (639 mg, 2.59 mmol) in chloroform (10 ml) was stirred for 2 hours at room temperature. The mixture was concentrated and the crude residue was applied to a silica gel (NH silica) column chromatography and eluted with dichloromethane/methanol (20:1) to furnish the title compound (433 mg, quant.) as a pale orange oil.

¹H NMR (300 MHz, CDCl₃) δ 1.43 (9H, s) 7.26-7.30 (1H, m), 7.73 (1H, d, J=8.1 Hz), 7.78 (1H, s), 7.85 (1H, dd, J=1.5, 8.8 Hz), 8.49 (1H, J=5.9 Hz), 8.67 (1Hd, J=8.8 Hz)

MS (ESI): m/z 202 (M+H)⁺.

### 6B) 6-TERT-BUTYLQUINOLINE-2CARBONITRILE

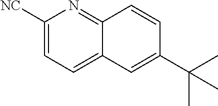

A mixture of the compound of Example 6A (310 mg, 1.54 mmol), trimethylsilylcyanide (458 mg, 4.62 mmol), trimethylamine (312 mg, 3.08 mmol) in acetonitrile (3 ml) was stirred for 15 minutes at 120° C. under microwave irradiation. The mixture was applied to a silica gel column chromatography and eluted with hexane/ethyl acetate (20:1) to furnish the title compound (295 mg, 91% yield) as a white solid.

¹H NMR (300 MHz, CDCl₃) δ 1.44 (9H, s), 7.68 (1H, d, J=8.8 Hz), 7.79 (1H, d, J=2.2 Hz), 7.94 (1H, d, J=2.2, 8.8 Hz), 8.11 (1H, d, J=8.8 Hz), 8.26 (1H, d, J=8.8 Hz)

MS (ESI): m/z 211 (M+H)⁺.

### 6C) 6-TERT-BUTYLQUINOLINE-2-CARBOXYLIC ACID

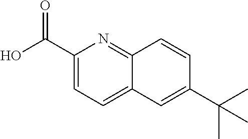

A solution of the compound of Example 6B (295 mg, 1.40 mmol) and 2M-aqueous sodium hydroxide (3 ml) in ethanol (4.5 ml) was stirred for 4 hours at reflux. The mixture was diluted with water (10 ml), neutralized by 2M-aqueous hydrochloride and extracted with ethyl acetate (30 ml). The organic layer was dried over sodium sulfate, filtrated, and concentrated in vacuo to furnish the title compound (313 mg, quant.) as a white solid.

¹H NMR (300 MHz, DMSO-d₆) δ 1.40 (9H, s), 7.93-7.97 (2H, m), 8.01-8.11 (2H, m), 8.41 (1H, d, J=8.1 Hz)

MS (ESI): m/z 230 (M+H)+.

### 6D) 6-TERT-BUTYL-N-((1R)-1-{3-METHYL-4[(METHYLSULFONYL)AMINO]PHENYL}-ETHYL)QUINOLINE-2-CARBOXAMIDE

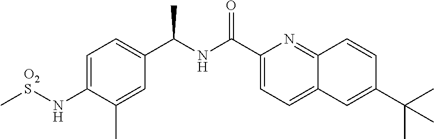

To a DMF (2 ml) solution of the compound of Example 6C (48 mg, 0.21 mmol), triethylamine (0.088 ml, 0.63 mmol) and the compound of Example 1D (55 mg, 0.21 mmol) was added HBTU (100 mg, 0.25 mmol) and the mixture was stirred for 2 hours at room temperature. Then, the reaction was quenched with saturated sodium bicarbonate aqueous solution, and the product was extracted with ethyl acetate which was dried over sodium sulfate. Then, filtration, evaporation, purification through silica gel column chromatography eluting with hexane/ethyl acetate (1:1) to furnish the title compound (32 mg, 35% yield) as a white solid.

¹H NMR (300 MHz, DMSO-d₆) [symbol] 1.41 (9H, s), 1.57 (3H, d, J=6.6 Hz), 2.29 (3H, s), 2.95 (3H, s), 5.16-5.21 (1H, m), 7.21-7.35 (3H, m), 7.97-8.16 (4H, m), 8.51 (1H, d, J=8.6 Hz), 9.01 (1H, brs), 9.07 (1H, d, J=8.6 Hz)

MS (ESI): m/z 440 (M+H)+.

### Example 7

### 6-TERT-BUTYL-N-((1R)-1-{3,5-DIFLUORO-4-[(METHYLSULFONYL)AMINO]PHENYL}-ETHYL)QUINOLINE-2-CARBOXAMIDE

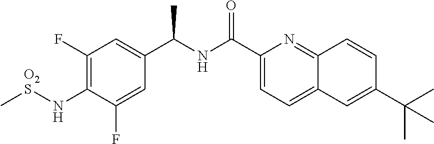

A DMF (2 ml) solution of the compound of Example 6C (115 mg, 0.5 mmol), triethylamine (0.20 ml, 0.15 mmol), the compound of Example 3D (143 mg, 0.5 mmol) and HBTU (228 mg, 0.6 mmol) was treated in the same procedure described in Example 1G. The crude residue was applied to a silica gel column chromatography and eluted with hexane/ethyl acetate (2:1 to 1:1) to furnish the title compound (131 mg, 57% yield) as a white solid.

¹H NMR (300 MHz, CDCl₃) [symbol] 1.45 (9H, s), 1.67 (3H, d, J=7.3 Hz), 3.20 (3H, s), 5.25-5.35 (1H, m), 6.04 (1H, s), 7.09 (2H, d, J=7.9 Hz), 7.80 (1H, s), 7.85-7.93 (1H, m), 8.07 (1H, d, J=9.2 Hz), 8.22-8.33 (2H, m), 8.52 (1H, d, J=7.9 Hz).

MS (ESI): m/z 440 (M+H)+.

### Example 8

### 2-TERT-BUTYL-N-((1R)-1-{3-METHYL-4-[(METHYLSULFONYL)AMINO]PHENYL}ETHYL)-QUINOLINE-6-CARBOXAMIDE

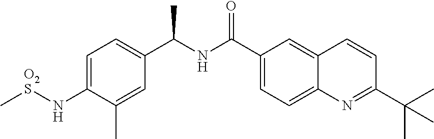

### 8A) METHYL 2-TERT-BUTYLQUINOLINE-6-CARBOXYLATE

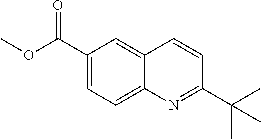

To a THF (20 ml) solution of methyl quinoline-6-carboxylate (984 mg, 5.26 mmol, J. Org. Chem. 2002, 67, 7890) was added t-butylmagnesium chloride in THF (15.8 ml, 1M solution) dropwise at −78° C. over 30 min. The mixture was stirred at −78° C. for 30 minutes and at −40° C. for 30 minutes, then at room temperature for 1 hour. The reaction was quenched with saturated ammonium chloride aqueous solution (100 ml) and extracted with ethyl acetate (100 ml×2) which was dried over sodium sulfate. Then, filtration, evaporation gave yellow oil, which was solved in THF (50 ml) and manganese dioxide (1.83 g 15.8 mmol) was added. After the mixture was stirred at room temperature for 2.5 hours, The precipitate was removed through a pad of celite and washed with ethyl acetate. The filtrate was concentrated and purified through silica gel column chromatography eluting with Hexane/Ethyl acetate (20:1) to furnish the title compound (348 mg, 27% yield) as a white solid.

¹H NMR (300 MHz, CDCl₃) δ1.48 (9H, s), 3.99 (3H, s), 7.59 (1H, d, J=8.8 Hz), 8.08 (1H, d, J=8.8 Hz), 8.17 (1H, d, J=8.8 Hz), 8.26 (1H, dd; J=2.2, 8.8 Hz), 8.55 (1H, d, J=2.2 Hz)

MS (ESI): m/z 244 (M+H)+.

### 8B) 2-TERT-BUTYLQUINOLINE-6-CARBOXYLIC ACID

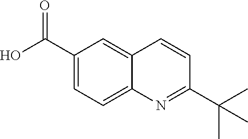

To a solution of the compound of Example 8A (347 mg, 1.43 mmol) in methanol (4 ml) and THF (4 ml) was added 2N aqueous sodium hydroxide (2 ml) at room temperature. The mixture was stirred at room temperature for 1.5 hours. Then evaporated, diluted with water (5 ml), and neutralized to pH 5˜6 by 2M aqueous hydrochloride. The formed precipitate was collected, washed with water to furnish the title compound (282 mg, 86% yield) as a white solid.

¹H NMR (300 MHz, CDCl₃) [symbol] 1.49 (9H, s), 7.62 (1H, d, J=8.8 Hz), 8.13 (1H, d, J=8.8 Hz), 8.20 (1H, d, J=8.8 Hz), 8.31-8.34 (1H, m), 8.64-8.66 (1H, m)

MS (ESI): m/z 230 (M+H)+.

### 8C) 2-TERT-BUTYL-N-((1R)-1-{3-METHYL-4-[(METHYLSULFONYL)AMINO]PHENYL}ETHYL)-QUINOLINE-6-CARBOXAMIDE

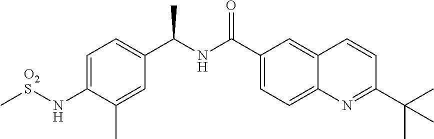

A DMF (0.5 ml) solution of the compound of Example 8B (8.0 mg, 0.035 mmol), triethylamine (0.015 ml, 0.11 mmol), the compound of Example 1D (18 mg, 0.07 mmol) and HBTU (20 mg, 0.053 mmol) was treated in accordance with the same procedure as described in Example 1G. The crude residue was applied to a silica gel column chromatography and eluted with hexane/ethyl acetate (1:2) to furnish the title compound (3.6 mg, 23% yield) as a white solid.

¹H NMR (300 MHz, CD₃OD) δ1.47 (9H, s), 1.61 (3H, d, J=7.3 Hz), 2.37 (3H, s), 2.96 (3H, s), 5.22-5.29 (1H, m), 7.28-7.39 (3H, m), 7.71 (1H, d, J=8.8 Hz), 8.06-8.13 (2H, m), 8.30 (1H, d, J=8.8 Hz), 8.36-8.38 (1H, m)

MS (ESI): m/z 440 (M+H)+.

### Example 9

### 6-ISOPROPYL-N-((1R)-1-{3-METHYL-4-[(METHYLSULFONYL)AMINO]PHENYL}ETHYL)-QUINOLINE-2-CARBOXAMIDE

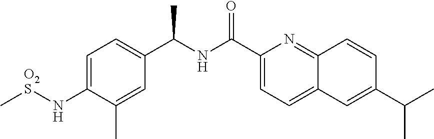

### 9A) 6-ISOPROPYLQUINOLINE 1-OXIDE

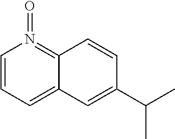

A chloroform (0.5 ml) solution of 6-isopropylquinoline (1.2 g, 7.0 mmol) and mCPBA (2.6 g, 10.5 mmol) was treated in the same procedure described in Example 6A. The crude residue was applied to a silica gel (NH silica) column chromatography and eluted with hexane/ethyl acetate (1:2 to 1:4) to furnish the title compound (1.23 g, 94% yield) as pale yellow oil.

¹H NMR (300 MHz, CDCl₃) δ1.35 (6H, d, J=7.3 Hz), 3.05-3.20 (1H, m), 7.25-7.30 (1H, m), 7.66-7.72 (3H, m), 8.48 (1H, d, J=5.9 Hz), 8.67 (1H, d, J=9.6 Hz)

MS (ESI): m/z 188 (M+H)+.

### 9B) 6-ISOPROPYLQUINOLINE-2-CARBONITRILE

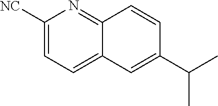

An acetonitrile (12 ml) solution of the compound of Example 9A (1.23 g, 6.62 mmol), trimethylsilylcyanide (1.97 g, 20.0 mmol) and triethylamine (1.85 ml, 13.2 mmol) was treated in the same procedure described in Example 6B. The crude residue was applied to a silica gel column chromatography and eluted with hexane/ethyl acetate (10:1) to furnish the title compound (1.27 g, 98% yield) as a yellow solid.

¹H NMR (300 MHz, CDCl₃) [symbol] 1.37 (6H, d, J=6.6 Hz), 3.10-3.20 (1H, m), 7.63-7.85 (3H, m), 8.30 (1H, d, J=8.8 Hz), 8.25 (1H, d, J=8.1 Hz)

MS (ESI): m/z 197 (M+H)+.

### 9C) 6-ISOPROPYLQUINOLINE-2-CARBOXYLIC ACID

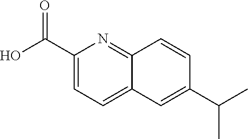

A solution of the compound of Example 9B (1.27 g, 6.47 mmol), 2M-aqueous sodium hydroxide (12 ml) in ethanol (30 ml) was treated in the same procedure described in Example 6C. Then evaporated, diluted with water (10 ml), neutralized to pH 5˜6 by 2M aqueous hydrochloride. The formed precipitate was collected, washed with water to furnish the title compound (1207 mg, 87% yield) as a white solid.

¹H NMR (270 MHz, DMSO-d₆) [symbol] 1.32 (6H, d, J=6.6 Hz), 3.05-3.15 (1H, m), 7.73 (1H, d, J=8.6 Hz), 7.80 (1H, s), 8.04-8.13 (2H, m), 8.35 (1H, d, J=7.9 Hz)

MS (ESI): m/z 216 (M+H)+.

### 9D) 6-ISOPROPYL-N-((1R)-1-{3-METHYL-4-[(METHYLSULFONYL)AMINO]PHENYL}ETHYL)-QUINOLINE-2-CARBOXAMIDE

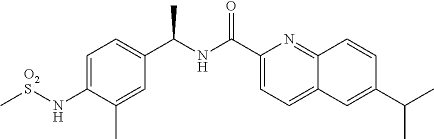

A DMF (4 ml) solution of the compound of Example 9C (91.5 mg, 0.425 mmol), triethylamine (0.178 ml, 1.28 mmol), the compound of Example 1D (113 mg, 0.425 mmol) and HBTU (193 mg, 0.510 mmol) was treated in the same procedure described in Example 1G. The crude residue was applied to a silica gel column chromatography and eluted with hexane/ethyl acetate (1:1) to furnish the title compound (111 mg, 61% yield) as a white solid.

¹H NMR (300 MHz, DMSO-d₆) [symbol] 132 (6H, d, J=6.6 Hz), 1.56 (3H, d, J=6.6 Hz), 2.29 (3H, s), 2.95 (3H, s), 3.09-3.18 (1H, m), 5.14-5.23 (1H, m), 7.21-7.35 (3H, m), 7.76-7.85 (1H, m), 7.88 (1H, s), 8.07-8.14 (2H, m), 8.48 (1H, d, J=8.8 Hz), 9.02 (1H, s), 9.09 (1H, d, J=8.8 Hz)

MS (ESI): m/z 426 (M+H)+.

### Example 10

### N-((1R)-1-{3-METHYL-4-[(METHYLSULFONYL)AMINO]PHENYL}ETHYL)-2-(TRIFLUOROMETHYL)QUINOLINE-6-CARBOXAMIDE

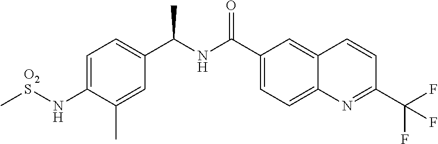

### 10A) 2-(TRIFLUOROMETHYL)QUINOLINE-6-CARBOXYLIC ACID

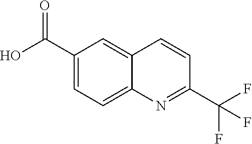

To a suspension of methyl quinoline-6-carboxylate 1-oxide (40 mg, 0.2 mmol, WO2006016548A1), trifluoromethyltrimethylsilane (84 mg, 0.6 mmol) in THF (2 ml) was added potassium tert-butoxide (73 mg, 0.6 mmol) portionwise at room temperature. The mixture was stirred at room temperature for 16 hours, then quenched with 1N-aqueous hydrochloride (10 ml) and extracted with ethyl acetate (20 ml). The organic layer was dried over sodium sulfate, filtrated and concentrated in vacuo to furnish the crude title compound (31.5 mg) as a orange solid.

MS (ESI): m/z 242 (M+H)+.

### 10B) N-((1R)-1-{3-METHYL-4-[(METHYLSULFONYL)AMINO]PHENYL}ETHYL)-2-(TRIFLUOROMETHYL)QUINOLINE-6-CARBOXAMIDE

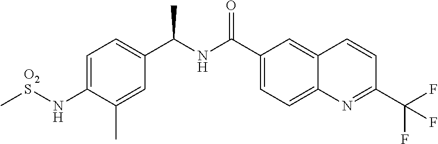

A DMF (1 ml) solution of the compound of Example 10A (31.5 mg, 0.13 mmol), triethylamine (0.054 ml, 0.39 mmol), the compound of Example 1D (34 mg, 0.13 mmol) and HBTU (59 mg, 0.15 mmol) was treated in the same procedure described in Example 10. The crude residue was applied to a silica gel column chromatography and eluted with hexane/ethyl acetate (1:1) to furnish the title compound (11 mg, 12% yield in 2 steps) as a white solid.

¹H NMR (300 MHz, CD₃OD) [symbol] 1.61 (3H, d, J=7.3 Hz), 2.38 (3H, s), 2.96 (3H, s), 5.23-5.30 (1H, m), 7.28-7.38 (3H, m), 7.93 (1H, d, J=8.8 Hz), 8.22-8.30 (2H, m), 8.55 (1H, s), 8.70 (1H, d, J=8.1 Hz)

MS (ESI): m/z 452 (M+H)+.

### Example 11

### 4-METHYL-N-((1R)-1-{3-METHYL-4-[(METHYLSULFONYL)AMINO]PHENYL}ETHYL)-7-(TRIFLUOROMETHYL)QUINOLINE-3-CARBOXAMIDE

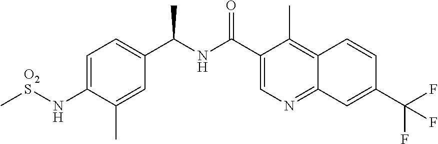

### 11A) ETHYL 4-METHYL-7-(TRIFLUOROMETHYL)QUINOLINE-3-CARBOXYLATE

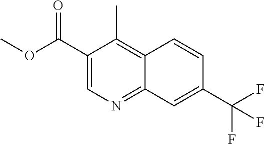

A mixture of ethyl 4-chloro-7-(trifluoromethyl)quinoline-3-carboxylate (304 mg, 1.0 mmol, Bioorganic & Medicinal Chemistry Letters 2004, 14, 1577), methylboronic acid (59.9 mg, 1.0 mmol), tetrakis(triphenylphosphine)palladium (58 mg, 0.05 mmol) and potassium carbonate (415 mg, 3 mmol) in dioxane (10 ml) was stirred at reflux for 16 hours. The mixture was quenched with saturated sodium bicarbonate aqueous solution, and the product was extracted with ethyl acetate which was dried over sodium sulfate. Then, filtration, evaporation, purification through silica gel column chromatography eluting with hexane/ethyl acetate (2:1) to furnish the title compound (90 mg, 32% yield) as a white solid.

¹H NMR (300 MHz, DMSO-d₆) δ1.39 (3H, t, J=7.3 Hz), 2.95 (3H, s), 4.42 (2H, q, J=7.3 Hz), 7.99 (1H, d, J=8.9 Hz), 8.41 (1H, s), 8.56 (1H, d, J=8.9 Hz), 9.22 (1H, s)

MS (ESI): m/z 284 (M+H)+.

### 11B) 4-METHYL-7-(TRIFLUOROMETHYL)QUINOLINE-3-CARBOXYLIC ACID

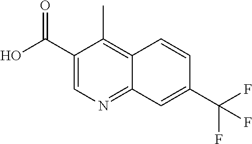

To a solution of the compound of Example 11A (90 mg, 0.32 mmol) in Methanol (4 ml) and THF (4 ml) was added 2M aqueous sodium hydroxide (1 ml) at room temperature. The mixture was stirred at 50° C. for 1 hour. Then evaporated, diluted with water (5 ml), neutralized to pH 5˜6 by 2M aqueous hydrochloride. The formed precipitate was collected, washed with water to furnish the title compound (50 mg, 62% yield) as a white solid.

¹H NMR (300 MHz, DMSO-d₆) δ 2.79 (3H, s), 7.97 (1H, d, J=8.8 Hz), 8.40 (1H, s), 8.54 (1H, d, J=8.8 Hz), 9.22 (1H, s),

MS (ESI) m/z 256 (M+H)+.

### 11C) 4-METHYL-N-((1R)-1-{3-METHYL-4-[(METHYLSULFONYL)AMINO]PHENYL}ETHYL)-7-(TRIFLUOROMETHYL)QUINOLINE-3-CARBOXAMIDE

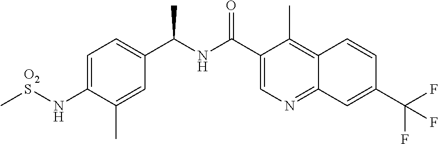

A DMF (1 ml) solution of the compound of Example 11B (50 mg, 0.20 mmol), triethylamine (0.082 ml, 0.59 mmol), the compound of Example 1D (52 mg, 0.20 mmol) and HBTU (89 mg, 0.24 mmol) was treated in the same procedure described in Example 1G. The crude residue was applied to a silica gel column chromatography and eluted with dichloromethane/methanol (20:1) to furnish the title compound (52 mg, 57% yield) as a white solid.

¹H NMR (300 MHz, DMSO-d₆) δ 1.47 (3H, d, J=6.6 Hz), 2.33 (3H, s), 2.71 (3H, s), 2.99 (3H, s), 5.13-5.23 (1H, m), 7.27-7.32 (3H, m), 7.96 (1H, d, J=8.8 Hz), 8.40 (1H, s), 8.45 (1H, d, J=8.8 Hz), 8.92 (1H, s), 9.04 (1H, s), 9.14 (1H, d, J=8.1 Hz)

MS (ESI): m/z 466 (M+H)+.

### Example 12

### 6-BROMO-N-((1R)-1-{3-METHYL-4-[(METHYLSULFONYL)AMINO]PHENYL}ETHYL)-QUINOLINE-2-CARBOXAMIDE

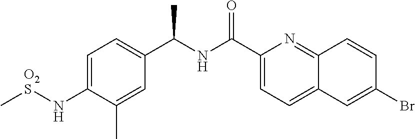

A DMF (10 ml) solution of 6-bromoquinoline-2-carboxylic acid (1000 mg, 4.0 mmol, Yakugaku Zasshi 1977, 97, 1022); triethylamine (1.66 ml, 12.0 mmol), the compound of Example 1D (1050 mg, 4.0 mmol) and HBTU (1810 mg, 4.8 mmol) was treated in the same procedure described in Example 1G. The mixture was quenched with saturated sodium bicarbonate aqueous solution (200 ml), then diluted with ethyl acetate/hexane (6:1) (350 ml). The precipitate was collected and washed with ethyl acetate (30 ml) to furnish the title compound (1268 mg, 69% yield) as a white solid.

¹H NMR (300 MHz, DMSO-d₆) δ 1.56 (3H, d, J=6.6 Hz), 2.28 (3H, s), 2.92 (3H, s), 5.13-5.22 (1H, m), 7.20-7.32 (3H, m), 7.95-8.05 (1H, m), 8.11-8.17 (2H, m), 8.41 (1H, s), 8.53 (1H, d, J=8.8 Hz), 9.01 (1H, brs), 9.13 (1H, d, J=8.1 Hz)

MS (ESI): m/z 463 (M+H)+.

### Example 13

### 6-TERT-BUTYL-N-((1R)-1-{2-FLUORO-5-METHYL-4-[(METHYLSULFONYL)AMINO]PHENYL}-ETHYL)-2-NAPHTHAMIDE

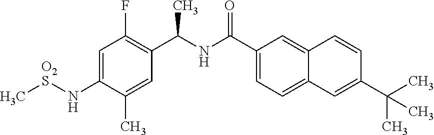

### 13A) N-(5-FLUORO-2-METHYLPHENYL)METHANESULFONAMIDE

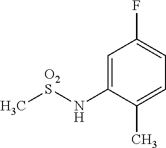

To a pyridine (20 ml) and DCM (40 ml) solution of 5-fluoro-2-methylaniline (3.5 g, 28 mmol), methanesulfonyl chloride (4.3 ml, 56 mmol) was added at room temperature and the mixture was stirred for 20 hours. The reaction was quenched with 2M sodium hydroxide aqueous solution and the aqueous layer was separated and washed with DCM. The layer was cooled to 0° C. and acidified to pH 2.0 using 2M HCl aqueous solution. The precipitates were collected, and the solvent evaporated in vacuo, to give the title compound (5.1 g, 90%). MS (ESI) m/z 202 (M−H)⁻

### 13B) N-(4-ACETYL-5-FLUORO-2-METHYLPHENYL)METHANESULFONAMIDE

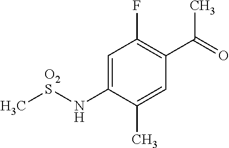

To a DCM (45 ml) suspension of aluminum trichloride (4.9 g, 36.9 mmol); acetyl chloride (1.9 g, 24.6 mmol) was slowly added at room temperature and the mixture was stirred for 20 minutes; then a dichloromethane (15 ml) solution of the compound of Example 13A (2.5 g, 12.3 mmol) was added to the mixture and the reaction was stirred for 2.5 hours at room temperature. The reaction mixture was poured into ice-water and the whole was extracted with DCM. The organic layer was dried over magnesium sulfate and the solvent evaporated to give the title compound (1.4 g, 46%).

¹H NMR (270 MHz, DMSO-d₆) δ 2.24-2.31 (3H, m), 2.54 (3H, d, J=4.6 Hz), 3.15 (3H, s), 7.27 (1H, d, J=132 Hz), 7.28 (1H, d, J=7.9 Hz), 9.54 (1H, brs).

### 13C) N-[4-((1R)-1-{[(R)-TERT-BUTYLSULFINYL]AMINO}ETHYL)-5-FLUORO-2-METHYL-PHENYL]METHANESULFONAMIDE

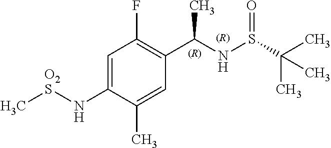

To a THF (5 ml) solution of the compound of Example 13B (1.4 g, 5.5 mmol) and (R)-(+)-2-methyl-2-propanesulfinylamide (1.0 g, 8.26 mmol), titanium(IV) ethoxide (5.0 ml, 21.9 mmol) was added under a nitrogen atmosphere and the mixture was subjected to microwave irradiation at 70° C. with stirring for 2.5 hours. After imine formation was confirmed with LC-MS (MS (ESI) m/z 347 (M−H)⁻, 349 (M+H)⁺), the mixture was cooled to 0° C. and sodium borohydride (707 mg, 18.7 mmol) was added and the reaction mixture was stirred for 2 hours at 0° C. The reaction mixture was partitioned with water and ethanol, then the mixture was stirred for 1 hour at room temperature. The mixture was filtered through a Celite pad, and the filtrate was evaporated and concentrated in vacuo to give the title compound (1.9 g, 99%).

MS (ESI) m/z 349 (M−H)⁻, 351 (M+H)⁺

### 13D) N-{4-[(1R)-1-AMINOETHYL]-5-FLUORO-2-METHYLPHENYL}METHANESULFONAMIDE HYDROCHLORIDE

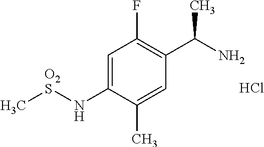

To the compound of Example 13C (1.9 g, 5.5 mmol) was added HCl-MeOH (2.0 M, 15.0 ml) and 1,4-dioxane (15.0 ml). The same procedure as described in Example 2D was performed to give the title compound (1.2 g, 74%) as white solids.

MS (ESI) m/z 245 (M−H)⁻.

### 13E) 6-TERT-BUTYL-N-((1R)-1-{2-FLUORO-5-METHYL-4-[(METHYLSULFONYL)AMINO]PHENYL}ETHYL)-2-NAPHTHAMIDE

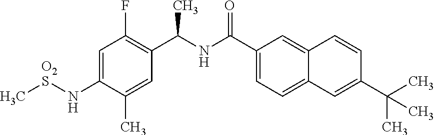

To a CH₂Cl₂ (5.0 ml) solution of the compound of Example 1F (100 mg, 0.44 mmol), thionyl chloride (1.0 ml) and DMAP (5.0 mg) were added and the mixture was stirred for 1 hour at room temperature. Then, solvent and thionyl chloride were removed under reduced pressure to give the white solid, which was used for further reaction without purification. To a CH₂Cl₂ (20 ml) solution of the compound of Example 13D (124 mg, 0.44 mmol), a CH₂Cl₂ (10 ml) solution of acid chloride prepared was added and the mixture was stirred for 1 hour at room temperature. Then, solvent was removed under reduced pressure to give the white solid which was crystallized from ethylacetate-hexane to give the white solid product in 58% yield.

¹H NMR (300 MHz, DMSO-d₆) δ 1.39 (9H, s), 1.51 (3H, s), 2.24 (3H, s), 3.01 (3H, s), 5.37-5.42 (1H, m), 7.09 (1H, d, J=11.74 Hz), 7.35 (1H, d, J=9.0 Hz), 7.72 (1H, d, J=7.3 Hz), 7.89-7.99 (3H, m), 8.46 (1H, m), 8.96 (1H, d, J=7.34 Hz), 9.25 (1H, s).

MS (ESI): m/z 457 (M+H)⁺.

### Example 14

### 6-TERT-BUTYL-N-((1R)-1-{2-FLUORO-5-METHYL-4-[(METHYLSULFONYL)AMINO]PHENYL}-ETHYL)QUINOLINE-2-CARBOXAMIDE

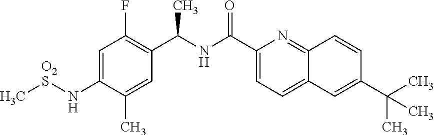

A CH₂Cl₂ (5.0 ml) solution of 6-(tert-butyl)quinoline-2-carboxylic acid (100 mg, 0.44 mmol), thionyl chloride (1.0 ml) and DMAP (5.0 mg) were added and the mixture was stirred for 1 hour at room temperature. Then, solvent and thionyl chloride were removed under reduced pressure to give the white solid, which was used for further reaction without purification. To a CH₂Cl₂ (20 ml) solution of the compound of Example 13D (124 mg, 0.44 mmol), a CH₂Cl₂ (10 ml) solution of acid chloride prepared was added and the mixture was stilled for 1 hour at room temperature. Then, solvent was removed under reduced pressure to give the white solid product in 33% yield.

¹H NMR (270 MHz, DMSO-d₆) δ 1.41 (9H, s), 1.57 (3H, d, J=7.25 Hz), 2.23 (3H, s), 3.01 (3H, s), 5.38-5.43 (1H, m), 7.10 (1H, d, J=11.87 Hz), 7.42 (1H, d, J=9.2 Hz), 7.99-8.13 (3H, m), 7.89-7.99 (3H, m), 8.53 (1H, d, J=8.5 Hz), 9.12 (1H, s), 9.15 (1H, s).

MS (ESI): m/z 458 (M+H)⁺.

### Example 15

### 2-TERT-BUTYL-N-((1R)-1-{3,5-DIFLUORO-4-[(METHYLSULFONYL)AMINO]PHENYL}ETHYL)-QUINOLINE-6-CARBOXAMIDE HYDROCHLORIDE

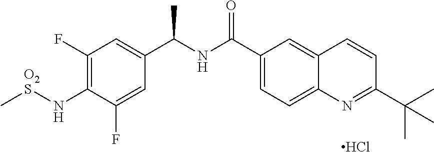

A DMF (1.5 ml) solution of the compound of Example 8B (80 mg, 0.35 mmol), triethylamine (0.15 ml, 1.1 mmol), the compound of Example 3D (100 mg, 0.35 mmol) and HBTU (159 mg, 0.42 mmol) was treated in the same procedure described in Example 1G. The crude residue was applied to a silica gel column chromatography and eluted with hexane/ethyl acetate (1:1). The obtained product was dissolved in 10% hydrochloride in methanol (5 ml) and stirred for 1 hour. The solvent was removed in vacuo and the residue was crystallized from methanol and ethyl acetate to furnish the title compound (69 mg, 40% yield) as a white solid.

¹H NMR (300 MHz, DMSO-d₆) δ 1.51-1.55 (12H, m), 3.06 (3H, s), 5.18-5.27 (1H, m), 7.29 (2H, d, J=8.8 Hz), 7.94 (1H, d, J=8.8 Hz), 8.30-8.39 (2H, m), 8.68-8.75 (2H, m), 9.23 (1H, d, J=7.3 Hz), 9.52 (1H, s).

MS (ESI): m/z 462 (M+H)+.

### Example 16

### 6-(1-HYDROXY-1-METHYLETHYL)-N-((1R)-1-{3-METHYL-4-[(METHYLSULFONYL)AMINO]-PHENYL}ETHYL)QUINOLINE-2-CARBOXAMIDE

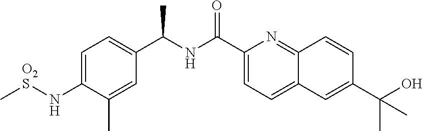

### 16A) 2-QUINOLIN-6-YLPROPAN-2-OL

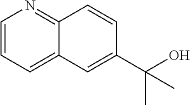

To a THF (10 ml) solution of 6-bromoqinoline (500 mg, 2.4 mmol) was added 1.6 M n-BuLi in hexane (1.65 ml, 2.64 mmol) dropwise at −78° C. and the mixture was stirred for 1 hour, then acetone (0.2 ml, 2.72 mmol) was added there at −78° C. After 1 hour, the mixture was quenched with small amount of methanol and purified through silica gel column chromatography eluting with hexane/ethyl acetate (1:1) to furnish the title compound (190 mg, 42% yield) as colorless oil.

¹H NMR (300 MHz, CDCl₃) δ 1.69 (6H, s), 2.04 (1H, s), 7.38-7.42 (1H, m), 7.83-7.86 (1H, m), 7.94 (1H, s), 8.08 (1H, d, J=8.8 Hz), 8.16 (1H, d, J=8.8 Hz), 8.88-8.90 (1H, m).

MS (ESI): m/z 188 (M+H)+.

### 16B) 2-(1-OXIDOQUINOLIN-6-YL)PROPAN-2-OL

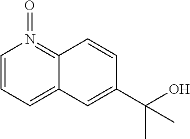

A mixture of the compound of Example 16A (190 mg, 1.0 mmol), mCPBA (350 mg, 1.5 mmol) in chloroform (5 ml) was treated in the same procedure described in Example 6A. The crude residue was applied to a silica gel (NH silica) column chromatography and eluted with dichloromethane/methanol (20:1) to furnish the title compound (145 mg, 70% yield) as a white solid.

¹H NMR (300 MHz, CDCl₃) [symbol] 1.67 (6H, s), 2.88 (1H, s), 7.24-7.29 (1H; m), 7.64 (1H, d, J=8.1 Hz), 7.78-7.81 (1H, m), 7.87 (1H, s), 8.45-8.52 (2H, m).

MS (ESI): m/z 204 (M+H)+.

### 16C) 6-{1-METHYL-1-[(TRIMETHYLSILYL)OXY]ETHYL}QUINOLINE-2-CARBONITRILE

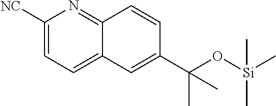

A mixture of the compound of Example 16B (145 mg, 0.71 mmol), trimethylsilylcyanide (211 mg, 2.13 mmol), trimethylamine (0.2 ml, 1.42 mmol) in acetonitrile (1.4 ml) was stirred for 15 minutes at 120° C. under microwave irradiation. Then the mixture was applied to a silica gel column chromatography and eluted with hexane/ethyl acetate (2:1) to furnish the 3:2 mixture of the title compound and 6-(1-hydroxy-1-methylethyl)quinoline-2-carbonitrile (194 mg) as a colorless oil, which was used in the next reaction without further purification.

MS (ESI): m/z 213, 285 (M+H)+.

### 16D) 6-(1-HYDROXY-1-METHYLETHYL)QUINOLINE-2-CARBOXYLIC ACID

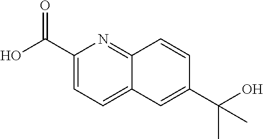

A solution of the compound of Example 16C (194 mg) and 2M-aqueous sodium hydroxide (1 ml) in ethanol (3 ml) was treated in the same procedure described in Example 6C to furnish the title compound (97 mg, 59% yield in 2 steps) as a white solid.

¹H NMR (300 MHz, CDCl₃) δ 1.71 (6H, s), 7.96-8.45 (5H, m).

MS (ESI): m/z 232 (M+H)+.

### 16E) 6-(1-HYDROXY-1-METHYLETHYL)-N-((1R)-1-{3-METHYL-4-[(METHYLSULFONYL)-AMINO]PHENYL}ETHYL)QUINOLINE-2-CARBOXAMIDE

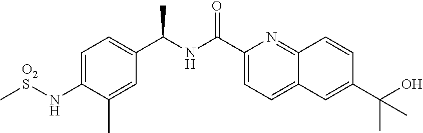

To a DMF (2 ml) solution of the compound of Example 16D (89 mg, 0.386 mmol), the compound of Example 1D (102 mg, 0386 mmol) and HBTU (176 mg, 0.463 mmol) was added triethylamine (0.16 ml, 1.16 mmol) and the mixture was stirred for 2 hours at room temperature. The same procedure as described in Example 1G was performed to furnish the title compound (156 mg, 91% yield) as a white solid.

¹H NMR (DMSO-d₆) δ 1.54 (6H, s), 1.56 (3H, d, J=7.3 Hz), 2.29 (3H, s), 2.96 (3H, s), 5.14-5.23 (1H, m), 5.32-5.33 (1H, m), 7.22-7.35 (3H, m), 7.95-8.14 (4H, m), 8.53 (1H, d, J=8.8 Hz), 9.01 (1H, s), 9.09 (1H, d, J=8.8 Hz).

MS (ESI) m/z 440 (M−H)⁻, 442 (M+H)⁺.

### Example 17

### 6-BROMONAPHTHALENE-2-CARBOXYLIC ACID [(R)-1-(4-METHANESULFONYLAMINO-3-METHYLPHENYL)ETHYL]AMIDE

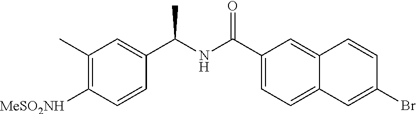

### 17A) 6-BROMONAPHTHALENE-2-CARBOXYLIC ACID

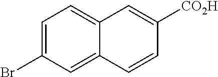

To a stirred solution of 6-bromonaphthalene-2-carboxylic acid methyl ester (2 g, 8 mmol) in tetrahydrofuran (66 mL) and ethanol (22 mL) was added a solution of lithium hydroxide (542 mg, 22 mmol) in water (22 mL). The reaction was stirred at 50° C. for 16 hours. After cooling, the organic solvents were removed by evaporation, and the aqueous residue was diluted with water (100 mL) then washed with EtOAc (2×50 mL). The aqueous layer was acidified using 1N HCl and the products were extracted with EtOAc (3×50 mL). The combined organics were washed with brine (100 mL), dried (MgSO₄), filtered and concentrated. Trituration with DCM gave the title compound (1.594 g, 80%) as an off-white solid.

¹H NMR (400 MHz, DMSO-d₆) δ 7.74 (dd, 1H, J=8.7 Hz, 1.9 Hz), 7.99-8.04 (m, 2H), 8.10 (d, 1H, J=8.8 Hz), 8.32 (s, 1H), 8.64 (s, 1H).

### 17B) 6-BROMONAPHTHALENE-2-CARBOXYLIC ACID [(R)-1-(4-METHANESULFONYLAMINO-3-METHYLPHENYL)ETHYL]AMIDE

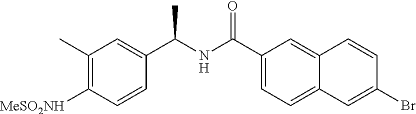

To a stirred solution of N-[44(R)-1-Aminoethyl)2-methylphenyl]methanesulfonamide (40 mg, 0.2 mmol) in anhydrous DMF (2 mL) was added a solution of 6-bromonaphthalene-2-carboxylic acid (53 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 0.71 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAC (3×50 mL). The combined organics were washed with brine (3 z 50 mL), dried (MgSO₄), filtered and concentrated. Flash chromatography (0 to 4% MeOH in DCM) gave the title compound (22 mg, 30%) as a white solid.

¹H NMR (400 MHz, DMSO-d₆) δ 1.49 (d, 3H, J=7.0 Hz), 2.30 (s, 3H), 2.95 (s, 3H), 5.14-5.21 (m, 1H), 7.21-7.31 (m, 3H), 7.71 (dd, 1H, J=8.7 Hz, 2.0 Hz), 7.95-8.03 (m, 3H), 8.28 (d, 1H, J=2.0 Hz), 8.50 (s, 1H), 8.97-9.01 (m, 2H).

LC/MS: m/z 463 (M+H)⁺; r.t.=4.39 min

### Example 18

### 18A) 6-FLUORONAPHTHALENE-2-CARBOXYLIC ACID [(R)-1-(4-METHANESULFONYL-AMINO-3-METHYLPHENYL)ETHYL]AMIDE

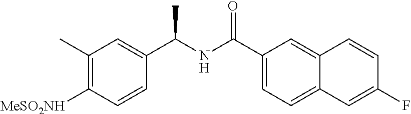

To a stirred solution of N-[4-((R)-1-aminoethyl)2-methylphenyl]methanesulfonamide (40 mg, 0.2 mmol) in anhydrous DMF (2 mL) was added a solution of 6-fluoronaphthalene-2-carboxylic acid (40 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in DCM) gave the title compound (31 mg, 40%) as a white solid.

¹H NMR (400 MHz, DMSO-d₆) δ 1.50 (d, 3H, J=7.1 Hz), 2.30 (s, 3H), 2.96 (s, 3H), 5.14-5.21 (m, 1H), 7.21-7.29 (m, 3H), 7.48-7.54 (m, 1H), 7.77-7.80 (m, 1H), 7.95-8.05 (m, 2H), 8.07-8.16 (m, 1H), 8.53 (s, 1H), 8.97 (d, 1H, J=7.9 Hz), 9.01 (s, 1H).

LC/MS: 401 m/z (M+H)⁺; r.t.=3.03 min

### Example 19

### 19A) NAPHTHALENE-2-CARBOXYLIC ACID [(R)-1-(4-METHANESULFONYLAMINO-3-METHYLPHENYL)ETHYL]AMIDE

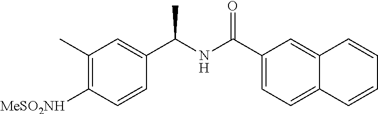

To a stirred solution of N-[4-((R)-1-aminoethyl)2-methylphenyl]methanesulfonamide (40 mg, 0.2 mmol) in anhydrous DMF (2 mL) was added a solution of naphthalene-2-carboxylic acid (36 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in DCM) gave the title compound (13 mg, 20%) as a white solid.

¹H NMR (400 MHz, DMSO-d₆) δ 1.51 (d, 3H, J=7.1 Hz), 2.30 (s, 3H), 2.96 (s, 3H), 5.14-5.22 (m, 1H), 7.22-7.30 (m, 3H), 7.57-7.64 (m, 2H), 7.94-8.05 (m, 4H), 8.50 (s, 1H), 8.96 (d, 1H, J=8.0 Hz), 9.01 (s, 1H).

LC/MS: m/z 383 (M+H)⁺; r.t.=2.97 min

### Example 20

### 20A) 6-METHOXYNAPHTHALENE-2-CARBOXYLIC ACID [(R)-1-(4-METHANESULFONYL-AMINO-3-METHYLPHENYL)ETHYL]AMIDE

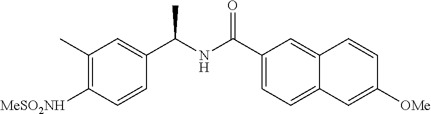

To a stirred solution of N-[4-((R)-1-aminoethyl)2-methylphenyl]methanesulfonamide (40 mg, 0.2 mmol) in anhydrous DMF (2 mL) was added a solution of 6-methoxynaphthalene-2-carboxylic acid (43 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122□L, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in DCM) gave the title compound (45 mg, 60%) as a white solid.

¹H NMR (400 MHz, DMSO-d₆) δ 1.51 (d, 3H, J=7.1 Hz), 2.30 (s, 3H), 2.96 (s, 3H), 3.90 (s, 3H), 5.14-5.21 (m, 1H), 7.21-7.26 (m, 3H), 7.29 (s, 1H), 7.38 (1H, d, J=2.5 Hz), 7.86-7.95 (m, 3H), 8.42 (s, 1H), 8.85 (d, 1H, J=7.9 Hz), 9.00 (s, 1H).

LC/MS: m/z 413 (M+H)⁺; r.t.=2.99 min

### Example 21

### 6-PYRROLIDIN-1-YL-NAPHTHALENE-2-CARBOXYLIC ACID [(R)-1-(4-METHANESULFONYL-AMINO-3-METHYLPHENYL)ETHYL]AMIDE

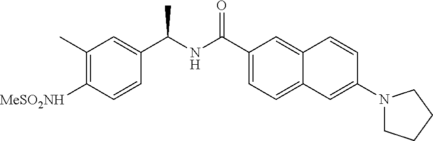

### 21A) 6-PYRROLIDIN-1-YL-NAPHTHALENE-2-CARBOXYLIC ACID METHYL ESTER

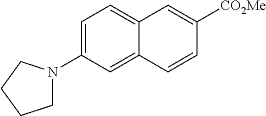

A flask containing 6-bromonaphthalene-2-carboxylic acid methyl ester (1 g, 4 mmol), palladium acetate (8.5 mg, 0.04 mmol), racemic BINAP (35 mg, 0.06 mmol) and cesium carbonate (1.721 g, 5.2 mmol) in anhydrous toluene (8 mL) was degassed with N₂ for 10 minutes. Pyrrolidine (0.38 mL, 4.5 mmol) was added, and the reaction was heated at 100° C. for 16 hours. After cooling, the reaction mixture was poured into saturated NaHCO₃ solution (100 ml) and extracted with EtOAc (3×50 ml). The combined organics were washed with brine (3×50 ml), dried (MgSO₄), filtered and concentrated. Flash chromatography (0 to 20% EtOAc in hexanes) gave the title compound (220 mg, 20%) as yellow crystals.

¹H NMR (400 MHz, CDCl₃) δ 2.07 (m, 4H), 3.52 (m, 4H), 3.95 (s, 3H), 6.72 (d, 1H, J=2.2 Hz), 7.01 (dd, 1H, J=9.0 Hz, 2.4 Hz), 7.59 (d, 1H, J=8.7 Hz), 7.77 (d, 1H, J=9.0 Hz), 7.91 (dd, 1H, J=8.7 Hz, 1.8 Hz), 8.43 (d, 1H, J=1.3 Hz).

LC/MS: m/z 256 (M+H)⁺; r.t.=3.92 min

### 21B) 6-PYRROLIDIN-1-YL-NAPHTHALENE-2-CARBOXYLIC ACID

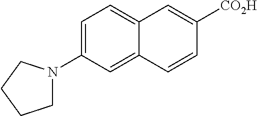

A solution of lithium hydroxide (56 mg, 2.35 mmol) in water (2.5 mL) was added to a stirred solution of 6-pyrrolidin-1-ylnaphthalene-2-carboxylic acid methyl ester (200 mg, 0.8 mmol) in tetrahydrofuran (7.5 mL) and ethanol (2.5 mL). The reaction was stirred at 50° C. for 72 hours. After cooling, the organic solvents were evaporated are the resulting aqueous solution was acidified with 2N HCl. After filtration, the filtrate was extracted with EtOAc (3×50 ml). The combined organics were washed with brine (2×50 ml), dried (MgSO₄), filtered and concentrated. Trituration using DCM/hexanes gave the title compound (80 mg, 40%) as yellow crystals.

¹H NMR (400 MHz, DMSO-d₆) δ 2.00 (t, 4H, J=6.4 Hz), 3.38 (t, 4H, J=6.4 Hz), 6.78 (d, 1H, J=1.6 Hz), 7.09 (dd, 1H, J=9.0 Hz, 2.2 Hz), 7.63 (d, 1H, J=8.6 Hz), 7.77 (dd, 1H, J=8.6 Hz, 1.6 Hz), 7.87 (4, 1H, J=9.0 Hz), 8.35 (s, 1H).

### 21C) 6-PYRROLIDIN-1-YL-NAPHTHALENE-2-CARBOXYLIC ACID [(R)-1-(4-METHANESULFONYLAMINO-3-METHYLPHENYL)ETHYL]AMIDE

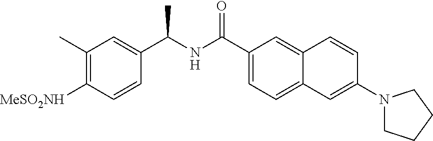

To a stirred solution of N-[4-((R)-1-aminoethyl)2-methylphenyl]methanesulfonamide (40 mg, 0.2 mmol) in anhydrous DMF (2 mL) was added a solution of 6-pyrrolidin-1-yl-naphthalene-2-carboxylic acid (50 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 5% MeOH in DCM) gave the title compound (34 mg, 40%) as an off-white powder.

¹H NMR (400 MHz, DMSO-d₆) δ 1.48 (d, 3H, J=7.1 Hz), 2.01 (t, 4H, J=6.5 Hz), 2.30 (s, 3H), 2.95 (s, 3H), 3.37 (t, 4H, J=6.5 Hz), 5.14-5.18 (m, 1H), 6.77 (d, 1H, J=1.8 Hz), 7.08 (dd, 1H, J=9.0 Hz, 2.3 Hz), 7.20-7.28 (m, 3H), 7.63 (d, 1H, J=8.7 Hz), 7.76-7.82 (m, 2H), 8.28 (s, 1H), 8.70 (d, 1H, J=8.0 Hz), 9.00 (s, 1H).

LC/MS: m/z 452 (M+H)⁺; r.t.=3.37 min

### Example 22

### 6-CYCLOPROPYLNAPHTHALENE-2-CARBOXYLIC ACID [(R)-1-(4-METHANESULFONYL-AMINO-3-METHYLPHENYL)ETHYL]AMIDE

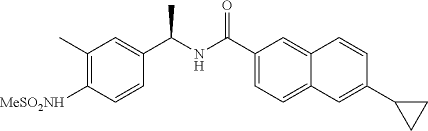

### 22A) 6-CYCLOPROPYLNAPHTHALENE-2-CARBOXYLIC ACID METHYL ESTER

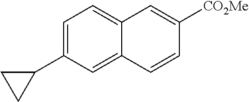

A flask containing 6-bromonaphthalene-2-carboxylic acid methyl ester (1.0 g, 3.7 mmol), cyclopropyl boronic acid (421 mg, 4.9 mmol), palladium acetate (42 mg, 0.02 mmol), tricyclohexylphosphine (106 mg, 0.04 mmol) and potassium phosphate (2.802 g, 13.2 mmol) in toluene (15 mL) and water (0.75 mL) was degassed with N₂ for 10 minutes. The reaction was heated at 100° C. for 1 hour. After cooling, the reaction mixture was poured into saturated NaHCO₃ solution (100 ml) and extracted with EtOAc (3×50 ml). The combined organics were washed with brine (3×50 ml), dried (MgSO₄), filtered and concentrated. Flash chromatography (0 to 10% EtOAc in hexanes) gave the title compound (270 mg, 30%) as an off-white solid.

¹H NMR (400 MHz, DMSO-d₆) δ 0.83-0.87 (m, 2H), 1.05-1.11 (m, 2H), 2.09-2.16 (m, 1H), 3.90 (s, 3H), 7.33 (dd, 1H, J=8.6 Hz, 1.8 Hz), 7.70 (s, 1H), 7.89-7.95 (m, 2H), 8.02 (d, 1H, J=8.6 Hz), 8.56 (s, 1H).

### 22B) 6-CYCLOPROPYLNAPHTHALENE-2-CARBOXYLIC ACID

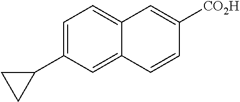

To a solution of 6-cyclopropylnaphthalene-2-carboxylic acid methyl ester (226 mg, 1 mmol) in tetrahydrofuran (9 mL) and ethanol (3 mL) was added a solution of lithium hydroxide (72 mg, 3 mmol) in water (3 mL). The reaction was stirred at 50° C. for 2 hours, then poured into 2N HCl and extracted with EtOAc (3×50 ml). The combined organics were washed with brine (2×100 ml), dried (MgSO₄), filtered and concentrated. Trituration using DCM/hexanes gave the title compound (150 mg, 67%) as a white solid.

¹H NMR (400 MHz, MeOH-d₄) δ 0.85-0.89 (m, 2H), 1.09-1.16 (m, 2H), 2.11-2.16 (m, 1H), 7.30 (dd, 1H, J=8.6 Hz, 1.7 Hz), 7.64 (s, 1H), 7.84 (d, 1H, J=8.6 Hz), 7.89 (d, 1H, J=8.6 Hz), 8.00 (dd, 1H, J=8.6 Hz, 1.7 Hz), 8.55 (s, 1H).

### 22C) 6-CYCLOPROPYLNAPHTHALENE-2-CARBOXYLIC ACID [(R)-1-(4-METHANE-SULFONYL-AMINO-3-METHYLPHENYL)ETHYL]AMIDE

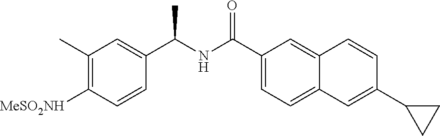

To a stirred solution of N-[4-((R)-1-aminoethyl)2-methylphenyl]methanesulfonamide (40 mg, 0.2 mmol) in anhydrous DMF (2 mL) was added a solution of 6-cyclopropylnaphthalene-2-carboxylic acid (45 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 5% MeOH in DCM) gave the title compound (8 mg, 10%) as a white solid.

¹H NMR (400 MHz, DMSO-d₆) δ 0.82-0.85 (m, 2H), 1.04-1.06 (dd, 2H, J=6.2 Hz, 2.1 Hz), 1.49 (d, 3H, J=7.0 Hz), 2.09-2.13 (m, 1H), 2.30 (s, 3H), 2.96 (s, 3H), 5.14-5.18 (m, 1H), 7.21-7.34 (m, 4H), 7.66 (s, 1H), 7.85-7.92 (m, 3H), 8.42 (s, 1H), 8.89 (d, 1H, J=8.1 Hz), 9.0 (s, 1H).

LC/MS: m/z 423 (M+H)⁺; r.t.=3.30 min

### Example 23

### 7-CHLORO-2-METHYL-QUINOLINE-3-CARBOXYLIC ACID [(R)-1-(4-METHANESULFONYL-AMINO-3-METHYLPHENYL)ETHYL]AMIDE

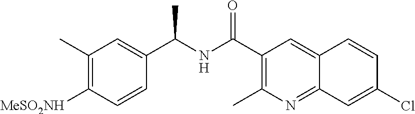

### 23A) 7-CHLORO-2METHYLQUINOLINE-3-CARBOXYLIC ACID

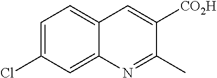

To a stirred solution of 7-chloro-2-methylquinoline-3-carboxylic acid ethyl ester (1.0 g, 4 mmol) in tetrahydrofuran (36 mL) and ethanol (12mL) was added a solution of lithium hydroxide (287 mg) in water (12 mL). The reaction was stirred at 50° C. overnight. After cooling, the organic solvents were evaporated and the aqueous solution was acidified to pH 7.0 with 2N HCl. The product was then extracted into EtOAc (2×100 ml) and the combined organics were washed with brine (100 ml), dried (MgSO₄), filtered and concentrated. Trituration with DCM/hexanes gave the title compound (100 mg, 10%) as an off-white solid.

¹H NMR (400 MHz, DMSO-d₆) δ 2.86 (s, 3H), 7.64 (d, 1H, J=7.4 Hz), 8.01 (s, 1H), 8.13 (d, 1H, J=8.7 Hz), 8.82 (s, 1H).

LC/MS: 220 m/z (M−H)⁻; r.t.=2.10 min

### 23B) 7-CHLORO-2-METHYL-QUINOLINE-3-CARBOXYLIC ACID [(R)-1-(4-METHANE-SULFONYLAMINO-3-METHYLPHENYL)ETHYL]AMIDE

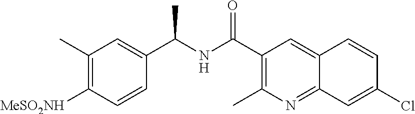

To a stirred solution of N-[4-((R)-1-aminoethyl)2-methylphenyl]methanesulfonamide (40 mg, 0.2 mmol) in anhydrous DMF (2 mL) was added a solution of 7-chloro-2-methyl-quinoline carboxylic acid (46 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 5% MeOH in DCM) gave the title compound (13 mg, 20%) as a white solid.

¹H NMR (400 MHz, DMSO-d₆) δ 1.45 (d, 3H, J=7.0 Hz), 2.32 (s, 3H), 2.64 (s, 3H), 2.98 (s, 3H), 5.14-5.18 (m, 1H), 7.26-7.35 (m, 3H), 7.64 (dd, 1H, J=8.7 Hz, 2.1 Hz), 8.02 (d, 1H, J=2.1 Hz), 8.04-8.11 (m, 1H), 8.39 (s, 1H), 9.03-9.09 (m, 2H).

LC/MS: m/z 432 (M+H)⁺; r.t.=2.67 min

### Example 24

### 6-MORPHOLIN-1-YL-NAPHTHALENE-2-CARBOXYLIC ACID [(R)-1-(4-METHANESULFONYL-AMINO-3-METHYLPHENYL)ETHYL]AMIDE

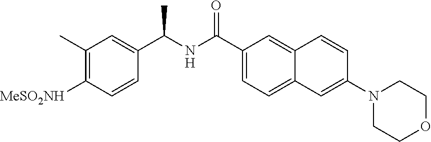

### 24A) 6-MORPHOLIN-1-YL-NAPHTHALENE-2-CARBOXYLIC ACID METHYL ESTER

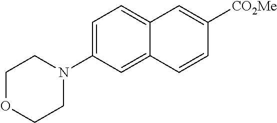

A flask containing 6-bromonaphthalene-2-carboxylic acid methyl ester (1 g, 4 mmol), palladium acetate (8.5 mg, 0.04 mmol), racemic BINAP (35 mg, 0.06 mmol) and cesium carbonate (1.721 g, 5.2 mmol) in anhydrous toluene (8 mL) was degassed with N₂ for 10 minutes. Morpholine (0.66 mL, 7.5 mmol) was added, and the reaction was heated at 100° C. for 16 hours. After cooling, the reaction mixture was poured into saturated NaHCO₃ solution (100 ml) and extracted with EtOAc (3×50 ml). The combined organics were washed with brine (3×50 ml), dried (MgSO₄), filtered and concentrated. Flash chromatography (0 to 20% EtOAc in hexanes) gave the title compound (506 mg, 50%) as yellow crystals.

¹H NMR (400 MHz, DMSO-d₆) δ 3.31 (t; 4H, J=4.7 Hz), 3.79 (t, 4H, J=4.7 Hz), 3.88 (s, 3H), 7.23 (d, 1H, J=1.8 Hz), 7.48 (dd, 1H, J=9.1 Hz, 2.2 Hz), 7.78 (d, 1H, J=8.6 Hz), 7.86 (dd, 1H, J=8.6 Hz, 1.6 Hz), 7.97 (d, 1H, J=9.1 Hz), 8.46 (s, 1H).

LC/MS: m/z 272 (M+H)⁺; r.t.=3.23 min

### 24B) 6-MORPHOLIN-1-YL-NAPHTHALENE-2-CARBOXYLIC ACID

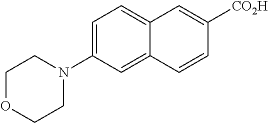

To a stirred solution of 6-morpholin-1-yl-naphthalene-2-carboxylic acid methyl ester (271 mg, 1 mmol) in tetrahydrofuran (15 mL) and ethanol (5 mL) was added a solution of lithium hydroxide (119 mg, 5 mmol) in water (5 mL). The reaction was stirred at 50° C. for 16 hours. After cooling the reaction mixture was diluted with water (100 mL) and then acidified to pH 7.0 with 2N HCl. The mixture was extracted with EtOAc (3×50 mL). The combined organics were dried (MgSO₄), filtered and concentrated to give the title compound (177 mg, 69%) as a yellow solid.

¹H NMR (400 MHz, DMSO-d₆) δ 3.30 (t, 4H, J=4.8 Hz), 3.79 (t, 4H, J=4.7 Hz), 7.23 (d, 1H, J=2.0 Hz), 7.46 (dd, 1H, J=9.1 Hz, 2.3 Hz), 7.76 (d, 1H, J=8.6 Hz), 7.84 (dd, 1H, J=8.6 Hz, 1.6 Hz), 7.94 (d, 1H, J=9.1 Hz), 8.43 (s, 1H).

LC/MS: m/z 258 (M+H)⁺; r.t.=2.58 min

### 24C) 6-MORPHOLIN-1-YL-NAPHTHALENE-2-CARBOXYLIC ACID [(R)-1-(4-METHANE-SULFONYLAMINO-3-METHYLPHENYL)ETHYL]AMIDE

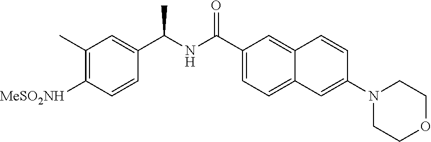

To a stirred solution of N-[4-((R)-1-aminoethyl)2-methylphenyl]methanesulfonamide (40 mg, 0.2 mmol) in anhydrous DMF (2 mL) was added a solution of 6-morpholin-1-yl-naphthalene-2-carboxylic acid (54 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in DCM) gave the title compound (15 mg, 20%) as a white solid.

¹H NMR (400 MHz, DMSO-d₆) δ 1.49 (d, 3H, J=7.0 Hz), 2.30 (s, 3H), 2.96 (s, 3H), 3.28 (t, 4H, J=4.8 Hz), 3.79 (t, 4H, J=4.8 Hz), 5.13-5.20 (m, 1H), 7.21-7.26 (m, 3H), 7.29 (s, 1H), 7.45 (dd, 1H, J=9.1 Hz, 2.4 Hz), 7.75 (d, 1H, J=8.7 Hz), 7.84-7.88 (m, 2H), 8.34 (s, 1H), 8.80 (d, 1H, J=8.0 Hz), 9.00 (s, 1H).

LC/MS: m/z 468 (M+H)⁺; r.t.=2.56 min

### Example 25

### 6-AZETIDIN-1-YL-NAPHTHALENE-2-CARBOXYLIC ACID [(R)-4-(4-METHANE-SULFONYLAMINO-3-METHYLPHENYL)ETHYL]AMIDE

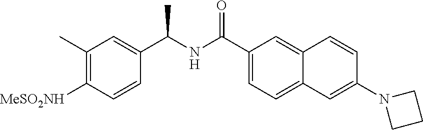

### 25A) 6-AZETIDIN-1-YL-NAPHTHALENE-2-CARBOXYLIC ACID METHYL ESTER

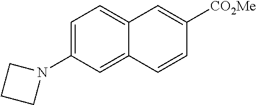

A flask containing 6-bromonaphthalene-2-carboxylic acid methyl ester (1 g, 4 mmol), palladium acetate (8.5 mg, 0.04 mmol), racemic BINAP (35 mg, 0.06 mmol) and cesium carbonate (1.721 g, 5.2 mmol) in anhydrous toluene (8 mL) was degassed with N₂ for 10 minutes. Azetidine (1 g, 20 mmol) was added, and the reaction was heated at 100° C. for 16 hours. After cooling, the reaction mixture was poured into saturated NaHCO₃ solution (100 ml) and extracted with EtOAc (3×50 ml). The combined organics were washed with brine (3×50 ml), dried (MgSO₄), filtered and concentrated. Flash chromatography (0 to 10% EtOAc in hexanes) gave the title compound (280 mg, 30%) as yellow crystals.

¹H NMR (400 MHz, DMSO-d₆) δ 2.34-2.41 (m, 2H), 3.86 (s, 3H), 3.98 (t, 4H, J=7.2 Hz), 6.67 (d, 1H, H=2.1 Hz), 6.88 (dd, 1H, J=8.8 Hz, 2.3 Hz), 7.68 (d, 1H, J=8.7 Hz), 7.81 (dd, 1H, J=8.7 Hz, 1.7 Hz), 7.91 (d, 1H, J=8.9 Hz), 8.42 (s, 1H).

LC/MS: m/z 242 (M+H)⁺; r.t.=3.63 min

### 25B) 6-AZETIDIN-1-YL-NAPHTHALENE-2-CARBOXYLIC ACID

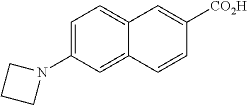 [0698] To a stirred solution of 6-azetidin-1-yl-naphthalene-2-carboxylic acid methyl ester (241 mg, 1 mmol) in tetrahydrofuran (15 mL) and ethanol (5 mL) was added a solution of lithium hydroxide (119 mg, 5 mmol) in water (5 mL). The reaction was stirred at 50° C. for 16 hours. After cooling the reaction mixture was diluted with water (100 mL) and then acidified to pH 7.0 with 2N HCl. The mixture was extracted with EtOAc (3×50 mL). The combined organics were dried (MgSO₄), filtered and concentrated to give the title compound (172 mg, 76%) as a yellow solid.

¹H NMR (400 MHz, DMSO-d₆) δ 2.34-2.41 (m, 2H), 3.97 (t, 4H, J=7.2 Hz), 6.67 (s, 1H), 6.87 (d, 1H, J=7.1 Hz), 7.66 (d, 1H, J=8.6 Hz), 7.80 (d, 1H, J=8.7 Hz), 7.89 (d, 1H, J=8.8 Hz), 8.38 (s, 1H).

LC/MS: m/z 228 (M+H)⁺; r.t.=2.92 min

### 25C) 6-AZETIDIN-1-YL-NAPHTHALENE-2-CARBOXYLIC ACID [(R)-1-(4-METHANE-SULFONYLAMINO-3-METHYLPHENYL)ETHYL]AMIDE

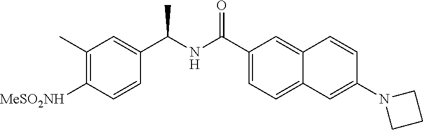

To a stirred solution of N-[4-((R)-1-aminoethyl)2-methylphenyl]methanesulfonamide (40 mg, 0.2 mmol) in anhydrous DMF (2 mL) was added a solution of 6-azetidin-1-yl-naphthalene-2-carboxylic acid (48 mg, 0.21 mmol), N-(3-dimethylaminopropyl)-N′-ethylcarbodiimide hydrochloride (40 mg, 0.21 mmol), 1-hydroxybenzotriazole hydrate (32 mg, 0.21 mmol), N,N-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 5% MeOH in DCM) gave the title compound (26 mg, 30%) as a white solid.

¹H NMR (400 MHz, DMSO-d₆) δ 1.48 (d, 3H, J=7.1 Hz), 2.30 (s, 3H), 232-2.38 (m, 2H), 2.95 (s, 3H), 3.95 (t, 4H, J=7.3 Hz), 5.14-5.20 (m, 1H), 6.67 (d, 1H, J=2.1 Hz), 6.87 (dd, 1H, J=8.8 Hz, 2.2 Hz), 7.20-7.27 (m, 3H), 7.65 (d, 1H, J=8.7 Hz), 7.80-7.83 (m, 2H), 8.31 (s, 1H), 8.74 (d, 1H, J=8.0 Hz), 9.00 (s, 1H).

LC/MS: m/z 438.1 (M+H)⁺; r.t.=3.09 min

### Example 26

### 6-PIPERIDIN-1-YL-NAPHTHALENE-2-CARBOXYLIC ACID [(R)-1-(4-METHANE-SULFONYL-AMINO-3-METHYLPHENYL)ETHYL]AMIDE

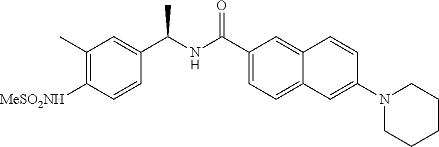

### 26A) 6-PIPERIDIN-1-YL-NAPHTHALENE-2-CARBOXYLIC ACID METHYL ESTER

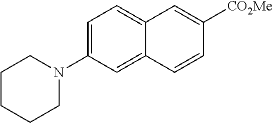

A flask containing 6-bromonaphthalene-2-carboxylic acid methyl ester (1 g, 4 mmol), palladium acetate (8.5 mg, 0.04 mmol), racemic BINAP (35 mg, 0.06 mmol) and cesium carbonate (1.721 g, 5.2 mmol) in anhydrous toluene (8 mL) was degassed with N₂ for 10 minutes. Piperidine (0.52 mL, 4.5 mmol) was added, and the reaction was heated at 100° C. for 16 hours. After cooling, the reaction mixture was poured into saturated NaHCO₃ solution (100 ml) and extracted with EtOAc (3×50 ml). The combined organics were washed with brine (3×50 ml), dried (MgSO₄), filtered and concentrated. Flash chromatography (0 to 10% EtOAc in Hexanes) gave the title compound (395 mg, 40%) as a cream solid.

¹H NMR (400 MHz, DMSO-d₆) δ 1.60-1.65 (m, 6H), 3.33-3.37 (m, 4H), 3.87 (s, 3H), 7.19 (d, 1H, J=2.2 Hz), 7.44 (dd, 1H, J=9.1 Hz, 2.5 Hz), 7.76 (d, 1H, J=8.7 Hz), 7.83 (dd, 1H, J=8.6 Hz, 1.7 Hz), 7.91 (d, 1H, J=9.2 Hz), 8.42 (s, 1H).

LC/MS: m/z 270 (M+H)⁺; r.t.=3.46 min

### 26B) 6-PIPERIDIN-1-YL-NAPHTHALENE-2-CARBOXYLIC ACID

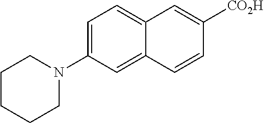

To a stirred solution of 6-piperidin-1-yl-naphthalene-2-carboxylic acid methyl ester (269 mg, 1 mmol) in tetrahydrofuran (15 mL) and ethanol (5 mL) was added a solution of lithium hydroxide (119 mg, 5 mmol) in water (5 mL). The reaction was stirred at 50° C. for 16 hours. After cooling the reaction mixture was diluted with water (100 mL) and then acidified to pH 7.0 with 2N HCl. The mixture was extracted with EtOAc (3×50 mL). The combined organics were dried (MgSO₄), filtered and concentrated to give the title compound (187 mg, 72%) as a yellow solid.

¹H NMR (400 MHz, DMSO-d₆) δ 1.60-1.65 (m, 6H), 3.34 (t, 4H, J=5.1 Hz), 7.18 (d, 1H, J=2.1 Hz), 7.42 (dd, 1H, J=9.2 Hz, 2.4 Hz), 7.72 (d, 1H, J=8.7 Hz), 7.82 (dd, 1H, 8.6 Hz, 1.6 Hz), 7.88 (d, 1H, 9.2 Hz), 8.39 (1H, s).

LC/MS: m/z 256 (M.+H)⁺; r.t.=2.43 min

### 26C) 6-PIPERIDIN-1-YL-NAPHTHALENE-2-CARBOXYLIC ACID [(R)-1-(4-METHANE-SULFONYLAMINO-3-METHYLPHENYL)ETHYL]AMIDE

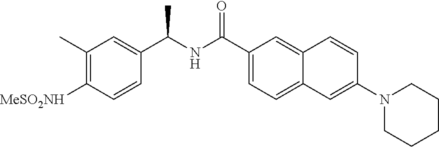

To a stirred solution of N-[4-((R)-1-aminoethyl)2-methylphenyl]methanesulfonamide (40 mg, 0.2 mmol) in anhydrous. DMF (2 mL) was added a solution of 6-piperidin-4-yl-naphthalene-2-carboxylic acid (54 mg, 0.21 mmol), N-(3-dimethylaminopropyl)-N′-ethylcarbodi-diisopropylethylamine (122 μL, 7.1 mmol) and 4-dimethylaminopyridine (1.1 mg, 0.008 mmol) in anhydrous DMF (2 mL). The reaction was stirred at room temperature for 16 hours, then poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in DCM) gave the title compound (4 mg, 5%) as a white solid.

¹H NMR (400 MHz, DMSO-d₆) δ 1.48 (d, 3H, J=7.0), 1.59-1.66 (m, 6H), 2.29 (s, 3H), 2.95 (s, 3H), 3.30 (s, 4H), 5.12-5.20 (m, 1H), 7.20-7.31 (m, 4H), 7.41 (dd, 1H, J=9.2 Hz, 2.4 Hz), 7.71 (d, 1H, J=8.7 Hz), 7.81-7.84 (m, 2H), 8.31 (s, 1H), 8.77 (d, 1H, J=8.0 Hz), 8.99 (s, 1H).

LC/MS: m/z 466 (M+H)⁺; r.t.=2.82 min

### Example 27

### 27A) N-((1R)-1-{3,5-DIFLUORO-4-[(METHYLSULFONYL)AMINO]PHENYL}ETHYL)-7-(TRIFLUOROMETHYL)QUINOLINE-3-CARBOXAMIDE

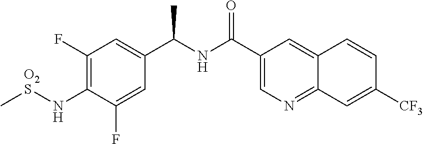

To a DMF (20 ml) solution of 7-(trifluoromethyl)quinoline-3-carboxylic acid (240 mg, 1.00 mmol), the compound of Example 3D (287 mg, 1.00 mmol) and HBTU (455 mg, 1.20 mmol) was added triethylamine (0.42 ml, 3.00 mmol) and the mixture was stirred for 2 hours at room temperature. The same procedure as described in Example 10 was performed to furnish the title compound (144 mg, 30% yield) as a white solid.

¹H NMR (270 MHz, DMSO-d₆) δ 1.53 (3H, d, J=7.3 Hz), 3.06 (3H, s), 5.17-5.31 (1H, s), 7.25-7.35 (2H, m), 7.96-8.03 (1H, m); 8.37-8.44 (1H, m), 8.46 (1H, s), 9.02-9.05 (1H, m), 9.30-9.37 (1H, m), 9.42-9.45 (1H, m), 9.51 (1H, br.s).

MS (ESI) m/z 472 (M−H)⁻, 474 (M+H)⁺.

### Example 28

### 28A) 6-ACETYL-N-((1R)-1-{3-METHYL-4-[(METHYLSULFONYL)AMINO]PHENYL}ETHYL)-QUINOLINE-2-CARBOXAMIDE

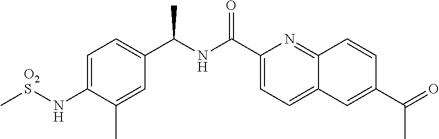

A suspension of the compound of Example 12 (80 mg, 0.35 mmol), palladium acetate (17 mg, 0.076 mmol), dppp (69 mg, 0.17 mmol), potassium carbonate (251 mg, 1.82 mmol) and butyl vinylether (758 mg, 7.6 mmol) in DMF (9 ml) and water (0.9 ml) was stirred at 130° C. for 30 minutes under microwave irradiation condition. Then the mixture was quenched with 2N-hydrochloride aqueous solution (5 ml) and stirred at room temperature for 1 hour. The mixture was diluted with water and extracted with ethyl acetate and the separated organic layer was dried over sodium sulfate, filtrated and concentrated under reduced pressure. The residue was purified through silica gel column chromatography eluting with ethyl acetate/hexane (1:1) to furnish the title compound (256 mg, 40% yield) as a white solid.

¹H NMR (300 MHz, DMSO-d₆) δ 1.58 (3H, d, J=6.6 Hz), (2.30 (3H, s), 2.75 (3H, s), 2.96 (3H, s), 5.16-5.25 (1H, m), 7.22-7.38 (3H, m), 8.20-8.35 (3H, m), 8.76 (1H, d, J=8.8 Hz), 8.84 (1H, s), 9.03 (1H, s), 9.23 (1H, d, J=8.1 Hz).

MS (ESI): m/z 426 (M+H)+.

### Example 29

### 6-TERT-BUTYL-N-((1R)-1-{3-METHYL-4-[(METHYLSULFONYL)AMINO]PHENYL}ETHYL)-QUINOLINE-3-CARBOXAMIDE

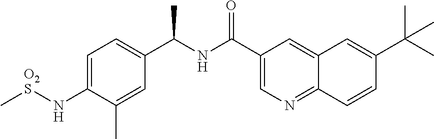

### 29A) ETHYL 6-TERT-BUTYLQUINOLINE-3-CARBOXYLATE

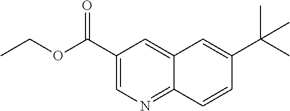

A mixture of ethyl 6-tert-butyl-4-chloroquinoline-3-carboxylate (2.57 g, 8.82 mmol) and triethylamine (2.46 ml, 17.6 mmol) in ethanol (100 ml) was hydrogenated over 5% palladium-carbon (250 mg) under balloon pressure for 10 hours. After the catalyst was filtered through a pad of celite and the filter cake was washed with methanol. The filtrate and washings were evaporated in vacuo and the residue was purified by column chromatography on silica gel with hexane/ethyl acetate (5:1) to furnish the title compound (2.02 g, 42% yield) as a slightly yellow oil.

¹H NMR (270 MHz, DMSO-d₆) δ 1.35-1.44 (12H, m), 4.36-4.47 (2H, m), 8.04-8.07 (2H, m), 8.15-8.18 (1H, m), 8.98-9.01 (1H, m), 9.25-9.28 (1H, m)

### 29B) 6-TERT-BUTYLQUINOLINE-3-CARBOXYLIC ACID

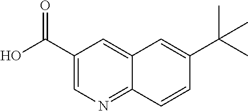

To a solution of the compound of Example 29A (2.02 g, 7.85 mmol) in ethanol (70 ml) was added 2M aqueous sodium hydroxide (15.7 ml) at room temperature. The mixture was stirred at 60° C. for 5 hours. Then the mixture was evaporated in vacuo, diluted with water (40 ml), neutralized to pH 5˜6 by 2M hydrochloride aqueous solution (8 ml). The precipitate solid was extracted with ethyl acetate and the organic solution was washed with brine, dried over sodium sulfate and concentrated in vacuo to give crude product, which was recrystallized from ethyl acetate and hexane to furnish the title compound (1.62 g, 90% yield) as a white solid.

¹H NMR (270 MHz, DMSO-d₆) [symbol] 1.41 (9H, s), 8.04 (2H, s), 8.12 (1H, s), 8.95 (1H, d, J=1.9 Hz), 9.26 (1H, d, J=1.9 Hz), 13.5 (1H, br.s).

### 29C) 6-TERT-BUTYL-N-((1R)-1-{3-METHYL-4-[(METHYLSULFONYL)AMINO]PHENYL}-ETHYL)QUINOLINE-3-CARBOXAMIDE

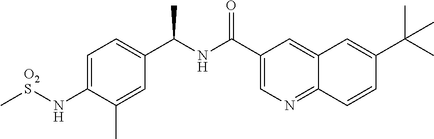

To a DMF (10 ml) solution of the compound of Example 29B (229 mg, 1.00 mmol), the compound of Example 1D (265 mg, 1.00 mmol) and HBTU (455 mg, 1.20 mmol) was added triethylamine (0.42 ml, 3.00 mmol) and the mixture was stirred for 5 hours at room temperature. The same procedure as described in Example 1G was performed to furnish the title compound (327 mg, 74% yield) as a white solid.

¹H NMR (270 MHz, DMSO-d₆) δ 1.41 (9H, s), 1.51 (3H, d, J=6.8 Hz), 2.31 (3H, s), 2.97 (3H, s), 5.12-5.25 (1H, m), 7.20-7.35 (3H, m), 7.95-8.07 (3H, m), 8.85 (1H, d, J=1.9 Hz), 9.01 (1H, s), 9.09 (1H, d, J=7.8 Hz), 9.22 (1H, d, J=1.9 Hz).

MS (ESI) m/z 438 (M−H)⁻, 440 (M+H)⁺.

### Example 30

### 6-(1-CYCLOPROPYL-1-HYDROXYETHYL)-N-((1R)-1-{3-METHYL-4-[(METHYL-SULFONYL)-AMINO]PHENYL}ETHYL)QUINOLINE-2-CARBOXAMIDE

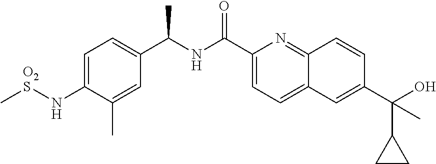

### 30A) 6-(1-CYCLOPROPYL-1-HYDROXYETHYL)-N-((1R)-1-{3-METHYL-4-[(METHYLSULFONYL)AMINO]PHENYL}ETHYL)QUINOLINE-2-CARBOXAMIDE

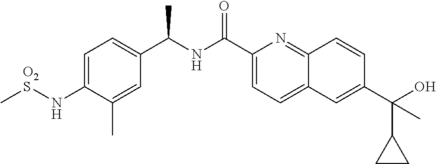

To a THF (10 ml) suspension of the compound of Example 28A (52 mg, 0.12 mmol) was added cyclopropylmagnesium bromide in 0.5M THF solution (1.22 ml, 0.611 mmol) at 0° C. dropwise over 15 minutes and then the mixture was stirred at room temperature for 3 hours. The reaction was quenched with saturated ammonium chloride aqueous solution (30 ml) and extracted with ethyl acetate. The organic layer was dried over sodium sulfate, filtrated, concentrated and purified through silica gel column chromatography eluting with ethyl acetate/hexane (1:1 to 2:1) and HPLC (used column was XTerra MS C18, 5 um, 30×50 mm) eluting with acetonitrile/0.01% ammonium aqueous solution (32:68 to 68:32) to furnish the title compound (6.0 mg, 11% yield) as a white solid.

¹H NMR (300 MHz, CDCl₃) [symbol] 0.45-0.68 (4H, m), 1.24-1.43 (1H, m), 1.59 (3H, s), 1.66 (3H, d, J=6.6 Hz); 1.82 (1H, s), 2.32 (3H, s), 3.01 (3H, s), 5.27-5.36 (1H, m), 6.25 (1H, s), 7.31-7.35 (2H, m), 7.43 (1H, d, J=8.8 Hz), 7.88-8.15 (3H, m), 8.26-8.35 (2H, m), 8.51 (1H, d, J=8.1 Hz).

MS (ESI): m/z 468 (M+H)⁺.

### Example 31

### N-((1R)-1-{3,5-DIFLUORO-4-[(METHYLSULFONYL)AMINO]PHENYL}ETHYL)-2-ISOPROPYL-QUINOLINE-6-CARBOXAMIDE

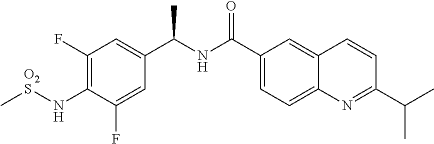

### 31A) METHYL 2-ISOPROPYLQUINOLINE-6-CARBOXYLATE

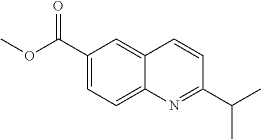

To a suspension of methyl quinoline-6-carboxylate (562 mg, 3 mmol, J. Org Chem. 2002, 67, 7890), isobutyric acid (396 mg, 4.5 mmol), silver nitrate (102 mg, 0.6 mmol) in 1M-sulfuric acid (3 ml) was added a solution of ammonium peroxodisulfate (1370 mg, 6 mmol) in water (3 ml) at 70° C. dropwise over 15 min. After the mixture was stirred at 70° C. for 1 hour, the reaction was quenched with saturated sodium bicarbonate aqueous solution (30 ml) extracted with ethyl acetate (30 ml×2). The organic layer was dried over sodium sulfate, concentrated and purified by silica gel column chromatography eluting with ethyl acetate/Hexane (1:50 to 1:20) to furnish the title compound (139 mg, 20% yield) as a white solid.

¹H NMR (300 MHz, CDCl₃) [symbol] 1.41 (6H, d, J=7.3 Hz), 3.23-3.35 (1H, m), 3.99 (3H, s), 7.41 (1H, d, J=8.8 Hz), 8.07 (1H, d, J=8.8 Hz), 8.18 (1H, d, J=8.1 Hz), 8.27 (1H, d, J=8.8 Hz), 8.56 (1H, s).

MS (ESI): m/z 230 (M+H)⁺.

### 31B) 2-ISOPROPYLQUINOLINE-6-CARBOXYLIC ACM

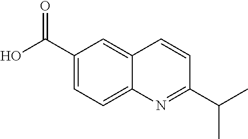

A methanol (3 ml) solution of the compound of Example 31A (139 mg, 0.61 mmol) and 2M-sodium hydroxide aqueous solution (1 ml, 2 mmol) was treated in the same procedure described in Example 8B to furnish the title compound (97 mg, 75% yield) as a white solid.

¹H NMR (300 MHz, CDCl₃) [symbol] 1.42 (6H, d, J=7.3 Hz), 3.29-3.39 (1H, m), 7.44 (1H, d, J=8.6 Hz), 8.15 (1H, d, J=9.2 Hz), 8.23 (1H, d, J=8.6 Hz), 8.35 (1H, d, J=9.2 Hz), 8.66 (1H, s).

MS (ESI): m/z 216 (M+H)⁺.

### 31C) N-((1R)-1-{3,5-DIFLUORO-4-[(METHYLSULFONYL)AMINO]PHENYL}ETHYL)-2-ISOPROPYLQUINOLINE-6-CARBOXAMIDE

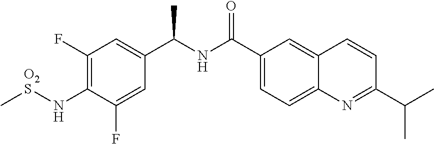

A acetonitrile (3 ml) solution of the compound of Example 31B (97 mg, 0.45 mmol), triethylamine (0.19 ml, 1.35 mmol), the compound of Example 3D (129 mg, 0.45 mmol) and HBTU (205 mg, 0.54 mmol) was treated in the same procedure described in Example 1G. The crude residue was applied to a silica gel column chromatography and eluted with hexane/ethyl acetate (1:1) and HPLC (used column was XTerra MS C18, 5 um, 30×50 mm) eluting with acetonitrile/0.01% ammonium aqueous solution (4:96 to 96:4) to furnish the title compound (43 mg, 22% yield) as a white, solid.

¹H NMR (300 MHz, CD₃OD) [symbol] 1.40 (6H, d, J=7.3 Hz), 1.60 (3H, d, J=6.6 Hz), 3.08 (3H, s), 3.23-3.35 (1H, m), 5.25 (1H, q, J=6.6 Hz), 7.16 (2H, d, J=8.8 Hz), 7.57 (1H, d, J=8.1 Hz), 8.06 (1H, d, J=8.8 Hz), 8.13-8.20 (1H, m), 8.38 (1H, d, J=8.8 Hz), 8.41-8.43 (1H, m).

MS (ESI): m/z 448 (M+H)⁺.

### Example 32

### N-((1R)-1-{2-FLUORO-5-METHYL-4-[(METHYLSULFONYL)AMINO]PHENYL}ETHYL)-2-(TRIFLUOROMETHYL)QUINOLINE-6-CARBOXAMIDE

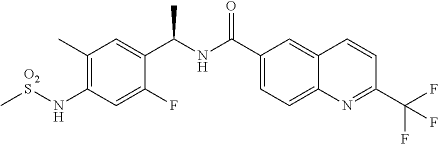

To a CH₂Cl₂ (20.0 ml) solution of carboxylic acid (70 mg, 0.29 mmol), thionyl chloride (1.0 ml) and DMAP (˜5.0 mg) were added and the mixture was stirred for 1 h at room temperature. Then, solvent and thionyl chloride were removed under reduced pressure to give the white solid, which was used for further reaction without purification. Then, to the pyridine (5 ml) solution of an amine (71 mg, 0.429 mmol), the CH₂Cl₂ (20 ml) solution of the acid chloride was added and the mixture was stirred for 1 h at room temperature. Then, the solvent was removed under reduced pressure to give the residue which was crystallized from ethyl acetate-hexane to give the title compound in 20% yield as a white solid ¹H NMR (300 MHz, CDCl₃) δ 1.67 (3H, d, J=7.3 Hz), 2.26 (3H, s), 3.05 (3H, s), 5.41 (1H, t, J=7.8 Hz), 6.24 (1H, s), 6.78 (1H, d, J=7.3 Hz), 7.23-7.33 (2a m), 7.76-7.83 (1H, m), 8.15 (1H, d, J=8.8 Hz), 8.28-8.48 (3H, m).

MS (ESI): m/z 470 (M+H)⁺.

### Example 33

### 7-TERT-BUTYL-N-((1R)-1-{3-METHYL-4-[(METHYLSULFONYL)AMINO]PHENYL}ETHYL)-QUINOLINE-3-CARBOXAMIDE

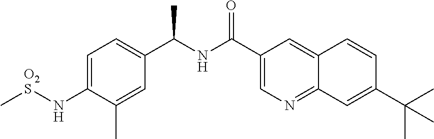

### 33A) ETHYL 7-TERT-BUTYL-4-CHLOROQUINOLINE-3-CARBOXYLATE

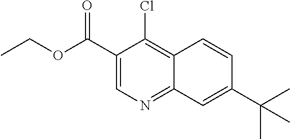

A mixture of ethyl 7-tert-butyl-4-oxo-1,4-dihydroquinoline-3-carboxylate (4.54 g, 16.61 mmol) in phosphorus oxychloride (60 ml) was heated at 120° C. for 3 hours. After the solvent was evaporated in vacuo, the residue was diluted with dichloromethane. The organic solution was poured into 27% ammonia water-ice carefully and stirred for 15 minutes. The aqueous layer was extracted with dichloromethane (×3) and the organic layer was washed with water, brine, dried over sodium sulfate and concentrate in vacuo to crude product. The crude product was purified by column chromatography on silica gel (350 g) with hexane-ethyl acetate (8:1-6:1) to furnish the title compound (4.82 g, 99% yield) as a colorless oil

¹H NMR (270 MHz, DMSO-d₆) δ 1.35-1.44 (3H, m), 1.42 (9H, s), 4.38-4.49 (2H, m), 7.97-8.06 (2H, m), 8.30-8.36 (1H, m), 9.15 (1H, s).

### 33B) ETHYL 7-TERT-BUTYLQUINOLINE-3-CARBOXYLATE

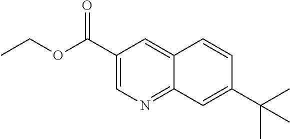

A mixture of the compound of Example 33A (2.06 g, 7.06 mmol) and triethylamine (1.97 ml, 21.2 mmol) in ethanol (70 ml) was hydrogenated over 5% palladium-carbon (300 mg) under balloon pressure for 1.5 hours. After the catalyst was filtered through a pad of celite and the filter cake was washed with methanol. The filtrate and washings were evaporated in vacuo and the residue was purified by column chromatography on silica gel with hexane/ethyl acetate (8:1) to furnish the title compound (1.68 g, 92.5% yield) as a yellow oil.

¹H NMR (270 MHz, CDCl₃) δ 1.43-1.51 (3H, m), 1.45 (9H, s), 4.48 (2H, q, J=7.0 Hz), 6.69-7.75 (1H, m), 7.85-7.91 (1H, m), 8.13 (1H, s), 8.79-8.82 (1H, m), 9.41-9.44 (1H, m).

### 33C) 7-TERT-BUTYLQUINOLINE-3-CARBOXYLIC ACID

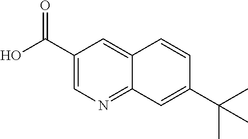

A mixture of the compound of Example 33B (1.63 g, 6.33 mmol) in 2M sodium hydroxide solution (12.67 mmol, 6.33 ml) and ethanol (50 ml) was heated at 75° C. for 2 hours. The same procedure as described in Example 1F was performed to give the title compound (1.27 g, 87.7% yield) as a white solid.

LM-MS retention time: 2.76 min (Neutral full range)

MS (ESI) m/z 228.2 (M−H)⁻, 230.2 (M+H)⁺.

### 33D) 7-TERT-BUTYL-N-((1R)-1-{3-METHYL-4-[(METHYLSULFONYL)AMINO]PHENYL}-ETHYL)QUINOLINE-3-CARBOXAMIDE

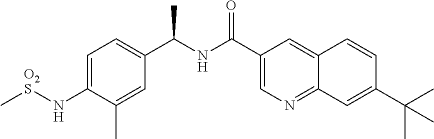

To a DMF (10 ml) solution of the compound of Example 1D (265 mg, 1.00 mmol), the compound of Example 33C (230 mg, 1.00 mmol) and HBTU (455 mg, 1.20 mmol) was added triethylamine (0.418 ml, 3.00 mmol) and the mixture was stirred for 4 hours at room temperature. The same procedure as described in Example 1G was performed to give the title compound (322 mg, 73.3% yield) as a white solid.

¹H NMR (270 MHz, DMSO-d₆) δ 1.42 (9H, s), 1.52 (3H, d, J=7.3 Hz), 2.31 (3H, s), 2.97 (3H, s), 5.13-5.26 (1H, m), 7.21-7.34 (3H, m), 7.79-7.86 (1H, m), 7.98 (1H, s), 8.01-8.07 (1H, m), 8.80-8.84 (1H, m), 9.01 (1H, s), 9.09-9.15 (1H, m), 9.26-9.30 (1H, m).

MS (ESI) m/z 438.25 (M−H)⁻, 440.23 (M+H)⁺.

### Example 34

### N-((1R)-1-{3-FLUORO-4-[(METHYLSULFONYL)AMINO]PHENYL}ETHYL)-7-(TRIFLUORO-METHYL)QUINOLINE-3-CARBOXAMIDE

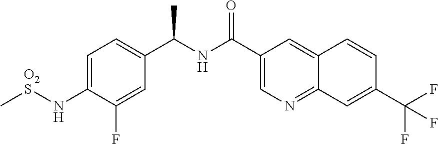

To a DMF (10 ml) solution of the compound of N-{4-[(1R)-1-aminoethyl]-2-fluorophenyl}methanesulfonamide hydrochloride (269 mg, 1.00 mmol), 7-(trifluoromethyl)quinoline-3-carboxylic acid (241 mg, 1.00 mmol) and HBTU (455 mg, 1.20 mmol) was added triethylamine (0.7 ml, 5.00 mmol) and the mixture was stirred for 5 hours at room temperature. The same procedure as described in Example 1G was performed to give the title compound (319 mg, 70.0% yield) as a white solid.

¹H NMR (270 MHz, DMSO-d₆) δ 1.53 (3H, d, J=6.8 Hz), 3.02 (3H, s), 5.17-5.31 (1H, m), 7.23-7.45 (3H, m), 7.94-8.02 (1H, m), 8.35-8.49 (2H, m), 9.01 (1H, s), 9.27-9.35 (1H, m), 9.42 (1H, s), 9.55 (1H, s).

MS (ESI) m/z 454.19 (M−H)⁻, 456.20 (M+H)⁺.

### Example 38

### 2-ISOPROPYL-N-((1R)-1-{3-METHYL-4-[(METHYLSULFONYL)AMINO]PHENYL}ETHYL)-QUINOLINE-6-CARBOXAMIDE

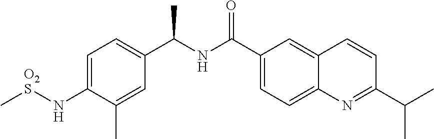

A DMF (4 ml) solution of the compound of Example 1D (92 mg, 0.35 mmol), triethylamine (0.15 ml, 1.1 mmol), the compound of Example 31B (75 mg, 0.35 mmol) and HBTU (159 mg, 0.42 mmol) was treated in the same procedure described in Example 1G. The crude residue was crystallized from ethyl acetate-hexane to furnish the title compound (100 mg, 67% yield) as a white solid.

¹H NMR (300 MHz, DMSO-d₆) [symbol] 1.31 (6H, d, J=8.8 Hz), 1.50 (3H, d, J=6.6 Hz), 2.30 (3H, s), 2.96 (3H, s), 3.19-3.30 (1H, m), 5.15-5.24 (1H, m), 7.21-7.32 (3H, m), 7.56 (1H, d, J=8.8 Hz), 7.99 (1H, d, J=8.8 Hz), 8.16 (1H, d, J=6.6 Hz), 8.39 (1H, d, J=8.8 Hz), 8.49 (1H, s), 8.98-9.00 (2H, m).

MS (ESI): m/z 426 (M+H)⁺.

### Example 36

### 7-TERT-BUTYL-N-((1R)-1-{3,5-DIFLUORO-4-[(METHYLSULFONYL)AMINO]PHENYL}ETHYL)-QUINOLINE-3-CARBOXAMIDE

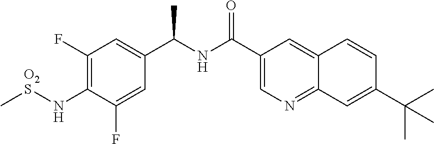

To a DMF (10 ml) solution of the compound of Example 3D (287 mg, 1.00 mmol), the compound of Example 33C (229 mg, 1.00 mmol) and HBTU (455 mg, 1.20 mmol) was added triethylamine (0.42 ml, 3.00 mmol) and the mixture was stirred for 4 hours at room temperature. The same procedure as described in Example 1G was performed to give the title compound (362 mg, 78.4% yield) as a white solid.

¹H NMR (270 MHz, DMSO-d₆) δ 1.42 (9H, s), 1.53 (3H, d, J=6.5 Hz), 3.06 (3H, s), 5.15-5.30 (1H, m), 7.24-7.33 (2H, m), 7.80-7.87 (1H, m), 7.97-8.01 (1H, m), 8.03-8.09 (1H, m), 8.82-8.87 (1H, m), 9.15-9.22 (1H, m), 9.27-9.30 (1H, m), 9.49 (1H, br.s).

MS (ESI) m/z 460.06 (M−H)⁻, 462.05 (M+H)⁺.

### Example 37

### N-((1R)-1-{2-FLUORO-5-METHYL-4-[(METHYLSULFONYL)AMINO]PHENYL}ETHYL)-2-ISOPROPYLQUINOLINE-6-CARBOXAMIDE

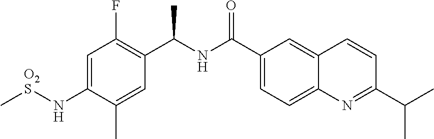

A DMF (3 ml) solution of the compound of Example 13D (100 mg, 0.35 mmol), triethylamine (0.15 ml, 1.06 mmol), the compound of Example 31B (76 mg, 0.35 mmol), HOBt.H₂O (60 mg, 0.39 mmol) and WSC (102 mg, 0.53 mmol) was treated in the same procedure described in Example 17B. The crude residue was applied to a silica gel column chromatography and eluted with hexane/ethyl acetate (2:3) to furnish the title compound (48 mg, 30% yield) as a white solid.

¹H NMR (300 MHz, DMSO-d₆) δ 1.40 (6H, d, J=7.3 Hz), 1.65 (3H, d, J=7.3 Hz), 2.25 (3H, s), 3.05 (3H, s), 3.23-3.30 (1H, m), 5.38-5.45 (1H, m), 6.22 (1H, brs), 6.72 (1H, d, J=8.1 Hz), 7.22-7.35 (2H, m), 7.41 (1H, d, J=8.8 Hz), 7.98-8.03 (1H, m), 8.09 (1H, d, J=8.8 Hz), 8.17 (1H, d, J=8.1 Hz), 8.25-8.27 (1H, m).

MS (ESI): m/z 444 (M+H)⁺.

### Example 38

### 7-ISOPROPYL-N-((1R)-1-{3-METHYL-4-[(METHYLSULFONYL)AMINO]PHENYL}ETHYL)-QUINOLINE-3-CARBOXAMIDE

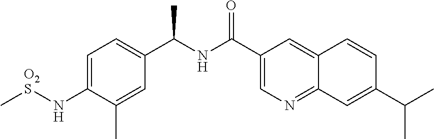

### 38A) ETHYL 4-CHLORO-7-ISOPROPYLQUINOLINE-3-CARBOXYLATE

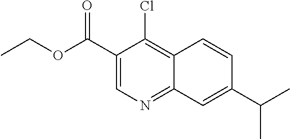

A mixture of ethyl 7-isopropyl-4-oxo-1,4-dihydroquinoline-3-carboxylate (5.00 g, 19.3 mmol) in phosphorus oxychloride (100 ml) was heated at 120° C. for 3 hours. The same procedure as described in Example 33A was performed to give the title compound (5.27 g, 98% yield) as a slightly yellow oil.

¹H NMR (270 MHz, CDCl₃) δ 1.38 (6H, d, J=7.3 Hz), 1.47 (3H, t, J=7.3 Hz), 3.10-3.23 (1H, m), 4.50 (2H, q, J=7.3 Hz), 7.58-7.65 (1H, m), 7.95-7.99 (1H, m), 8.34 (1H, d, J=8.4 Hz), 9.19 (1H, s).

MS (ESI) m/z 278.02 (M+H)⁺.

### 38B) ETHYL 7-ISOPROPYLQUINOLINE-3-CARBOXYLATE

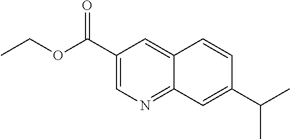

A mixture of the compound of Example 38A (5.20 g, 19 mmol) and triethylamine (7.83 ml, 56.2 mmol) in ethanol (150 ml) was hydrogenated over 5% palladium-carbon (600 mg) at room temperature under balloon pressure for total 3 hours. The same procedure as described in Example 33B was performed to give the title compound (3.97 g, 86% yield) as a slightly yellow oil.

¹H NMR (270 MHz, CDCl₃) δ 1.38 (6H, d, J=6.6 Hz), 1.47 (3H, t, J=7.0 Hz), 3.10-3.22 (1H, m), 4.48 (2H. q, J=7.0 Hz), 7.51-7.57 (1H, m), 7.88 (1H, d, J=8.1 Hz), 7.98-8.01 (1H, m), 8.79-8.83 (1H, m), 9.41-9.44 (1H, m).

### 38C) 7-ISOPROPYLQUINOLINE-3-CARBOXYLIC ACID

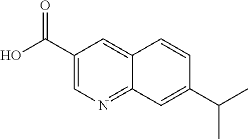

The solution of the compound of Example 38B (3.96 g, 16.3 mmol) in ethanol (100 ml) and 2M sodium hydroxide aqueous solution (16.3 ml, 32.6 mmol) was heated at 80° C. for 3 hours. The same procedure as described in Example 1F was performed to give the title compound (3.25 g, 93% yield) as a white solid.

¹H NMR (270 MHz, DMSO-d₆) δ 1.33 (6H, d, J=6.5 Hz), 3.10-3.24 (1H, m), 7.62-7.70 (1H, m), 7.92 (1H, br.s), 8.12 (1H, d, J=8.6 Hz), 8.90-8.95 (1H, m), 9.26-9.33 (1H, m)

### 38D) 7-ISOPROPYL-N-((1R)-1-{3-METHYL-4-[(METHYLSULFONYL)AMINO]PHENYL}ETHYL)-QUINOLINE-3-CARBOXAMIDE

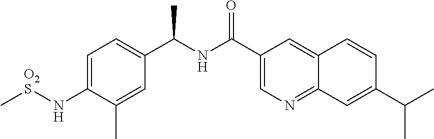

To a DMF (10 ml) solution of the compound of Example 1D (265 mg, 1.00 mmol), the compound of Example 38C (215 mg, 1.00 mmol) and HBTU (455 mg, 1.20 mmol) was added triethylamine (0.418 ml, 3.00 mmol) and the mixture was stirred for 4 hours at room temperature. The same procedure as described in Example 1G was performed to give the title compound (310 mg, 72.7% yield) as a white solid.

¹H NMR (270 MHz, DMSO-d₆) [symbol] 1.33 (6H, d, J=6.5 Hz), 1.52 (3H, d, J=7.3 Hz), 2.31 (3H, s), 2.97 (3H, s), 3.08-3.23 (1H, m), 5.13-5.27 (1H, m), 7.21-7.34 (3H, m), 7.61-7.68 (1H, m), 7.90 (1H, s), 8.00-8.07 (1H, m), 8.80-8.84 (1H, m), 9.01 (1H, s), 9.08-9.15 (1H, m), 9.25-9.29 (1H, m).

MS (ESI) m/z 424.24 (M−H)⁻, 426.19 (M+H)⁺.

### Example 39

### N-((1R)-1-{3,5-DIFLUORO-4-[(METHYLSULFONYL)AMINO]PHENYL}ETHYL)-7-ISOPROPYL-QUINOLINE-3-CARBOXAMIDE

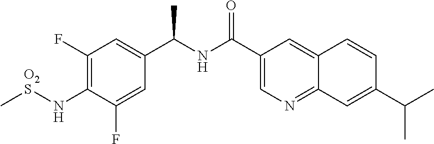

To a DMF (10 ml) solution of the compound of Example 3D (286 mg, 1.00 mmol), the compound of Example 38C (215 mg, 1.00 mmol) and HBTU (378 mg, 1.00 mmol) was added triethylamine (0.42 ml, 3.00 mmol) and the mixture was stirred for 4 hours at room temperature. The same procedure as described in Example 1a was performed to give the title compound (351 mg, 78.6% yield) as a white solid.

¹H NMR (270 MHz, DMSO-d₆) δ1.33 (6H, d, J=7.3 Hz), 1.52 (3H, d, J=7.3 Hz), 3.06 (3H, s), 3.09-3.23 (1H, m), 5.15-5.30 (1H, m), 7.24-7.33 (2H, m), 7.62-7.69 (1H, m), 7.91 (1H, s), 8.02-8.09 (1H, m), 8.82-8.87 (1H, m), 9.14-9.21 (1H, m), 9.26-9.30 (1H, m), 9.50 (1H, s)

MS (ESI) m/z 446.22 (M−H)⁻, 448:13 (M+H)⁺.

### Example 40

### 6-TERT-BUTYL-N-((1R)-1-{4-METHYL-5-[(METHYLSULFONYL)AMINO]PYRIDIN-2-YL}ETHYL)-2-NAPHTHAMIDE

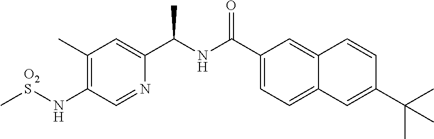

### 40A) N-(6-CHLORO-4-METHYLPYRIDIN-3-YL)METHANESULFONAMIDE

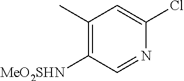

A mixture of 3-amino-6-chloro-4-picoline (2.0 g, 14.0 mmol) and methanesulfonyl chloride (1.93 g, 16.8 mmol) in pyridine (140 ml) was stirred for 2 hours at room temperature. The resulting mixture was quenched with 2 M HCl aqueous solution and diluted with EtOAc. The separated organic phase was washed with 2 M HCl aqueous solution, dried over magnesium sulfate, concentrated to give crude. It was diluted with EtOAc and extracted with 2 M sodium hydroxide aqueous solution. The separated basic phase was acidified with 2 M HCl aqueous solution to give precipitates, which were collected and rinsed with water, dried in vacuo to afford the title compound (2.42 g, 78%) as a solid.

¹H NMR (DMSO-d₆) δ 2.33 (3H, s), 3.05 (3H, s), 7.47 (1H, s), 8.24 (1H, s), 9.44 (1H, brs).

MS (ESI): m/z 221 (M+H)⁺, 219 (M−H)⁻.

### 40B) N-(6-CYANO-4-METHYLPYRIDIN-3-YL)METHANESULFONAMIDE

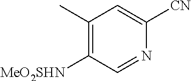

A test tube suitable for microwave use was charged with the compound of Example 40A (2.42 g, 10.9 mmol), zinc cyanide (1.61 g, 13.7 mmol) and tetrakis(triphenylphosphine)palladium(0) (1.27 g, 1.09 mmol) in DMF (10.9 ml). The mixture was subjected to microwave irradiation at 100° C. with stirring for 30 minutes. Then, the mixture was diluted with toluene/EtOAc (1:10) and the precipitates were filtered off. The filtrate was basified with 2 M sodium hydroxide aqueous solution. And then the separated aqueous phase was acidified with 2M HCl aqueous solution, extracted with EtOAc (×2), dried over magnesium sulfate, concentrated. The residue was recrystallized from EtOAc/Hexane to give crystals. A mixture of the obtained crystals and zinc cyanide (1.28 g, 10.9 mmol) and tetrakis(triphenylphosphine)palladium(0) (0.64 g, 0.55 mmol) in DMF (10.9 ml) was subjected to microwave irradiation at 100° C. with stirring for 30 minutes. It was irradiated at 110° C. with stirring for an additional 20 minutes. The resulting mixture was treated with same manner as above to afford the title compound (792 mg, 34%) as pale red solids.

¹H NMR (DMSO-d₆) δ 2.37 (3H, s), 3.17 (3H, s), 7.99 (1H, s), 8.61 (1H, s), 9.82 (1H, s).

MS (ESI): m/z 212 (M+H)⁺, 210 (M−H)⁻.

### 40C) N-(6-ACETYL-4-METHYLPYRIDIN-3-YL)METHANESULFONAMIDE

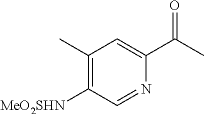

To a solution of the compound of Example 40B (1.57 g, 7.43 mmol) in THF (37.2 ml) was added dropwise a THF solution of methyl magnesium bromide (27.2 ml, 22.3 mmol) at 0° C. with stirring. After being stirred for 0.5 hours at 0° C., the mixture was stirred for 1.5 hours at room temperature. The reaction mixture was quenched with brine (5 ml), and then diluted with EtOAc and MeOH. To this solution was added NaCl and stirred for 1 hour. The obtained yellow clear solution was filtered through a pad of celite with EtOAc and MeOH. The filtrate was concentrated and purified by silica gel chromatography eluting with EtOAc/hexane (3:2) to afford the title compound (1.27 g, 75%) as orange brown solids.

¹H NMR (300 MHz, DMSO-d₆) δ 2.38 (3H, s), 2.59 (3H, s), 3.12 (3H, s), 7.86 (1H, s), 8.56 (1H, s), 9.65 (1H, brs).

MS (ESI): m/z 229 (M+H)⁺, 227 (M−H)⁻.

### 40D) N-[4-METHYL-6-((1R)-1-{[(1R)-1-PHENYLETHYL]AMINO}ETHYL)PYRIDIN-3-YL]METHANESULFONAMIDE

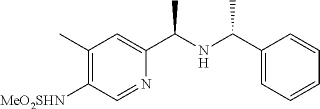

A mixture of the compound of Example 40C (1.27 g, 5.56 mmol), (R)-1-phenylethanamine (0.81 g, 6.68 mmol), titanium (IV) chloride (30 ml) and tetrahydrofuran (30 ml) was stirred at room temperature for 4 hours. The obtained imine solution was added to a solution of sodium borohydride (630 mg, 16.7 mmol) in MeOH (63 ml) at −8° C. After 3 h the reduction was not complete, so an additional sodium borohydride (630 mg, 16.7 mmol) was added and stirred for 7.5 hours at room temperature. The obtained mixture was quenched with water (100 ml) and stirred for 1 hour, filtered through a pad of celite with EtOAc, the separated aqueous phase was extracted with EtOAc. The combined organic phases were dried over magnesium sulfate, concentrated to give yellow amorphous (1.89 g, 85% d.e. by HPLC-UV). It was purified by silica gel chromatography eluting with acetone/hexane (1:1), then purified by preparative thin layer chromatography (Merck, silica gel 60 F254, 1 mm) eluting with acetone/hexane (1:1) to give the title compound (747 mg, 40% yield, 98% d.e. by HPLC-UV) as yellow oil.

¹H NMR (300 MHz, DMSO-d₆) [symbol] 1.12-1.22 (6H, m), 2.33 (3H, s), 3.03 (3H, s), 3.28-3.52 (2H, m), 7.09-7.43 (6H, m), 8.30 (1H, s), 9.25 (1H, s)

MS (ESI): m/z 334 (M+H)⁺, 332 (M−H).

### 40E) N-{6-[(1R)-1-AMINOETHYL]-4METHYLPYRIDIN-3-YL}METHANESULFONAMIDE

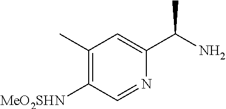

To a mixture of the compound of Example 40D (747 mg, 2.24 mmol) in EtOH (22 ml) was added 10% Pd—C (187 mg) and ammonium formate (4.24 g, 67 mmol) at room temperature under N₂. The resulting mixture was stirred for 2 hours at 65° C. An additional 10% Pd—C (63 mg) and ammonium formate (1.30 g, 21 mmol) was added and stirred for 0.5 hours at 65° C. The reaction mixture was cooled to room temperature and filtered through a celite pad. The filtrate was concentrated and purified by amino bounded silica gel chromatography eluting with MeOH/DCM (1:10), then recrystallized from MeOH/EtOAc/hexane to afford the title compound (225 mg, 44% yield) as white solids.

¹H NMR (300 MHz, DMSO-d₆) δ 1.31 (3H, d, J=6.6 Hz), 2.23 (3H, s), 2.86 (3H, s), 3.99-4.10 (1H, m), 5.61 (2H, brs), 7.24 (1H, s), 8.30 (1H, s).

MS (ESI): m/z 230 (M+H)⁺, 228 (M−H)⁻.

### 40F) 6-TERT-BUTYL-N-((1R)-1-{4-METHYL-5-[(METHYLSULFONYL)AMINO]PYRIDIN-2-YL}ETHYL)-2-NAPHTHAMIDE

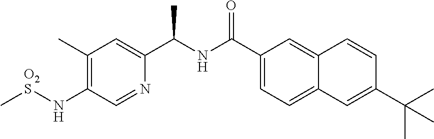

To a DMF (5.0 ml) solution of the compound of Example 40E (100 mg, 0.436 mmol), the compound of Example 1F (99.6 mg, 0.436 mmol) and HBTU (198 mg, 0.523 mmol) was added triethylamine (0.18 ml, 1.31 mmol) and the mixture was stirred for 5 hours at room temperature. The same procedure as described in Example 1G was performed to give the title compound (158 mg, 82.4% yield) as a white solid.

¹H NMR (270 MHz, DMSO-d₆) δ 1.40 (9H, s), 1.54 (3H, d, J=7.3 Hz), 2.33 (3H, s), 3.04 (3H, s), 5.15-5.30 (1H, m), 7.36 (1H, s), 7.69-7.75 (1H, m), 7.88-8.01 (4H, m), 8.37 (1H, s), 8.50 (1H, s), 8.91-8.97 (1H, m), 9.31 (1H, s).

MS (ESI) m/z 438.27 (M−H)⁻, 440.20 (M+H)⁺.

### Example 41

### 7-TERT-BUTYL-N-((1R)-1-{4-[(METHYLSULFONYL)AMINO]PHENYL}ETHYL)QUINOLINE-3-CARBOXAMIDE

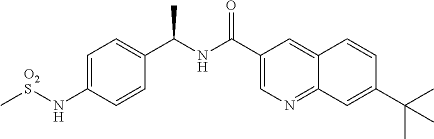

To a DMF (3.0 ml) solution of N-{4-[(1R)-1-aminoethyl]phenyl}methanesulfonamide hydrochloride (200 mg, 0.798 mmol), the compound of Example 33C (183 mg, 0.798 mmol) and HBTU (363 mg, 0.957 mmol) was added triethylamine (0.33 ml, 2.39 mmol) and the mixture was stirred for 5 hours at room temperature. The same procedure as described in Example 1G was performed to give the title compound (178 mg, 52.4% yield) as a white solid.

¹H NMR (270 MHz, DMSO-d₅) δ 1.42 (9H, s), 1.52 (3H, d, J=7.3 Hz), 2.97 (3H, s), 5.15-5.30 (1H, m), 7.16-7.23 (2H, m), 7.39-7.45 (2H, m), 7.79-7.86 (1H, m), 7.96-8.08 (2H, m), 8.82 (1H, s), 9.09-9.16 (1H, m), 9.27 (1H, s), 9.68 (1H, s),

MS (ESI) m/z 423.92 (M−H)⁻, 425.94 (M+H)⁺.

### Example 42

### 7-TERT-BUTYL-4-METHYL-N-((1R)-1-{3-METHYL-4-[(METHYLSULFONYL)AMINO]PHENYL}-ETHYL)QUINOLINE-3-CARBOXAMIDE

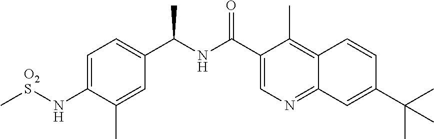

### 42A) ETHYL 7-TERT-BUTYL-4-METHYLQUINOLINE-3-CARBOXYLATE

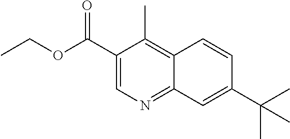

A mixture of the compound of Example 33A (500 mg, 1.71 mmol), 50% w/w methylboronic acid (0.719 ml, 2.57 mmol), tetrakis(triphenylphosphine)palladium (198 mg, 0.17 mmol) and potassium carbonate (710 mg, 5.14 mmol) in anhydrous DMF (15 ml) was heated at 140° C. for 16 hours. The same procedure as described in Example 11A was performed to give the title compound (425 mg, 91.4% yield) as a white solid.

¹H NMR (270 MHz, DMSO-d₆) δ 1.34-1.43 (3H, m), 1.40 (9H, s), 2.90 (3H, s), 4.33-4.45 (2H, m), 7.80-7.98 (2H, m), 8.22 (1H, d, J=8.4 Hz), 9.08 (1H, s)

MS (ESI) m/z 272.27 (M+H)⁺.

### 42B) 7-TERT-BUTYL-4-METHYLQUINOLINE-3-CARBOXYLIC ACID

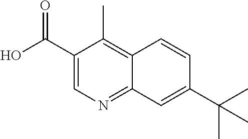

The solution of the compound of Example 42A (420 mg, 1.55 mmol) in ethanol (10 ml) and 2M sodium hydroxide aqueous solution (1.55 ml, 3.10 mmol) was heated at 80° C. for 3 hours. After the solvent was evaporated in vacuo, the residue was dissolved with water and the aqueous layer was neutralized with 2M hydrochloric acid aqueous solution with ice-cooling. The aqueous layer was extracted with ethyl acetate (×3) and the combined solution was washed with brine, dried over sodium sulfate and concentrated in vacuo to give crude product, which was recrystallized from ethyl acetate-hexane to furnish the title compound (280 mg, 74.4% yield) as a white solid.

¹H NMR (270 MHz, DMSO-d₆) δ 1.41 (9H, s), 2.94 (3H, s), 7.79-7.86 (1H, m), 7.93-7.96 (1H, m), 8.23 (1H, d, J=8.6 Hz), 9.11 (1H, s), 13.4 (1H, br.s)

### 42C) 7-TERT-BUTYL-4-METHYL-N-((1R)-1-{3-METHYL-4-[(METHYLSULFONYL)-AMINO]PHENYL}ETHYL)QUINOLINE-3-CARBOXAMIDE

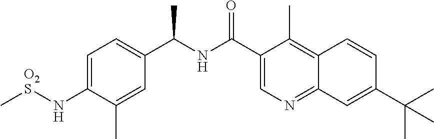

To a DMF (10 ml) solution of the compound of Example 1D (200 mg, 0.755 mmol), the compound of Example 42B (184 mg, 0.755 mmol) and HBTU (344 mg, 0.906 mmol) was added triethylamine (0.316 ml, 2.27 mmol) and the mixture was stirred, for 5 hours at room temperature. The same procedure as described in Example 1G was performed to give the title compound (283 mg, 82.6% yield) as a white solid.

¹H NMR (270 MHz, DMSO-d₆) δ 1.41 (9H, s), 1.47 (3H, d, J=7.3 Hz), 2.33 (3H, s), 2.65 (3H, s), 2.98 (3H, s), 5.10-5.24 (1H, m), 7.25-7.33 (3H, m), 7.77-7.83 (1H, m), 7.91-7.94 (1H, m), 8.09-8.15 (1H, m), 8.74 (1H, s), 8.97-9.05 (2H, m)

MS (ESI) m/z 452.16 (M−H)⁻, 454.11 (M+H)⁺.

### Example 43

### N-((1R)-1-{2-FLUORO-5-METHYL-4-[(METHYLSULFONYL)AMINO]PHENYL}ETHYL)-7-(TRIFLUOROMETHYL)QUINOLINE-3-CARBOXAMIDE

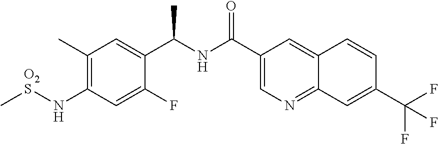

To a CH₂Cl₂ (20.0 ml) solution of 7-(trifluoromethyl)quinoline-3-carboxylic acid (300 mg, 1.24 mmol), thionyl chloride (1.0 ml) and DMAP (15.2 mg, 0.124 mmol) were added and the mixture was stirred for 1 h at room temperature. Then, solvent and thionyl chloride were removed under reduced pressure to give the white solid, which was used for further reaction without purification. Then, to the pyridine (5 ml) solution of an amine (Example 13D) (352 mg, 1.24 mmol), the CH₂Cl₂ (20 ml) solution of the acid chloride was added and the mixture was stirred for 1 h at room temperature. The same procedure as described in Example 32 was performed to give the title compound (152 mg, 26% yield) as a white solid.

¹H NMR (300 MHz, CDCl₃) [symbol] 2.09 (3H, s), 2.26 (3H, s), 3.03 (3H, s), 5.41 (1H, t, J=7.4 Hz), 7.12 (1H, d, J=11.0 Hz), 7.39 (1H, d, J=8.8 Hz), 8.13 (1H, d, J=11.0 Hz), 8.31 (1H, d, J=8.8 Hz), 8.66 (1H, s), 9.06 (1H, s), 9.21-9.44 (3H, m).

MS (ESI): m/z 470 (M+H)⁺.

### Example 44

### 2-ETHOXY-N-((1R)-1-{3-METHYL-4-[(METHYLSULFONYL)AMINO]PHENYL}ETHYL)-QUINOLINE-6-CARBOXAMIDE

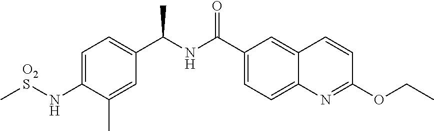

### 44B) 2-ETHOXYQUINOLINE-6-CARBOXYLIC ACID HYDROCHLORIDE

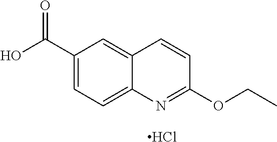

To a suspension of the compound of tert-butyl 2-ethoxyquinoline-6-carboxylate (86 mg, 0.315 mmol) in THF (2 ml) was added concentrated hydrochloride (0.5 ml) and the mixture was stirred at room temperature for 16 hours. The reaction mixture was concentrated and co-evaporated with toluene to furnish the title compound (102 mg, quant.) as a white solid.

¹H NMR (270 MHz, DMSO-d₆) δ 1.38 (3H, t, J=7.3 Hz), 4.48 (2H, q, J=7.3 Hz), 7.06 (1H, d, J=9.2 Hz), 7.79 (1H, d, J=8.6 Hz), 8.12 (10, d, J=9.3 Hz), 8.39 (1H, d, J=8.6 Hz), 8.54 (1H, s).

MS (ESI): m/z 218 (M+H)⁺.

### 44C) 2-ETHOXY-N-((1R)-1-{3-METHYL-4-[(METHYLSULFONYL)AMINO]PHENYL}ETHYL)-QUINOLINE-6-CARBOXAMIDE

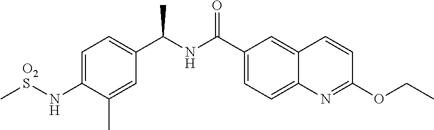

A DMF (3 ml) solution of the compound of Example 1D (104 mg, 0.32 mmol), triethylamine (0.13 ml, 0.95 mmol), the compound of Example 44B (80 mg, 0.32 mmol) and HBTU (143 mg, 0.38 mmol) was treated in the same procedure described in Example 1G. The crude residue was applied to a silica gel column chromatography and eluted with hexane/ethyl acetate (2:1) to furnish the title compound (33 mg, 25% yield) as a white solid.

¹H NMR (270 MHz, DMSO-d₆) [symbol] 1.39 (3H, t, J=7.3 Hz), 1.50 (3H, d, J=7.3 Hz), 2.31 (3H, s), 2.96 (3H, s) 4.49 (2H, q, J=7.3 Hz), 5.13-5.23 (1H, m), 7.06 (1H, d, J=8.6 Hz), 7.21-7.30 (3H, m), 7.80 (1H, d, J=8.6 Hz), 8.11-8.16 (1H, m), 8.32 (1H, d, J=9.2 Hz); 8.44 (1H, s), 8.92 (1H, d, J=7.9 Hz), 9.00 (1H, s).

MS (ESI): m/z 428 (M+H)⁺.

### Example 45

### (R)—N-(1-(2-FLUORO-5-METHYL-4-(METHYLSULFONAMIDO)PHENYL)ETHYL)-6-CYCLOPROPYL-2-NAPHTHAMIDE

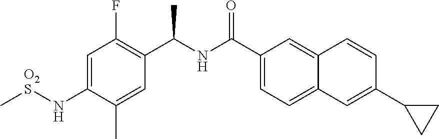

To a DMF (3 ml) solution of the compound of Example 13D (66.6 mg, 0.236 mmol), Example 22B (50 mg, 0.24 mmol), N-ethyl-N′-(3-dimethylaminopropyl)-carbodiimide hydrochloride (67.7 mg, 0.353 mmol), and HOBt hydrate (10.8 mg, 0.071 mmol) was added triethylamine (0.0985 ml, 0.707 mmol) and the mixture was stirred for 24 hours at room temperature. Then the reaction was diluted with ethyl acetate-toluene (1:1, 50 ml) and washed 2M hydrochloric aqueous solution, water, saturated aqueous sodium bicarbonate solution, water and brine. The organic layer was dried over sodium sulfate and concentrated in vacuo to give crude product. The crude product was purified by column chromatography on silica gel with ethyl acetate-hexane (1:1) to furnish the title compound as white solid (24.9 mg, 24% yield).

¹H NMR (270 MHz, DMSO-d₆) [symbol] 0.71-0.93 (2H, m), 0.98-1.17 (2H, m), 1.49 (3H, d, J=7.3 Hz), 2.04-2.19 (1H, m), 2.24 (3H, s), 3.00 (3H, s), 5.26-5.54 (1H, m), 7.08 (1H, d, J=11.9 Hz), 7.22-7.43 (2H, m), 7.67 (1H, s), 7.80-8.00 (3H, m), 8.43 (1H, s), 8.92 (1H, br.d, J=7.3 Hz), 9.18 (1H, br.s).

MS (ESI) m/z 439 (M−H)⁻, 441 (M+H)⁺.

### Example 46

### 2-CYCLOPROPYL-N-((1R)-1-{3,5-DIFLUORO-4-[(METHYLSULFONYL)AMINO]PHENYL}-ETHYL)QUINOLINE-6-CARBOXAMIDE

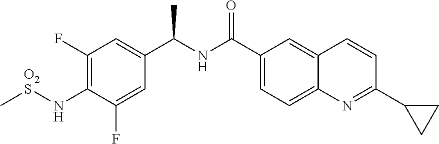

### 46A) TERT-BUTYL 2-CYCLOPROPYLQUINOLINE-6-CARBOXYLATE

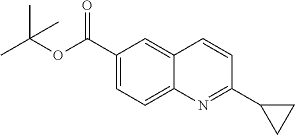

To a solution of tert-butyl quinoline-6-carboxylate (1.91 g, 8.33 mmol, WO2005080373A1) in THF (2 ml) was added cyclopropyl magnesiumbromide (45 ml, 22 mmol, 0.5M in THF solution) at 0° C. dropwise. The mixture was stirred at room temperature for 16 hours, then further amount of cyclopropyl magnesiumbromide (20 ml, 10 mmol, 0.5 M in THF solution) was added and the mixture was stirred at room temperature for 24 hours. Then to the mixture was added a cerium ammonium nitrate (6.85 g, 12.5 mmol) at room temperature. After stirring for 30 minutes, the reaction was filtrated and the filtrate was diluted with saturated sodium bicarbonate aqueous solution, and the product was extracted with ethyl acetate which was dried over sodium sulfate. Then, filtration, evaporation, purification through silica gel column chromatography eluting with hexane/ethyl acetate (100:1 to 20:1) to furnish the title compound (263 mg, 12% yield) as colorless oil.

¹H NMR (300 MHz, CDCl₃) [symbol] 1.12-1.29 (4H, m), 1.65 (9H, s), 2.20-2.30 (1H, m), 7.24 (1H, d, J=8.8 Hz), 7.95 (1H, d, J=8.8 Hz), 8.08 (1H, d, J=8.8 Hz), 8.17-8.21 (1H, m), 8.44-8.46 (1H, m).

MS (ESI): m/z 270 (M+H)⁺.

### 46B) 2-CYCLOPROPYLQUINOLINE-6-CARBOXYLIC ACID HYDROCHLORIDE

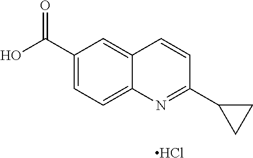

A mixture of the compound of Example 46A (261 mg, 0.97 mmol) and 4N-hydrochloride in dioxane (4 ml) was stirred at room temperature for 16 hours. The reaction mixture was concentrated to furnish the title compound (254 mg, quant.) as a white solid.

¹H NMR (270 MHz, DMSO-d₆) δ 1.10-1.50 (4H, m), 2.30-2.75 (1H, m), 7.62 (1H, d, J=8.1 Hz), 8.26-8.38 (2H, m), 8.78 (1H, s), 8.84-8.88 (1H, m).

MS (ESI): m/z 214 (M+H)⁺.

### 46C) 2-CYCLOPROPYL-N-((1R)-1-{3,5-DIFLUORO-4-[(METHYLSULFONYL)AMINO]PHENYL}ETHYL)QUINOLINE-6-CARBOXAMIDE

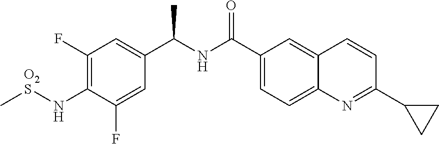

A DMF (1 ml) solution of the compound of Example 3D (57 mg, 0.20 mmol), triethylamine (0.08 ml, 0.60 mmol), the compound of Example 46B (50 mg, 0.20 mmol) and HBTU (91 mg, 0.24 mmol) was treated in the same procedure described in Example 1G. The crude residue was applied to a silica gel column chromatography and eluted with hexane/ethyl acetate (1:1) to furnish the title compound (26 mg, 29% yield) as a white solid.

¹H NMR (300 MHz, DMSO-d₆) [symbol] 1.08-1.15 (4H, m), 1.49 (3H, d, J=6.6 Hz), 2.29-2.36 (1H, m), 3.04 (3H, s), 5.16-5.24 m), 7.25 (2H, d, J=8.8 Hz), 7.50 (1H, d, J=8.8 Hz), 7.89 (1H, d, J=8.8 Hz), 8.12 (1H, d, J=8.8 Hz), 8.32 (1H, d, J=8.8 Hz), 8.47 (1H, s), 9.03 (1H, d, J=7.3 Hz), 9.49 (1H, s).

MS (ESI): m/z 446 (M+H)⁺.

### Example 47

### (R)—N-(1-(2-FLUORO-5-METHYL-4-(METHYLSULFONAMIDO)PHENYL)ETHYL)-6-(TRIFLUOROMETHYL)-2-NAPHTHAMIDE

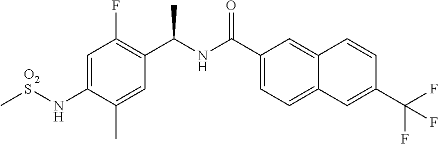

To a DMF (3 ml) solution of the compound of Example 13D (51.3 mg, 0.208 mmol), 6-(trifluoromethyl)-2-naphthoic acid (prepared according to Synthesis 2005, 791-797; 50 mg, 0.210 mmol), WSC (59.9 mg, 0.312 mmol), and HOBt hydrate (9.6 mg, 0.0625 mmol) was added triethylamine (0.087 ml, 0.625 mmol) and the mixture was stirred for 24 hours at room temperature. Then the reaction was diluted with ethyl acetate-toluene (1:1, 50 ml) and washed 2M hydrochloric aqueous solution, water, saturated aqueous sodium bicarbonate solution, water and brine. The organic layer was dried over sodium sulfate and concentrated in vacuo to give crude product. The crude product was purified by column chromatography on silica gel with ethyl acetate-hexane (1:1) to furnish the title compound as a white solid (23.2 mg, 24% yield).

¹H NMR (270 MHz, DMSO-d₆) [symbol] 1.51 (3H, d, J=6.6 Hz), 2.26 (3H, s), 3.03 (3H, s), 5.30-5.50 (1H, m), 7.10 (1H, d, J=11.2 Hz), 7.37 (1H, d, J=8.6 Hz), 7.84 (1H, d, J=9.9 Hz), 8.09 (1H, d, J=8.6 Hz), 8.24 (1H, d, J=8.6 Hz), 8.30 (1H, d, J=9.2 Hz), 8.50 (1H, s), 8.62 (1H, s), 9.11 (1H, d, J=7.3 Hz), 9.19 (1H, br.s).

MS (ESI) m/z 467 (M−H)⁻, 469 (M+H)⁺.

### Example 48

### N-((1R)-1-{3,5-DIFLUORO-4-[(METHYLSULFONYL)AMINO]PHENYL}ETHYL)-2-(1-METHYL-CYCLOPROPYL)QUINOLINE-6-CARBOXAMIDE

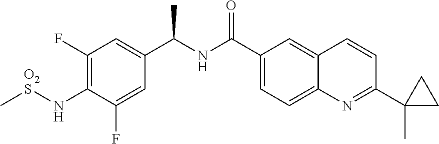

### 48A) 6-BROMO-N-METHOXY-N-METHYLQUINOLINE-2-CARBOXAMIDE

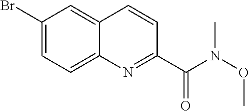

A DMF (1 ml) solution of 6-bromoquinoline-2-carboxylic acid (4000 mg, 15.9 mmol, US2005165049A1), triethylamine (6.64 ml, 47.6 mmol), N,O-dimethylhydroxyamine hydrochloride (1860 mg, 19.0 mmol) and HBTU (6620 mg, 17.5 mmol) was treated in the same procedure described in Example 1G. The crude residue was applied to a silica gel column chromatography and eluted with hexane/ethyl acetate (4:1) to furnish the title compound (4.29 g, 92% yield) as a orange solid.

¹H NMR (300 MHz, CDCl₃) [symbol] 3.47 (3H, s), 3.80 (3H, s), 7.68-7.80 (1H, brs), 7.81-7.85 (1H, m), 8.00-8.06 (2H, m), 8.17 (1H, d, J=8.1 Hz).

MS (ESI): m/z 295, 297 (M+H)⁺.

### 48B) 1-(6-BROMOQUINOLIN-2-YL)ETHANONE

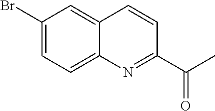

To a solution of the compound of Example 48A (4.29 g, 14.5 mmol) in THF (100 ml) was added methyl magnesiumbromide (18.2 ml, 17.4 mmol, 0.96M in THF solution) at 0° C. dropwise and the mixture was stirred at 0° C. for 1 hour. Then, the mixture was quenched with saturated ammonium chloride aqueous solution (50 ml) and water (200 ml). After stirring for 30 min, the product was extracted with ethyl acetate which was dried over sodium sulfate. Then, filtration, evaporation, purification through silica gel column chromatography eluting with hexane/ethyl acetate (4:1) to furnish the title compound (3.47 g, 96% yield) as a white solid.

¹H NMR (300 MHz, CDCl₃) [symbol] 2.66 (3H, s), 7.83-7.88 (1H, m), 8.02-8.20 (4H, m).

MS (ESI): m/z 250, 252 (M+H)⁺.

### 48C) 6-BROMO-2-ISOPROPENYLQUINOLINE

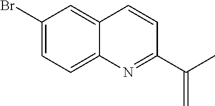

A suspension of (methyl)triphenylphosphonium bromide (2.86 g, 8.0 mmol) in THF (20 ml) was added potassium butoxide (897 mg, 8.0 mmol) in THF dropwise at 0° C. A solution of the compound of Example 48B in THF (5 ml) was added there at room temperature and the mixture was stirred for 2 hours. Then the reaction was quenched with water, and the product was extracted with ethyl acetate which was dried over sodium sulfate. Then, filtration, evaporation, purification through silica gel column chromatography eluting with hexane/ethyl acetate (100:1) to furnish the title compound (791 mg, 79% yield) as a pale yellow solid.

¹H NMR (300 MHz, CDCl₃) [symbol] 2.34 (3H, s), 5.51 (1H, s), 5.93 (1H, s), 7.69-7.77 (2H, m), 7.94-8.02 (3H, m).

MS (ESI): m/z 248, 250 (M+H)⁺.

### 48D) 6-BROMO-2-(1-METHYLCYCLOPROPYL)QUINOLINE

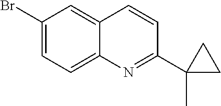

To a suspension of trimethyl sulfoxonium iodide (945 mg, 4.3 mmol) in DMSO (10 ml) was added potassium butoxide (482 mg, 4.3 mmol) at room temperature. After 1 hour, a solution of the compound of Example 48C (710 mg, 2.9 mmol) in THF (2 ml) was added there. The mixture was stirred at room temperature for 5 hours. Then the mixture was quenched with saturated sodium bicarbonate aqueous solution, and the product was extracted with ethyl acetate which was dried over sodium sulfate. Then, filtration, evaporation, purification through silica gel column chromatography eluting with hexane/ethyl acetate (100:1) to furnish the title compound (338 mg, 23% yield) as colorless oil which was including the compound of Example 48C in about 20%.

¹H NMR (300 MHz, CDCl₃) [symbol] 0.85-1.40 (4H, m), 1.62 (3H, s), 7.40 (1H, d, J=8.8 Hz), 7.70-8.03 (4H, m).

MS (ESI): m/z 262, 264 (M+H)⁺.

### 48 E) METHYL 2-(1-METHYLCYCLOPROPYL)QUINOLINE-6-CARBOXYLATE

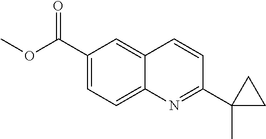

A mixture of the compound of Example 48D (338 mg, 1.29 mmol), triethylamine (0.27 ml, 1.93 mmol), 1,3-bis(diphenylphosphino)propane (40 mg, 0.1 mmol), palladium acetate (14.5 mg, 0.065 mmol) and methanol (2 ml) in DMF (4 ml) was stirred at reflux under carbon monoxide (1 atm) for 16 hours. Then the reaction was quenched with saturated sodium bicarbonate aqueous solution and the product was extracted with ethyl acetate which was dried over sodium sulfate. Then, filtration, evaporation, purification through silica gel column chromatography eluting with hexane/ethyl acetate (20:1) to furnish the title compound (126 mg, 21% yield) as a white solid which was including the compound derived from 48C in about 25%.

¹H NMR (300 MHz, CDCl₃) [symbol] 0.90-0.98 (2H, m), 1.38-1.45 (2H, m), 1.63 (3H, s), 3.98 (3H, s), 7.43 (1H, d, J=8.8 Hz), 7.99 (1H, d, J=8.8 Hz), 8.10-8.54 (3H, m).

MS (ESI): m/z 242 (M+H)⁺.

### 48F) 2-(1-METHYLCYCLOPROPYL)QUINOLINE-6-CARBOXYLIC ACID

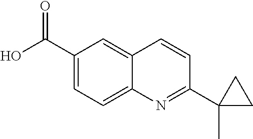

A methanol (1 ml) and THF (1 ml) solution of the compound of Example 48E (127 mg, 0.53 mmol) and 2M-sodium hydroxide aqueous solution (0.53 ml, 1.1 mmol) was treated in the same procedure described in Example 8B to furnish the title compound (125 mg, quant.) as a white solid.

¹H NMR (300 MHz, CDCl₃) [symbol] 0.94-0.08 (2H, m), 1.41-1.45 (2H, m), 1.65 (3H, s), 7.47 (1H, d, J=8.6 Hz), 8.04 (1H, d, J=9.2 Hz), 8.15-8.35 (2H, m), 8.61-8.64 (1H, m).

MS (ESI): m/z 228 (M+H)⁺.

### 48G) N-((1R)-1-{3,5-DIFLUORO-4-[(METHYLSULFONYL)AMINO]PHENYL}ETHYL)-2-(1-METHYLCYCLOPROPYL)QUINOLINE-6-CARBOXAMIDE

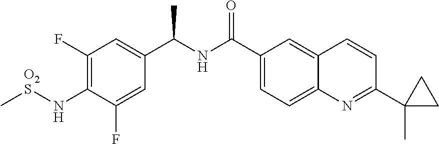

A DMF (1 ml) solution of the compound of Example 3D (38 mg, 0.13 mmol), triethylamine (0.056 ml, 0.40 mmol), the compound of Example 48F (30 mg, 0.13 mmol) and HBTU (60 mg, 0.16 mmol) was treated in the same procedure described in Example 1G. The crude residue was applied to a silica gel column chromatography and eluted with hexane/ethyl acetate (3:2) and HPLC (used column was XTerra MS C18, 5 um, 30×50 mm) eluting with acetonitrile/0.01% ammonium aqueous solution (32:68 to 68:32).to furnish the title compound (19 mg, 31% yield) as a white solid.

¹H NMR (300 MHz, DMSO-d₆) δ 0.94-0.96 (2H, m), 1.31-133 (2H, m), 1.50 (3H, d, J=6.6H), 1.60 (3H, s), 3.04 (3H, s), 5.15-5.25 (1H, m), 7.26 (2H, d, J=8.8 Hz), 7.54 (1H, d, J=8.8 Hz), 7.92 (1H, d, J=8.8 Hz), 8.11-8.16 (1H, m), 8.37 (1H, d, J=8.8 Hz), 8.48 (1H, s), 9.04 (1H, d, J=8.1 Hz), 9.48 (1H, brs).

MS (ESI): m/z 460 (M+H)⁺.

### Example 49

### N-((1R)-1-{3-METHYL-4-[(METHYLSULFONYL)AMINO]PHENYL}ETHYL)-2-(2,2,2-TRIFLUORO-1,1-DIMETHYLETHYL)QUINOLINE-6-CARBOXAMIDE

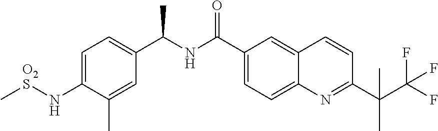

### 49A) 2-(6-BROMOQUINOLIN-2-YL)-1,1,1-TRIFLUOROPROPAN-2-OL

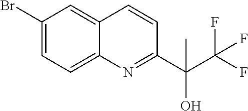

A DMF (5 ml) solution of the compound of Example 48B (129 mg, 0.52 mmol), (trifluoromethyl)trimethylsilane (220 mg, 1.55 mmol) and tetrabutylammonium fluoride (13.5 mg, 0.052 mmol) was stirred at 100° C. for 2 hours. Then the mixture was cooled to room temperature and added 1N-hydrochloride aqueous solution (2 ml). After 4 hours, the mixture was quenched with saturated sodium bicarbonate aqueous solution, and the product was extracted with ethyl acetate which was dried over sodium sulfate. Then, filtration, evaporation and purification through silica gel column chromatography eluting with hexane/ethyl acetate (4:1) furnished the title compound (175 mg, quant.) as a white solid.

¹H NMR (300 MHz, CDCl₃) [symbol] 1.81 (3H, s), 6.51 (1H, s), 7.64 (1H, d, J=8.1 Hz), 7.66-7.89 (1H, m), 8.00-8.12 (2H, m), 8.21 (1H, d, J=8.8 Hz).

MS (ESI): m/z 320, 322 (M+H)⁺.

### 49B) 1-(6-BROMOQUINOLIN-2-YL)-2,2,2-TRIFLUORO-1-METHYLETHYL METHANE-SULFONATE

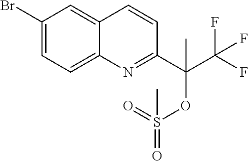

To a solution of the compound of Example 49A (1.93 g, 6.03 mmol) in THF (20 ml) was added sodium hydride (241 mg, 7.23 mmol) portionwise at 0° C. and the mixture was stirred at room temperature for 1 hour. A solution of methanesulfonyl chloride (829 mg, 7.23 mmol) in THF (2 ml) was added there at 0°. Then the reaction mixture was stirred at room temperature for 16 hours. The mixture was quenched with saturated sodium bicarbonate aqueous solution, and the product was extracted with ethyl acetate which was dried over sodium sulfate. Then, filtration, evaporation, purification through silica gel column chromatography eluting with hexane/ethyl acetate (15:1 to 5:1) to furnish the title compound (1.11 g, 46% yield) as a white solid.

¹H NMR (300 MHz, CDCl₃) [symbol] 2.45 (3H, s), 3.24 (3H, s), 7.81-7.86 (2H, m), 7.96-8.05 (2H, m), 8.17 (1H, d, J=8.8 Hz).

MS (ESI): m/z 397, 399 (M+H)⁺.

### 49C) 6-BROMO-2-(2,2,2-TRIFLUORO-1,1-DIMETHYLETHYL)QUINOLINE

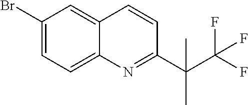

A suspension of the compound of Example 49B (1.40 g, 3.52 mmol) in cyclohexane (14 ml) was added trimethylaluminum (14 ml, 14 mmol, 1.03M in hexane solution) at room temperature, and the mixture was stirred at room temperature for 16 hours. The reaction was carefully quenched with saturated sodium bicarbonate aqueous solution (10 ml), brine (10 ml) and diluted with ethyl acetate (100 ml). After the mixture was, stirred for 30 minutes, formed precipitate was removed by celite and washed with ethyl acetate. The filtrate was concentrated and purified through silica gel column chromatography eluting with hexane only to furnish the title compound (951 mg, 85% yield) as colorless oil.

¹H NMR (300 MHz, CDCl₃) [symbol] 1.72 (6H, s), 7.66 (1H, d, J=8.8 Hz), 7.75-7.80 (1H, m), 7.96-8.00 (2H, m), 8.06 (1H, d, J=8.8 Hz).

MS (EST): m/z 318, 320 (M+H)⁺.

### 49D) METHYL 2-(2,2,2-TRIFLUORO-1,1-DIMETHYLETHYL)QUINOLINE-6-CARBOXYLATE

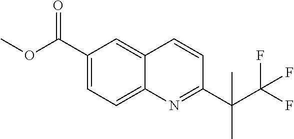

A mixture of the compound of Example 49C (950 mg, 3.0 mmol), triethylamine (1.25 ml, 9.0 mmol), 1,3-bis(diphenylphosphino)propane (123 mg, 0.3 mmol), palladium acetate (67 mg, 0.3 mmol) and methanol (4.8 ml) in DMF (10 ml) was stirred at reflux under carbon monoxide (1 atm) for 16 hours. Then the reaction was quenched with saturated sodium bicarbonate aqueous solution and the product was extracted with ethyl acetate which was dried over sodium sulfate. Then, filtration, evaporation, purification through silica gel column chromatography eluting with hexane/ethyl acetate (25:1) to furnish the title compound (777 mg, 88% yield) as a white solid.

¹H NMR (300 MHz, CDCl₃) [symbol] 1.74 (6H, s), 4.00 (3H, s), 7.71 (1H, d, J=8.8 Hz), 8.14 (1H, d, J=8.8 Hz), 8.25 (1H, d, J=^{.}8.8 Hz), 8.28-8.32 (1H, m), 8.58-8.59 (1H, m).

MS (ESI): m/z 298 (M+H)⁺.

### 49E) 2-(2,2,2-TRIFLUORO-1,1-DIMETHYLETHYL)QUINOLINE-6-CARBOXYLIC ACID

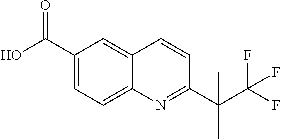

A methanol (6 ml) and THF (6 ml) solution of the compound of Example 49D (777 mg, 2.6 mmol) and 2M-sodium hydroxide aqueous solution (2.6 ml, 5.2 mmol) was treated in the same procedure described in Example 8B to furnish the title compound (735 mg, 99% yield) as a white solid.

¹H NMR (300 MHz, CDCl₃) δ 1.75 (6H, s), 7.74 (1H, d, J=8.8 Hz), 8.19 (1H, d, J=8.8 Hz), 8.29 (1H, d, J=8.8 Hz), 8.35-8.40 (1H, m), 8.69-8.70 (1H, m).

MS (ESI): m/z 284 (M+H)⁺.

### 49F) N-((1R)-1-{3-METHYL-4-[(METHYLSULFONYL)AMINO]PHENYL}ETHYL)-2-(2,2,2-TRIFLUORO-1,1-DIMETHYLETHYL)QUINOLINE-6-CARBOXAMIDE

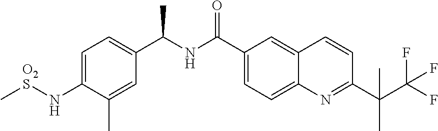

A DMF (2 ml) solution of the compound of Example 1D (47 mg, 0.18 mmol), triethylamine (0.074 ml, 0.53 mmol), the compound of Example 49E (50 mg, 0.18 mmol) and HBTU (80 mg, 0.21 mmol) was treated in the sane procedure described in Example 1G. The crude residue was applied to a silica gel column chromatography and eluted with hexane/ethyl acetate (2:1) to furnish the title compound (59 mg, 68% yield) as a white solid.

¹H NMR (300 MHz, DMSO-d₆) [symbol] 1.50 (3H, d, J=6.6 Hz), 1.71 (6H, s), 2.30 (3H, s), 2.95 (3H, s), 5.15-5.23 (1H, m), 7.21-7.31 (3H, m), 7.87 (1H, d, J=8.8 Hz), 8.08 (1H, d, J=8.8 Hz), 8.23 (1H, d, J=9.5 Hz), 8.53-8.57 (2H, m), 9.00-9.09 (2H, m).

MS (ESI): m/z 494 (M+H)⁺.

### Example 50

### (R)—N-(1-(2-FLUORO-5-METHYL-4-(METHYLSULFONAMIDO)PHENYL)ETHYL)-6-(1-METHYL-CYCLOPROPYL)-2-NAPHTHAMIDE

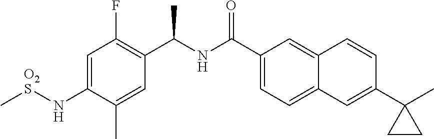

### 50A) METHYL 6-(PROP-1-EN-2-YL)-2-NAPHTHOATE

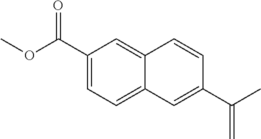

A suspension of methyl triphenylphosphonium bromide (2.41 g, 6.74 mmol) in THF (20 ml) was added dropwise potassium tert-butoxide (756 mg, 6.74 mmol) in THF (20 ml) at 0° C., and the mixture was stirred at room temperature for 1.5 hours. Then, methyl 6-acetyl-2-naphthoate (J. Org. Chem, 1990, 55, 319-324, 769 mg, 3.37 mmol) in THF (5 ml) was added there at room temperature, and the resulting mixture was stirred at room temperature for 2 hours. The reaction was quenched with water (100 ml) and extracted with ethyl acetate-hexane (1:2). The organic layer was dried over sodium sulfate and concentrated in vacuo. The crude material was purified by silica gel column chromatography, eluting with ethyl acetate-hexane (0:100 to 1:20) to give 0.67 g (88% yield) of the title compound as white solid.

¹H NMR (270 MHz, CDCl₃) [symbol] 2.28 (3H, s), 3.99 (3H, s), 5.26 (1H, s), 5.58 (1H, s), 7.74 (1H, d, J=8.6 Hz), 7.82-7.97 (3H, m), 8.05 (1H, d, J=8.6 Hz), 8.58 (1H, s).

### 50B) METHYL 6-(1-METHYLCYCLOPROPYL)-2-NAPHTHOATE

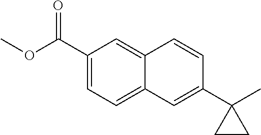

Diethylzinc (1.0 M solution in hexane, 6.30 ml, 6.30 mmol) was added to a solution of the compound of Example 50A (0.57 g, 2.5 mmol) in 1,2-dichloroethane (25 ml) at 0° C. Diiodomethane (1.01 ml, 12.6 mmol was then added dropwise to the solution and the resultant mixture was stirred at 60° C. for 20 hours. The reaction mixture was cooled to room temperature, diluted with saturated aqueous ammonium chloride solution (30 ml), and the mixture was extracted with dichloromethane (30 ml×3 times). The combined organic layer was washed with saturated aqueous sodium bicarbonate (50 mL) and brine (50 mL), and the organic layer was dried over sodium sulfate Removal of the solvent gave a residue, which was chromatographed on a column of silica gel eluting with ethyl acetate-hexane (1:20) to give 0.91 g of the title compound as white solid. This crude product as used for the next step without further purification.

¹H NMR (270 MHz, CDCl₃) [symbol] 0.75-0.95 (2H, m), 0.95-1.13 (2H, m), 1.52 (3H, s), 3.97 (3H, s), 7.41 (1H, d, J=9.9 Hz), 7.74 (1H, s), 7.82 (1H, d, J=7.8 Hz), 7.86 (1H, d, J=8.6 Hz), 8.04 (1H, d, J=8.6 Hz), 8.56 (1H, s).

### 50C) 6-(1-METHYLCYCLOPROPYL)-2-NAPHTHOIC ACID

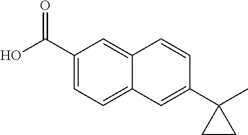

A mixture of the compound of Example 50B (crude 0.91 g, 2.5 mmol) and 2M sodium hydroxide solution (3.8 ml) in methanol (7.6 ml) was heated at 60° C. for 2 hours. After cooling to room temperature, the mixture was washed with diethyl ether (100 ml). The aqueous layer was acidified to pH<3 with 2M hydrochloric acid solution and the mixture was extracted with dichloromethane-methanol (10:1, 150 ml×3 times). The combined organic layer was dried over sodium sulfate and concentrated in vacuo to give 0.444 g (78% yield in 2 steps) of the title compound as white solid.

¹H NMR (300 MHz, DMSO-d₆) [symbol] 0.77-0.92 (2H, m), 0.95-1.11 (2H, m), 1.49 (3H, s), 7.42 (1H, d, J=8.8 Hz), 7.84 (1H, s), 7.90-7.97 (2H, m), 8.01 (1H, d, J=8.8 Hz), 8.54 (1H, s).

MS (ESI): 225 (M−H)⁻.

### 50D) (R)—N-(1-(2-FLUORO-5-METHYL-4-(METHYLSULFONAMIDO)PHENYL)ETHYL)-6-(1-METHYLCYCLOPROPYL)-2-NAPHTHAMIDE

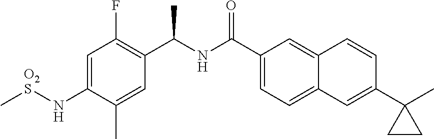

To a DMF (3 ml) solution of the compound of Example 13D (62.5 mg, 0.221 mmol), Example 50C (50 mg, 0.22 mmol), WSC (63.5 mg, 0.331 mmol), and HOBt hydrate (10.2 mg, 0.0663 mmol) was added triethylamine (0.092 ml, 0.663 mmol) and the mixture was stirred for 24 hours at room temperature. Then the reaction was diluted with ethyl acetate-toluene (1:1, 50 ml) and washed 2M hydrochloric aqueous solution, water, saturated aqueous sodium bicarbonate solution, water and brine. The organic layer was dried over sodium sulfate and concentrated in vacuo to give crude product. The crude product was purified by column chromatography on silica gel with ethyl acetate-hexane (1:1) to furnish the title compound as a white solid (16.4 mg, 16% yield).

¹H NMR (270 MHz, DMSO-d₆) [symbol] 0.81-0.92 (2H, m), 0.96-1.05 (2H, m), 1.42-1.58 (6H, m), 2.24 (3H, s), 3.01 (3H, s), 5.26-5.52 (1H, m), 7.09 (1H, d; J=11.9 Hz), 7.35 (1H, d, J=8.6 Hz), 7.41 (1H, d, J=8.6 Hz), 7.82 (1H, s), 7.88-8.01 (3H, m), 8.44 (1H, s), 8.94 (1H, d, J=7.3 Hz), 9.20 (1H, br.s).

MS (ESI) m/z 453 (M−H)⁻, 455 (M+H)⁺.

### Example 51

### N-((1R)-1-{3-METHYL-4-[(METHYLSULFONYL)AMINO]PHENYL}ETHYL)-6-(TRIFLUORO-METHYL)-2-NAPHTHAMIDE

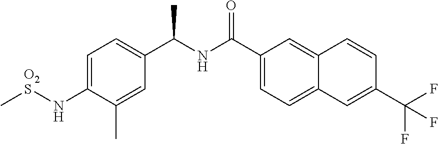

To a DMF (3.0 ml) solution of the compound of Example 1D (55.1 mg, 0.208 mmol), 6-(trifluoromethyl)-2-naphthoic acid (prepared according to Synthesis 2005, 791-797; 50 mg, 0.208 mmol) and HBTU (94.7 mg, 0.252 mmol) was added triethylamine (0.0871 ml, 0.625 mmol) and the mixture was stirred for 10 hours at room temperature. The same procedure as described in Example 1G was performed to give the title compound (59.6 mg, 63.5% yield) as a white solid.

¹H NMR (270 MHz, DMSO-d₆) δ 1.52 (3H, d, J=6.5 Hz), 2.31 (3H, s), 2.97 (3H, s), 5.13-5.26 (1H, m), 7.20-7.35 (3H, m), 7.81-7.87 (1H, m), 8.06-8.13 (1H, m), 8.20-8.32 (2H, m), 8.50 (1H, s), 8.61 (1H, s), 8.99-9.10 (2H, m).

MS (ESI) m/z 449.11 (M−H)⁻, 451.03 (M+H)⁺.

### Example 52

### (R)—N-(1-(2-CHLORO-5-METHYL-4-(METHYLSULFONAMIDO)PHENYL)ETHYL)-6-CYCLOPROPYL-2-NAPHTHAMIDE

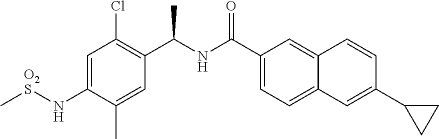

### 52A) N-(5-CHLORO-2-METHYLPHENYL)METHANESULFONAMIDE

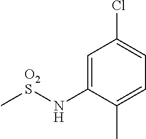

To a pyridine (3.0 ml, 37.1 mmol) and dichloromethane (40 ml) solution of 2-chloro-5-methylaniline (5.00 g, 35.3 mmol), methanesulfonyl chloride (2.73 ml, 35.3 mmol) was added at room temperature and the mixture was stirred for 20 hours. The reaction was diluted with ethyl acetate (250 ml), washed with 2M hydrochloric acid aqueous solution (100 ml), saturated aqueous sodium bicarbonate solution (100 ml), brine (100 ml), and the organic layer was dried over sodium sulfate. Removal of the solvent gave crude product (white solid). The white solid was diluted with dichloromethane (50 ml), filtered, washed with dichloromethane (50 ml×3 times), and dried in vacuo to give the title compound (7.56 g, 98%) as white solid.

¹H NMR (270 MHz, DMSO-d₆) [symbol] 2.28 (3H, s), 3.02 (3H, s), 7.21 (1H, d, J=7.9 Hz), 7.29 (1H, d, J=8.5 Hz), 7.33 (1H, s), 9.25 (1H, br.s).

MS (ESI) m/z 218 (M−H)⁻

### 52B) N-(4-ACETYL-5-CHLORO-2-METHYLPHENYL)METHANESULFONAMIDE

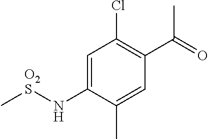

To a dichloromethane (50 ml) solution of the compound of Example 52A (5.35 g, 24.4 mmol) was added aluminum trichloride (8.12 g, 60.9 mmol) and acetyl chloride (2.60 ml, 36.5 mmol) was slowly added at 0° C. and the mixture was stirred at room temperature for 24 hours. The reaction mixture was quenched with 2M hydrochloric acid solution (150 ml) and the whole was extracted with ethyl acetate (300 ml). The organic layer was washed with saturated aqueous sodium bicarbonate (150 ml) and brine (150 ml), dried over sodium sulfate, filtered and evaporated. The crude product was chromatographed on a column of silica gel eluting with ethyl acetate-hexane (1:2 to 1:1) to give the title compound (5.85 g, 92%) as white solid.

¹H NMR (270 MHz, DMSO-d₆) δ 2.30 (3H, s), 2.56 (3H, s), 3.10 (3H, s), 7.43 (1H, s), 7.65 (1H, s), 9.47 (1H, brs).

MS (ESI) m/z 260 (M−H)⁻

### 52C) N-[4-((1R)-1-{[(R)-TERT-BUTYLSULFINYL]AMINO}ETHYL)-5-CHLORO-2-METHYL-PHENYL]METHANESULFONAMIDE

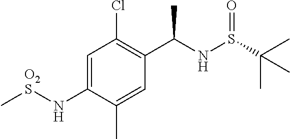

To a THF (54.2 ml) solution of the compound of Example 52B (6.77 g, 25.9 mmol) and (R)-(+)-2-methyl-2-propanesulfinylamide (3.45 g, 28.5 mmol), titanium(IV) ethoxide (54.2 ml, 0.259 mol) was added under a nitrogen atmosphere and the mixture was refluxed with stirring for 16 hours. After imine formation was confirmed with LC-MS, the mixture was cooled to r.t. and the imine solution was added dropwise to a suspension of sodium borohydride (2.94 g, 77.6 mmol) in THF (50 mL) at 0° C. under nitrogen atmosphere. After stifling at room temperature for 5 hours, the reaction mixture was partitioned with water and ethanol, and then, the mixture was stirred for 1 hour at room temperature. The mixture was filtered through a Celite pad, and the filtrate was evaporated and concentrated in vacuo. The crude product was chromatographed on a column of silica gel eluting with ethyl acetate-hexane (2:1) gave the title compound as diastereomer mixture (4.08 g). The mixture was recrystallized from ethyl acetate (150 ml) to give the title compound (1.95 g, 20%) as white solid.

¹H NMR (270 MHz, CDCl₃) δ 1.24 (9H, s), 1.50 (3H, d, J=6.6 Hz), 2.29 (3H, s), 3.06 (3H, s), 3.59 (1H, d, J=5.3 Hz), 4.73-5.03 (1H, m), 6.27 (1H, br.s), 7.28 (1H, s), 7.48 (1H, s).

MS (ESI) m/z 365 (M−H)⁺, 367 (M+H)⁺

### 52D) N-{4-[(1R)-1-AMINOETHYL]-5-CHLORO-2-METHYLPHENYL}METHANESULFONAMIDE HYDROCHLORIDE

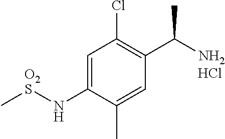

To the compound of Example 52C (1.30 g, 3.54 mmol) was added hydrochloric acid methanolic (10%, 15 ml). The reaction mixture was evaporated and dried in vacuo to give the title compound (1.06 g, 100%) as a white solid.

¹H NMR (270 MHz, DMSO-d₆) δ 1.49 (3H, d, J=72 Hz), 2.31 (3H, s), 3.05 (3H, s), 4.62 (1H, m), 7.61 (1H, s), 7.68 (1H, s), 8.66 (3H, br.s).

MS (ESI) m/z 261 (M−H)⁻.

### 52E) (R)—N-(1-(2-CHLORO-5-METHYL-4-(METHYLSULFONAMIDO)PHENYL)ETHYL)-6-CYCLOPROPYL-2-NAPHTHAMIDE

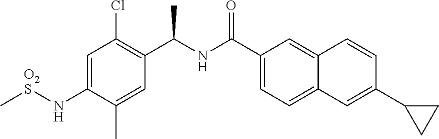

To a DMF (3 ml) solution of the compound of Example 52D (50 mg, 0.17 mmol), Example 22B (35.5 mg, 0.167 mmol), WSC (48 mg, 0.251 mmol), and HOBt hydrate (7.7 mg, 0.050 mmol) was added triethylamine (0.070 ml, 0.501 mmol) and the mixture was stirred for 20 hours at room temperature. Then the reaction was diluted with ethyl acetate-toluene (1:1, 50 ml) and washed 2M hydrochloric aqueous solution, water, saturated aqueous sodium bicarbonate solution, water, and brine. The organic layer was dried over sodium sulfate and concentrated in vacuo to give crude product. The crude product was purified by column chromatography on silica gel with ethyl acetate-hexane (1:1) to furnish the title compound as a white solid (38.9 mg, 51% yield).

¹H NMR (270 MHz, DMSO-d₆) [symbol] 0.76-0.91 (2H, m), 0.99-1.13 (2H, m), 1.47 (3H, d, J=7.3 Hz), 2.04-2.19 (1H, m), 2.29 (3H, s), 3.02 (3H, s), 5.32-5.54 (1H, m), 7.25-7.37 (2H, m), 7.45 (1H, 7.68 (1H, s), 7.81-8.01 (3H, m), 8.44 (1H, s), 8.99 (1H, d, J=7.3 Hz), 9.21 (1H, br.s).

MS (ESI) m/z 455 (M−H)⁻, 457 (M+H)⁺.

### Example 53

### (R)—N-(1-(2-CHLORO-5-METHYL-4-(METHYLSULFONAMIDO)PHENYL)ETHYL)-7-(TRIFLUOROMETHYL)QUINOLINE-3-CARBOXAMIDE

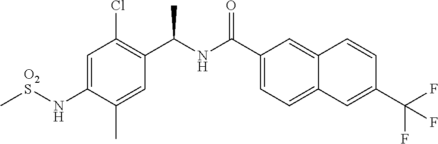

To a DMF (3 ml) solution of the compound of Example 52D (50 mg, 0.17 mmol), 7-trifluoromethyl-quinoline-3-carboxylic acid (40.3 mg, 0.167 mmol), WSC (48 mg, 0.251 mmol), and HOBt hydrate (7.7 mg, 0.050 mmol) was added triethylamine (0.070 ml, 0.501 mmol) and the mixture was stirred for 20 hours at room temperature. Then the reaction was diluted with ethyl acetate-toluene (1:1, 50 ml) and washed saturated aqueous sodium bicarbonate solution, water and brine. The organic layer was dried over sodium sulfate and concentrated in vacuo to give crude product. The crude product was purified by column chromatography on silica gel with ethyl acetate-hexane (2:1) to furnish the title compound as a white solid (39.1 mg, 48% yield).

¹H NMR (270 MHz, DMSO-d₆) [symbol] 1.50 (3H, d, J=7.2 Hz), 2.29 (3H, s), 3.03 (3H, s), 5.35-5.62 (1H, m), 7.34 (1H, s), 7.47 (1H, s), 8.13 (1H, d, J=9.2 Hz), 8.31 (1H, d, J=8.6 Hz); 8.66 (1H, s), 9.07 (1H, s), 9.23 (1H, s), 9.33 (1H, d, J=7.2 Hz), 9.46 (1H, d, J=2.6 Hz).

MS (ESI) m/z 484 (M−H)⁻, 486 (M+H)⁺.

### Example 54

### N-((1R)-1-{4-METHYL-5-[(METHYLSULFONYL)AMINO]PYRIDIN-2-YL}ETHYL)-6-(TRIFLUOROMETHYL)-2-NAPHTHAMIDE

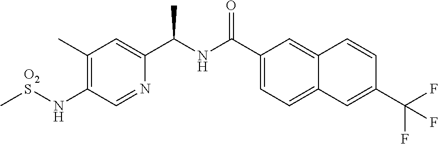

To a DMF (3.0 ml), solution of the N-[6-[(1R)-1-aminoethyl]-4-methylpyridin-3-yl]methanesulfonamide (prepared by an analogous method described for compound 2E) (50 mg, 0.22 mmol), 6-(trifluoromethyl)-2-naphthoic acid (52.4 mg, 0.22 mmol) and HBTU (99.2 mg, 0.262 mmol) was added triethylamine (0.091 ml, 0.654 mmol) and the mixture was stirred for 6 hours at room temperature. The same procedure as described in Example 1G was performed to give the title compound (68.1 mg, 70% yield) as a white solid.

¹H NMR (270 MHz, DMSO-d₆) δ 1.55 (3H, d, J=7.0 Hz), 2.33 (3H, s), 3.04 (3H, s), 5.17-5.30 (1H, m), 7.37 (1H, s), 7.80-7.88 (1H, m), 8.08-8.15 (1H, m), 8.21-8.33 (2H, m), 8.38 (1H, s), 8.50 (1H, s), 8.65 (1H, s), 9.05-9.12 (1H, m), 9.31 (1H, s).

MS (ESI) m/z 450.20 (M−H)⁻, 452.12 (M+H)⁺.

### Example 55

### N-((1R)-1-{2-FLUORO-5-METHYL-4-[(METHYLSULFONYL)AMINO]PHENYL}ETHYL)-2-(2,2,2-TRIFLUORO-1,1-DIMETHYLETHYL)QUINOLINE-6-CARBOXAMIDE

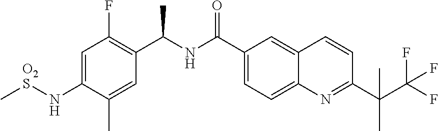

A DMF (3.5 ml) solution of the compound of Example 13D (87 mg, 0.35 mmol), triethylamine (0.15 ml, 1.1 mmol), the compound of Example 49E (100 mg, 0.35 mmol), HOBt.hydrate (5.4 mg, 0.035 mmol) and N,N′-dicyclohexylcarbodiimide (80 mg, 0.39 mmol) was stirred at room temperature for 16 hours and at 120° C. for 5 hours. The mixture was quenched with saturated sodium bicarbonate aqueous solution, and the product was extracted with ethyl acetate which was dried over sodium sulfate. Then, filtration, evaporation, purification through silica gel column chromatography eluting with hexane/ethyl acetate (1:1) and HPLC (used column was XTerra MS C18, 5 um, 30×50 mm) eluting with acetonitrile/0.01% ammonium aqueous solution (4:96 to 96:4) to furnish the title compound (17.3 mg, 10% yield) as a white solid.

¹H NMR (300 MHz, DMSO-d₆) [symbol] 1.50 (3H, d, J=7.3 Hz), 1.72 (6H, s), 2.24 (3H, s), 3.01 (3H, s), 5.36-5.44 (1H, m), 7.09 (1H, d, J=11.1 Hz), 7.36 (1H, d, J=8.1 Hz), 7.88 (1H, d, J=8.8 Hz), 8.09 (1H, d, J=8.1 Hz), 8.23 (1H, d, J=8.8 Hz), 8.54-8.59 (2H, m), 9.10 (1H, d, J=7.3 Hz), 9.20 (1H, brs).

MS (ESI): m/z 512 (M+H)⁺.

### Example 56

### (R)—N-(1-(2-FLUORO-5-METHYL-4-(METHYLSULFONAMIDO)PHENYL)ETHYL)-6-(TRIFLUOROMETHYL)QUINOLINE-2-CARBOXAMIDE

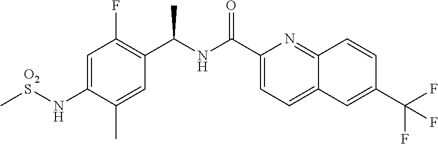

To a DMF (2 ml) solution of the compound of Example 13D (35.2 mg, 0.12 mmol), 6-(trifluoromethyl)quinoline-2-carboxylic acid (WO2005/033082, 30 mg, 0.12 mmol), WSC (35.8 mg, 0.187 mmol), and HOBt hydrate (5.7 mg, 0.037 mmol) was added triethylamine (0.052 ml, 0.373 mmol) and the mixture was stirred for 20 hours at room temperature. Then the reaction was diluted with ethyl acetate-toluene (1:1, 100 ml) and washed 2M hydrochloric aqueous solution, water, saturated aqueous sodium bicarbonate solution, water and brine. The organic layer was dried over sodium sulfate and concentrated in vacuo to give crude product. The crude product was purified by column chromatography on silica gel with ethyl acetate-hexane (1:2) to furnish the title compound as a white solid (9.2 mg, 16% yield).

¹H NMR (270 MHz, DMSO-d₆) [symbol] 1.58 (3H, d, J=7.2 Hz), 2.25 (3H, s), 3.02 (3H, s), 5.31-5.58 (1H, m), 7.11 (1H, d, J=11.9 Hz), 7.45 (1H, d, J=7.9 Hz), 8.14 (1H, d, J=8.6 Hz), 8.25 (1H, d, J=8.6 Hz), 8.41 (1H, d, J=9.3 Hz), 8.66 (1H, s), 8.79 (1H, d, J=8.6 Hz), 9.20 (1H, br.s), 9.29 (1H, br.d, J=8.0 Hz).

MS (ESI) m/z 468 (M−H)⁻, 470 (M+H)⁺.

### Example 57

### (R)—N-(1-(2-CHLORO-5-METHYL-4-(METHYLSULFONAMIDO)PHENYL)ETHYL)-6-(TRIFLUOROMETHYL)QUINOLINE-2-CARBOXAMIDE

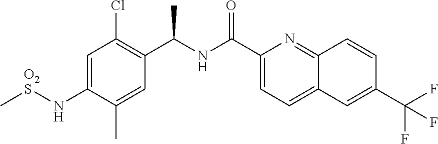

To a DMF (3 ml) solution of the compound of Example 52D (50 mg, 0.17 mmol), 6-(trifluoromethyl)quinoline-2-carboxylic acid (WO2005/033082, 40.3 mg, 0.167 mmol), WSC (48 mg, 0.251 mmol), and HOBt hydrate (7.7 mg, 0.050 mmol) was added triethylamine (0.070 ml, 0.501 mmol) and the mixture was stirred for 20 hours at room temperature. Then the reaction was diluted with ethyl acetate-toluene (1:1, 50 ml) and washed 2M hydrochloric aqueous solution, water, saturated aqueous sodium bicarbonate solution, water and brine. The organic layer was dried over sodium sulfate and concentrated in vacuo to give crude product. The crude product was purified by column chromatography on silica gel with ethyl acetate-hexane (1:2) to furnish the title compound as a white solid (65.6 mg, 81% yield).

¹H NMR (270 MHz, DMSO-d₆) [symbol] ppm. 1.56 (3H, d, J=6.6 Hz), 2.26 (3H, s), 3.02 (3H, s), 5.30-5.63 (1H, m), 7.33 (1H, s), 7.53 (1H, s), 8.15 (1H, d, J=9.3 Hz), 8.24 (1H, d, J=8.5 Hz), 8.43 (1H, d, J=8.6 Hz); 8.66 (1H, s), 8.79 (1H, d, J=8.6 Hz), 9.23 (1H, s), 9.40 (1H, br.d, J=8.6 Hz).

MS (ESI) m/z 484 (M−H)⁻.

### Example 58

### (R)—N-(1-(4-METHYL-5-(METHYLSULFONAMIDO)PYRIDIN-2-YL)ETHYL)-6-(1-METHYL-CYCLOPROPYL)-2-NAPHTHAMIDE

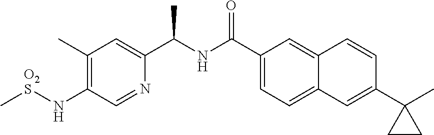

To a DMF (3 ml) solution of the compound of Example 40E (60 mg, 0.26 mmol), triethylamine (0.109 ml, 0.785 mmol), the compound of Example 50C (59.2 mg, 0.262 mmol), and HBTU (109 mg, 0.288 mmol) was treated in the same procedure described in Example 2G. The crude residue was applied to a silica gel column chromatography and eluted with hexane/ethyl acetate (1:2 to 1:4) and HPLC (used column was XTerra MS C18, 5 um, 30×50 mm) eluting with acetonitrile/0.01% ammonium aqueous solution (basic 4_{—}40, 4:96 to 40:60) to furnish the title compound (59 mg, 52% yield) as white solid.

¹H NMR (270 MHz, DMSO-d₆) [symbol] ppm 0.82-0.92 (2H, m), 0.95-1.07 (2H, m), 1.50 (3H, s), 1.54 (3H, d, J=6.6 Hz), 2.32 (3H, s), 3.03 (3H, s), 5.22 (1H, m), 7.35 (1H, s), 7.41 (1H, d, J=8.6 Hz), 7.82 (1H, s), 7.88-8.01 (3H, m), 8.37 (1H, s), 8.48 (1H, s), 8.92 (1H, d, J=7.9 Hz), 9.29 (1H, br s).

MS (ESI): m/z 438 (M+H)⁺, 436 (M−H)⁺.

### Example 62

### QUINOLINE-3-CARBOXYLIC ACID [(R)-1-(4-METHANESULFONYLAMINO-3-METHYL-PHENYL)-ETHYL]-AMIDE

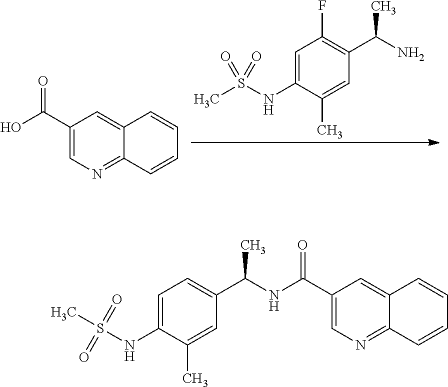

The compound is prepared in a similar manner as Example 63C by condensing the acid (100 mg, 0.065 mmol) with the appropriate amine (131 mg, 0.057 mmol) to give the title compound (14 mg, 6%).

m/z=384.2 (M+1), r.t. 2.39 min.

¹H NMR (400 MHz; d₆-DMSO) δ 9.29 (1H, d), 9.15 (1H, d), 9.01 (1H, s), 8.87 (1H, d), 8.09 (2H, t), 7.85 (1H, ddd), 7.69 (1H, ddd), 7.31 (1H, s), 7.28-7.22 (2H, m), 5.12-5.08 (1H, m), 2.98 (3H, s), 2.35 (3H, s), 1.51 (3H, d).

### Example 63

### 4-PYRROLIDIN-1-YL-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

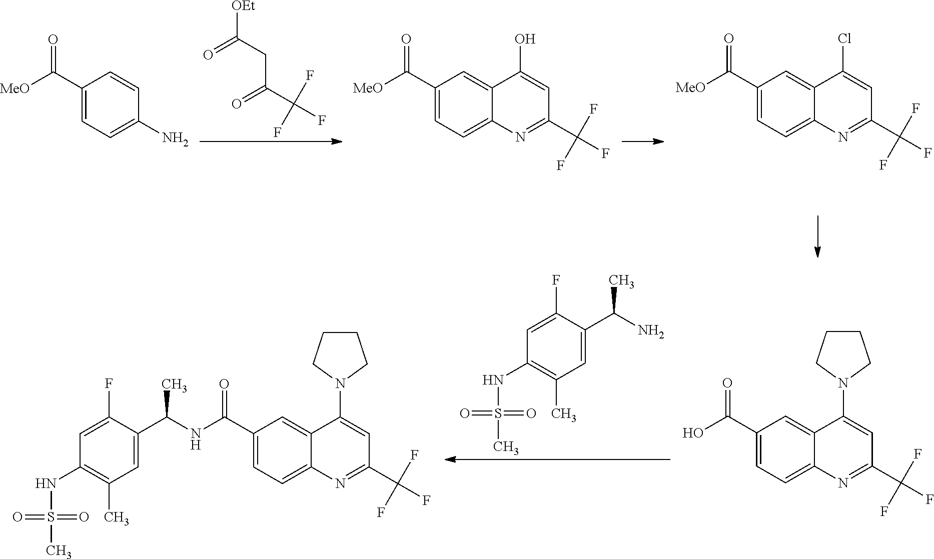

### 63A) METHYL 4-CHLORO-2-(TRIFLUOROMETHYL)QUINOLINE-6-CARBOXYLATE

A mixture of, 4-aminobenzoic acid methyl ester (1000 mg, 7 mmol) and 4,4,4-trifluoro-3-oxo-butanoic acid ethyl ester (1.1 mL, 7.5 mmol) in polyphosphoric acid (10 mL) were heated at 125° C. overnight. The reaction was cooled and water was added. The precipitate formed was filtered off to give the quinoline derivative, which was used without further purification into the next step.

m/z=271.9 (M+1), r.t. 2.48 min.

The above quinoline obtained was heated in neat POCl₃ (20 mL) at 110° C. for 20 h. The reaction mixture was cooled and poured carefully into a mixture of NH₄OH-ice water. The pH of the aqueous layer was acidified to pH 5 by adding 1N HCl and extracted with EtOAc. The combined organic layers were dried (MgSO₄), concentrated in vacuo and the residue was triturated with MeOH/Et₂O/hexane to give the title product as a beige solid (2800 mg, 32%).

m/z=290.0 (M+1), r.t. 3.77 min.

¹H NMR (400 MHz; CD₃OD) δ 8.91 (1H, d), 8.38 (1H, d), 8.19 (1H, d), 8.04 (1H, s), 3.93 (3H, s).

### 63B) 4-(PYRROLIDIN-1-YL)-2-(TRIFLUOROMETHYL)QUINOLINE-6-CARBOXYLIC ACID

A solution of methyl 1-chloro-2-(trifluoromethyl)quinoline-6-carboxylate (200 mg, 0.70 mmol), cesium fluoride (105 mg, 0.69 mmol), triethylamine (193 μL, 1.38 mmol) and pyrrolidine (57 μL, 0.68 mmol) in 4 mL of DMSO were heated in the microwave at 150° C. for 16 h. The reaction mixture was dissolved in EtOAc and the organic layer was washed with brine. The combined organic layers were dried (MgSO₄), filtered and concentrated in vacuo to give a mixture of the desired ester and the corresponding acid that was used without further purification in the next step. m/z=325.0 (M+1), r.t. 2.74 min.

The above mixture was dissolved in 15 mL MeOH and 5 mL of water, and Lithium hydroxide (100 mg, 4 mmol) was added. The reaction mixture was heated to reflux for 30 min. The solvents were removed in vacuo and the solid obtained was suspended in water. 1N HCl was added until pH 4 and the precipitated formed was filtered off to give the title compound (120 mg, 57%). m/z=311.0 (M+1), r.t. 2.44 min. ¹H NMR (400 MHz; d₆-DMSO) δ 9.07 (1H, d), 8.14 (1H, dd), 7.90 (1H, d), 6.84 (1H, s), 3.81-3.77 (4H, m), 2.10-2.02 (4H, m).

### 63C) (R)—N-(1-(2-FLUORO-5-METHYL-4-(METHYLSULFONAMIDO)PHENYL)ETHYL)-4-PYRROLIDIN-2-(TRIFLUOROMETHYL)QUINOLINE-6-CARBOXAMIDE

To a solution of 4-pyrrolidino-2-(trifluoromethyl)quinoline-6-carboxylic acid (50 mg, 0.20 mmol) in 40 mL CH₂Cl₂, oxalyl chloride (28 μL, 0.33 mmol) was added followed by one drop of N,N-dimethylformamide. The reaction mixture was stirred at room temperature for 1.5 hr. The volatiles were removed in vacuo and the residue was dissolved in 10 mL CH₂Cl₂. To this mixture, a solution of N-[4-((R)-1-aminoethyl)-5-fluoro-2-methylphenyl]methane-sulfonamide hydrochloride (46 mg, 0.16 mmol) and triethylamine (70 μL, 0.5 mmol) in 10 mL of CH₂Cl₂ was added followed by addition of more triethylamine (70 μL, 0.5 mmol). After stirring at room temperature for 3 hr, the reaction mixture was washed with water and extracted with CH₂Cl₂. The combined organic layers were dried (MgSO₄), filtered and concentrated in vacuo. PTLC using EtOAc as an eluent gave the title product (26 mg, 26%). m/z=539.9 (M+1), r.t. 2.86 min. ¹H NMR (400 MHz; d₆-DMSO) δ 9.22 (1H, bs), 9.12 (1H, d), 8.92 (1H, d), 8.17 (1H, dd), 7.92 (1H, d), 7.36 (1H, d), 7.12 (1H, d), 6.79 (1H, s), 5.42-5.33 (1H, m), 3.84 (4H, bs), 3.02 (3H, s), 2.25 (3H, s), 2.08 (4H, bs), 1.50 (3H, d).

### Example 64

### (R)-6-FLUORO-N-(1-(2-FLUORO-5-METHYL-4-(METHYLSULFONAMIDO)PHENYL)ETHYL)-7-(TRIFLUOROMETHYL)QUINOLINE-3-CARBOXAMIDE

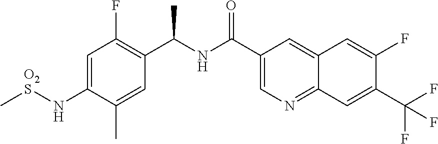

### 64A) ETHYL 6-FLUORO-4-HYDROXY-7-(TRIFLUOROMETHYL)QUINOLINE-3-CARBOXYLATE

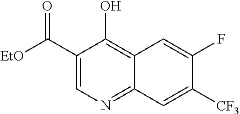

A mixture of 3-trifluoromethyl-4-fluoroaniline (15 g, 84 mmol, purchased from Wako) and diethylethoxymethylene malonate (22.8 mL, 113 mmol) was heated slowly as follows; 60° C. for 10 minutes, 90° C. for 15 minutes, 140° C. for 90 minutes. After ethanol was removed in vacuo, the residue was solidified upon standing. This solid was added portionwise to a boiling diphenyl ether (278 mL) at 250° C., and the resulting dark yellow solution was stirred at this temperature for 90 minutes. After being cooled to room temperature, the white solid began to precipitate out. This solid material was filtered, and washed with ethyl acetate-hexanes 2:1 (ca. 500 mL) to give a crude title compound as a white solid (3.38 g, 13% yield). This crude product was used for the next step without further purification.

¹H NMR (270 MHz, DMSO-d₆) [symbol] 1.30 (3H, t, J=7.3 Hz), 4.25 (2H, q, J=7.3 Hz), 7.98-8.16 (1H, m), 8.73 (1H, s), 12.59 (1H, br s).

MS (ESI) m/z 302 (M−H)⁻, 304 (M+H)⁺.

### 64B) ETHYL 4-CHLORO-6-FLUORO-7-(TRIFLUOROMETHYL)QUINOLINE-3-CARBOXYLATE

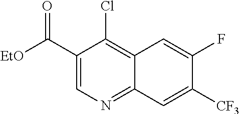

A mixture of Example 64A (3.38 g, 11.1 mmol) and phosphorous oxychloride (20.8 mL, 223 mmol) was heated under reflux for 120 minutes. After phosphorous oxychloride was removed in vacuo, the residue was diluted in dichloromethane and poured into crashed ice-25% ammonia water mixture portionwise. The aqueous layer was extracted with dichloromethane 3 times. The combined organic extracts were washed with brine, dried over sodium sulfate and concentrated. The residue was chromatographed on a column of silica gel eluting with ethyl acetate-hexane (1:5) gave the title compound (3.38 g, 94%) as white solid.

¹H NMR (270 MHz, CDCl₃) [symbol] 1.48 (3H, t, J=7.3 Hz), 4.54 (2H; q, J=7.3 Hz), 8.19 (1H, d, J=11.2 Hz), 8.49 (1H, d, J=6.6 Hz), 8.25 (1H, s).

MS (ESI) m/z 322 (M+H)⁺.

### 64C) ETHYL 6-FLUORO-7-(TRIFLUOROMETHYL)QUINOLINE-3-CARBOXYLATE

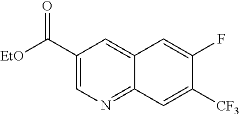

A mixture of Example 64B (3.38 g, 10.5 mmol), 5% palladium on activated carbon (338 mg), triethylamine (2.93 mL, 21.0 mmol) and ethanol (50 mL) was hydrogenated (1 atm, balloon) at room temperature for 90 minutes. The reaction mixture was filtered over a pad of celite, and the filtrate was evaporated. The residue was chromatographed on a column of silica gel eluting with ethyl acetate-hexane (1:10 to 1:5) gave the title compound (2.94 g, 97%) as yellow solid.

¹H NMR (270 MHz, CDCl₃) [symbol] 1.48 (3H, t, J=7.3 Hz), 4.51 (2H, q, J=7.3 Hz), 7.72 (1H, d, J=9.9 Hz), 8.51 (1H, d, J=7.3 Hz), 8.84 (1H, br s), 9.51 (1H, br s).

MS (ESI) m/z 288 (M+H)⁺.

### 64D) 6-FLUORO-7-(TRIFLUOROMETHYL)QUINOLINE-3-CARBOXYLIC ACID

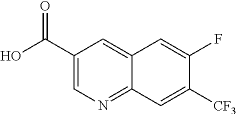

To a solution of the Example 64C (2.94 g, 10.2 mmol) in ethanol (4 ml) was added 2N aqueous sodium hydroxide (10.2 mL, 20.5 mmol) at room temperature. The mixture was stirred at 60° C. for 2 hours. Then the reaction mixture was neutralized to pH 5˜6 by 2N aqueous hydrochloride. The formed precipitate was collected, washed with water to furnish the title compound (2.52 g, 95%) as a white solid.

¹H NMR (270 MHz, DMSO-d₆) [symbol] 8.32 (1H, d, J=11.2 Hz), 8.51 (1H; d, J=7.3 Hz), 9.01 (1H, s), 9.42 (1H, s)

MS (ESI): m/z 260 (M+H)⁺258 (M−H)⁺.

### 64E) (R)-6-FLUORO-N-(1-(2-FLUORO-5-METHYL-4-(METHYLSULFONAMIDO)PHENYL)-ETHYL)-7-(TRIFLUOROMETHYL)QUINOLINE-3-CARBOXAMIDE

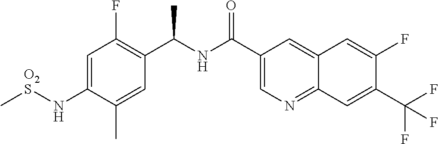

To a DMF (2 ml) solution of the compound of Example 13D (49 mg, 0.172 mmol), triethylamine (0.072 ml, 0.518 mmol), the compound of Example 64D (45 mg, 0.1.73 mmol), and HBTU (72 mg, 0.190 mmol) was treated in the same procedure described in Example 1G. The crude residue was applied to a silica gel column chromatography and eluted with hexane/ethyl acetate (1:1) and HPLC (used column was XTerra MS C18, 5 um, 30×50 mm) eluting with acetonitrile/0.01% ammonium aqueous solution (basic 32_{—}68, 32:68 to 68:32) to furnish the title compound (24 mg, 29% yield) as white solid.

¹H NMR (270 MHz, DMSO-d₆) [symbol] 1.52 (3H, d, J=7.3 Hz), 2.26 (3H, s), 3.02 (3H, s), 5.40 (1H, m), 7.11 (1H, d, J=11.9 Hz), 7.37 (1H, d, J=8.6 Hz), 8.29 (1H, d, J=8.2 Hz), 8.55 (1H, d, J=8.6 Hz), 8.94 (1H, s), 9.24 (1H, br s), 9.35 (1H, d, J=7.3 Hz), 9.39 (1H, s).

MS (ESI): m/z 488 (M+H)⁺, 486 (M−H)⁺.

### Example 65

### (R)-6-CHLORO-N-(1-(2-FLUORO-5-METHYL-4-(METHYLSULFONAMIDO)PHENYL)ETHYL)-7-(TRIFLUOROMETHYL)QUINOLINE-3-CARBOXAMIDE

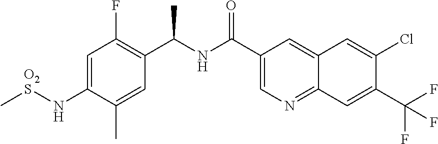

### 65A) ETHYL 6-CHLORO-4-HYDROXY-7-(TRIFLUOROMETHYL)QUINOLINE-3-CARBOXYLATE

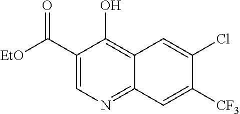

The title compound was prepared by the same procedure of Example 64A using 3-trifluoromethyl-4-chloroaniline instead of 3-trifluoromethyl-4-fluoroaniline.

¹H NMR (270 MHz, DMSO-d₆) [symbol] 1.30 (3H, t, J=7.3 Hz), 4.25 (2H, q, J=73 Hz), 8.16 (1H, s), 8.73 (1H, s), 12.60 (1H, br s).

MS (ESI) m/z 318 (M−H)⁻, 320 (M+H)⁺.

### 65B) ETHYL 4-BROMO-6-CHLORO-7-(TRIFLUOROMETHYL)QUINOLINE-3-CARBOXYLATE

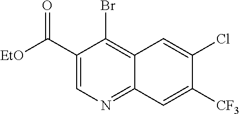

A mixture of Example 65A (2.00 g, 6.26 mmol), phosphorous oxybromide (5.38 g, 18.8 mmol) and N,N-dimethylformamide (40 mL) was stirred at 70° C. for 2 hours. After phosphorous oxychloride was removed in vacuo, the residue was diluted in dichloromethane and poured onto crashed ice carefully. The mixture was diluted with saturated aqueous sodium bicarbonate (300 mL), extracted with dichloromethane 3 times (total 150 mL). The combined organic extracts were dried over sodium sulfate and concentrated. The residue was chromatographed on a column of silica gel eluting with ethyl acetate-hexane (1:5) gave the title compound (2.15 g, 90%) as white solid.

¹H NMR (270 MHz, CDCl₃) [symbol] 1.48 (3H, t, J=7.3 Hz), 4.54 (2H, q, J=7.3 Hz), 8.53 (1H, s), 8.56 (1H, s), 9.14 (1H, s).

MS (ESI) m/z 384, 382 (M+H)⁺.

### 65C) ETHYL 6-CHLORO-7-(TRIFLUOROMETHYL)QUINOLINE-3-CARBOXYLATE

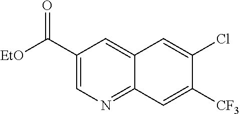

A mixture of Example 6513 (2.15 g, 5.63 mmol), 5% palladium on activated carbon (215 mg), triethylamine (1.57 mL, 11.30 mmol) and ethanol (56 mL) was hydrogenated (1 atm, balloon) at room temperature for 55 minutes. The reaction mixture was filtered over a pad of celite, and the filtrate was evaporated. The residue was chromatographed on a column of silica gel eluting with ethyl acetate-hexane (1:10 to 1:5) gave the mixture of the title compound and des-diCl derivative (ethyl 7-(trifluoromethyl)quinoline-3-carboxylate). The mixture was recrystallized from hexane (50 mL) gave the title compound (0.61 g, 35%) as white solid.

¹H NMR (270 MHz, CDCl₃) [symbol] 1.48 (3H, t, J=7.3 Hz), 4.52 (2H, q, J=7.3 Hz), 8.10 (1H, s), 8.57 (1H, s), 8.81 (1H, d, J=2.0 Hz), 9.53 (1H, d, J=2.0 Hz).

MS (ESI) m/z 304 (M+H)⁺.

### 65D) 6-CHLORO-7-(TRIFLUOROMETHYL)QUINOLINE-3-CARBOXYLIC ACID

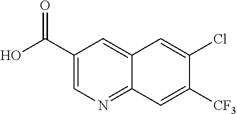

The title carboxylic acid was prepared by the same procedure of Example 64D to give the title compound (595 mg, quant) as white solid.

¹H NMR (270 MHz, DMSO-d₆) [symbol] 8.57 (1H, s), 8.67 (1H, s), 9.09 (1H, d, J=2.0 Hz), 9.44 (1H, d, J=2.0 Hz)

MS (ESI): m/z 276 (M+H)⁺ 274 (M−H)⁺.

### 65E) (R)-6-CHLORO-N-(1-(2-FLUORO-5-METHYL-4-(METHYLSULFONAMIDO)PHENYL)ETHYL)-7-(TRIFLUOROMETHYL)QUINOLINE-3-CARBOXAMIDE

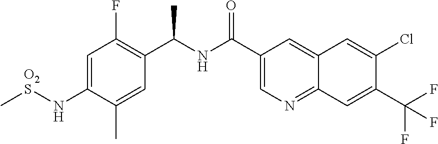

To a DMF (2 ml) solution of the compound of Example 13D (60 mg, 0.201 mmol), triethylamine (0.084 ml, 0.604 mmol), the compound of Example 65D (56 mg, 0.201 mmol), and HBTU (84 mg, 0.221 mmol) was treated in the same procedure described in Example 1G. The crude residue was applied to a silica gel column chromatography and eluted with hexane/ethyl acetate (1:1) to furnish the title compound (20 mg, 20% yield) as white solid.

¹H NMR (270 MHz, DMSO-d₆) [symbol] 1.52 (3H, d, J=6.6 Hz), 2.26 (3H, s), 3.02 (3H, s), 5.40 (1H, m), 7.11 (1H, d, J=11.9 Hz), 7.37 (1H, d, J=7.9 Hz), 8.58 (2H, s), 8.92 (1H, s), 9.36 (1H, d, J=7.3 Hz), 9.44 (1H, d, J=2.0 Hz). The amide N—H peak was not observed.

MS (ESI): m/z 504 (M+H)⁺, 502 (M−H)⁺.

### Example 66

### 7-BROMO-[1,5]NAPHTHYRIDINE-3-CARBOXYLIC ACID [(R)-1-(2-FLUORO-4-METHANE-SULFONYLAMINO-5-METHYL-PHENYL)-ETHYL]-AMIDE

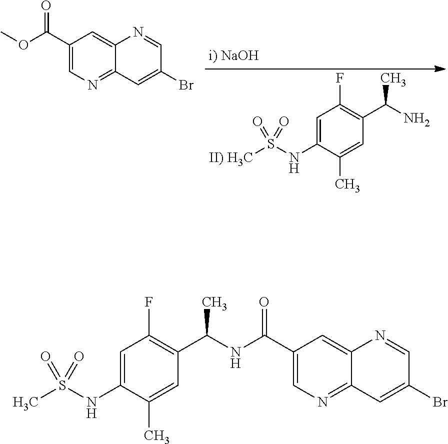

66A) To a solution of methyl 7-bromo-1,5-naphthyridine-3-carboxylate (200 mg; 0.70 mmol) in Methanol (3 mL, 70 mmol), 1N Sodium hydroxide in water (3 mL, 3 mmol) was added and the reaction was stirred at room temperature 2 h. The reaction mixture was neutralized with 1N HCl, dried (MgSO₄), filtered and evaporated. The crude residue was used in the next step without further purification. m/z=255.11 (M+1), r.t. 2.56 min.

66B) A solution of the crude acid, N-[4-((R)-1-aminoethyl)-5-fluoro-2-methylphenyl]methanesulfonamide hydrochloride (35 mg, 0.14 mmol), N,N,N′,N′-tetramethyl-O-(7-azabenzotriazol-1-yl)uronium hexafluorophosphate (100 mg, 0.30 mmol) and N,N-diisopropylethylamine (0.084 mL, 0.48 mmol) in N,N-Dimethylformamide (1.1 mL, 15 mmol) was stirred at room temperature 16 hr. The reaction mixture was quenched with 1N HCl, neutralized with triethylamine and concentrated in vacuo. The residue was purified by prep HPLC (25-65 method) to give the title product (7.4 mg, 10%) as a tan solid. m/z=483.1 (M+1), r.t. 2.70 min. ¹H NMR (400 MHz; d₆-DMSO-d₆) δ 9.39 (1H, d), 9.35 (1H, d), 9.19 (1H, d), 9.02-8.95 (2H, m), 8.90-8.84 (1H, m), 7.34 (1H, d), 7.08 (1H, d), 5.42-5.35 (1H, m), 2.97 (3H, s), 2.22 (3H, s), 1.51 (3H, d).

### Example 67

### QUINOXALINE-2-CARBOXYLIC ACID [(R)-1-(2-FLUORO-4-METHANESULFONYLAMINO-5-METHYLPHENYL)ETHYL]AMIDE

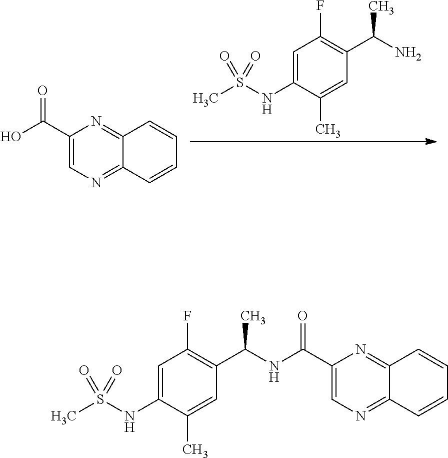

To a stirred solution of quinoxaline-2-carboxylic acid (26 mg, 0.15 mmol), N-[4-((R)-1-aminoethyl)-5-fluoro-2-methylphenyl]methanesulfonamide hydrochloride (35 mg, 0.12 mmol), and N,N,N′,N′-tetramethyl-O-(7-azabenzotriazol-1-yl)uronium hexafluorophosphate (56 mg, 0.15 mmol) in N,N-dimethylformamide (0.5 mL) was added N,N-diisopropylethylamine (80 mg, 0.6 mmol). A catalytic amount of DMAP was added, and the reaction was stirred at room temperature for 16 hours. The reaction mixture was concentrated down to a solid. The crude product was suspended in MeOH and filtered, and the filtrate purified by HPLC to give the title compound (25.0 mg, 49%) as an off-white solid. m/z=403.1 (M+1), r.t. 2.83 min. ¹H NMR (400 MHz; d₆-DMSO) δ 9.43 (1H, s), 9.39 (1H, d), 8.30-8.26 (1H, m), 8.22-8.18 (1H, m), 8.03-7.97 (2H, m), 7.45 (1H, d), 7.11 (1H, d), 5.49-5.39 (1H, m), 3.02 (3H, s), 2.24 (3H, s), 1.57 (3H, d).

### Example 68

### (R)—N-(1-(2-FLUORO-5-METHYL-4-(METHYLSULFONAMIDO)PHENYL)ETHYL)-2-(PYRROLIDIN-1-YL)QUINOLINE-6-CARBOXAMIDE

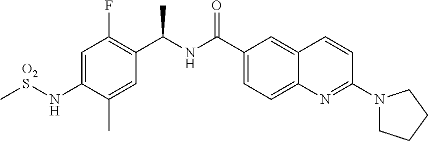

### 68A) ETHYL 2-(PYRROLIDIN-1-YL)QUINOLINE-6-CARBOXYLATE

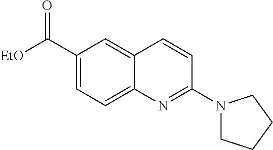

A mixture of the compound of Example 69A (200 mg, 0.714 mmol) and pyrrolidine (254 mg, 3.56 mmol) in ethanol (7 ml) was stirred at 50° C. for 2 hours. The reaction mixture was evaporated to remove the solvents, and the residue was chromatographed on a column of silica gel (ethyl acetate-hexane=1:10 to 1:3) as eluent to give the title compound (119 mg, 62%) as white solid.

¹H NMR (270 MHz, CDCl₃) [symbol] 1.42 (3H, t, J=6.6 Hz), 1.97-2.17 (4H, m), 3.50-3.78 (4H, m), 4.40 (2H, q, J=6.6 Hz), 6.73 (1H, d, J=9.2 Hz), 7.67 (1H, d, J=9.2 Hz), 7.88 (1H, d, J=9.2 Hz), 8.12 (1H, dd, J=2.0 Hz, 9.2 Hz), 8.33 (1H, d, J=2.0 Hz).

MS (ESI): m/z 271 (M+H)⁺.

### 68B) 2-(PYRROLIDIN-1-YL)QUINOLINE-6-CARBOXYLIC ACID

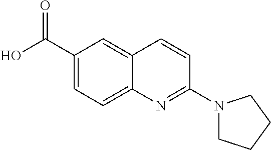

A mixture of the compound of Example 68A (119 mg, 0.425 mmol)and 2M sodium hydroxide solution (0.43 ml, 0.85 mmol) in ethanol (2 ml) was treated in the same procedure described in Example 8B. The aqueous mixture was concentrated and dried in vacuo to give the title compound as white solid (including sodium chloride). These crude products were used for the next step without purification.

MS (ESI): m/z 243 (M+H)⁺, 241 (M−H)⁺.

### 68C) (R)—N-(1-(2-FLUORO-5-METHYL-4-(METHYLSULFONAMIDO)PHENYL)ETHYL)-2-(PYRROLIDIN-1-YL)QUINOLINE-6-CARBOXAMIDE

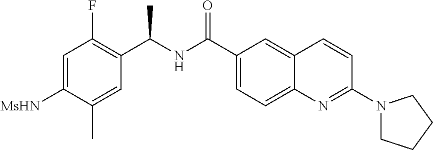

To a DMF (2 ml) solution of the compound of Example 13D (60 mg, 0.212 mmol), triethylamine (64.5 mg, 0.638 mmol), the compound of Example 68B (crude 0.212 mmol), and HBTU (97 mg, 0.255 mmol) was treated in the same procedure described in Example 1G. The crude residue was applied to a silica gel column chromatography and eluted with hexane/ethyl acetate (3:1 to 5:1) to furnish the title compound (70 mg, 70% yield for 2 steps) as white solid.

¹H NMR (270 MHz, DMSO-d₆) [symbol] 1.48 (3H, d, J=7.25 Hz), 1.90-2.08 (4H, m), 2.25 (3H, s), 3.02 (3H, s), 3.50-3.65 (4H, m), 5.37 (1H, m), 6.94 (1H, d, J=9.2 Hz), 7.09 (1H, d, J=1.1.9 Hz), 7.36 (1H, d, J=8.6 Hz), 7.55 (1H, d, J=8.6 Hz), 7.98 (1H, dd, J=2.0 Hz, 8.6 Hz), 8.07 (1H, d, J=9.2 Hz), 8.27 (1H, d, J=2.0 Hz), 8.79 (1H, d, J=7.3 Hz), 9.17 (1H, br s).

MS (ESI) m/z 471 (M+H)⁺, 469 (M−H)⁺.

### Example 69

### (R)-2-(DIMETHYLAMINO)-N-(1-(2-FLUORO-5-METHYL-4-(METHYLSULFONAMIDO)-PHENYL)ETHYL)QUINOLINE-6-CARBOXAMIDE

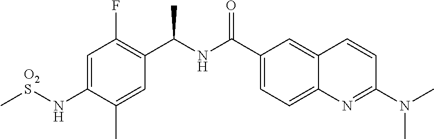

### 69A) ETHYL 2-(OR 4-)BROMOQUINOLINE-6-CARBOXYLATE

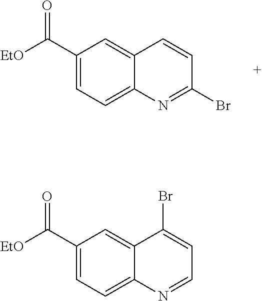

To a mixture of ethyl quinoline-6-carboxylate 1-oxide (4.00 g, 18.0 mmol, Bioorg. Med. Chem. 2005, 1487-1496), DCM (36 ml) and phosphorous oxybromide (10.6 g, 36.8 mmol) was stirred at 50° C. for 1 hours. The reaction mixture was cooled to 0° C., poured onto crashed ice and 25% ammonia solution (50 ml), and stirred for further 3 hours. The mixture was extracted with DCM (150 ml×3 times), dried over sodium sulfate and concentrated in vacuo. The crude material was purified by silica gel column chromatography, eluting with ethyl acetate-hexane (1:2) to give 2.41 g (47% yield) of the title compounds (2:1 mixture) as white solid. These compounds were used for the next step without further purification.

¹H NMR (270 MHz, CDCl₃) [symbol] 1.45 (3H, t, J=7.3 Hz), 4.47 (2H, q, J=7.3 Hz), 7.60 (0.611, d, J=9.2 Hz), 8.03-8.18 (1.6H, m), 8.30-8.34 (0.6H, m), 8.34-8.38 (0.3H, m), 8.43 (0.3H, d, J=2.6 Hz), 8.52 (0.3H, d, J=2.0 Hz), 8.58 (0.6H, d, J=2.0 Hz), 9.00 (0.3H, d, J=2.6 Hz).

MS (ESI): m/z 282 (M+H)⁺

### 69B) ETHYL 2-(DIMETHYLAMINO)QUINOLINE-6-CARBOXYLATE

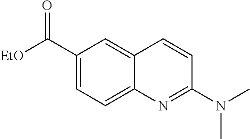

A mixture of dimethylamine hydrochloride (116 mg, 1.42 mmol) and triethylamine (0.199 ml, 1.42 mmol) in DMF (2.5 ml) was stirred at room temperature for 0.5 hours. Then, the compound of Example 69A (133 mg, 0.475 mmol) in DMF (2.5 ml) was added, and the resulting mixture was stirred at room temperature for further 20 hours. The reaction mixture was diluted with toluene-ethyl acetate (1:1, 150 ml), washed with saturated aqueous sodium bicarbonate (50 ml), water (50 ml) and brine (50 ml). The organic layer was dried over sodium sulfate. Removal of the solvent gave a residue, which was chromatographed on a column of silica gel (ethyl acetate-hexane=1:5 to 1:3) as eluent to give the title compound (51 mg, 44% yield) as white solid.

¹H NMR (270 MHz, CDCl₃) [symbol] 1.42 (3H, t, J=7.3 Hz), 3.27 (6H, s), 4.40 (2H, q, J=7.3 Hz), 6.93 (1H, d, J=9.5 Hz), 7.67 (1H, d, J=8.80 Hz), 7.93, (1H, d, J=8.8 Hz), 8.13 (1H, d, J=8.8 Hz), 8.35 (1H, s).

MS (ESI): m/z 245 (M+H)⁺

### 69C) 2-(DIMETHYLAMINO)QUINOLINE-6-CARBOXYLIC ACID

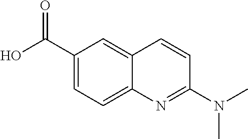

A mixture of the compound of Example 69B (51 mg, 0.208 mmol) and 2M sodium hydroxide solution (0.208 ml, 0.417 mmol) in ethanol (3 ml) was heated at 60° C. for 2 hours. After cooling to room temperature, and the mixture was neutralized with 2M hydrochloric acid solution. The aqueous mixture was concentrated and dried in vacuo to give 143 mg of the title compound as white solid (including sodium chloride). This crude product was used for the next step without purification.

MS (ESI): m/z 217 (M+H)⁺, 215 (M−H)⁺.

### 69D) (R)-2-(DIMETHYLAMINO)-N-(1-(2-FLUORO-5-METHYL-4-(METHYLSULFONAMIDO)-PHENYL)ETHYL)QUINOLINE-6-CARBOXAMIDE

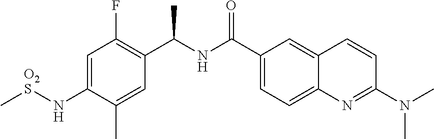

To a DMF (2 ml) solution of the compound of Example 13D (45 mg, 0.208 mmol), triethylamine (0.087 ml, 0.624 mmol), the compound of Example 69C (crude 143 mg, 0.208 mmol), and HBTU (87 mg, 0.229 mmol) was treated in the same procedure described in Example 1G. The crude residue was applied to a silica gel column chromatography and eluted with hexane/ethyl acetate (1:2 to 1:3) to furnish the title compound (74 mg, 80% yield) as white solid.

¹H NMR (270 MHz, DMSO-d₆) [symbol] 1.47 (3H, d, J=6.6 Hz), 2.24 (3H, s), 3.01 (3H, s), 3.19 (6H, s), 5.37 (1H, m), 7.08 (1H, d, J=11.2 Hz), 7.14 (1H, d, J=9.2 Hz), 7.35 (1H, d, J=8.6 Hz), 7.55 (1H, d, J=8.6 Hz), 7.99 (1H, d, J=9.2 Hz), 8.09 (1H, d, J=9.2 Hz), 8.27 (1H, br s), 8.79 (1H, d, J=7.9 Hz), 9.17 (1H, br s).

MS (ESI) m/z 445 (M+H)⁺, 443 (M−H)⁻.

### Example 70

### 4-PIPERIDIN-1-YL-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

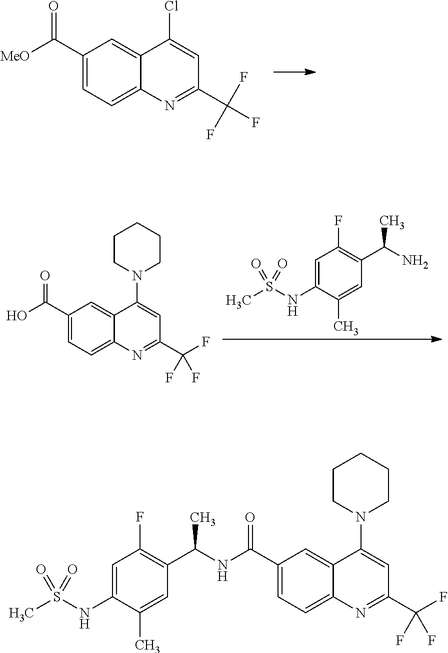

### 70A) 4-(PIPERIDIN-1-YL)-2-(TRIFLUOROMETHYL)QUINOLINE-6-CARBOXYLIC ACID

A solution of ethyl 4-(piperidin-1-yl)-2-(trifluoromethyl)quinazoline-6-carboxylate (50 mg, 0.14 mmol) and lithium hydroxide (100 mg, 0.40 mmol) in 15 mL MeOH and 5 mL of water were heated to reflux for 1 hr. The reaction mixture was cooled and acidified to pH 1 with 1 N HCl and the solvents were removed in vacuo. Flash chromatography (0 to 50% MeOH in EtOAc) gave the title compound (42 mg, 95%). m/z=325.1 (M+1), r.t. 3.33 min. ¹H NMR (400 MHz; d₆-DMSO) [symbol] 13.41 (1H, bs), 8.67 (1H, d), 8.22 (1H, dd), 8.07 (1H, d), 7.27 (1H, s), 3.40-3.35 (4H, m), 1.85 (4H, bs), 1.70-1.63 (2H, m).

### 70B) 4-PIPERIDIN-1-YL-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (100 mg, 0.30 mmol) with the appropriate amine (104 mg, 0.368 mmol) to give the title compound (130 mg, 70%). m/z=552.7 (M+1), r.t. 3.55 min. ¹H NMR (400 MHz; d₆-DMSO) δ 9.22-9.20 (2H, m), 8.50 (1H, d), 8.24 (1H, dd), 8.10 (1H, d), 7.37 (1H, d), 7.25 (1H, s), 7.12 (1H, d), 5.44-5.37 (1H, m), 3.35-3.27 (4H, m), 3.02 (3H, s), 2.25 (3H, s), 1.89-1.78 (4H, m), 1.72-1.61 (2H, m), 1.48 (3H, d).

### Example 71

### ISOQUINOLINE-3-CARBOXYLIC ACID [(R)-1-(2-FLUORO-4-METHANESULFONYLAMINO-5-METHYLPHENYL)ETHYL]AMIDE

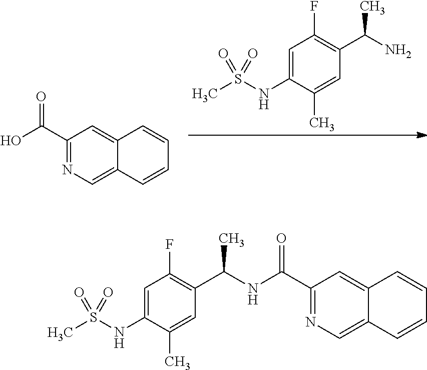

To a stirred solution of isoquinoline-3-carboxylic acid (26 mg, 0.15 mmol), N-[4-((R)-1-aminoethyl)-5-fluoro-2-methylphenyl]methanesulfonamide hydrochloride (35 mg, 0.12 mmol), and N,N,N′,N′-tetramethyl-O-(7-azabenzotriazol-1-yl)uronium hexafluorophosphate (56 mg, 0.15 mmol) in N,N-dimethylformamide (0.5 g) was added N,N-diisopropylethylamine (80 mg, 0.62 mmol). A catalytic amount of DMAP was added and the reaction was stirred for 16 hours at room temperature. The reaction mixture was concentrated and purified by flash chromatography (0 to 5% MeOH in CH₂Cl₂). The resulting product was shaken with water and extracted with EtOAc (3×). The combined organics were dried (MgSO₄), filtered and concentrated to give the title compound (36.8 mg, 74%) as a white solid. m/z=402.0 (M+1), r.t. 3.01 min. ¹H NMR (400 MHz; d₆-DMSO) [symbol] 9.42 (1H, s), 9.21 (1H, s), 916 (1H, 8.53 (1H, s), 8.27 (1H, d), 8.19 (1H, d), 7.87 (1H, t), 7.84 (1H, t), 7.43 (1H d), 7.10 (1H, d), 5.49-5.40 (1H, m), 3.19 (3H, s), 2.23 (3H, s), 1.54 (3H, d).

### Example 72

### 4-MORPHOLIN-4-YL-2-TRIFLUOROMETHYL-QUINOLINE-6-CARBOXYLIC ACID [(R)-1-(2-FLUORO-4-METHANESULFONYLAMINO-5-METHYL-PHENYL)ETHYL]-AMIDE

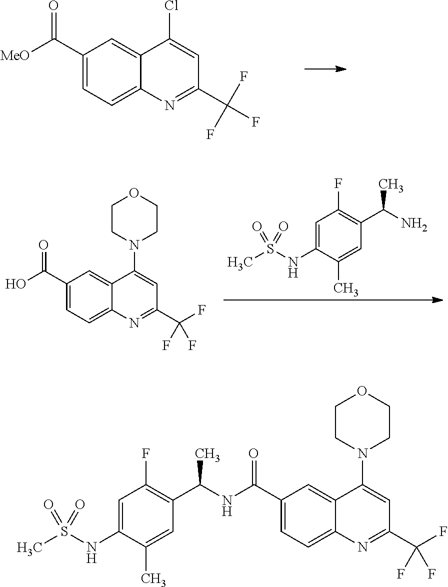

### 72A) 4-(MORPHOLIN-1-YL)-2-(TRIFLUOROMETHYL)QUINOLINE-6-CARBOXYLIC ACID

The compound is prepared in a similar manner as Example 70A by hydrolysis of the ester (200 mg, 0.60 mmol) in basic media to give the title compound (180 mg, 80%). m/z=326.6 (M+1), r.t. 2.75 min. ¹H NMR (400 MHz; d₆-DMSO) δ 13.5 (1H, bs), 8.69 (1H, d), 8.25 (1H, dd), 8.14 (1H, d), 7.35 (1H, s), 3.56-3.60 (4H, m), 2.43-2.52 (4H, m).

### 72B) 4-MORPHOLIN-1-YL-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (23 mg, 0.070 mmol) with the appropriate amine (20 mg, 0.070 mmol) to give the title compound (17 mg, 47%). m/z=555.4 (M+1), r.t. 3.06 min. ¹H NMR (400 MHz; d₆-acetone) δ 8.55 (1H, d), 8.28 (1H, d), 8.14 (1H, dd), 7.98 (1H, d), 7.93 (1H, s), 7.32 (1H, d), 7.22 (1H, s), 7.11 (1H, d), 5.45-5.38 (1H, m), 3.89-3.82 (4H, m), 3.31-3.26 (4H, m), 2.92 (3H, s), 2.23 (3H, s), 1.48 (3H, d).

### Example 73

### 4-(4,4-DIFLUORO-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

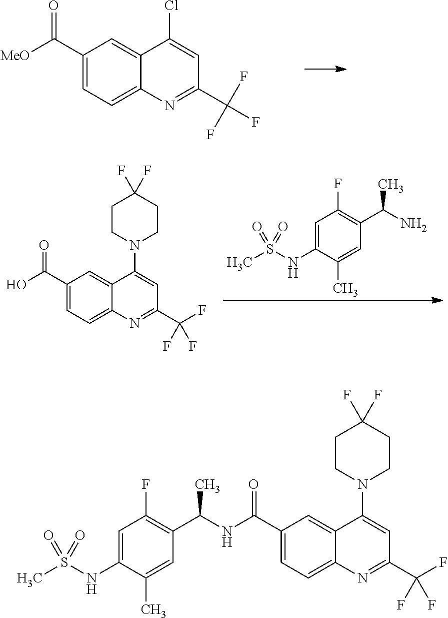

### 73A) 4-(4,4-DIFLUORO-PIPERIDIN-1-YL)-2-(TRIFLUOROMETHYL)QUINOLINE-6-CARBOXYLIC ACID

The compound is prepared in a similar manner as Example 63B by reaction of the chloroquinoline ester (200 mg, 0.70 mmol) with the appropriate amine (109 mg, 0.70 mmol), followed by hydrolysis in basic media to give the title compound (230 mg, 92%). m/z=360.5 (M+1), r.t. 2.88 min.

¹H NMR (400 MHz; d₆-DMSO) δ 8.69 (1H, d), 8.25 (1H, dd), 8.14 (1H, d), 7.41 (1H, s), 3.56-3.40 (4H, m), 2.33-2.26 (4H, m).

### 73B) 4-(4,4-DIFLUORO-PIPERIDIN-1-YL-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (50 mg, 0.10 mmol) with the appropriate amine (40 mg, 0.10 mmol) to give the title compound (5 mg, 5%). m/z=589.2 (M+1), r.t. 3.41 min. ¹H NMR (400 MHz; d₆-DMSO) δ 9.22-9.20 (2H, m), 8.54 (1H, d), 8.27 (1H, dd), 8.12 (1H, d), 7.41 (1H, s), 7.37 (1H, d), 7.10 (1H, d), 5.42-5.35 (1H, m), 3.55-3.43 (4H, m), 3.05 (3H, s), 2.25-2.39 (4H, m), 2.21 (3H, s), 1.48 (3H, d).

### Example 74

### N-((1R)-1-{3-(HYDROXYMETHYL)-4-[(METHYLSULFONYL)AMINO]PHENYL}ETHYL)-6-(TRIFLUOROMETHYL)-2-NAPHTHAMIDE

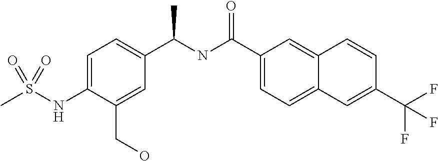

### 74A) ETHYL 5-((1R)-1-{[(R)-TERT-BUTYLSULFINYL]AMINO}ETHYL)-2-[(METHYL-SULFONYL)AMINO]BENZOATE

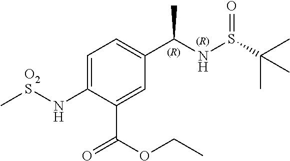

To a mixture of methyl 5-acetyl-2-[(methylsulfonyl)amino]benzoate (13.2 g, 49 mmol, PCT Int. Appl. WO2005003084), titanium (IV) ethoxide (100 ml) and THF (100 ml) was added (R)-(+)-2-methylpropane-2-sulfinamide (5.9 g, 49 mmol) and the mixture was stirred for 16 hours at 80° C. The mixture was cooled to room temperature and then to 0° C. before it was added dropwise into a 0° C. solution of sodium borohydride (7.4 g, 195 mmol). The mixture was stirred at 0° C. for 3 hours and then warmed to room temperature. The reaction was quenched with methanol and stirred for 30 minutes. Then to the mixture water was added. After stirring for 10 minutes, the resulting suspension was filtered through a celite pad and the filtered calm was washed with ethyl acetate. The filtrate was concentrated under reduced pressure to give a residue, which was applied to a silica gel chromatography column and eluted with a volume mixture of dichloromethane and ethyl acetate (1/1) to afford 4.3 g (23% yield) of the title compound as pale yellow solids.

¹H NMR (270 MHz. CDCl₃) δ 1.24 (9H, s), 1.43 (3H, t, J=6.8 Hz), 1.53 (3H, d, J=6.6 Hz), 3.07 (3H, s), 3.39 (1H, br.s), 4.41 (2H, q, J=6.8 Hz), 4.55 (1H, m), 7.56 (1H, dd, J=8.6, 2.0 Hz), 7.74 (1H, d, J=9.2 Hz), 8.06 (1H, d, J=2.0 Hz), 10.49 (1H, br.s). MS (ESI): m/z 391 [M+H]⁺, 389 [M−H]⁻.

### 74B) ETHYL 5-[(1R)-1-AMINOETHYL]-2-[(METHYLSULFONYL)AMINO]BENZOATE

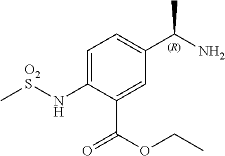

To a solution of the compound of Example 74A (4.3 g, 11 mmol) in MeOH (30 ml) was added 10% hydrogenchloride-methanols solution (30 ml). The mixture was then treated according to the procedure described in Example 13D to afford 3.1 g (87% yields) of the title compound as white solids.

¹H NMR (270 MHz, DMSO-d₆) δ 1.34 (3H, t, J=7.3 Hz), 1.49 (3H, d, J=7.3 Hz), 3.19 (3H, s), 4.36 (2H, q, J=7.3 Hz), 4.45 (1H, m), 7.61 (1H, d, J=8.6 Hz), 7.75 (1H, dd, J=8.6, 2.0 Hz), 8.09 (1H, d, J=2.0 Hz), 8.35 (2H, br.s), 10.14 (1H, br.s).

### 74C) ETHYL 2-[(METHYLSULFONYL)AMINO]-5-((1R)-1-{[6-(TRIFLUOROMETHYL)-2-NAPHTHOYL]AMINO}ETHYL)BENZOATE

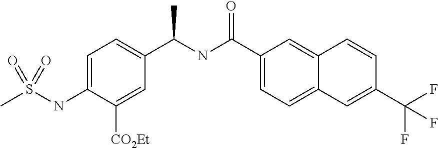

A stirred solution of Example 74B (amine) (202 mg, 0.625 mmol), 6-(trifluoromethyl)-2-naphthoic acid (150 mg, 0.625 mmol), HBTU (284 mg, 0.749 mmol) and triethylamine (190 mg, 0.26 ml, 1.87 mmol) in anhydrous N,N-dimethylformamide (5 mL) was treated in the same procedure described in Example 1G to furnish the title compound (221 mg, 70%) as a white solid.

MS (ESI): m/z 509.14 [M+H]⁺, 507.22 [M−H]⁻.

### 74D) N-((1R)-1-{3-(HYDROXYMETHYL)-4-[(METHYLSULFONYL)AMINO]PHENYL}ETHYL)-6-(TRIFLUOROMETHYL)-2-NAPHTHAMIDE

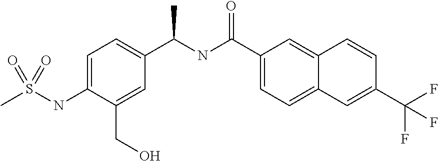

To a stirred solution of Example 74C (220 mg, 0.433 mmol) in dry THF (10 ml) was added lithium aluminium hydride (33 mg) in one portion at room temperature. After 3 hours at 40° C., the mixture was quenched with 2M hydrochloric acid solution (ca. 20 ml) and the precipitate was filtered through a pad of celite. The filter cake was washed with methanol and the filtrate and washings were evaporated in vacuo. The aqueous solution was extracted with dichloromethane (×3) and the organic layer was washed with brine, dried over sodium sulfate and concentrated in vacuo to give the crude product. The crude product was purified by column chromatography on amine-silica gel (150 g) with dichloromethane-methanol (25:1) to give 00110141-0051-000 (white solid), which was recrystallized with ethyl acetate-hexane to furnish the title compound (120 mg, 60%) as a white solid.

¹H NMR (270 MHz. CDCl₃) δ 1.53 (3H, d, J=6.6 Hz), 2.99 (3H, s), 4.62 (2H, s), 5.16-5.30 (1H, m), 7.24-7.38 (2H, m), 7.55 (1H, s), 7.80-7.87 (1H, m), 8.05-8.32 (3H, m), 8.50 (1H, s), 8.61 (1H, s), 9.09-9.16 (1H, m).

MS (ESI): m/z 465.15 [M−H]⁻.

### Example 75

### (R)—N-(1-(2-FLUORO-5-METHYL-4-(METHYLSULFONAMIDO)PHENYL)ETHYL)-2-(PIPERIDIN-1-YL)QUINOLINE-6-CARBOXAMIDE

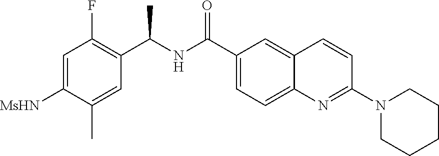

### 75A) ETHYL 2-(PIPERIDIN-1-YL)QUINOLINE-6-CARBOXYLATE

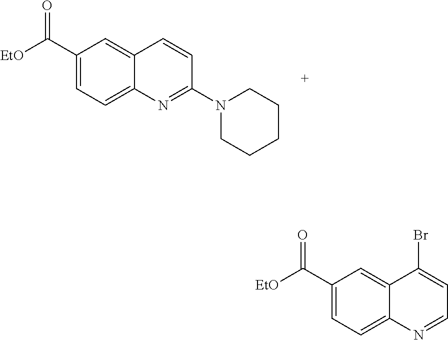

A mixture of the compound of Example 69A (133 mg, 0.475 mmol) and piperidine (121 mg, 1.42 mmol) in DMF (2 ml) was stirred at room temperature for 20 hours. The reaction mixture was diluted with toluene-ethyl acetate (1:1, 150 ml), washed with saturated aqueous sodium bicarbonate (50 mL), water (50 mL) and brine (50 mL). The organic layer was dried over sodium sulfate, filtered and evaporated. The residue was chromatographed on a column of silica gel (ethyl acetate-hexane-1:10 to 1:5) as eluent to give the 120 mg of ethyl 2-(piperidin-1-yl)quinoline-6-carboxylate (including ethyl 4-bromoquinoline-6-carboxylate) as white solid. This mixture was used for the next step without further purification.

MS (ESI) m/z 285 (M+H)⁺.

### 75B) 2-(PIPERIDIN-1-YL)QUINOLINE-6-CARBOXYLIC ACID

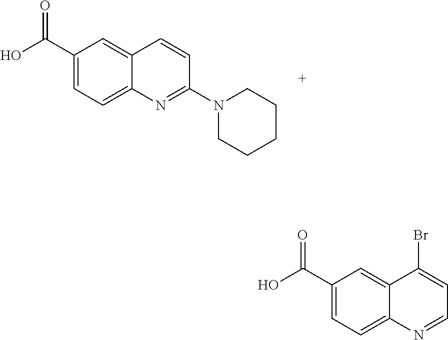

A mixture of the compound of the product of Example 75A (crude 120 mg) and 2M sodium hydroxide solution (0.42 ml, 0.842 mmol) in ethanol (3 ml) was treated in the same procedure described in Example 1G. The aqueous mixture was concentrated and dried in vacuo to give the title compound as white solid (including 4-bromoquinoline-6-carboxylic acid and sodium chloride). These crude products were used for the next step without purification.

MS (ESI): m/z 257 (M+H)⁺, 255 (M−H)⁺.

### 75C) (R)—N-(1-(2-FLUORO-5-METHYL-4-(METHYLSULFONAMIDO)PHENYL)ETHYL)-2-(PIPERIDIN-1-YL)QUINOLINE-6-CARBOXAMIDE

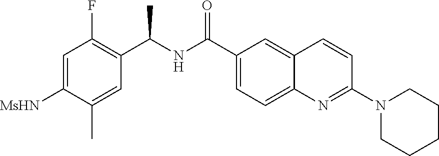

To a DMF (2 ml) solution of the compound of Example 13D (59 mg, 0.210 mmol), triethylamine (0.088 ml, 0.630 mmol), the compound of Example 75B (crude 0.210 mmol), and HBTU (88 mg, 0.231 mmol) was treated in the same procedure described in Example 1G. The crude residue was applied to a silica gel column chromatography and eluted with hexane/ethyl acetate (1:1 to 1:2) and HPLC (used column was XTerra MS C18, 5 um, 30×50 mm) eluting with acetonitrile/0.01% ammonium aqueous solution (basic 32_{—}68, 32:68 to 68:32) to furnish the title compound (29.6 mg, 30% yield) as white solid.

¹H NMR (270 MHz, DMSO-d₆) [symbol] 1.47 (3H, d, J=7.25 Hz), 1.52-1.75 (6H, m), 2.24 (3H, s), 3.01 (3H, s), 3.68-3.83 (4H, m), 5.37 (1H, m), 7.08 (1H, d, J=11.9 Hz), 7.28 (1H, d, J=9.2 Hz), 7.34 (1H, d, J=8.6 Hz) 7.53 (1H, d, J=8.6 Hz), 7.99 (1H, dd, J=2.0 Hz, 8.6 Hz), 8.07 (1H, d, J=9.2 Hz), 8.25 (1H, d, J=2.0 Hz), 8.80 (1H, d, J=7.3 Hz), 9.17 (1H, br s).

MS (ESI) m/z 485 (M+H)⁺, 483 (M−H)⁺.

### Example 76

### 2-TRIFLUOROMETHYL-4-(4-TRIFLUOROMETHYL-PIPERIDIN-1-YL)-QUINOLINE-6-CARBOXYLIC ACID [(R)-1-(2-FLUORO-4-METHANESULFONYLAMINO-5-METHYL-PHENYL)-ETHYL]-AMIDE

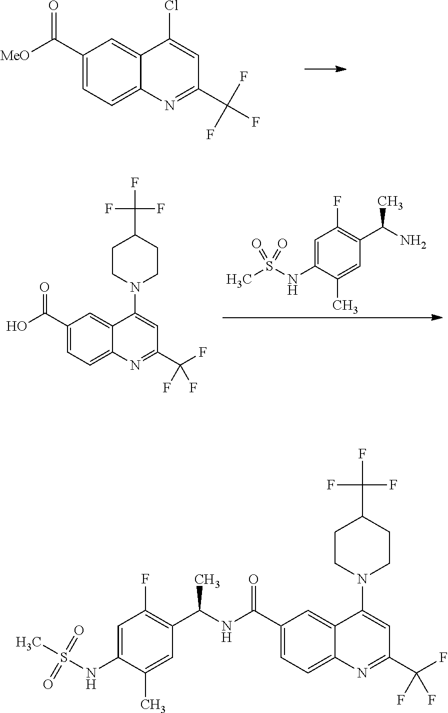

### 76A) 2-TRIFLUOROMETHYL-4-(4-TRIFLUOROMETHYL-PIPERIDIN-1-YL)-QUINOLINE-6-CARBOXYLIC ACID

The compound is prepared in a similar manner as Example 63B by reaction of the chloroquinoline ester (200 mg, 0.76 mmol) with the appropriate amine (130 mg, 0.68 mmol), followed by hydrolysis in basic media to give the title compound (50 mg, 19%). m/z=392.6 (M+1), r.t. 3.11 min. ¹H NMR (400 MHz; d₆-DMSO) δ 8.60 (1H, bs), 8.23 (s, 2H), 8.03 (1H, d), 7.28 (1H, s), 3.81-3.75 (2H, m), 3.18-3.05 (2H, m), 2.75-2.62 (1H, m), 2.11-1.99 (1H, m), 1.99-1.80 (m, 1H).

### 76B) 2-TRIFLUOROMETHYL-4-(4-TRIFLUOROMETHYL-PIPERIDIN-1-YL)-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (50 mg, 0.10 mmol) with the appropriate amine (44 mg, 0.16 mmol) to give the title compound (34 mg, 40%). m/z=62.1.4 (M+1), r.t. 3.52 min. ¹H NMR (400 MHz; d₆-DMSO) δ 9.25 (1H, d), 9.20 (1H, s), 8.50 (1H, d), 8.26 (1H, dd), 8.13 (1H, d), 7.38 (1H, d), 7.28 (1H, s), 7.11 (1H, d), 5.45-5.38 (1H, m), 3.85-3.73 (2H, m), 3.18-3.05 (2H, m), 3.05 (3H, s), 2.75-2.62 (1H, m), 2.26 (3H, s), 2.11-1.99 (2H, m), 1.99-1.80 (2H, m), 1.47 (3H, d).

### Example 78

### 4-[4-(2-HYDROXYETHYL)PIPERAZIN-1-YL]-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

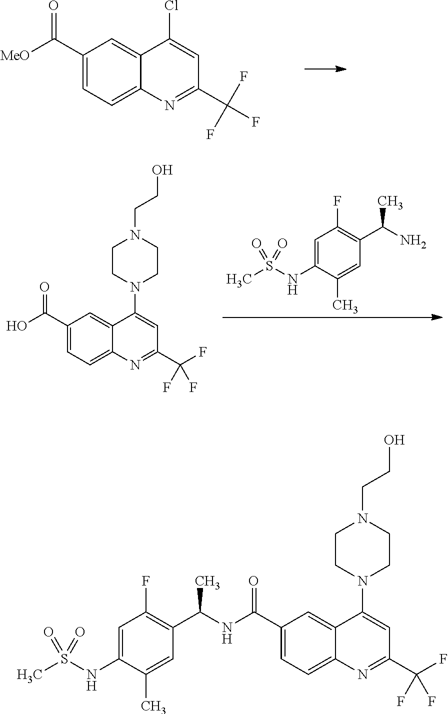

### 78A) 4-[4-(2-HYDROXYETHYL)PIPERAZIN-1-YL]-2-TRIFLUOROMETHYLQUINOLINE-6-CARBOXYLIC ACID

To a microwave vial containing methyl 4-chloro-2-(trifluoromethyl)quinoline-6-carboxylate (100 mg, 0.3 mmol), palladium acetate (0.78 mg, 0.0034 mmol), rac-BINAP (3.2 mg, 0.0052 mmol), cesium carbonate (157.5 mg, 0.48 mmol) and 1-piperazineethanol (67.4 mg, 0.52 mmol) was added anhydrous N,N-dimethylformamide (1 mL). The reaction was heated in the microwave at 120° C. for 5 minutes. The reaction mixture was poured into brine (50 mL) and extracted with EtOAc (3×40 mL). The combined organics were washed with brine (3×20 mL), dried (MgSO₄), filtered and concentrated. The material was dissolved in THF (6 mL) and EtOH (2 mL). 1N Lithium hydroxide in water (2 mL) was added, and the reaction stirred at 50° C. for 1 hour. The reaction mixture was neutralized with 2N HCl and evaporated onto silica. Flash chromatography (0 to 10% MeOH in CH₂Cl₂) gave the title compound (50 mg, 40%) as an off-white solid. m/z=370.3 (M+1), r.t. 1.95 min. ¹H NMR (400 MHz; d₆-DMSO) δ 11.4 (1H, bs), 8.65 (1H, d), 8.28 (1H, dd), 8.18 (1H, dd), 7.45 (1H, s), 5.42 (1H, bs), 3.92-3.30 (12H, m).

### 78B) 4-[4-(2-HYDROXYETHYL)PIPERAZIN-1-YL]-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4-[4-(2-hydroxyethyl)piperazin-1-yl]-2-trifluoromethylquinoline-6-carboxylic acid (28 mg, 0.076 mmol) was added a solution containing N,N,N′,N′-tetramethyl-O-(7-azabenzotriazol-1-yl)uronium hexafluorophosphate (29.1 mg, 0.076 mmol), N,N-diisopropylethylamine (26 mL, 0.15 mmol) and 4-dimethylaminopyridine (0.93 mg, 0.008 mmol) in anhydrous N,N-dimethylformamide (1.5 mL). After stirring for 5 minutes, a solution of N-[4-((R)-1-aminoethyl)-5-fluoro-2-methylphenyl]methanesulfonamide hydrochloride (25.9 mg, 0.092 mmol) and N,N-diisopropylethylamine (13 μL, 0.076 mmol) in anhydrous N,N-dimethylformamide (1 mL) was added. The reaction was stirred at room temperature for 16 hours. The reaction mixture was poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in CH₂Cl₂) gave the title compound (6 mg, 10%) as an off-white solid. m/z=598.4 (M+1), r.t. 2.18 min.

¹H NMR (400 MHz; d_{b}-DMSO) δ 9.22 (2H, d), 8.52 (1H, d), 8.26 (1H, dd), 8.13 (1H, d), 7.38 (1H, d), 7.29 (1H, s), 7.11 (1H, d), 5.44-5.37 (1H, m), 4.49 (1H, t), 3.58 (2H, q), 3.39 (4H, bs), 3.02 (3H, s), 2.74 (4H, bs), 2.55 (2H, m), 2.26 (3H, s), 1.50 (3H, d).

### Example 79

### 4-((S)-2-HYDROXYMETHYLPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

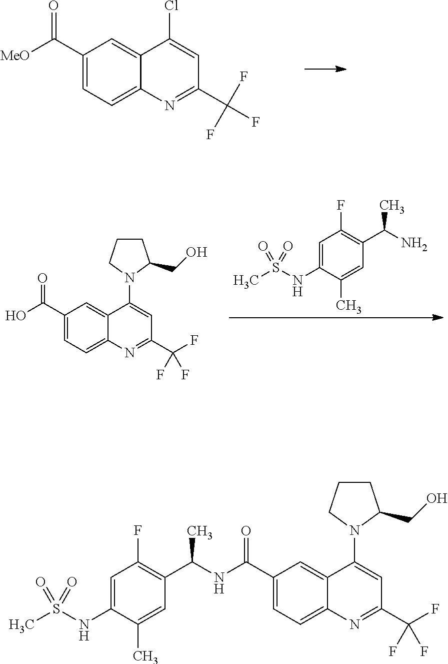

### 79A) 4-((S)-2-HYDROXYMETHYLPYRROLIDIN-1-YL)-2-TRIFLUOROMETHYLQUINOLINE-6-CARBOXYLIC ACID

To a microwave vial containing methyl 4-chloro-2-(trifluoromethyl)quinoline-6-carboxylate (100 mg, 0.3 mmol), palladium acetate (0.78 mg, 0.0034 mmol), rac-BINAP (3.2 mg, 0.0052 mmol), cesium carbonate (157.5 mg, 0.48 mmol) and L-prolinol (52.4 mg, 0.52 mmol) was added anhydrous N,N-dimethylformamide (1 mL). The reaction was heated in the microwave at 120° C. for 5 minutes. The reaction mixture was poured into brine (50 mL) and extracted with EtOAc (3×40 mL). The combined organics were washed with brine (3×20 mL), dried (MgSO₄), filtered and concentrated. The crude material was dissolved in THF (6 mL) and EtOH (2 mL). 1N Lithium hydroxide in water (2 mL) was added, and the reaction stirred at 50° C. for 1 hour. The reaction mixture was neutralized with 2N HCl and evaporated onto silica. Flash chromatography (0 to 10% MeOH in CH₂Cl₂) gave the title compound (40 mg, 30%) as a pale green solid. m/z=341.5 (M+1), r.t. 2.52 min.

### 79B) 4-((S)-2-HYDROXYMETHYLPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4-((S)-2-hydroxymethylpyrrolidin-1-yl)-2-trifluoromethylquinoline-6-carboxylic acid (30 mg, 0.09 mmol) was added a solution containing N,N,N′,N′-tetramethyl-O-(7-azabenzotriazol-1-yl)uronium hexafluorophosphate (33.52 mg, 0.09 mmol), N,N-diisopropylethylamine (31 μL, 0.18 mmol) and 4-dimethylaminopyridine (1.1 mg, 0.008 mmol) in anhydrous N,N-dimethylformamide (1.5 mL). After stirring for 5 minutes, a solution of N-[4-((R)-1-aminoethyl)-5-fluoro-2-methylphenyl]methanesulfonamide hydrochloride (29.9 mg, 0.11 mol) and N,N-diisopropylethylamine (16 μL, 0.09 mmol) in anhydrous N,N-dimethylformamide (1 mL) was added. The reaction was stirred at room temperature for 16 hours. The reaction mixture was poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in CH₂Cl₂) gave the title compound (7 mg, 10%) as a white solid. m/z=569.5 (M+1), r.t. 3.02 min. ¹H NMR (400 MHz; d₆-DMSO) δ 9.19 (1H, s), 9.06 (1H, d), 8.79 (1H, d), 8.21 (1H, dd), 7.96 (1H, d), 7.34 (1H, d), 7.10 (1H, d), 7.03 (1H, s), 5.53-5.35 (1H, m), 4.91 (1H, t), 4.32-4.28 (1H, m), 4.21-4.15 (1H, m), 3.79 (1H, t), 3.61-3.49 (2H; m), 3.03 (3H, s), 2.25 (3H, s), 2.24-2.18 (1H, m), 2.03-1.97 (2H, m), 1.71-1.64 (1H, m), 1.50 (3H, s).

### Example 80

### 4-(4-METHYL-PIPERAZIN-1YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

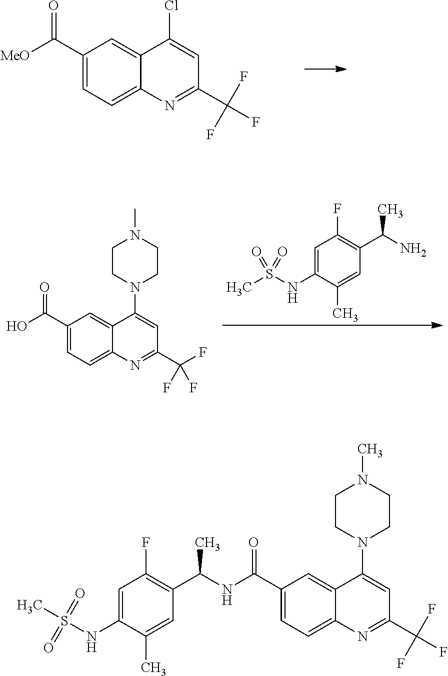

### 80A) 4-(4-METHYL-PIPERAZIN-1-YL)-2-(TRIFLUOROMETHYL)QUINOLINE-6-CARBOXYLIC ACID

The compound is prepared in a similar manner as Example 70A by hydrolysis of the ester (200 mg, 0.60 mmol) and in basic media to give the title compound (180 mg, 80%). m/z=326.6 (M+1), r.t. 2.75 min. ¹H NMR (400 MHz; d₆-DMSO) δ 8.63 (1H, s), 8.29 (1H, d), 8.14 (1H, d), 7.94 (1H, s), 7.22 (1H, s), 3.37 (4H, bs), 2.63 (4H, bs), 2.30 (3H, s).

### 80B) 4-(4-METHYL-PIPERAZIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (55 mg, 0.16 mmol) with the appropriate amine (55 mg, 0.19 mmol) to give the title compound (8 mg, 8%). m/z=567.5 (M+1), r.t. 1.96 min. ¹H NMR (400 MHz; d₆-DMSO) δ 9.22-9.21 (2H, m), 8.51 (1H, d), 8.25 (1H, dd), 8.13 (1H, d), 7.38 (1H, d), 7.25 (1H, s), 7.07 (1H, d), 5.44-5.38 (1H, m), 3.43-3.33 (4H, m), 3.05 (3H, s), 2.65 (4H, bs), 2.35 (3H, s), 2.26 (3H, s), 1.47 (3H, d).

### Example 81

### ((R)—N-(1-(2-FLUORO-5-METHYL-4-(METHYLSULFONAMIDO)PHENYL)ETHYL)-2-MORPHOLINOQUINOLINE-6-CARBOXAMIDE

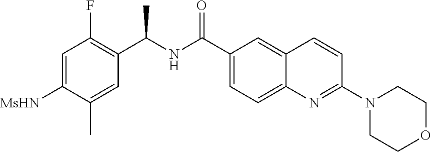

### 81A) ETHYL 2-MORPHOLINOQUINOLINE-6-CARBOXYLATE

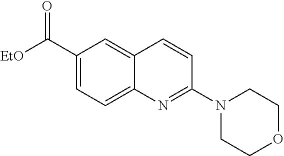

A mixture of the compound of Example 69A (245 mg, 0.875 mmol) and morpholine (114 mg, 1.31 mmol) in DMF (7 ml) was stirred at room temperature for 20 hours. The reaction mixture was treated in the same procedure described in Example 75A. The crude product was chromatographed on a column of silica gel (ethyl acetate-hexane=1:5 to 1:2) as eluent to give the title compound (82 mg, 32%) as white solid.

¹H NMR (270 MHz, CDCl₃) 1.43 (3H, t, J=6.6 Hz), 3.71-3.98 (8H, m), 4.42 (2H, q, J=6.6 Hz), 7.00 (1H, d, J=9.2 Hz), 7.69 (1H, d, J=9.2 Hz), 7.99 (1H, d, J=9.2 Hz), 8.16 (1H, d, J=8.6 Hz), 8.38 (1H, s).

MS (ESI): m/z 287 (M+H)⁺.

### 81B) 2-MORPHOLINOQUINOLINE-6-CARBOXYLIC ACID

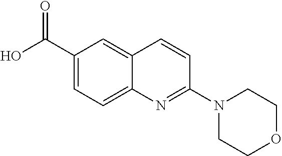

A mixture of the compound of Example 81A (82 mg, 0.425 mmol) and 2M sodium hydroxide solution (0.33 ml, 0.67 mmol) in ethanol (3 ml) was treated in the same procedure described in Example 69C. The aqueous mixture was concentrated and dried in vacuo to give the title compound as white solid (including sodium chloride). These crude products were used for the next step without purification.

¹H NMR (270 MHz, DMSO-d₆) [symbol] 3.73 (8H, s), 7.31 (1H, d, J=9.2 Hz), 7.58 (1H, d, J=8.6 Hz), 8.01 (1H, dd, J=1.3 Hz, 8.6 Hz), 8.22 (1H, d, J′=9.2 Hz), 8.38 (1H, d, J=2.0 Hz), 12.80 (1H, br s).

MS (ESI): m/z 259 (M+H)⁺, 257 (M−H)⁺.

### 81C) ((R)—N-(1-(2-FLUORO-5-METHYL-4-(METHYLSULFONAMIDO)PHENYL)ETHYL)-2-MORPHOLINOQUINOLINE-6-CARBOXAMIDE

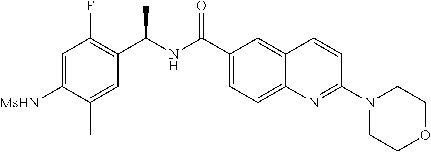

To a DMF (2 ml) solution of the compound of Example 13D (24 mg, 0.084 mmol), triethylamine (0.0351 ml, 0.252 mmol), the compound of Example 81B (24 mg, 0.084 mmol), and HBTU (35 mg, 0.092 mmol) was treated in the same procedure described in Example 1G. The crude residue was applied to a silica gel column chromatography and eluted with hexane/ethyl acetate (1:1 to 1:2) to furnish the title compound (34 mg, 83%) as white solid.

¹H NMR (270 MHz, DMSO-d₆) [symbol] 1.48 (3H, d, J=6.6 Hz), 2.25 (3H, s), 3.02 (3H, s), 3.72 (8H, s), 5.38 (1H, m), 7.09 (1H, d, J=11.9 Hz), 7.30 (1H, d, J=9.2 Hz), 7.36 (1H, d, J=8.6 Hz), 7.59 (1H, d, J=8.6 Hz), 8.02 (1H, dd, J=2.0 Hz, 9.2 Hz), 8.15 (1H, d, J=9.2 Hz), 8.30 (1H, d, J=2.0 Hz), 8.84 (1H, d, J=7.3 Hz), 9.18 (1H, br s).

MS (ESI) m/z 487 (M+H)⁺, 485 (M−H)⁺.

### Example 82

### 82A) N-((1R)-1-{3-METHYL-4-[(METHYLSULFONYL)AMINO]PHENYL}ETHYL)-7-(TRIFLUOROMETHYL)-2-NAPHTHAMIDE

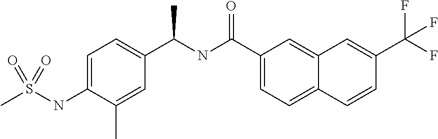

A solution of 7-(trifluoromethyl)-2-naphthoic acid (120 mg, 0.5 mmol), Example 13D (132 mg, 0.5 mmol), HBTU (227 mg, 0.6 mmol) and triethylamine (152 mg, 0.21 ml, 1.50 mmol) in anhydrous N,N-dimethylformamide (10 ml) was treated in the same procedure described in Example 1G to furnish the title compound (158.8 mg, 70%) as a white solid.

¹H NMR (270 MHz, CDCl₃) δ 1.51 (3H, d, J=7.2 Hz), 2.31 (3H, s), 2.97 (3H, s), 5.13-5.27 (1H, m), 7.21-7.33 (3H, m), 7.82-7.89 (1H, m), 8.10-8.26 (3H, m), 8.52 (1H, s), 8.69 (1H, s), 8.99-9.06 (2H, m).

MS (ESI): m/z 451.12 [M+H]⁺, 449.17 [M−H]⁻.

### Example 84

### 4-PIPERIDIN-1-YL-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

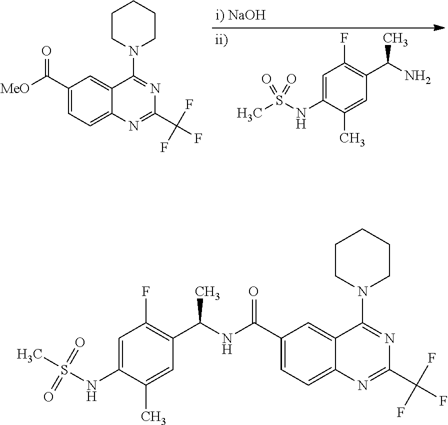

### 84A) 4-PIPERIDIN-1-YL-2-TRIFLUOROMETHYL-QUINAZOLINE-6-CARBOXYLIC ACID

The compound is prepared in a similar manner as Example 70A by hydrolysis of the ester (50 mg, 0.10 mmol) in basic media to give the title compound (45 mg, 100%). m/z=326.4 (M+1), r.t. 3.22 min. ¹H NMR (400 MHz; d₆-DMSO) δ 8.69 (1H, d), 8.33 (1H, dd), 7.75 (1H, d), 3.83 (4H, s), 1.74 (6H, bs).

### 84B) 4-PIPERIDIN-1-YL-2-TRIFLUOROMETHYL-QUINAZOLINE-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (45 mg, 0.14 mmol) with the appropriate amine (51 mg, 0.18 mmol) to give the title compound (24 mg, 31%). m/z=554.5 (M+1), r.t. 3.42 min. ¹H NMR (400 MHz; d₆-DMSO) δ 9.25 (1H, s), 9.14 (1H, d), 8.49 (1H, d), 8.33 (1H, dd), 7.95 (1H, d), 7.34, (1H, d), 7.09 (1H, d), 5.41-5.33 (1H, m), 3.90 (s, 4H), 3.02 (3H, s), 2.25 (3H, s), 1.78 (6H, s), 1.50 (3H, d).

### Example 85

### 4-[4-(3-CHLORO-PHENYL)-PIPERAZIN-1-YL]-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

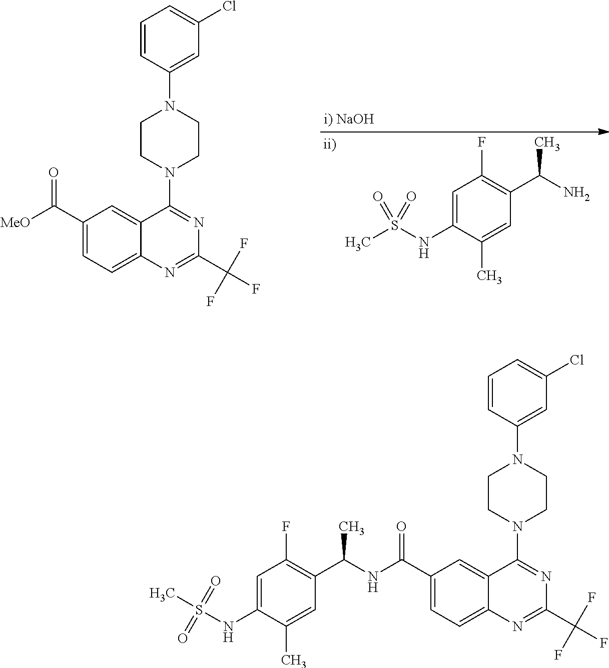

The compound is prepared in a similar manner as Example 66 by hydrolysis of the corresponding ester (25 mg, 0.054 mmol) and condensing the acid obtained with the appropriate amine (17 mg, 0.060 mmol) to give the title compound (17 mg, 47%). m/z=665.4 (M+1), r.t. 4.10 min. ¹H NMR (400 MHz; d₆-DMSO) δ 9.20 (1H, s), 9.16 (1H, d), 8.62 (1H, d), 8.39 (1H, dd), 8.01 (1H, d), 7.34, (1H, d), 7.15 (1H, t), 7.02 (1H, d), 6.97 (1H, t), 6.91 (1H, dd), 6.81 (1H, dd), 5.41-5.33 (1H, m), 4.13 (s, 4H), 3.53-3.48 (4H, m), 3.01 (3H, s), 2.25 (3H, s), 1.50 (3H, d)

### Example 86

### 4-(2,6-DIMETHYL-MORPHOLIN-4-YL)-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

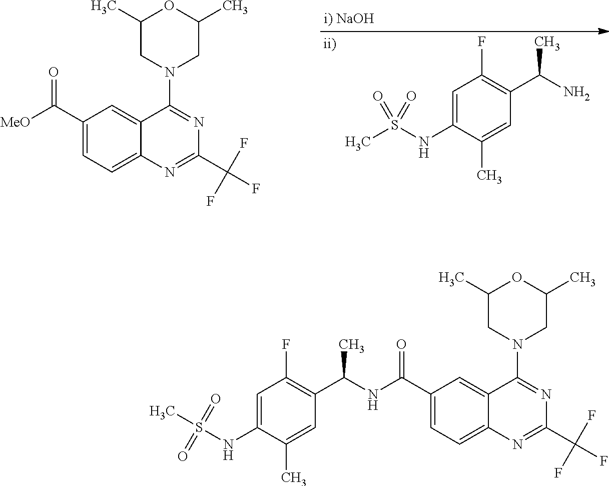

To a solution of ethyl 4-(2,6-dimethylmorpholino)-2-(trifluoromethyl)quinazoline-6-carboxylate (25 mg, 0.065 mmol) in tetrahydrofuran (0.5 mL, 6 mmol), 1 N of sodium hydroxide in water (0.21 mL) was added and the reaction was stirred at room temperature 4 h. The reaction mixture was neutralized with 1N HCl, dried (MgSO₄), filtered and evaporated. The crude residue was used in the next step without further purification. m/z=356.0 (M+r.t. 3.40 min.

A solution of the crude acid, N-[4-((R)-1-aminoethyl)-5-fluoro-2-methylphenyl]methanesulfonamide hydrochloride (17 mg, 0.059 mmol), N,N,N′,N′-tetramethyl-O-(7-azabenzotriazol-1-yl)uronium hexafluorophosphate (40 mg, 0.1 mmol) and N,N-diisopropylethylamine (0.010 mL, 0.059 mmol) in N,N-dimethylformamide (0.4 mL, 5 mmol) was stirred at room temperature for 16 hr. The reaction mixture was quenched with 1N HCl, neutralized with triethylamine and concentrated in vacuo. The residue was purified by prep HPLC (25-55 method) to give the title product (10 mg, 30%) as a tan solid. m/z=584.5 (M+1), r.t. 3.61 min. ¹H NMR (400 MHz; d₆-DMSO) δ 9.20 (1H, bs); 9.14 (1H, d), 8.51 (1H, d), 8.39 (1H, dd), 7.99 (1H, d), 7.33, (1H, d), 7.09 (1H, d), 5.44-5.37 (1H, m), 4.42-4.48 (2H, m), 3.70-3.83 (2H, m), 3.15-3.05 (2H, m), 3.01 (3H, s), 2.25 (3H, s), 1.49 (3H, d), 1.71 (6H, d).

### Example 87

### 7-CYCLOPROPYL-[1.5]NAPHTHYRIDINE-3-CARBOXYLIC ACID [(R)-1-(2-FLUORO-4-METHANESULFONYLAMINO-5-METHYL-PHENYL)-ETHYL]-AMIDE

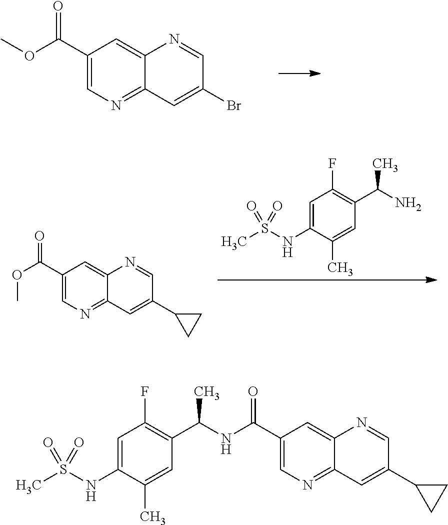

### 87A) ETHYL 7-CYCLOPROPYL-1,5-NAPHTHYRIDINE-3-CARBOXYLATE

A solution of ethyl 7-bromo-1,5-naphthyridine-3-carboxylate (50 mg, 0.2 mmol), cyclopropylboronic acid (2.0E1 mg, 0.23 mmol), potassium phosphate (130 mg, 0.62 mmol) in a mixture of toluene (2 mL, 20 mmol) and water (0.03 mL, 2 mmol) was degassed with nitrogen for 10 minutes. Then, palladium acetate (20 mg, 0.09 mmol) was added, and the reaction was heated at 100° C. for 1 hr in the microwave. After cooling, the reaction mixture was poured into saturated NaHCO₃ and extracted with EtOAc. The combined organics were washed with brine, dried (MgSO₄), filtered and concentrated. Flash Chromatography (10 to 50% EtOAc in Hexane) gave the title product as a tan solid (28 mg, 60%). m/z=243.5 (M+1), r.t. 3.35 min. ¹H NMR (400 MHz; CDCl₃) δ 9.47 (1H, d), 9.00 (1H, d), 8.90 (1H, d), 7.97 (1H, d), 4.46 (q, 2H), 2.19-2.13 (1H, m), 1.44 (t, 3H), 1.26-1.18 (2H, m), 0.98-0.93 (2H, m).

### 87B) (R)-7-CYCLOPROPYL-N-(1-(2-FLUORO-5-METHYL-4-(METHYLSULFONAMIDO)-PHENYL)ETHYL)-1,5-NAPHTHYRIDINE-3-CARBOXAMIDE

The compound is prepared in a similar manner as Example 66 by condensing the appropriate amine (43 mg, 0.15 mmol) with the corresponding acid (30 mg, 0.14 mmol). (11 mg, 0.024 mmol, 17%). m/z=443.3 (M+1), r.t. 2.96 min. ¹H NMR (400 MHz; d₆-DMSO) δ 9.31 (1H, d), 9.28 (1H, d), 9.19 (1H, bs), 8.95 (1H, dm), 8.85 (1H, bs), 8.07 (1H, d), 7.36 (1H, d), 7.09 (1H, d), 5.42-5.35 (1H, m), 3.01 (3H, s), 2.26 (3H, s), 1.51 (3H, d), 1.20-1.15 (2H, m), 1.04-1.00 (2H, m).

### Example 88

### 4-[4-(2-METHOXY-PHENYL)-PIPERAZIN-1-YL]-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

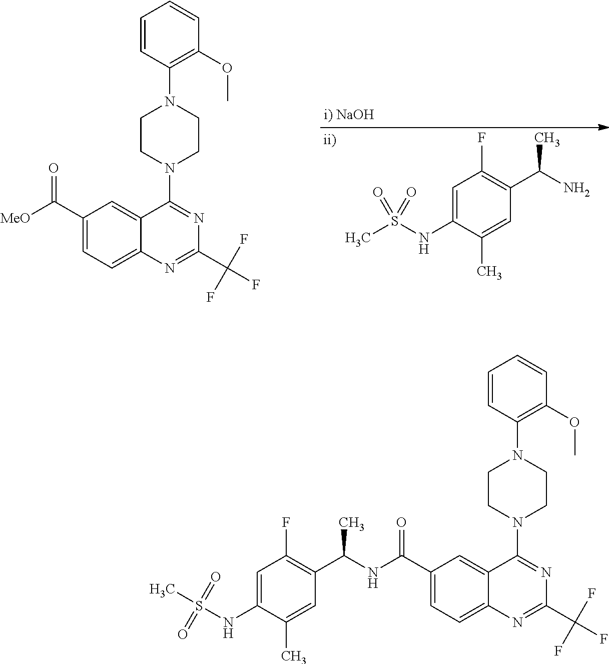

The compound is prepared in a similar manner as Example 66 by hydrolysis of the corresponding ester (25 mg, 0.054 mmol) and condensing the acid obtained with the appropriate amine (17 mg, 0.060 mmol) to give the title compound (18 mg, 47%). m/z=661.6 (M+1), r.t. 3.81 min. ¹H NMR (400 MHz; d₆-DMSO) δ 9.11 (1H, d), 8.57 (1H, s), 8.35 (dm, 1H), 7.99 (1H, d), 7.25 (1H, bs), 7.04-6.99 (m, 5H), 5.40-5.33 (1H, m), 4.14 (s, 4H), 3.82 (3H, s), 3.21 (s, 4H), 2.87 (3H, s), 2.15 (3H, s), 1.48 (3H, d).

### Example 90

### 4-((R)-3-HYDROXYPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

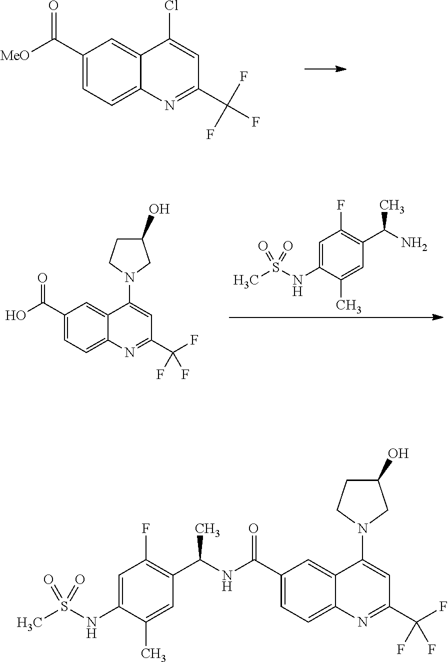

### 90A) 4-((R)-3-HYDROXYPYRROLIDIN-1-YL)-2-TRIFLUOROMETHYLQUINOLINE-6-CARBOXYLIC ACID

To a microwave vial containing methyl 4-chloro-2-(trifluoromethyl)quinoline-6-carboxylate (300 mg, 1 mmol), palladium acetate (2.3 mg, 0.01 mmol), rac-BINAP (9.7 mg, 0.016 mmol), cesium carbonate (472.5 mg, 1.45 mmol) and (R)-(+)-3-hydroxypyrrolidine (135 mg, 1.55 mol) was added anhydrous N,N-dimethylformamide (4 mL). The reaction was heated in the microwave at 120° C. for 15 minutes. The reaction mixture was poured into brine (50 mL) and extracted with EtOAc (3×40 mL). The combined organics were washed with brine (3×20 mL), dried (MgSO₄), filtered and concentrated. The crude material was dissolved in THF (6 mL) and EtOH (2 mL). 1N Lithium hydroxide in water (2 mL) was added, and the reaction stirred at 50° C. for 1 hour. The reaction mixture was neutralized with 2N HCl and evaporated onto silica. Flash chromatography (0 to 10% MeOH in CH₂Cl₂) gave the title compound (200 mg, 60%) as an off-white solid. m/z=327.5 (M+1), r.t. 2.30 min. ¹H NMR (400 MHz; d₆-DMSO) δ 9.04 (1H, d), 8.16 (1H, d), 7.96 (1H, d), 6.84 (1H, s), 4.48 (1H, t), 4.24-3.78 (3H, m), 3.62 (1H, d), 2.12-2.01 (2H, m).

### 90B) 4-((R)-3-HYDROXYPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4-4(R)-3-hydroxypyrrolidin-1-yl)-2-trifluoromethylquinoline-6-carboxylic acid (50 mg, 0.15 mmol) was added a solution containing N,N,N′,N′-tetramethyl-O-(7-azabenzotriazol-1-yl)uronium hexafluorophosphate (58.3 mg, 0.15 mmol), N,N-diisopropylethylamine (50 μL, 0.3 mmol) and 4-dimethylaminopyridine (1.87 mg, 0.015 mmol) in anhydrous N,N-dimethylformamide (1.5 mL). After stirring for 5 minutes, a solution of N-[4-((R)-1-aminoethyl)-5-fluoro-2-methylphenyl]methanesulfonamide hydrochloride (52 mg, 0.18 mmol) and N,N-diisopropylethylamine (25 μL, 0.15 mmol) in anhydrous N,N-dimethylformamide (1 mL) was added. The reaction was stirred at room temperature for 16 hours. The reaction mixture was poured into saturated. NaHCO₃ solution (50 mL) and extracted with EtOAc (3×50mL). The combined organics were washed with brine (3×50 mL), dried (MgSO₄), filtered and concentrated. Flash chromatography (0 to 5% MeOH in CH₂Cl₂) gave the title compound (24 mg, 22%) as a white solid. m/z=355.2 (M+1), r.t. 2.82 min. ¹H NMR (400 MHz; d₆-DMSO) δ 9.19 (1H, s), 9.10 (1H, d), 8.90 (1H, d), 8.21 (1H, dd), 7.96 (1H, d), 7.33 (1H, d), 7.09 (1H, d), 6.81 (1H, s), 5.41-5.46 (1H, m), 5.15 (1H, d), 4.47 (1H, bs), 4.05-3.96 (3H, m), 3.81-335 m), 3.62 (1H, d), 3.02 (3H, s), 2.25 (3H, s), 2.09-1.98 (2H, m), 1.49 (3H, d).

### Example 91

### 4-((S)-3-HYDROXY-PYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

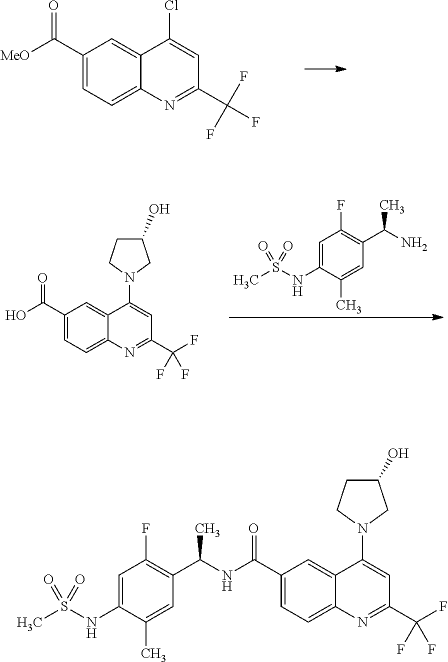

### 91A) 4-((S)-3-HYDROXYPYRROLIDIN-1-YL)-2-TRIFLUOROMETHYLQUINOLINE-6-CARBOXYLIC ACID

To a microwave vial containing methyl 4-chloro-2-(trifluoromethyl)quinoline-6-carboxylate (300 mg, 1 mmol), palladium acetate (2.3 mg, 0.010 mmol), rac-BINAP (9.7 mg, 0.016 mmol), cesium carbonate (472.5 mg, 1.45 mmol) and (S)-pyrrolidin-3-ol (135.4 mg, 1.55 mmol) was added anhydrous N,N-dimethylformamide (4 mL). The reaction was heated in the microwave at 120° C. for 15 minutes. The reaction mixture was poured into brine (50 mL) and extracted with EtOAc (3×40 mL). The combined organics were washed with brine (3×20 mL), dried (MgSO₄), filtered and concentrated. The crude material was dissolved in THF (6 mL) and EtOH (2 mL). 1N Lithium hydroxide in water (2 mL) was added, and the reaction stirred at 50° C. for 1 hour. The reaction mixture was neutralized with 2N HCl and evaporated onto silica. Flash chromatography (0 to 10% MeOH in CH₂Cl₂ over 60 minutes) gave the title compound (40 mg, 10%) as a white solid. m/z=327.4 (M+1), r.t. 2.31 min. ¹H NMR (400 MHz; d₆-DMSO) δ 9.05 (1H, d), 8.17 (1H, dd), 7.97 (1H, d), 6.86 (1H, s), 4.48 (1H, bs), 4.08 (1H, dd), 4.00-3.93 (1H, m), 3.85-3.78 (1H, m), 3.64 (1H, d), 2.14-1.99 (2H, m).

### 91A) 4-((S)-3-HYDROXYPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4-((S)-3-hydroxypyrrolidin-1-yl)-2-trifluoromethylquinoline-6-carboxylic acid (26 mg, 0.08 mmol) was added a solution containing N,N,N′,N′-tetramethyl-O-(7-azabenzotriazol-1-yl)uronium hexafluorophosphate (30.3 mg, 0.08 mmol), N,N-diisopropylethylamine (28 μL, 0.16 mmol) and 4-dimethylaminopyridine (0.9 mg, 0.008 mmol) in anhydrous N,N-dimethylformamide (1.5 mL). After stirring for 5 minutes, a solution of N-[4-((R)-1-aminoethyl)-5-fluoro-2-methylphenyl]methanesulfonamide hydrochloride (27 mg, 0.096 mmol) and N,N-diisopropylethylamine (14 μL, 0.08 mmol) in anhydrous N,N-dimethylformamide (1 mL) was added. The reaction was stirred at room temperature for 16 hours. The reaction mixture was poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 5% MeOH in CH₂Cl₂) gave the title compound (19.1 mg, 71%) as an off-white solid. m/z=555.3 (M+1), r.t. 2.74 min. ¹H NMR (400 MHz; d₆-DMSO) δ 9.20 (1H, s), 9.12 (1H, d), 8.91 (1H, s), 8.17 (1H, dd), 7.96 (1H, d), 7.37 (1H, d), 7.10 (1H, d), 6.81 (1H, s), 5.43-5.37 (1H, m), 5.17 (1H, d), 4.48 (1H, bs), 4.07-4.03 (2H, m), 3.78 (1H, t), 3.60 (1H, d), 3.02 (3H, s), 2.26 (3H, s), 2.11-1.99 (2H, m), 1.49 (2H, d).

### Example 92

### 4-(3,3-DIFLUOROAZETIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)-ETHYL]AMIDE

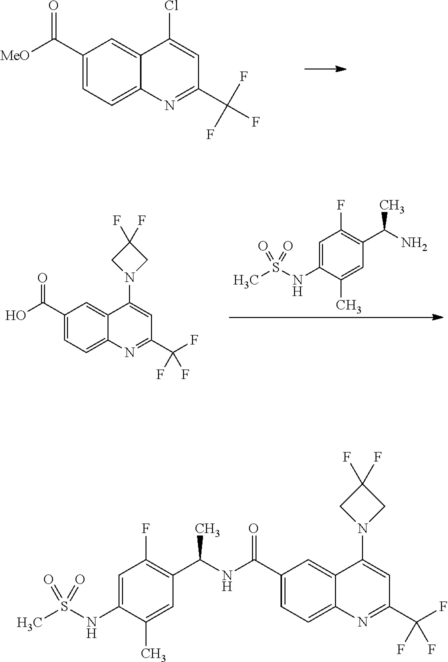

### 92A) 4-(3,3-DIFLUOROAZETIDIN-1-YL)-2-TRIFLUOROMETHYLQUINOLINE-6-CARBOXYLIC ACID

To a microwave vial containing methyl 4-chloro-2-(trifluoromethyl)quinoline-6-carboxylate (300 mg, 1 mmol), palladium acetate (2.3 mg, 0.010 mmol), rac-BINAP (9.7 mg, 0.016 mmol), cesium carbonate (472.5 mg, 1.45 mmol) and 3,3-difluoroazetidine hydrochloride (201.2 mg, 1.55 mmol) was added anhydrous N,N-dimethylformamide (4 mL). The reaction was heated in the microwave at 120° C. for 15 minutes. The reaction mixture was poured into brine (50 mL) and extracted with EtOAc (3×40 mL). The combined organics were washed with brine (3×20 mL), dried (MgSO₄), filtered and concentrated. The crude material was dissolved in THF (9 mL) and EtOH (3 mL). 1N Lithium hydroxide in water (3 mL) was added, and the reaction stirred at 50° C. for 1 hour. The reaction mixture was neutralized with 2N HCl and evaporated onto silica. Flash chromatography (0 to 10% MeOH in CH₂Cl₂ over 60 minutes) gave the title compound (40 mg, 10%) as a white solid. m/z=333.3 (M+1), r.t. 3.25 min. ¹H NMR (400 MHz; d₆-DMSO) δ 8.60 (1H, d), 8.21 (1H, dd), 8.03 (1H, dd), 6.81 (1H, s), 5.03 (4H, s).

### 92B) 4(3,3-DIFLUOROAZETIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4-(3,3-difluoroazetidin-1-yl)-2-trifluoromethylquinoline-6-carboxylic acid (42 mg, 0.13 mmol) was added a solution containing N,N,N′,N′-tetramethyl-O-(7-azabenzotriazol-1-yl)uronium hexafluorophosphate (48 mg, 0.13 mmol), N,N-diisopropylethylamine (45 μL, 0.26 mmol) and 4-dimethylaminopyridine (1.5 mg, 0.013 mmol) in anhydrous N,N-dimethylformamide (1.5 mL). After stirring for 5 minutes, a solution of N-[4-((R)-1-aminoethyl)-5-fluoro-2-methylphenyl]methanesulfonamide hydrochloride (42.9 mg, 0.15 mmol) and N,N-diisopropylethylamine (23 μL, 0.08 mmol) in anhydrous N,N-dimethylformamide (1 mL) was added. The reaction was stirred at room temperature for 16 hours. The reaction mixture was poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in CH₂Cl₂) gave the title compound (33.1 mg, 44%) as a white solid. m/z=561.4 (M+1), r.t. 3.50 min. ¹H NMR (400 MHz; d₆-DMSO) δ 9.20 (1H, s), 9.12 (1H, d), 8.46 (1H, d), 8.20 (1H, dd), 8.04 (1H, d), 7.36 (1H, d), 7.10 (1H, d), 6.83 (1H, d), 5.40-5.34 (1H, m), 5.06 (4H, t), 3.02 (1H, s), 2.25 (1H, s), 1.51 (1H, d).

### Example 93

### 4-((R)-2-HYDROXYMETHYLPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

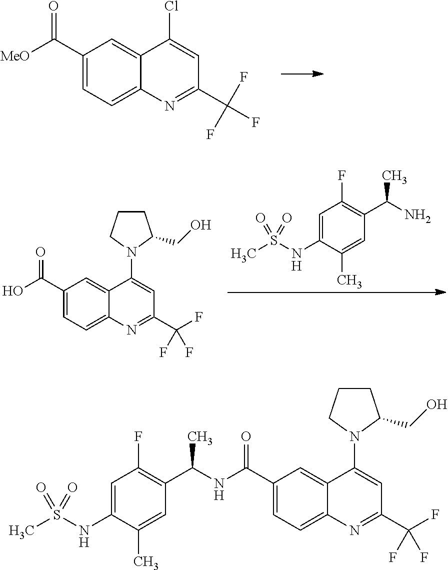

### 93A) 4-((R)-2-HYDROXYMETHYLPYRROLIDIN-1-YL)-2-TRIFLUOROMETHYLQUINOLINE-6-CARBOXYLIC ACID

To a microwave vial containing methyl 4-chloro-2-(trifluoromethyl)quinoline-6-carboxylate (300 mg, 1 mmol), palladium acetate (2.3 mg, 0.001 mmol), rac-BINAP (9.7 mg, 0.0016 mmol), cesium carbonate (472.5 mg, 1.45 mmol) and (R)-(−)-2-pyrrolidinemethanol (157.1 mg, 15.5 mmol) was added anhydrous N,N-dimethylformamide (4 mL). The reaction was heated in the microwave at 120° C. for 5 minutes. The reaction mixture was poured into brine (50 mL) and extracted with EtOAc (3×40 mL). The combined organics were washed with brine (3×20 mL), dried (MgSO₄), filtered and concentrated. The material was dissolved in THF (9 mL) and EtOH (3 mL). 1N Lithium hydroxide in water (3 mL) was added, and the reaction stirred at 50° C. for 1 hour. The reaction mixture was neutralized with 2N HCl and evaporated onto silica. Flash chromatography (0 to 10% MeOH in CH₂Cl₂) gave the title compound (200 mg, 50%) as an off-white solid. m/z=341.1 (M+1), r.t. 2.52 min. ¹H NMR (400 MHz; d₆-DMSO) δ 8.90 (1H, d), 8.17 (1H, dd), 8.00 (1H, d), 7.10 (1H, s), 4.40-4.35 (1H, m), 4.13-4.06 (1H, m), 3.88-3.82 (1H, m), 3.57 (2H, m), 2.21-1.96 (3H, m), 1.83-1.74 (1H, m).

### 93B) 4-((R)-2-HYDROXYMETHYLPYRROLIDIN-1-YL)-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4((R)-2-hydroxymethylpyrrolidin-1-yl)-2-trifluoromethylquinoline-6-carboxylic acid (50 mg, 0.1 mmol) was added a solution containing N,N,N′,N′-tetramethyl-O-(7-azabenzotriazol-1-yl)uronium hexafluorophosphate (55.9 mg, 0.15 mmol), N,N-diisopropylethylamine (52 μL, 0.30 mmol) and 4-dimethylaminopyridine (1.8 mg, 0.015 mmol) in anhydrous N,N-dimethylformamide (1.5 mL). After stirring for 5 minutes, a solution of N-[4-((R)-1-aminoethyl)-5-fluoro-2-methylphenyl]methanesulfonamide hydrochloride (50.0 mg, 0.18 mmol) and N,N-diisopropylethylamine (26 μL, 0.15 mmol) in anhydrous N,N-dimethylformamide (1 mL) was added. The reaction was stirred at room temperature for 16 hours. The reaction mixture was poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 4% MeOH in CH₂Cl₂) gave the title compound (30.5 mg, 30%) as a cream solid. m/z=569.4 (M+1), r.t. 3.01 min. ¹H NMR (400 MHz; d₆-DMSO) δ 9.19 (1H, s), 9.08 (1H, d), 8.80 (1H, s), 8.18 (1H, dd), 7.97 (1H, d), 7.35 (1H, d), 7.09 (1H, d), 7.04 (1H, s), 5.41-5.36 (1H, m), 4.91 (1H, t), 4.33-4.29 (1H, m), 4.13-4.08 (1H, m), 3.79 (1H, t), 3.63-3.56 (1H, m), 3.53-3.48 (1H, m), 3.02 (3H, s), 2.24 (3H, s), 2.21-2.18 (1H, m), 2.03-1.98 (2H, m), 1.84-1.77 (1H, m), 1.49 (3H, d).

### Example 94

### 4-(TETRAHYDRO-PYRAN-4-YLOXY)-2-TRIFLUOROMETHYL-QUINOLINE-6CARBOXYLIC ACID [(R)-1-(2-FLUORO-4-METHANESULFONYLAMINO-5-METHYL-PHENYL)-ETHYL]-AMIDE

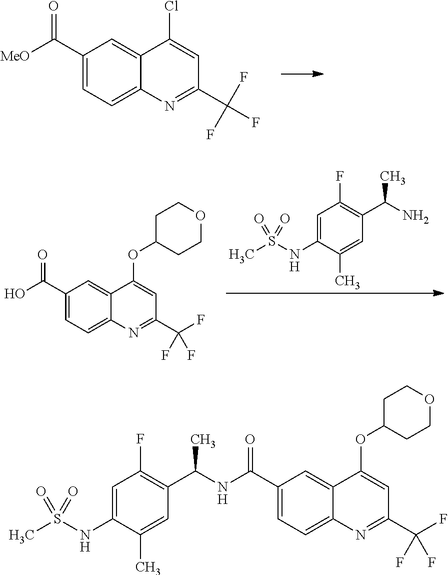

### 94A) 4-(TETRAHYDRO-PYRAN-4-YLOXY)-2-(TRIFLUOROMETHYL)QUINOLINE-6-CARBOXYLIC ACID

To a suspension of 60% Sodium hydride (210 mg, 5.2 mmol) in 20 mL of N,N-dimethylformamide, tetrahydro-2H-pyran-4-ol (500 uL, 5 mmol;) was added and the reaction mixture stirred at room temperature for 10 min. To this mixture methyl 4-chloro-2-(trifluoromethyl)quinoline-6-carboxylate (500 mg, 2 mmol) was added. After stirring at 100 C for 3 hr, the reaction was cooled down, dissolved in EtOAc and washed with H₂O. The combined organic layers were dried (MgSO₄), filtered and concentrated in vacuo to give a mixture of the desired ester and the corresponding acid that was used without further purification into the next step.

The above mixture was dissolved in 15 mL MeOH and 5 mL of water, and Lithium hydroxide (250 mg, 10 mmol) was added. The reaction mixture was heated to reflux for 30 min. Silica gel was added to the reaction mixture and the solvents were removed in vacuo by Flash Chromatography (0 to 50% MeOH in EtOAc) gave the title product (28 mg, 60%). m/z=342.3 (M+1), r.t. 2.86 min. ¹H NMR (400 MHz; d₆-DMSO) δ 8.73 (1H, d), 8.37 (1H, dd), 7.99 (1H, d), 7.35 (1H, s), 5.18-5.28 (1H, m), 3.94-3.84 (2H, m), 3.65-3.58 (2H, m), 2.19-2.08 (2H, m), 1.85-1.75 (2H, m).

### 94B) 4-(TETRAHYDRO-PYRAN-4-YLOXY)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (100 mg, 0.30 mmol) with the appropriate amine (99 mg, 0.35 mmol) to give the title compound (38 mg, 20%). m/z=570.2 (M+1), r.t. 3.11 min. ¹H NMR (400 MHz; d₆-DMSO) δ 9.24 (1H, d), 9.15 (1H, s), 8.70 (1H, d), 8.22 (1H, dd), 8.04 (1H, d), 7.49 (1H, s), 7.36 (1H, d), 7.10 (1H, d), 5.44-5.38 (1H, m), 5.21-5.32 (1H, m), 3.96-3.90 (2H, m), 3.70-3.58 (2H, m), 3.05 (3H, s), 2.22 (3H, s), 2.28-2.18 (2H, m), 1.89-1.75 (2FI, m) 1.47 (3H, d).

### Example 95

### 4-(4-HYDROXY-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

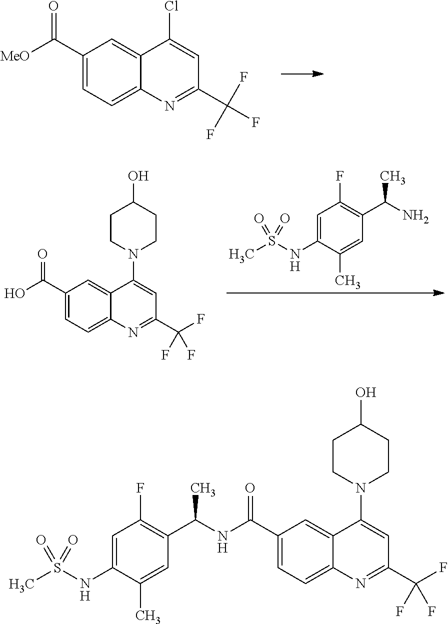

### 95A) 4-(4-HYDROXY-PIPERIDIN-1-YL)-2-(TRIFLUOROMETHYL)QUINOLINE-6-CARBOXYLIC ACID

A solution of methyl 4-chloro-2-(trifluoromethyl)quinoline-6-carboxylate (300 mg, 1.00 mmol), cesium carbonate (1010 mg, 3.10 mmol), palladium acetate (23 mg, 0.10 mmol) and 4-hydroxypiperidine (210 mg, 2.10 mmol) in 4 mL of N,N-dimethylformamide were heated in the microwave at 120° C. for 6 min. The reaction mixture was dissolved in EtOAc and the organic layer was washed with brine. The combined organic layers were dried (MgSO₄), filtered and concentrated in vacuo to give a mixture of the desired ester and the corresponding acid that was used without further purification into the next step. m/z=355.1 (M+1), r.t. 2.94 min.

The above mixture was dissolved in 305 mL MeOH and 5 mL of water, and lithium hydroxide (200 mg, 8 mmol) was added. The reaction mixture was heated to reflux for 30 min. Silica gel was added to the reaction mixture and the solvents were removed in vacuo by Flash Chromatography (0 to 50% MeOH in EtOAc) and gave the title product (200 mg, 57%). m/z=341.7 (M+1), r.t. 2.55 min. ¹H NMR (400 MHz; d₆-DMSO) δ 8.62 (1H, d), 8.24 (1H, dd), 7.92 (1H, d), 7.18 (1H, s), 4.92 (1H, bs), 3.84-3.74 (1H, m), 3.60-3.54 (2H, m), 3.37 (1H, bs), 3.18-3.05 (2H, m), 2.09-1.95 (2H, m), 1.79-1.62 (2H, m).

### 95B) 4-(4-HYDROXY-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (100 mg, 0.30 mmol) with the appropriate amine (99 mg, 0.35 mmol) to give the title compound (16 mg, 9%). m/z=568.5 (M+1), r.t. 3.24 min. ¹H NMR (400 MHz; d₆-DMSO) δ 9.22 (1H, d), 9.06 (1H, bs), 8.51 (1H, d), 8.26 (1H, dd), 8.13 (1H, d), 7.36 (1H, d), 7.23 (1H, s), 7.08 (1H, d), 5.44-5.38 (1H, m), 4.87 (1H, d), 3.82-3.77 (1H, m), 3.69-3.55 (2H, m), 3.23-3.13 (2H, m), 3.05 (3H, s), 2.26 (3H, s), 2.0 (2H, bs), 1.61-1.78 (2H, m), 1.47 (3H, d).

### Example 96

### 4-{6-[(R)-1-(2-FLUORO-4-METHANESULFONYLAMINO-5-METHYL-PHENYL)-ETHYLCARBAMOYL]-2-TRIFLUOROMETHYL-QUINAZOLIN-4-YL}-PIPERAZINE-1-CARBOXYLIC ACID ETHYL ESTER

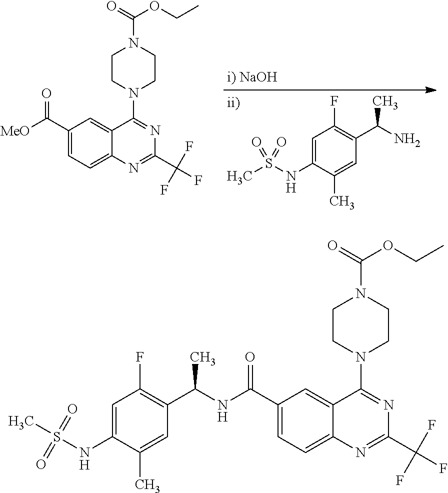

The compound is prepared in a similar manner as Example 66 by hydrolysis of the corresponding ester (25 mg, 0.059 mmol) and condensing the acid obtained with the appropriate amine (28 mg, 0.10 mmol) to give the title compound (10 mg, 30%). m/z=627.1 (M+1), r.t. 3.23 min. ¹H NMR (400 MHz; d₆-DMSO) δ 9.20 (1H, bs), 9.15 (1H, d), 8.55 (1H, d), 8.36 (1H, dd), 8.00 (1H, d), 7.35 (1H, d), 7.09 (1H, d), 5.4-5.34 (1H, m), 4.09 (2H, q), 4.00-4.03 (4H, m), 3.64 (4H, bs), 3.02 (3H, s), 2.25 (3H, s), 1.50 (3H, d), 1.20 (3H, t).

### Example 97

### 4-CYCLOHEXYLAMINO-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

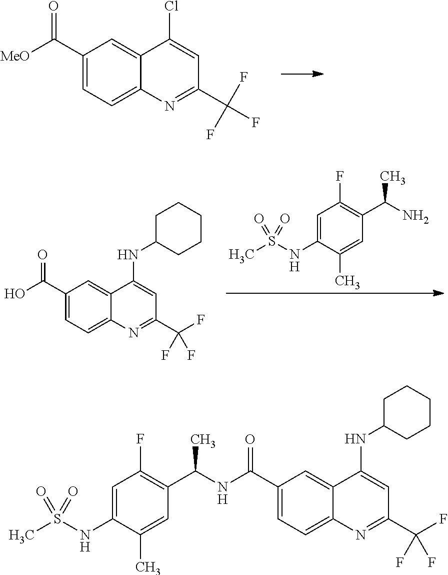

### 97A) 4-CYCLOHEXYLAMINO-2-TRIFLUOROMETHYLQUINOLINE-6-CARBOXYLIC ACID

To a microwave vial containing methyl 4-chloro-2-(trifluoromethyl)quinoline-6-carboxylate (300 mg, 1 mmol), palladium acetate (2.3 mg, 0.010 mmol), rac-BINAP (9.7 mg, 0.016 mmol), cesium carbonate (472.5 mg, 1.45 mmol) and cyclohexanamine (154.1 mg, 1.55 mol) was added anhydrous N,N-dimethylformamide (4 mL). The reaction was heated in the microwave at 120° C. for 15 minutes. The reaction mixture was poured into brine (50 mL) and extracted with EtOAc (3×40 mL). The combined organics were washed with brine (3×20 mL), dried (MgSO₄), filtered and concentrated. The material was dissolved in THF (9 mL) and EtOH (3 mL). 1N Lithium hydroxide in water (3 mL) was added, and the reaction stirred at 50° C. for 1 hour. The reaction mixture was neutralized with 2N HCl and evaporated onto silica. Flash chromatography (0 to 10% MeOH in CH₂Cl₂) gave the title compound (50 mg, 10%) as an off-white solid. m/z=339.2 (M+1), r.t. 3.58 min. ¹H NMR (400 MHz; d₆-DMSO) δ 9.15 (1H, d), 8.17 (1H, dd), 7.93 (1H, d), 6.90 (1H, s), 3.73-3.69 (1H, m), 1.99-1.88 (2H, m), 1.82-1.63 (3H, m), 1.54-1.39 (4H, m), 1.30-1.26 (1H, m).

### 97B) 4-CYCLOHEXYLAMINO-2-TRIFLUOROMETHYLQUINOLINE-6-CARBOXYLIC ACID [(R)-1-(2-FLUORO-4-METHANESULFONYLAMINO-5-METHYLPHENYL)ETHYL]AMIDE

To a vial containing 4-cyclohexylamino-2-trifluoromethylquinoline-6-carboxylic acid (45 mg, 0.13 mmol) was added a solution containing N,N,N′,N′-tetramethyl-O-(7-azabenzotriazol-1-yl)uronium hexafluorophosphate (50.6 mg, 0.13 mmol), N,N-diisopropylethylamine (45 μL, 0.26 mmol) and 4-dimethylaminopyridine (1.6 mg, 0.013 mmol) in anhydrous N,N-dimethylformamide (1.5 mL). After stirring for 5 minutes, a solution of N-[4-((R)-1-aminoethyl)-5-fluoro-2-methylphenyl]methane-sulfonamide hydrochloride (45.1 mg, 0.16 mmol) and N,N-diisopropylethylamine (23 μL, 0.13 mmol) in anhydrous N,N-dimethylformamide (1 mL) was added. The reaction was stirred at room temperature for 16 hours. The reaction mixture was poured into saturated NaHCO₃ solution (50 mL) and extracted with EtOAc (3×50 mL). The combined organics were washed with brine (3×50 mL), dried (MgSO₄), filtered and concentrated. Flash chromatography (0 to 3% MeOH in CH₂Cl₂) gave the title compound (17.4 mg, 22%) as a white solid. m/z=567.4 (M+1), r.t. 3.74 min. ¹H NMR (400 MHz; d₆-DMSO) δ 9.19 (1H, s), 8.98 (1H, d), 8.70 (1H, s), 8.15 (1H, dd), 7.92 (1H, d), 7.66 (1H, d), 7.36 (1H, d), 7.09 (1H, d), 6.82 (1H, s), 5.41-5.35 (1H, m), 3.35-3.32 (1H, m), 3.02 (3H, s), 2.24 (3H, s), 1.96 (2H, bs), 1.80-1.76 (2H, m), 1.68-1.63 (1H, m), 1.50 (3H, s), 1.44-1.39 (4H, m), 1.22-1.16 (1H, m).

### Example 98

### 7-PYRROLIDIN-1-YL-[1,5]NAPHTHYRIDINE-3-CARBOXYLIC ACID [(R)-1-(2-FLUORO-4-METHANESULFONYLAMINO-5-METHYL-PHENYL)-ETHYL]-AMIDE

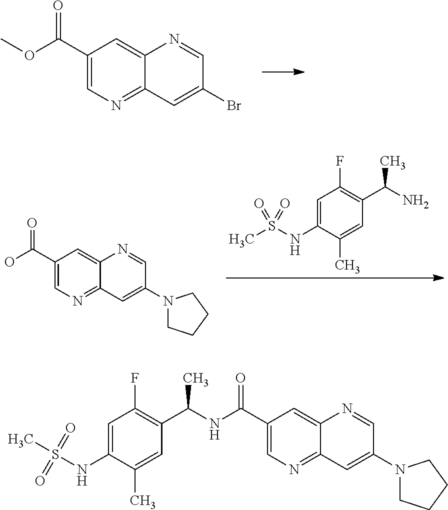

### 98A) ETHYL 7-(PYRROLIDIN-1-YL)-1,5-NAPHTHYRIDINE-3-CARBOXYLATE

To a solution of ethyl 7-bromo-1,5-naphthyridine-3-carboxylate (100 mg, 0.36 mmol) and 2,2′-Bis(diphenylphosphino)-1,1′-binaphthyl (3.5 mg, 0.0056 mmol) and cesium carbonate (120 mg, 0.36 mmol) in toluene (4 mL, 40 mmol) was degassed with nitrogen for 10 minutes. Then, pyrrolidine (0.046 mL, 0.55 mmol) and dichlorobis(tri-o-tolylphosphine)palladium(II) (1.0 mg, 0.0013 mmol) was added, and the reaction was heated at 150° C. for 1 hr in the microwave. The reaction mixture was concentrated in vacuo and purified by column chromatography (Hex:EtOAc, 20-60%) to give the pyrrolidine product (75 mg, 78%) as a yellow solid. m/z=272.3 (M+1), r.t. 2.61 min. ¹H NMR (400 MHz; CDCl₃) δ 9.32 (1H, d), 8.85 (1H, s), 8.66 (1H, d), 7.22 (1H, s), 4.46 (q, 2H), 3.55-3.52 (2H, m), 2.17-2.04 (2H, m), 1.44 (t, 3H).

### 98B) 7-PYRROLIDIN-1-YL-[1,5]NAPHTHYRIDINE-3-CARBOXYLIC ACID [(R)-1-(2-FLUORO-4-METHANESULFONYLAMINO-5-METHYL-PHENYL)-ETHYL]-AMIDE

The compound is prepared in a similar manner as Example 66 by hydrolysis of the corresponding ester (38 mg, 0.14 mmol) and condensing the acid obtained with the appropriate amine (43 mg, 0.15 mmol) to give the title compound (14 mg, 21%). m/z=472.4 (M+1), r.t. 2.25 min. ¹H NMR (400 MHz; dd-DMSO) δ 9.19 (1H, bs), 9.15 (1H, d), 9.06 (1H, d), 8.74 (2H, d), 7.34 (1H, d), 7.12 (1H, d), 7.08 (1H, d), 5.40-5.32 (1H, m), 3.51-3.46 (4H, m), 2.99 (3H, s), 2.24 (3H, s), 2.07-2.01 (4H, m), 1.49 (3H, d).

### Example 99

### 4-(4-HYDROXYMETHYL-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

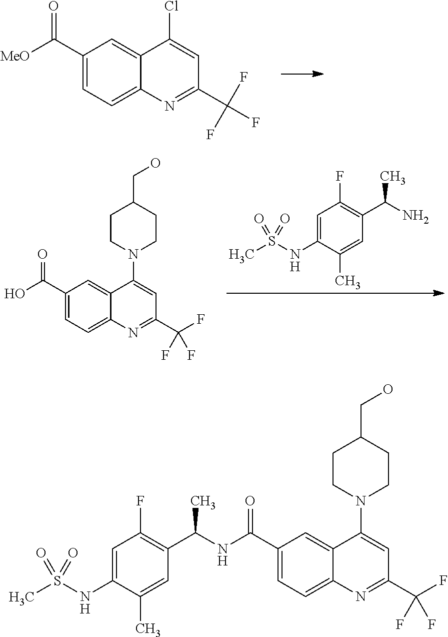

### 99A) 4-(4-HYDROXYMETHYL-PIPERIDIN-1-YL)-QUINOLINE-6-CARBOXYLIC ACID

The compound is prepared in a similar manner as Example 95A by reaction of the chloroquinoline ester (300 mg, 1.00 mmol) with the appropriate amine (400 mg, 3.00 mmol), followed by hydrolysis in basic media to give the title compound (260 mg, 70%). m/z=355.3 (M+1), r.t. 2.67 min. ¹H NMR (400 MHz; d₆-DMSO) δ 8.60 (1H, bs), 8.48 (1H, d), 8.25 (1H, dd), 7.92 (1H, d), 7.18 (1H, s), 3.71-3.60 (2H, m), 3.41 (2H, d), 2.93 (2H, t), 3.40 (1H, bs), 1.91-1.83 (m 2H), 1.69-1.60 (1H, m), 1.57-1.43 (2H, m).

### 99B) 4-(4-HYDROXYMETHYL-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (100 mg, 0.30 mmol) with the appropriate amine (92 mg, 0.32 mmol) to give the title compound (17 mg, 10%). m/z=583.32 (M+1), r.t. 2.96 min. ¹H NMR (400 MHz; d₆-DMSO) δ 9.21-9.20 (2H, m), 8.50 (1H, d), 8.25 (1H, dd), 8.11 (1H, d), 7.37 (1H, d), 7.29 (1H, s), 7.12 (1H, d), 5.44-5.38 (1H, m), 4.59 (1H, t), 3.75 (2H, d), 3.39 (2H, t), 3.35-3.28 (2H, m), 3.05-2.94 (2H, m), 3.07 (3H, s), 2.22 (3H, s), 1.94-1.83 (2H, m), 1.47 (3H, d).

### Example 100

### 7-PYRAZOL-1-YL-[1,5]NAPHTHYRIDINE-3-CARBOXYLIC ACID [(R)-1-(2-FLUORO-4-METHANESULFONYLAMINO-5-METHYL-PHENYL)-ETHYL]-AMIDE

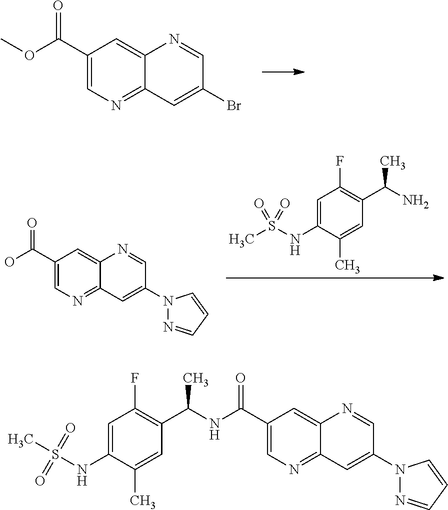

### 100A) ETHYL 7(1H-PYRAZOL-1-YL)-1,5-NAPHTHYRIDINE-3-CARBOXYLATE

A solution of ethyl 7-bromo-1,5-naphthyridine-3-carboxylate (85 mg, 0.30 mmol), cesium carbonate (148 mg, 0.454 mmol), 1H-pyrazole (31.4 mg, 0.461 mmol) and copper(I) iodide (14 mg, 0.076 mmol) in N,N-dimethylformamide (2 mL, 30 mmol) was heated at 150° C. for 1 hr in the microwave. The reaction mixture was filtered, dissolved in EtOAc and washed with 1N HCl. The organic layers were dried (MgSO4), concentrated in vacuo and purified by column chromatography (Hex:EtOAc, 20-40%) to give the pyrazole product (15 mg, 18%) as a white solid. m/z=—268.9 (M+1), r.t. 2.75 min. ¹H NMR (400 MHz; CDCl₃) δ 9.70 (1H, d), 9.55 (1H, d), 9.08 (1H, d), 8.58 (1H, d), 8.18 (1H, d), 7.88 d), 6.63 (1H, dd), 4.51 (q, 2H), 1.48 (t, 3H).

### 100B) 7-PYRAZOL-1-YL-[1,5]NAPHTHYRIDINE-3-CARBOXYLIC ACID [(R)-1-(2-FLUORO-4-METHANESULFONYL AMINO-5-METHYL-PHENYL)-ETHYL]-AMIDE

The compound is prepared in a similar manner as Example 66 by hydrolysis of the corresponding ester (17 mg, 0.063 mmol) and condensing the acid obtained with the appropriate amine (20 mg, 0.070 mmol) to give the title compound (4 mg, 10%). m/z=469.4 (M+1), r.t. 2.88 min. ¹H NMR (400 MHz; d₆-DMSO) δ 9.76 (1H, d), 9.41 (1H, d), 9.34 (1H, d), 9.20 (1H, bs), 8.98-9.01 (1H, m), 8.94 (2H, d), 8.85 (1H, d), 7.96 (d, 1H), 7.39 (1H, d), 7.10 (1H, d), 6.73 (1H, dd), 5.45-5.36 (1H, t), 3.02 (3H, s), 2.26 (3H, s), 1.53 (3H, d).

### Example 101

### N-((1R)-1-{2-FLUORO-5-METHYL-4-[(METHYLSULFONYL)AMINO]PHENYL}ETHYL)-6-(2,2,2-TRIFLUORO-1-HYDROXY-1-METHYLETHYL)-2-NAPHTHAMIDE

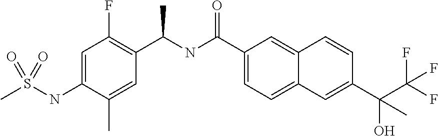

### 101A) CARBOXYLIC ACID 1: 2-(6-BROMO-2-NAPHTHYL)-1,1,1-TRIFLUOROPROPAN-2-OL

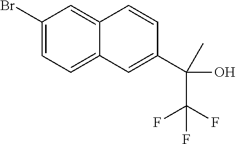

To a DMF (25 ml) solution of 1-(6-bromo-2-naphthyl)ethanone (2.5 g, 10.0 mmol, Tetrahedron Letters (2001), 42(2), 265-266), trifluoromethyl trimethyl silane (2.14 g, 15.1 mmol) and lithium acetate (33.1 mg, 0.5 mmol) were added and the mixture was stirred for 12 hrs at room temperature. Then, the reaction was partitioned with sodium acetate aqueous solution and ethylacetate. The organic layer was dried over sodium sulfate and filtrated. Then, evaporation gave the crude residue which was treated with hydrogen chloride and methanol with stirring for 5 hrs. Then, evaporation gave the crude residue which was purified through silica gel column chromatography eluting with hexane:ethyl acetate (5:1) to give the title compound as colorless oil in 83% yield.

¹H NMR (300 MHz, CDCl₃) [symbol] 2.50 (1H, s), 7.58 (1H, d, J=8.8 Hz); 771-7.81 (3H, m), 8.04 (2H, d, J=8.9 Hz).

### 101B) METHYL 6(2,2,2-TRIFLUORO-1-HYDROXY-1-METHYLETHYL)-2-NAPHTHOATE

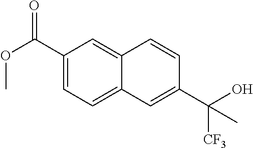

To a DMA (25 ml) and methyl alcohol (1 ml) solution of the compound of 101A (1.0 g, 3.1 mmol), trifluoromethyl trimethyl silane (2.14 g, 15.1 mmol) and lithium acetate (33.1 mg, 0.5 mmol), palladium acetate (70.0 mg, 0.31 mmol), diphenylohosphino propane (129 mg, 0.31 mmol) and triethylamine (951 mg, 9.4 mmol) were added and the mixture was stirred for 12 hrs at 100° C. under CO gas condition (balloon pressure). Then, the reaction was partitioned with water and ethyl acetate. The organic layer was dried over sodium sulfate and filtrated. Then, evaporation gave the crude residue which was purified through silica gel column chromatography eluting with hexane:ethyl acetate (5:1) to give the colorless oil in 50% yield.

¹H NMR (300 MHz, DMSO-d₆) [symbol] 1.81 (3H, s), 3.93 (3H, s), 6.85 (1H, s), 7.81-8.00 (1H, m), 8.11-8.26 (4H, m), 8.66 (1H, s).

### 101C) 6-(2,2,2-TRIFLUORO-1-HYDROXY-1-METHYLETHYL)-2-NAPHTHOIC ACID

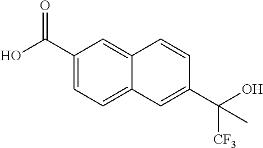

To an ethyl alcohol (30 ml) solution of the compound of 101B (1.16 g, 3.1 mmol), sodium hydroxide aqueous solution (2M) (15 ml) was added and the mixture was stirred for 5 hrs at room temperature. Then, the reaction was acidified with hydrogen chloride aqueous solution (20 ml) and the product was extracted with ethyl acetate and dried over sodium sulfate. Then filtration, evaporation gave the title compound as a white solid in 90% yield.

¹H NMR (300 MHz, DMSO-d₆) [symbol] 1.81 (3H, s), 6.85 (1H, s), 7.82 (1H, d, J=9.2 Hz), 7.99-825 (4H, m), 8.62 (1H, s), 12.9 (1H, brs).

### 101D) N-((1R)-1-{2-FLUORO-5-METHYL-4-[(METHYLSULFONYL)AMINO]PHENYL}ETHYL)-6-(2,2,2-TRIFLUORO-1-HYDROXY-1-METHYLETHYL)-2-NAPHTHAMIDE

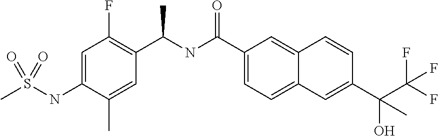

To a DMF (100 ml) solution of the compound of 101C (100 mg, 0.35 mmol), HBTU (133 mg, 0.35 mmol) and triethylamine (107 mg, 1.06 mmol) were added and the mixture was stirred for 0.2 hour at 50° C. Then, amine 13D (99.5 mg, 0.35 mmol) was portioned to this reaction and the mixture was stirred for 12 h at 50° C. Then, the reaction was quenched with saturated sodium bicabonate and the product was extracted with ethyl acetate. After the usual purification, the title compound was furnished as a white solid in 31% yield.

¹H NMR (300 MHz, DMSO-d₆) [symbol] 1.65 (3H, d, J=7.3 Hz), 1.90 (3H, s), 2.25 (3H, s), 2.66 (1H, brs), 3.05 (3H, s), 5.38-5.46 (1H, m), 6.22 (1H, brs), 6.74 (1H, d, J=8.8 Hz), 7.22 (1H, s), 7.32 (1H, s), 7.72-7.83 (1H, m), 7.86-7.97 (3H, m), 8.11 (1H, s), 8.28 (1H, s).

### Example 102

### 4-(4-PYRROLIDIN-1-YL-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

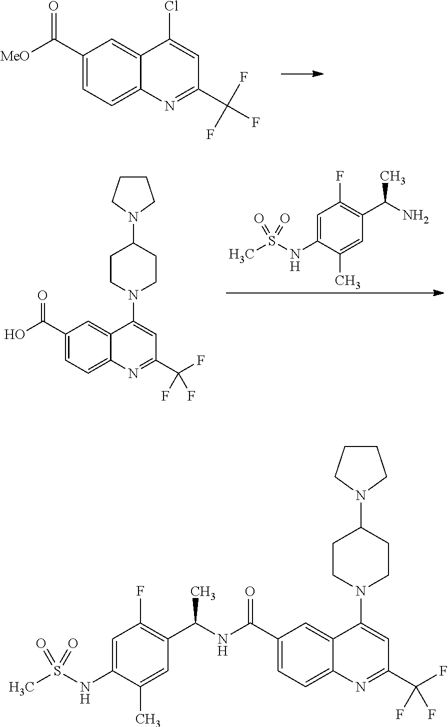

### 102A) 4-(4-PYRROLIDIN-1-YL-PIPERIDIN-1-YL)-QUINOLINE-6-CARBOXYLIC ACID

The compound is prepared in a similar manner as Example 95A by reaction of the chloroquinoline ester (300 mg, 1.00 mmol) with the appropriate amine (320 mg, 3.00 mmol), followed by hydrolysis in basic media to give the title compound (260 mg, 70%). m/z=394.3 (M+1), r.t. 1.91 min. ¹H NMR (400 MHz; d₆-DMSO) δ 8.55 (1H, d), 8.25 (1H, dd), 7.90 (1H, d), 7.16 (1H, s), 3.71-3.60 (2H, m), 3.38 (1H, bs), 3.03 (2H, t), 2.25 (3H, bs), 2.28-2.22 (1H, m), 2.09-2.02 (2H, m), 1.80-1.67 (6H, m).

### 102B) 4-(4-PYRROLIDIN-1-YL-PIPERIDIN-1-YL)-2-TRIFLUOROMETHYL-QUINOLINE-6-CARBOXYLIC ACID [(R)-1-(2-FLUORO-4-METHANESULFONYLAMINO-5-METHYL-PHENYL)-ETHYL]-AMIDE

The compound is prepared in a similar manner as Example 63C by condensing the acid (100 mg, 0.20 mmol) with the appropriate amine (86 mg, 0.30 mmol) to give the title compound (30 mg, 20%). m/z=622.0 (M+1), r.t. 2.08 min. ¹H NMR (400 MHz; d₆-DMSO) δ 9.23 (1H, d), 8.52 (1H, d), 8.25 (1H, dd), 8.13 (1H, d), 7.37 (1H, d), 7.36 (1H, s), 7.10 (1H, d), 5.44-5.38 (1H, m), 3.80-3.72 (2H, m), 3.13-3.03 (2H, m), 3.05 (3H, s), 2.75 (3H, bs), 2.22 (3H, s), 2.18-2.08 (2H, m), 1.85-1.79 (6H, m) 1.47 (3H, d).

### Example 103

### 4-(3-HYDROXY-PROPYLAMINO)-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

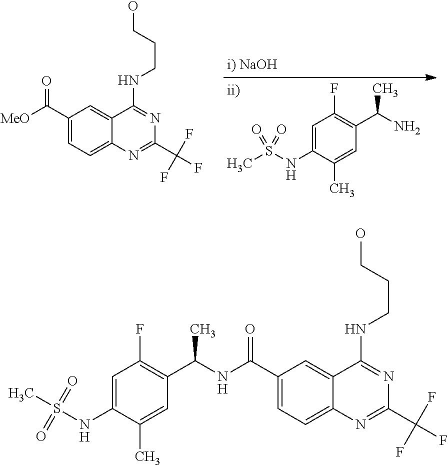

The compound is prepared in a similar manner as Example 66 by hydrolysis of the corresponding ester (25 mg, 0.073 mmol) and condensing the acid obtained with the appropriate amine (23 mg, 0.080 mmol) to give the title compound (9 mg, 20%). m/z=544.5 (M+1), r.t. 3.10 min. ¹H NMR (400 MHz; d₆-DMSO) δ 9.10 (1H, t), 9.01 (1H, d), 8.83 (1H, d), 8.30 (1H, dd), 7.89 (1H, d), 7.34 (1H, d), 7.08 (1H, d), 5.41-5.35 (1H, m), 4.53 (1H, t), 3.61 (2H, q), 3.51 (2H, q), 3.00 (3H, s), 2.23 (3H, s), 1.84-1.79 (2H, m), 1.50 (3H, d).

### Example 104

### 4-(3-MORPHOLIN-4-YL-PROPYLAMINO)-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

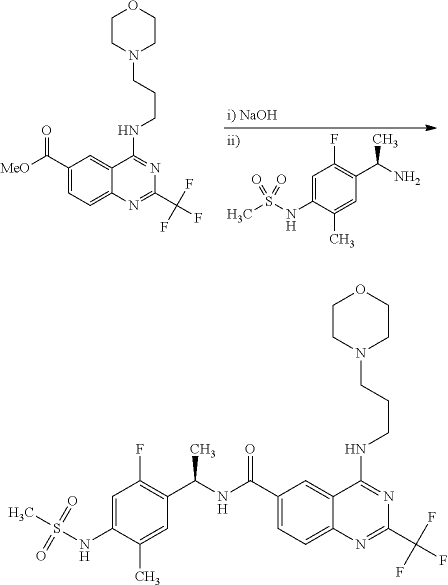

The compound is prepared in a similar manner as Example 66 by hydrolysis of the corresponding ester (25 mg, 0.061 mmol) and condensing the acid obtained with the appropriate amine (19 mg, 0.067 mmol) to give the title compound (16 mg, 43%). m/z=613.2 (M+1), r.t. 2.19 min. ¹H NMR (400 MHz; d₆-DMSO) δ 9.23 (1H, bs), 9.10 (1H, t), 9.01 (1H, d), 8.83 (1H, d), 8.31 (1H, dd), 7.89 (1H, d), 7.33 (1H, d), 7.08 (1H, d), 5.37 (1H, t), 3.61 (q, 2H), 3.55 (q, 4H), 3.00 (3H, s), 2.35 (6H, bs), 2.23 (3H, s), 1.84-1.79 (2H, m), 1.50 (3H, d).

### Example 105

### 6-PYRAZOL-1-YL-NAPHTHALENE-2-CARBOXYLIC ACID [(R)-1-(2-FLUORO-4-METHANESULFONYLAMINO-5-METHYL-PHENYL)-ETHYL]-AMIDE

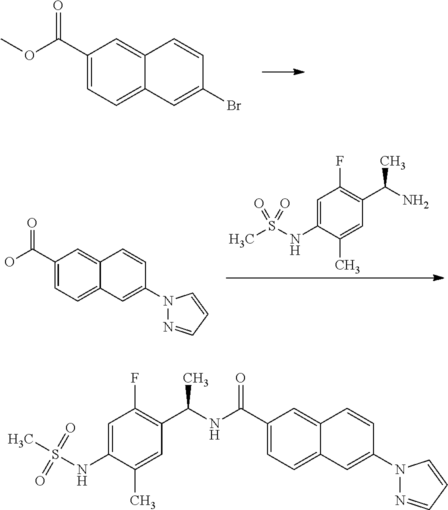

### 105A) METHYL 6-(1H-PYRAZOL-1-YL)-2-NAPHTHOATE

The compound is prepared in a similar manner as Example 66 by reaction of 6-Bromonaphthalene-2-carboxylic acid methyl ester (3.0E2 mg, 1.1 mmol) and 1H-pyrazole (31.4 mg, 0.46 mmol) to give the title compound (15 mg, 18%). m/z=253.5 (M+1), r.t. 3.59 min. ¹H NMR (400 MHz; CDCl₃) δ 8.62 (1H, bs), 8.16 (1H, d), 8.12-8.09 (2H, m), 8.05 (1H, d), 6.55 (1H, t), 3.99 (3H, s).

### 105B) 6-PYRAZOL-1-YL-NAPHTHALENE-2-CARBOXYLIC ACID [(R)-1-(2-FLUORO-4-METHANESULFONYLAMINO-5-METHYL-PHENYL)-ETHYL]-AMIDE

The compound is prepared in a similar manner as Example 66 by hydrolysis of the corresponding ester (35 mg, 0.14 mmol) and condensing the acid obtained with the appropriate amine (43 mg, 0.15 mmol) to give the title compound (3 mg, 4%). m/z=467.5 (M+1), 3.37 min. ¹H NMR (400 MHz; d₆-DMSO) δ 8.99 (1H, d), 8.69 (1H, d), 8.53 (1H, bs), 8.42 (1H, bs), 8.20-8.14 (2H, m), 8.06-7.99 (2H, m), 7.84 (1H, d), 7.31 (1H, d), 7.06 (1H, d), 6.63 (1H, dd), 5.42-5.35 (1H, m), 2.94 (3H, s), 2.21 (3H, s), 1.50 (3H, d).

### Example 106

### 4-(4-BENZYL-PIPERIDIN-1-YL)-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

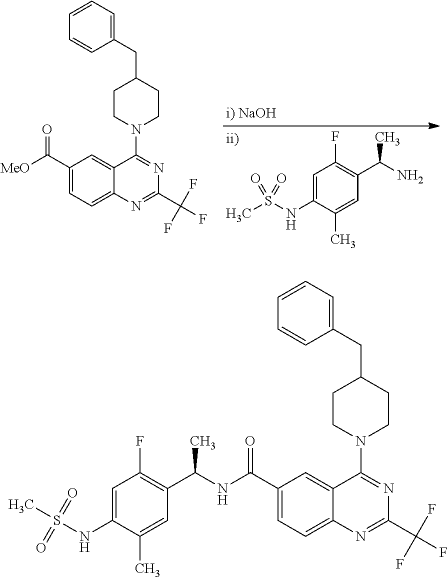

The compound is prepared in a similar manner as Example 66 by hydrolysis of the corresponding ester (25 mg, 0.056 mmol) and condensing the acid obtained with the appropriate amine (18 mg, 0.062 mmol) to give the title compound (12 mg, 33%). m/z=644.6 (M+1), 4.20 min. ¹H NMR (400 MHz; d₆-DMSO) δ 9.18 (1H, bs), 9.13 (1H, d), 8.49 (1H, d), 8.34 (1H, dd), 7.95 (1H, d), 7.34-7.28 (m, 3H), 7.23-7.18 (m, 3H), 7.09 (1H, d), 5.39-5.32 (1H, m), 4.55-4.47 (2H, m), 3.33-3.24 (2H, m), 3.00 (3H, s), 2.60 (2H, d), 2.22 (3H, s), 1.97 (1H, bs), 1.77 (2H, bs), 1.49 (3H, d), 1.40-1.44 (2H, m).

### Example 108

### N-((1R)-1-{2-FLUORO-5-METHYL-4-[(METHYLSULFONYL)AMINO]PHENYL}ETHYL)-2-(1-METHYLCYCLOPROPYL)QUINOLINE-6-CARBOXAMIDE

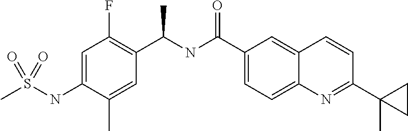

### 108A) 6-BROMO-2-ISOPROPENYLQUINOLINE

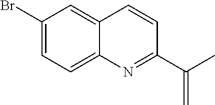

To a stirred suspension of (methyl)triphenylphosphonium bromide (2000 mg, 5.60 mmol) in dry THF (15 ml) was added a solution of potassium t-butoxide (628 mg, 5.60 mmol) in dry THF (10 ml) at ice-cooling. After 2 hours at room temperature, to this was added a solution of 1-(6-bromoquinolin-2-yl)ethanone (Example 48B) (700 mg, 2.80 mmol) in dry THF (15 ml) at ice-cooling. After 3 hours at ambient temperature, the mixture was quenched with water and extracted with ethyl acetate (×2). The combined solution was washed with brine, dried over sodium sulfate and concentrated in vacuo to give crude product, which was purified by column chromatography on silica gel (250 g) with hexane-ethyl acetate (10:1) to furnish the title compound (661 mg, 95%) as a tan solid.

¹H NMR (270 MHz. CDCl₃) δ 2.34 (3H, s), 5.50 (1H, s), 5.93 (1H, s), 7.65-7.78 (2H, m), 7.88-8.03 (3H, m). MS (ESI): m/z 248.11, 250.14 [M+H]⁺.

### 108B) METHYL 2-ISOPROPENYLQUINOLINE-6-CARBOXYLATE

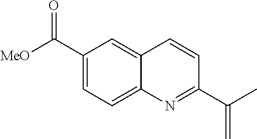

A mixture of 6-bromo-2-isopropenylquinoline (200 mg, 1.45 mmol), palladium acetate (18.1 mg, 0.081 mmol), 1,3-bis(diphenylphosphino)propane (33 mg, 0.081 mmol), triethylamine (245 mg, 2.42 mmol˜0.337 ml) and methanol (1.03 g, 1.31 ml˜32.2 mmol) in dry DMF (2.5 ml) was heated at 80° C. under carbon monooxide gas (balloon) for overnight (15 hours). The mixture was diluted with ethyl acetate-toluene (8:1) (159 ml) and the precipitate was filtered through a pad of celite. The organic layer was washed with water (×2), brine, dried over sodium sulfate and concentrated in vacuo to give the crude product. The crude product was purified by column chromatography on silica gel (150 g) with hexane-ethyl acetate (15:1) to furnish the title compound (150 mg, 82%) as dark yellow solid.

¹H NMR (270 MHz. CDCl₃) δ 2.36 (3H, s), 3.99 (3H, s), 5.53-5.57 (1H, m), 5.98 (1H, s), 7.73-7.78 (1H, m), 8.08-8.31 (3H, m), 8.54-8.56 (1H, m)

MS (ESI): m/z 228.21 [M+H]⁺.

### 108C) METHYL 2-(1-METHYLCYCLOPROPYL)QUINOLINE-6-CARBOXYLATE

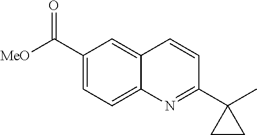

To a stirred suspension of trimethylsulfoxonium iodide (435 mg, 2.06 mmol) in dimethylsulfoxide-THF (3 ml-2 ml) was added potassium t-butoxide (231 mg, 2.06 mmol) in one portion at ambient temperature. After 30 min. at same temperature, to this (colorless solution) was added a solution of methyl 2-isopropenylquinoline-6-carboxylate (312 mg, 1.37 mmol) in THF (3 ml) at room temperature. The mixture was stirred at room temperature for 40 min then 1 hour at 60° C. The mixture was quenched with water and diluted with ethyl acetate-toluene (8:1) (90 ml). The organic solution was separated and washed with water (×2), brine, dried over sodium sulfate and concentrated in vacuo to crude product. The crude product was purified by column chromatography on silica gel (250 g) with hexane-ethyl acetate (10:1) to furnish the title compound (225 mg, 68%) as a white solid.

¹H NMR (270 MHz. CDCl₃) δ 0.91-0.98 (2H, m), 1.38-1.45 (2H, m), 1.64 (3H, s), 3.98 (3H, s), 7.42-7.48 (1H, m), 7.97-8.27 (3H, m), 8.50-8.55 (1H, m)

MS (ESI): m/z 242.15 [M+H]⁺.

### 108D) 2-(1-METHYLCYCLOPROPYL)QUINOLINE-6-CARBOXYLIC ACID

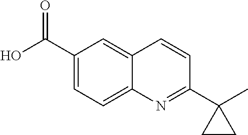

A solution of 108C (225 mg, 0.93 mmol) and 2M sodium hydroxide solution (2 ml. 4 mmol) in methanol (10 ml) was heated at 60° C. for 2 hours. After the solvent was evaporated in vacuo, the residue was dissolved with water. The aqueous solution was neutralized with 2M hydrochloric acid solution (2 ml) and the precipitate white solid was extracted with ethyl acetate (×3). The combined solution was washed with brine, dried over sodium sulfate and concentrated in vacuo to give crude white solid, which was recrystallized from ethyl acetate and hexane to furnish the title compound (177 mg, 84%) as a white solid.

MS (ESI): m/z 228.15 [M+H]⁺, 226.13 [M−H]⁻.

### 108E) N-((1R)-1-{2-FLUORO-5-METHYL-4-[(METHYLSULFONYL)AMINO]PHENYL}ETHYL)-2-(1-METHYLCYCLOPROPYL)QUINOLINE-6-CARBOXAMIDE

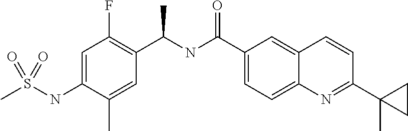

A solution of 2-(1-methylcyclopropyl)quinoline-6-carboxylic acid (187 mg, 0.66 mmol), Example 13D (150 mg, 0.66 mmol), HBTU (227 mg, 0.6 mmol) and triethylamine (200 mg, 0.28 ml, 1.98 mmol) in anhydrous N,N-dimethylformamide (10 ml) was treated in the same procedure described in Example 1G to furnish the title compound (251 mg, 84%) as a white solid.

¹H NMR (270 MHz. CDCl₃) δ 0.92-0.99 (2H, m), 1.30-1.37 (2H, m), 1.50 (3H, d, J=7.2 Hz); 1.60 (3H, s), 2.26 (3H, s), 3.03 (3H, s), 5.33-5.47 (1H, m), 7.06-7.14 (1H, m), 7.34-7.41 (1H, m), 7.51-7.58 (1H, m), 7.89-7.96 (1H, m), 8.11-8.18 (1H, m), 8.33-8.40 (1H, m), 8.49 (1H, s), 8.98-9.05 (1H, m), 9.19 (1H, s).

MS (ESI): m/z 456.15 [M+H]⁺, 454.21 [M−H]⁻.

### Example 110

### (R)-2-(4,4-DIFLUOROPIPERIDIN-1-YL)-N-(1-(2-FLUORO-5-METHYL-4-(METHYLSULFONAMIDO)PHENYL)ETHYL)QUINOLINE-6-CARBOXAMIDE

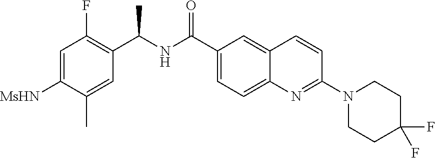

### 110A) ETHYL 2-(4,4-DIFLUOROPIPERIDIN-1-YL)QUINOLINE-6-CARBOXYLATE

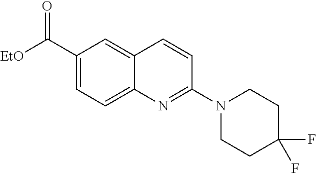

A mixture of the compound of Example 69A (200 mg, 0.714 mmol) and 4,4-difluoropiperidine (225 mg, 1.43 mmol) in EtOH (7 ml) was stirred at 60° C. for 24 hours. The reaction mixture was evaporated to remove the solvents, and the residue was chromatographed on a column of silica gel (ethyl acetate-hexane=1:5 to 1:1) as eluent to give the title compound (65 mg, 28%) as white solid.

¹H NMR (270 MHz, CDCl₃) [symbol] 1.43 (3H, t, J=7.3 Hz), 1.97-2.24 (4H, m), 3.87-4.05 (4H, m), 4.41 (2H, q, J=7.3 Hz), 7.06 (1H, d, J=9.2 Hz), 7.68 (1H, d, J=8.6 Hz), 7.99 (1H, d, J=9.2 Hz), 8.16 (1H, d, J=8.6 Hz), 8.37 (1H, s).

MS (ESI): m/z 321 (M+H)⁺.

### 110B) 2-(4,4-DIFLUOROPIPERIDIN-1-YL)QUINOLINE-6-CARBOXYLIC ACID

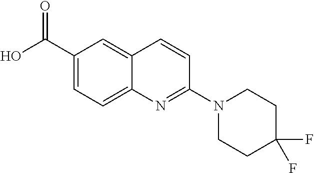

A mixture of the compound of Example 110A (65 mg, 0.203 mmol) and 2M sodium hydroxide solution (0.203 ml, 0.406 mmol) in ethanol (2 ml) was treated in the same procedure described in Example 69C. The aqueous mixture was concentrated and dried in vacuo to give the title compound as white solid (including sodium chloride). These crude products were used for the next step without purification.

MS (ESI): m/z 293 (M+H)⁺, 291 (M−H)⁺.

### 110C) (R)-2-(4,4-DIFLUOROPIPERIDIN-1-YL)-N-(1-(2-FLUORO-5-METHYL-4-(METHYLSULFONAMIDO)PHENYL)ETHYL)QUINOLINE-6-CARBOXAMIDE

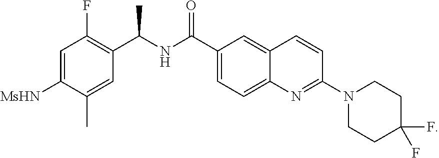

To a DMF (2 ml) solution of the compound of Example 13D (57 mg, 0.203 mmol), triethylamine (0.085 ml, 0.609 mmol), the compound of Example 110B (59 mg, 0.203 mmol), and HBTU (85 mg, 0.223 mmol) was treated in the same procedure described in Example 1G. The crude residue was purified by a silica gel column chromatography eluted with hexane/ethyl acetate (1:1 to 1:2) and recrystallized from diethylether-hexane to furnish the title compound (77 mg, 73%) as white solid.

¹H NMR (270 MHz, DMSO-d₆) 1.48 (3H, d, J=7.3 Hz), 1.93-2.17 (4H, m), 2.25 (3H, s), 3.02 (3H, s), 3.84-3.99 (4H, m), 5.38 (1H, m), 7.09 (1H, d, J=11.9 Hz), 7.36 (1H, d, J=7.9 Hz), 7.40 (1H, d, J=9.2 Hz), 7.61 (1H, d, J=8.6 Hz), 8.03 (1H, dd, J=2.0 Hz, 8.6 Hz), 8.17 (1H, d, J=9.2 Hz), 8.30 (1H, d, J=2.0 Hz), 8.85 (1H, d, J=7.9 Hz), 9.18 (1H, s).

MS (ESI) m/z 521 (M+H)⁺, 519 (M−H)⁺.

### Example 111

### 6-(5-TRIFLUOROMETHYL-PYRAZOL-1-YL)-NAPHTHALENE-2-CARBOXYLIC ACID [(R)-1-(2-FLUORO-4-METHANESULFONYLAMINO-5-METHYL-PHENYL)-ETHYL]-AMIDE

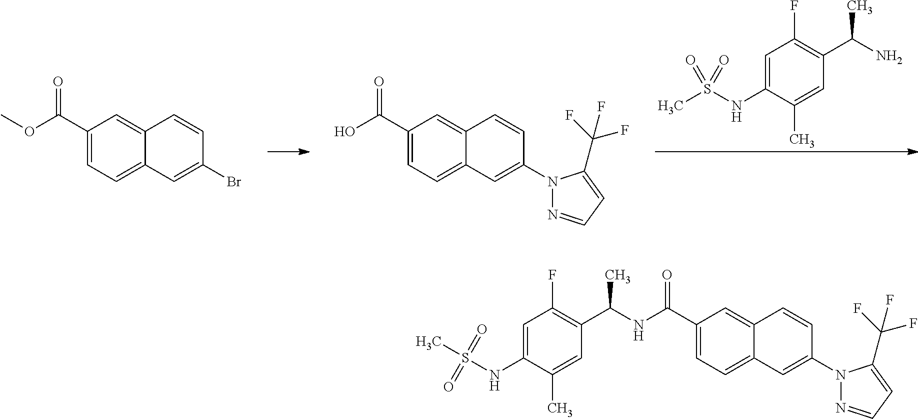

### 111A) METHYL 6-(5-(TRIFLUOROMETHYL)-1H-PYRAZOL-1-YL)-2-NAPHTHOATE

The compound is prepared in a similar manner as Example 100A by reaction of 6-bromonaphthalene-2-carboxylic acid methyl ester (400 mg, 1.5 mmol) and 3-(trifluoromethyl)pyrazole (310 mg, 2.3 mmol) to give the title compound (115 mg, 24%). m/z=253.5 (M+1); r.t. 3.44 min. ¹H NMR (400 MHz; CDCl₃) δ 8.64 (1H, bs), 8.20 (1H, s), 8.14 (1H, d), 8.13 (1H, s), 8.09 (1H, d), 7.96 (1H, s), 7.94 (1H, s), 6.80 (1H, t), 4.00 (3H; s).

### 111B) 6-(5-TRIFLUOROMETHYL-PYRAZOL-1-YL)-NAPHTHALENE-2-CARBOXYLIC ACID [(R)-1-(2-FLUORO-4-METHANESULFONYLAMINO-5-METHYL-PHENYL)-ETHYL]-AMIDE

The compound is prepared in a similar manner as Example 66 by hydrolysis of the corresponding ester (44 mg, 0.14 mmol) and condensing the acid obtained with the appropriate amine (43 mg, 0.15 mmol) to give the title compound (7 mg, 9%). m/z=535.2 (M+1), r.t. 3.79 min. ¹H NMR (400 MHz; d₆-DMSO) δ 9.19 (1H, s), 9.04 (1H, d), 8.93-8.91 (1H, m), 8.57 (1H, bs), 8.51 (1H, d), 8.25 (1H, d), 8.18-8.15 (1H, m), 8.14 (1H, d), 8.03 (1H, dd), 7.37 (1H, d), 7.14 (1H, d), 7.10 (1H, d), 5.44-5.38 (1H, m), 5.32 (1H, t), 3.02 (3H, s), 2.25 (3H, s), 1.46 (3H, d).

### Example 112

### 4-MORPHOLIN-4-YL-2-TRIFLUOROMETHYL-QUINAZOLINE-6-CARBOXYLIC ACID [(R)-1-(2-FLUORO-4-METHANESULFONYLAMINO-5-METHYL-PHENYL)-ETHYL]-AMIDE

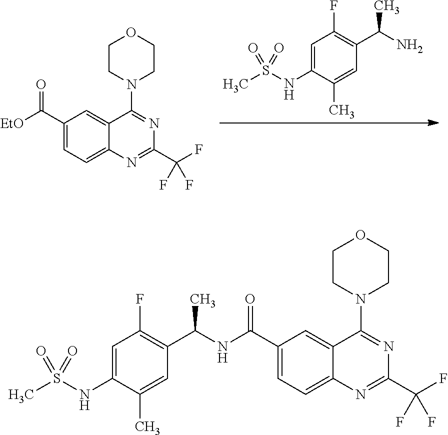

The compound is prepared in a similar manner as Example 66 by hydrolysis of the corresponding ester (25 mg, 0.070 mmol) and condensing the acid obtained with the appropriate amine (22 mg, 0.077 mmol) to give the title compound (15 mg, 38%). m/z=556.3 (M+1), 3.41 min. ¹H NMR (400 MHz; d₆-DMSO) δ 9.26 (1H, bs), 9.15 (1H, d), 8.53 (1H, d), 8.36 (1H, dd), 8.00 (1H, d), 7.33 (1H, d), 7.09 (1H, d), 5.40-5.32 (1H, m), 4.00-3.98 (4H, m), 3.80-3.77 (4H; m), 3.01 (3H, s), 2.24 (3H, s), 1.49 (3H, d).

### Example 114

### (R)-2-(DIETHYLAMINO)-N-(1-(2-FLUORO-5-METHYL-4-(METHYLSULFONAMIDO)PHENYL)-ETHYL)QUINOLINE-6-CARBOXAMIDE

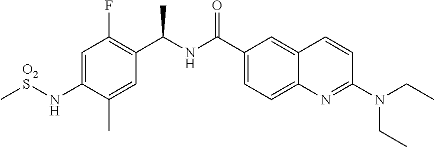

### 114A) ETHYL 2-(DIETHYLAMINO)QUINOLINE-6-CARBOXYLATE

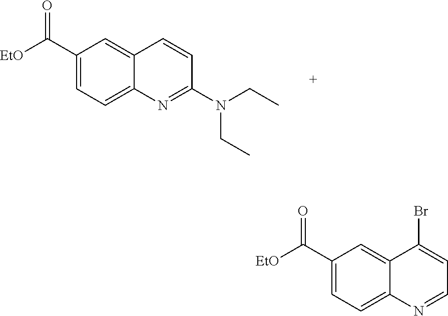

A mixture of the compound of Example 69A (200 mg, 0.714 mmol) and diethylamine (104 mg, 1.43 mmol) in ethanol (2 ml) was stirred at 60° C. for 2 hours. The reaction mixture was evaporated to remove the solvents, and the residue was chromatographed on a column of silica gel (ethyl acetate-hexane=1:5 to 1:2) as eluent to give the 196 mg of ethyl 2-(diethylamino)quinoline-6-carboxylate (including ethyl 4-bromoquinoline-6-carboxylate) as white solid. This mixture was used for the next step without further purification.

MS (ESI) m/z 273, 281, 283 (M+H)⁺⁻.

### 114B) 2-(DIETHYLAMINO)QUINOLINE-6-CARBOXYLIC ACID

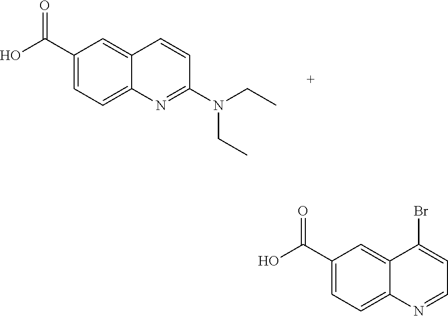

A mixture of the compound of the product of Example 114A and 2M sodium hydroxide solution (1 ml) in ethanol (2 ml) was treated in the same procedure described in Example 75B. The aqueous mixture was concentrated and dried in vacuo to give the title compound as white solid (including 4-bromoquinoline-6-carboxylic acid and sodium chloride). These crude products were used for the next step without purification.

MS (ESI): m/z 245 (M+H)⁺, 250, 252 (M−H)⁺.

### 114C) (R)-2-(DIETHYLAMINO)-N-(1-(2-FLUORO-5-METHYL-4-(METHYLSULFONAMIDO)-PHENYL)ETHYL)QUINOLINE-6-CARBOXAMIDE

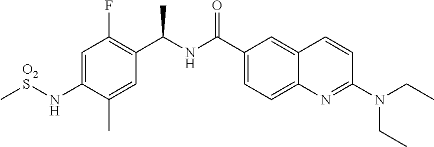

To a DMF (2 ml) solution of the compound of Example 13D (102 mg, 0.360 mmol), triethylamine (0.151 ml, 1.08 mmol), the compound of Example 114B (crude 0.360 mmol), and HBTU (150 mg, 0.396 mmol) was treated in the same procedure described in Example 1G. The crude residue was applied to a silica gel column chromatography and eluted with hexane/ethyl acetate (1:1 to 1:2) and HPLC (used column was XTerra MS C18, 5 um, 30×50 mm) eluting with acetonitrile/0.01% ammonium aqueous solution (basic 32_{—}96, 32:68 to 96:4) to furnish the title compound (8.0 mg, 5% yield for 3 steps) as a white solid.

¹H NMR (270 MHz, DMSO-d₆) [symbol] 1.18 (6H, t, J=6.6 Hz), 1.48 (3H, d, J=7.3 Hz), 2.24 (3H, s), 3.01 (3H, s), 3.65 (4H, q, J=6.6 Hz), 5.38 (1H, m), 7.06 (1H, s), 7.10 (1H, d, J=2.6 Hz), 7.35 (1H, d, J=8.6 Hz), 7.52 (1H, d, J=8.6 Hz), 7.99 (1H, d, J=8.6 Hz), 8.06 (1H, d, J=9.2 Hz), 8.26 (1H, br s), 8.79 (1H, d, J=7.3 Hz). the amide N—H peak was not observed.

MS (ESI) m/z 473 (M+H)⁺, 471 (M−H)⁺.

### Example 115

### (R)—N-(1-(2-FLUORO-5-METHYL-4-(METHYLSULFONAMIDO)PHENYL)ETHYL)-2-(4-(TRIFLUOROMETHYL)PIPERIDIN-1-YL)QUINOLINE-6-CARBOXAMIDE

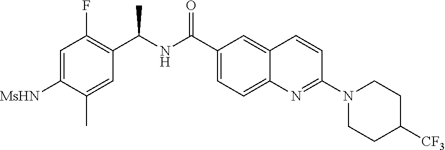

### 115A) ETHYL 2(4-(TRIFLUOROMETHYL)PIPERIDIN-1-YL)QUINOLINE-6-CARBOXYLATE

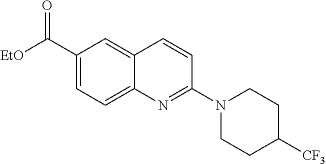

A mixture of the compound of Example 69A (200 mg, 0.714 mmol) and 4-(trifluoromethyl)piperidine (271 mg, 1.43 mmol) in EtOH (7 ml) was stirred at 60° C. for 24 hours. The reaction mixture was evaporated to remove the solvents, and the residue was chromatographed on a column of silica gel (ethyl acetate-hexane=1:5 to 1:2) as eluent to give the title compound (30 mg, 12%) as white solid.

¹H NMR (270 MHz, CDCl₃) [symbol] 1.43 (3H, t, J=6.6 Hz), 1.66 (2H, dq, J=4.0 Hz, 12.5 Hz), 2.02 (2H, br d, J=15.3 Hz), 2.24-2.51 (1H, m), 2.98 (2H, br t, J=13.5 Hz), 4.41 (2H, q, J=7.3 Hz), 4.75 (2H, br d, J=12.6 Hz), 7.03 (1H, d, J=9.2 Hz), 7.67 (1H, d, J=9.2 Hz), 7.96 (1H, d, J=9.2 Hz), 8.15 (1H, dd, J=2.0 Hz, 8.6 Hz), 8.35 (1H, d, J=2.0 Hz).

MS (ESI): m/z 353 (M+H)⁺.

### 115B) 2-(4-(TRIFLUOROMETHYL)PIPERIDIN-1-YL)QUINOLINE-6-CARBOXYLIC ACID

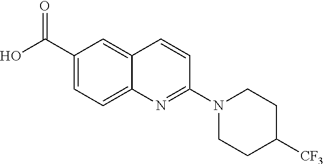

A mixture of the compound of Example 115A (30 mg, 0.085 mmol) and 2M sodium hydroxide solution (1 ml) in ethanol (2 ml) was treated in the same procedure described in Example 69C. The aqueous mixture was concentrated and dried in vacuo to give the title compound as white solid (including sodium chloride). These crude products were used for the next step without purification.

MS (ESI): m/z 325 (M+H)⁺.

### 115C) (R)—N-(1-(2-FLUORO-5-METHYL-4-(METHYLSULFONAMIDO)PHENYL)ETHYL)-2-(4-(TRIFLUOROMETHYL)PIPERIDIN-1-YL)QUINOLINE-6-CARBOXAMIDE

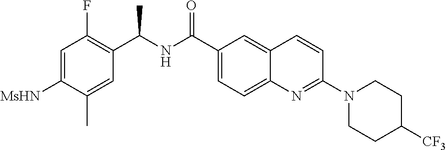

To a DMF (2 ml) solution of the compound of Example 13D (24 mg, 0.085 mmol), triethylamine (0.036 ml, 0.255 mmol), the compound of Example 115B (28 mg, 0.085 mmol), and HBTU (36 mg, 0.094 mmol) was treated in the same procedure described in Example 1G. The crude residue was purified by a silica gel column chromatography eluted with hexane/ethyl acetate (1:1 to 1:2) and washed with diethyl ether-hexane (10 ml) to furnish the title compound (25 mg, 53%) as white solid.

¹H NMR (270 MHz, DMSO-d₆) [symbol] 1.32-1.57 (5H, m, including 3H, d, J=6.6 Hz, 1.48 ppm), 1.94 (2H, br d, J=11.2 Hz), 2.63-2.80 (1H, m), 2.90-3.11 (5H, m, including 3H, s, 3.01 ppm) 4.72 (2H, br d, J=13.8 Hz), 5.38 (1H, m), 7.08 (1H, d, J=11.2 Hz), 7.29-7.40 (2H, m), 7.58 (1H, d, J=8.6 Hz), 8.02 (1H, dd, J=1.3 Hz, 8.6 Hz), 8.13 (1H, d, J=9.2 Hz), 8.29 (1H, br s), 8.84 (1H, d, J=7.9 Hz), 9.17 (1H, br s).

MS (ESI) m/z 553 (M+H)⁺, 551 (M−H)⁺.

### Example 116

### N-((1R)-1-{2-FLUORO-5-METHYL-4-[(METHYLSULFONYL)AMINO]PHENYL}ETHYL)-6-(2,2,2-TRIFLUORO-1-METHOXY-1-METHYLETHYL)-2-NAPHTHAMIDE

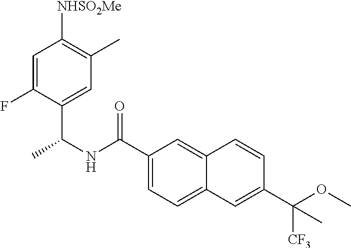

### 116A) METHYL 6-(2,2,2-TRIFLUORO-1-METHOXY-1-METHYLETHYL)-2-NAPHTHOATE

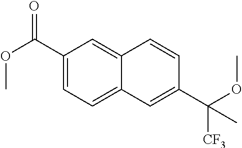

To a THF solution of the 101B (0.45 g, 1.5 mmol), sodium hydride (80 mg, 2.2 mmol) was added and the mixture was stirred for 30 minutes at 0° C. Then, methyl iodide (642 mg, 4.5 mmol) was added to the mixture and additional stirring was allowed for 3 hrs. Then, the product was extracted with ethyl acetate and dried over sodium sulfate. Then filtration, evaporation, purification through silica gel column chromatography eluting with hexane:ethyl acetate=4:1 to give the title compound as a white solid in 58% yield.

¹H NMR (300 MHz, DMSO-d₆) [symbol] 1.31 (3H, s), 2.02 (3H, s), 5.81-5.84 (1H, m), 6.11-6.14 (1H, m), 6.23-6.33 (3H, m), 6.77 (1H, s).

### 116B) 6-(2,2,2-TRIFLUORO-1-METHOXY-1-METHYLETHYL)-2-NAPHTHOIC ACID

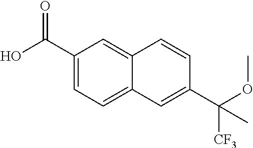

The title compound was prepared by the same procedure of Step 101C using the compound of 116A instead of the compound of 101B to give the title compound in 98% yield as a white solid.

¹H NMR (300 MHz, DMSO-d₆) [symbol] 1.91 (3H, s), 7.71-7.74 (1H, m), 8.01-8.21 (4H, m), 8.64 (1H, s), 13.2 (1H, brs).

### 116C) N-((1R)-1-{2-FLUORO-5-METHYL-4-[(METHYLSULFONYL)AMINO]PHENYL}ETHYL)-6-(2,2,2-TRIFLUORO-1-METHOXY-1-METHYLETHYL)-2-NAPHTHAMIDE (PF-04530505-00)

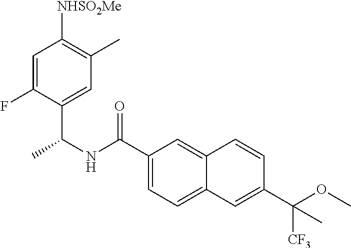

To a DMF (50 ml) solution of carboxylic acid 116B (60 mg, 0.21 mmol), HBTU (133 mg, 0.35 mmol) and triethylamine (107 mg, 1.06 mmol) were added and the mixture was stirred for 0.2 hour at 50 deg. Then, amine 13D (99.5 mg, 0.35 mmol) was portioned to this reaction and the mixture was stirred for 12 h at 50 deg. Then, the reaction was quenched with saturated sodium bicabonate and the product was extracted with ethyl acetate. After the evaporation, the crude residue was purified through silica gel column chromatography eluting with hexane/ethyl acetate=4/1 to give the title compound as a white solid in 32% yield.

¹H NMR (300 MHz, DMSO-d₆) 1.65 (3H, d, J=6.6 Hz), 1.90 (3H, s), 2.25 (3H, s), 3.05 (3H, s), 3.29 (3H, s), 4.09-4.16 (1H, m), 5.36-5.41 (1H, brs), 6.18 (1H, brs), 6.72-6.74 (1H, m), 7.32-7.99 (6H, m), 8.29 (1H, s).

### General Method for Automated Parallel LC-MS Purification of Libraries

The libraries were purified using a Perkin Elmer API100 mass spectrometer coupled to Shimadzu LC pumps. The chromatographic method employed was 10-100% gradient of acetonitrile to water over 8 minutes at a flow rate of 6 ml'per minute. The column used was a 10×50 mm YMC C18 and the compounds were collected using a Gilson 204 fraction collector.

Following the methods described above and the appropriate reagents, starting materials and purification methods known to those skilled in the art, the amide compounds of this invention were or can be prepared.

The synthetic and biological examples presented herein are offered to illustrate this invention and are not to be construed in any way as limiting the scope of this invention. In the examples below, all temperatures are in degrees Celsius (unless otherwise indicated).

The compounds that have been prepared in accordance with the invention are presented in Table 1, below. The syntheses of these representative compounds were carried out in accordance with the methods set forth above, and activity of the compounds was measured by percent inhibition in a calcium uptake assay, the details of which are described below.

### Calcium Uptake Assay.

Functional activity of compounds against the VR1 receptor was determined by measuring changes in intracellular calcium in HEK 293 cells expressing hVR1. Compounds were examined for their ability to inhibit agonist-induced calcium influx. Dual wavelength ratiometric dye, Fura2, was used as an indicator of relative levels of [Ca²⁺] in a 96-well format using a Flex Station®, Molecular Devices.

### Cell Line and Culture Conditions:

hVR1 was cloned into a pcDNA5/TO vector from Invitrogen and stably transformed into T-REx HEK 293 cell line from Invitrogen. HEK 293 cells expressing hVR1 were grown to confluency (24 hours culture) on PDL-coated, plastic 96-well black-walled plates, in the presence of DMEM medium containing 5% PenStrep, 5% Glutamax, 200 μg/mL Hygromycin, 5 μg/mL Blasticidin and 10% heat inactivated FBS. Twenty-four hours prior to assay, cells were transferred to DMEM media containing 1 μg/mL doxycycline. Prior to the assay, cells were loaded with 5 μg/mL Fura-2 (Molecular Probes) in saline solution (130 mM NaCl, 3 mM KCl, 1 mM CaCl₂, 0.6 mM MgCl₂, 10 mM HEPES, 10 mM glucose and 50 mM sucrose pH 7.4) at 37° C. for 40 minutes. The dye was then aspirated and replaced with 100 μL saline before commencement of the assay in Flex Station®.

### Agonist Concentration and Compound Dilutions:

The agonist EC₅₀ was determined at the start of the assay and compound IC₅₀ experiments were run using an agonist concentration equal to its EC₅₀ as stimulus. The agonists used were capsaicin (EC₅₀=2.5 nM) and protons (saline solution plus 10 mM citric acid buffered to pH 5.7 with HCl). Compounds were tested at concentrations ranging from 10 nM to 3.3 μM.

The assay consists of two stages: a pre-treatment phase followed by a treatment phase. 50 μl of a compound solution was added to the cells (Pre-treatment). In some instances, following pre-treatment, 50 μl of the test compound in a saline solution at pH 5.1 was added (Treatment). Compounds were tested as follows: For the pre-treatment phase, 50 μL of 3× concentration of test compound in saline is added to cells containing 100 μL of saline to achieve a final concentration of x. For the treatment phase, at a determined time after pre-treatment, 50 μL of test compound plus agonist solution is added to cells at the relevant concentrations.

Recordings were made at 4 second intervals at wavelengths of 340 nm and 380 nm and the fluorescence ratio analyzed. Responses were measured as peak fluorescence ratio after compound-agonist addition minus baseline fluorescence ratio prior to treatment and were calculated using the SoftMaxPro software from Molecular Devices. Percent inhibition was calculated as follows:

$$Percentage inhibition = 1 - \frac{( Compound Response - Control Response )}{( Agonist Response - Control Response )} \times 100$$

### Acid Stimulation Assay:

The Acid-induced changes in the intracellular calcium concentration were monitored using FDSS 6000 (Hamamatsu Photonics, Japan), a fluorometric imaging system. The cell suspension in resting buffer (HBSS supplemented with 10 mM HEPES, pH 7.4) was pre-incubated with varying concentrations of the test compounds or resting buffer (buffer control) for 15 minutes at room temperature under dark conditions. The cells were automatically added to the stimulating solution (HBSS supplemented with MES, final assay buffer pH5.8) by the FDSS 6000. The IC₅₀ values of VR1 antagonists were determined from one half of the increase demonstrated by buffer control samples after acidic stimulation, and the results obtained with representative compounds of the invention, prepared according to the methods described herein, are set forth in Table 1, below.

TABLE 1
AMIDE COMPOUNDS

| | | | | IC₅₀ |
|---|---|---|---|---|
| | | MW | MW | (nM) |
| ID | Structure | (Calcd) | (Obs) | Low pH |
| 1 | 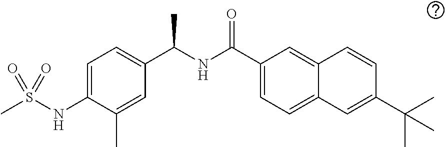 | 438.59 | | 3 |
| 2 | 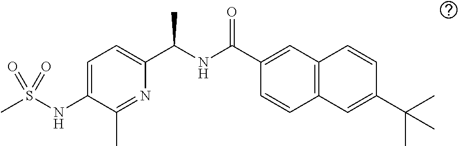 | 439.58 | | 4 |
| 3 | 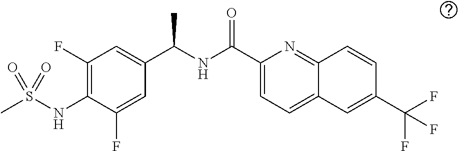 | 473.42 | | 4 |
| 4 | 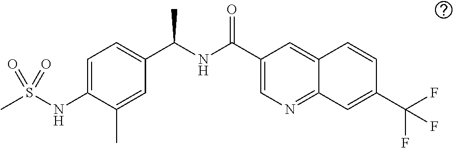 | 451.47 | | 100 |
| 5 | 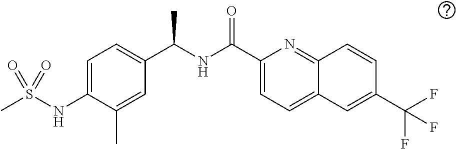 | 451.47 | | |
| 6 | 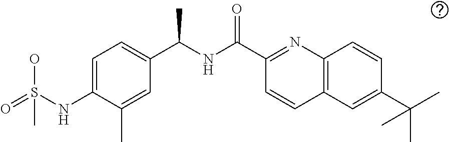 | 439.58 | | |
| 7 | 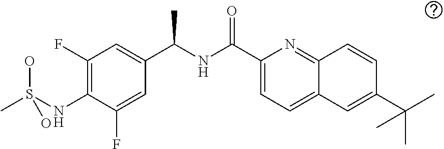 | 461.53 | | 1 |
| 8 | 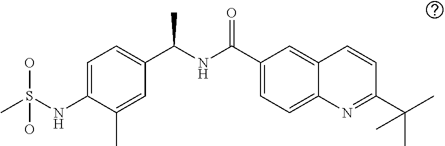 | 439.58 | | |
| 9 | 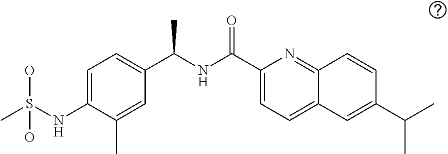 | 425.55 | | 714 |
| 10 | 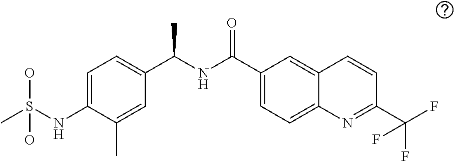 | 451.47 | | |
| 11 | 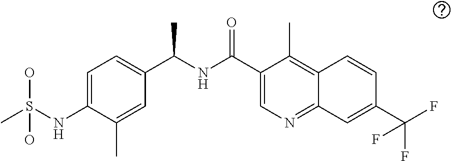 | 465.49 | | 240 |
| 12 | 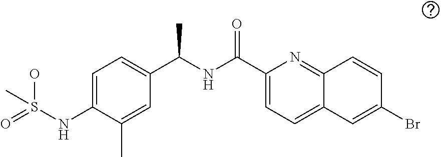 | 462.37 | | 444 |
| 13 | 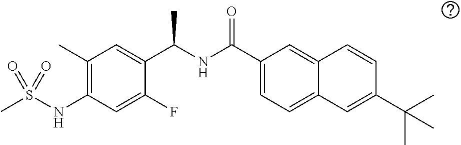 | 456.58 | | 127 |
| 14 | 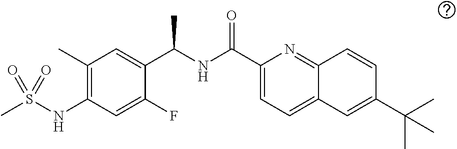 | 457.57 | | 288 |
| 15 | 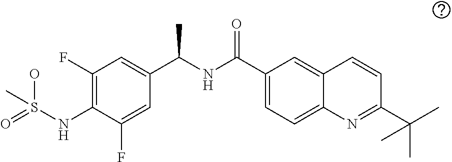 | 461.53 | | 24 |
| 16 | 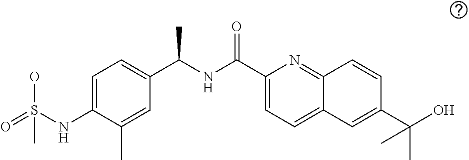 | 441.55 | | 71 |
| 17 | 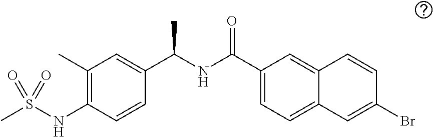 | 461.38 | | 14 |
| 18 | 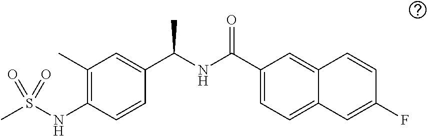 | 400.47 | | 83 |
| 19 | 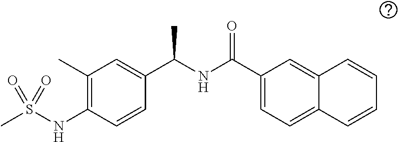 | 382.48 | | 63 |
| 20 | 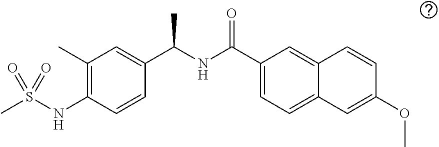 | 412.51 | | 8 |
| 21 | 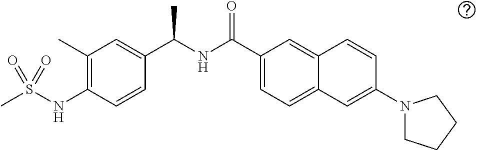 | 451.59 | | 44 |
| 22 | 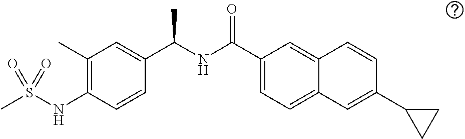 | 422.55 | | 3 |
| 23 | 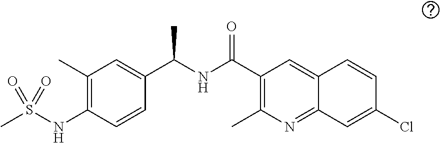 | 431.94 | | 379 |
| 24 | 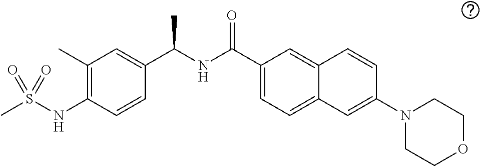 | 467.59 | | 1000 |
| 25 | 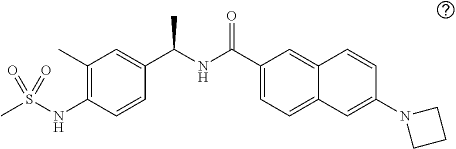 | 437.56 | | 1000 |
| 26 | 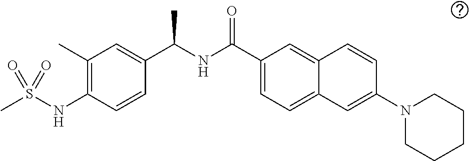 | 465.61 | | 1000 |
| 27 | 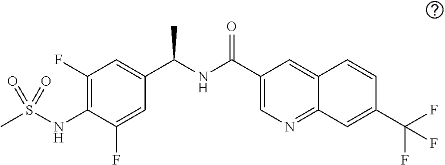 | 473.42 | | 105 |
| 28 | 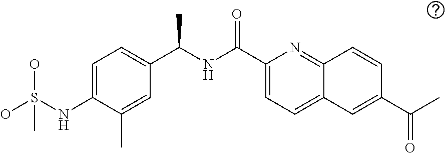 | 425.51 | | 187 |
| 29 | 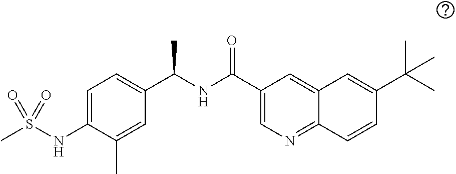 | 439.58 | | 231 |
| 30 | 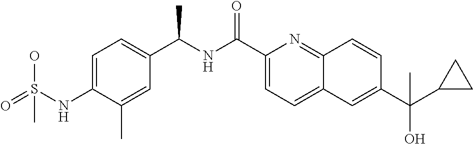 | 467.59 | | |
| 31 | 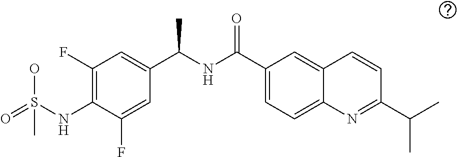 | 447.5 | | 47 |
| 32 | 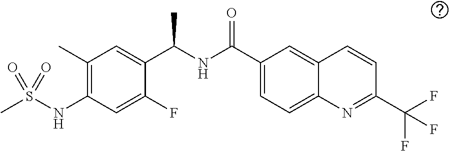 | 469.46 | | 6 |
| 33 | 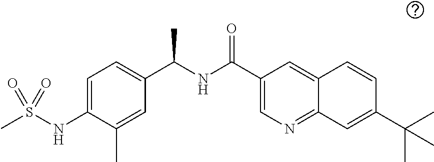 | 439.58 | | 56 |
| 34 | 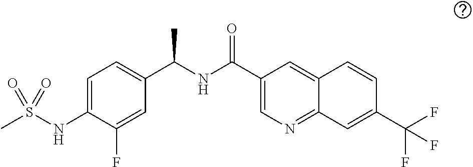 | 455.43 | | 26 |
| 35 | 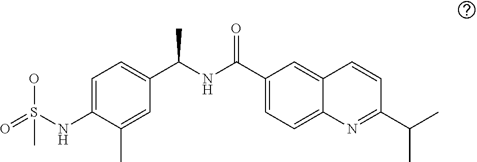 | 425.55 | | 5 |
| 36 | 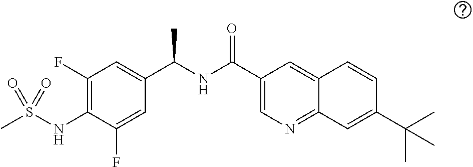 | 461.53 | | |
| 37 | 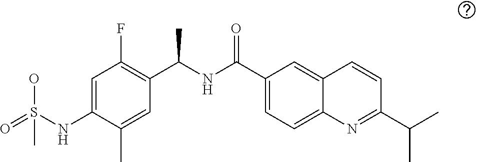 | 443.54 | | |
| 38 | 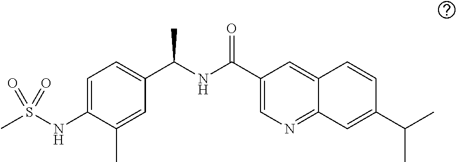 | 425.55 | | |
| 39 | 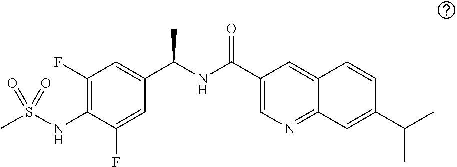 | 447.5 | | 13 |
| 40 | 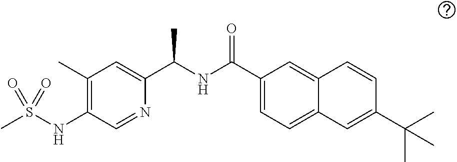 | 439.58 | | 31 |
| 41 | 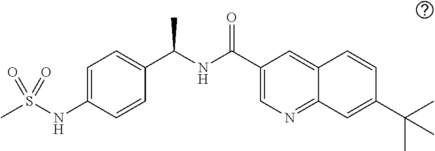 | 425.55 | | 29 |
| 42 | 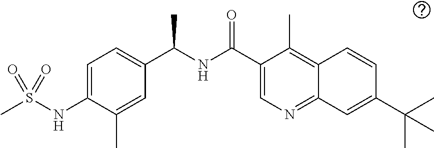 | 453.6 | | 532 |
| 43 | 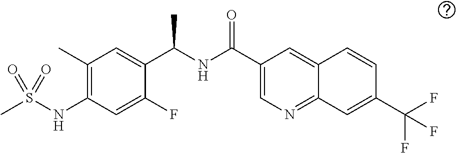 | 469.46 | | 30 |
| 44 | 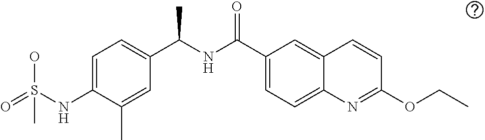 | 427.52 | | 15 |
| 45 | 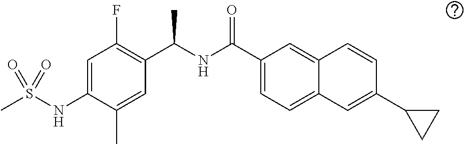 | 440.54 | | |
| 46 | 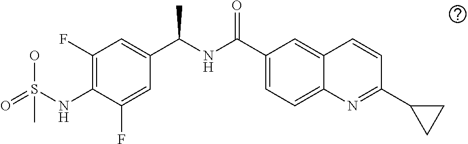 | 445.49 | | |
| 47 | 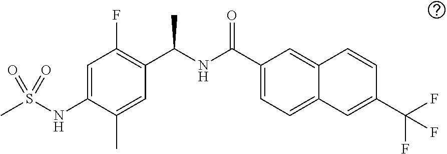 | 468.47 | | |
| 48 | 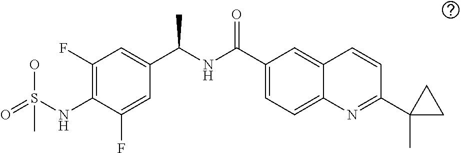 | 459.51 | | |
| 49 | 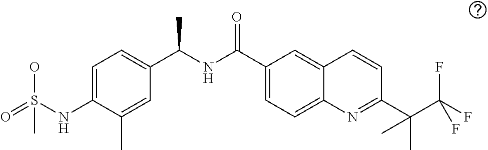 | 493.55 | | |
| 50 | 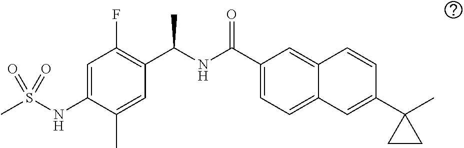 | 454.56 | | |
| 51 | 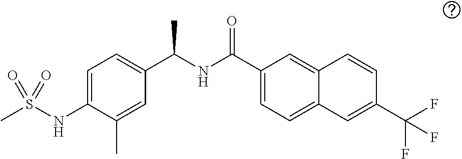 | 450.48 | | 1 |
| 52 | 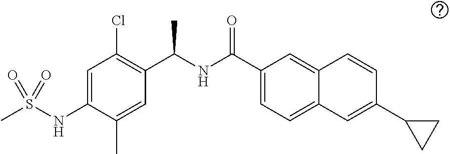 | 456.99 | | 22 |
| 53 | 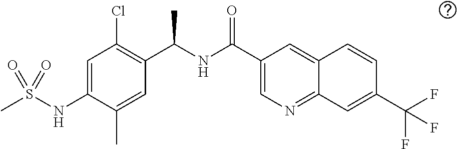 | 485.91 | | 36 |
| 54 | 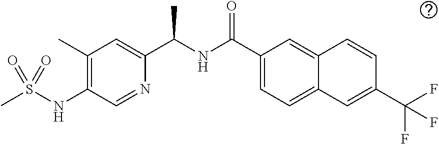 | 451.47 | | 12 |
| 55 | 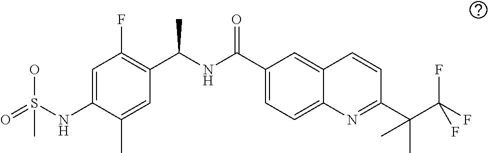 | 511.54 | | |
| 56 | 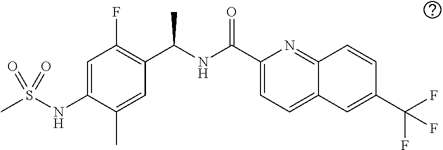 | 469.46 | | |
| 57 | 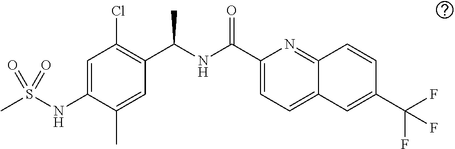 | 485.91 | | 58 |
| 58 | 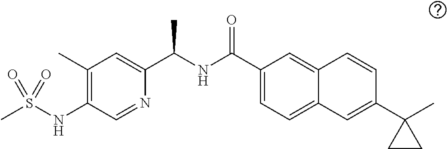 | 437.56 | | 7 |
| 59 | 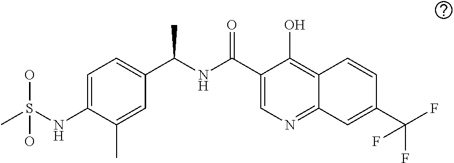 | 467.47 | | |
| 60 | 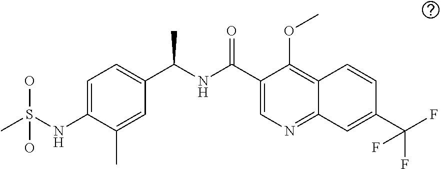 | 481.49 | | 1000 |
| 61 | 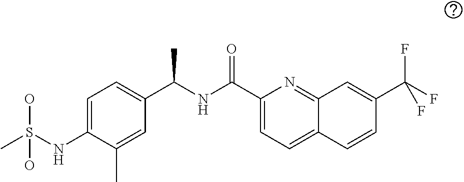 | 451.47 | | |
| 62 | 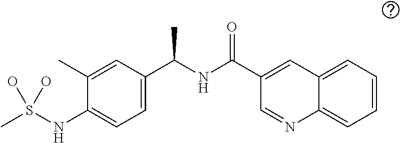 | 383.47 | 384.2 | |
| 63 | 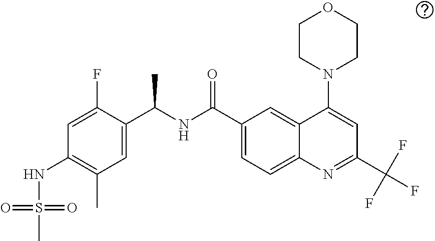 | 554.56 | 554.7 | 16 |
| 64 | 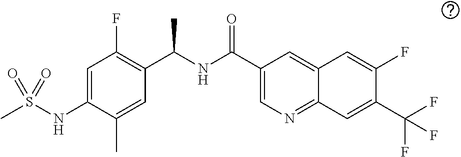 | 487.45 | | |
| 65 | 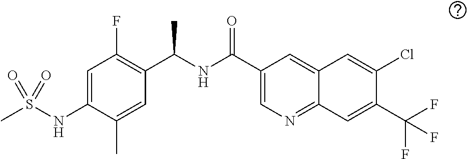 | 503.9 | | |
| 66 | 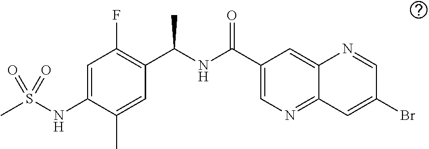 | 481.34 | 483.1 | 1000 |
| 67 | 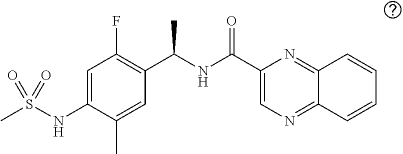 | 402.45 | 403.1 | 1000 |
| 68 | 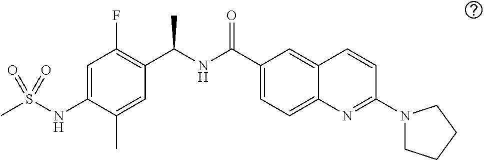 | 470.57 | | 293 |
| 69 | 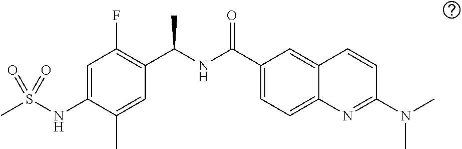 | 444.53 | | |
| 70 | 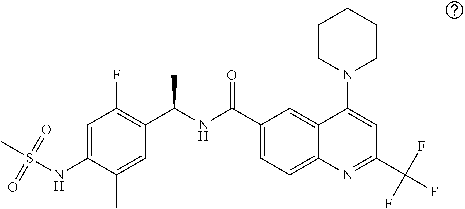 | 552.59 | 552.7 | 26 |
| 71 | 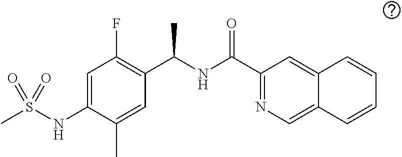 | 401.46 | 402.2 | 1000 |
| 72 | 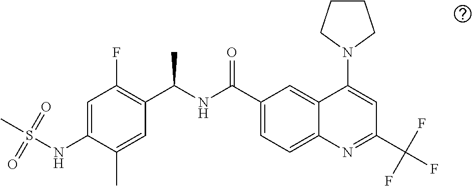 | 538.56 | 539.9 | 31 |
| 73 | 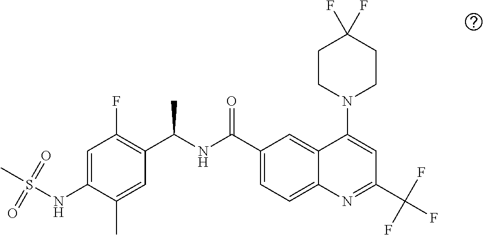 | 588.57 | 589.2 | 29 |
| 74 | 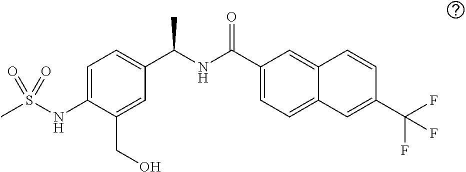 | 466.48 | | 13 |
| 75 | 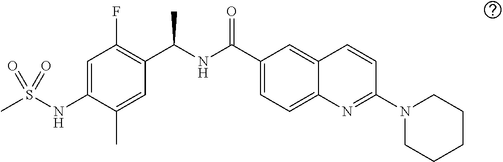 | 484.59 | | |
| 76 | 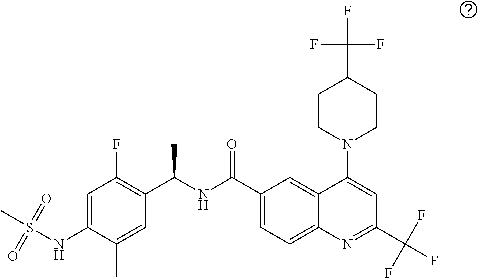 | 620.59 | 621.4 | 48 |
| 77 | 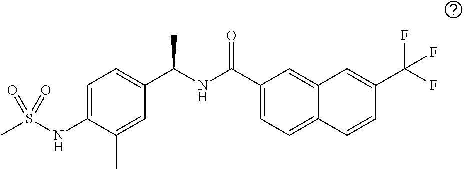 | 450.48 | | 4 |
| 78 | 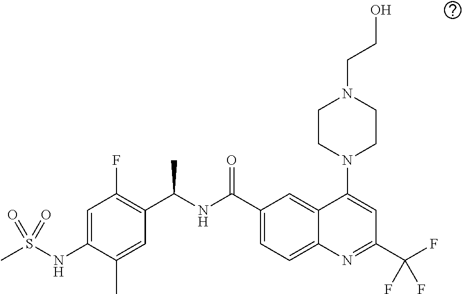 | 597.63 | 598.4 | 1000 |
| 79 | 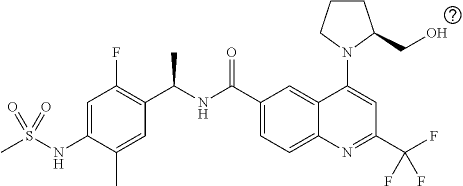 | 568.59 | 569.5 | 1000 |
| 80 | 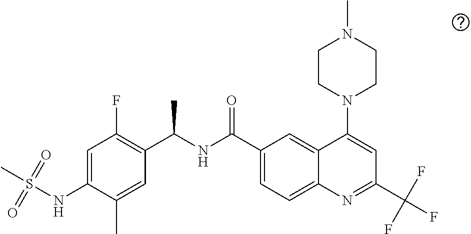 | 567.61 | 567.3 | 1000 |
| 81 | 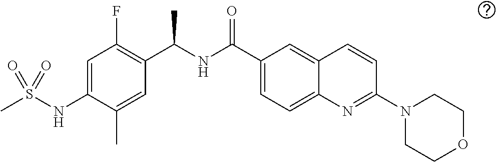 | 486.57 | | 748 |
| 82 | 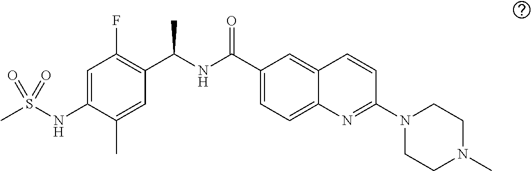 | 499.61 | | 1000 |
| 83 | 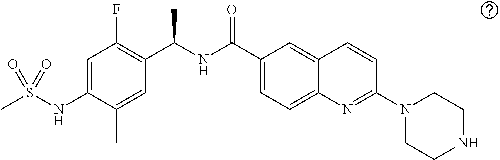 | 485.58 | | 1000 |
| 84 | 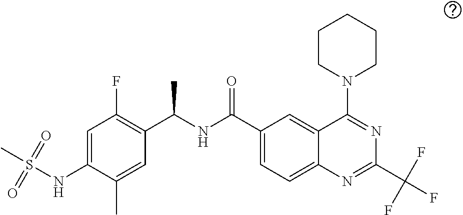 | 553.58 | 554.5 | 51 |
| 85 | 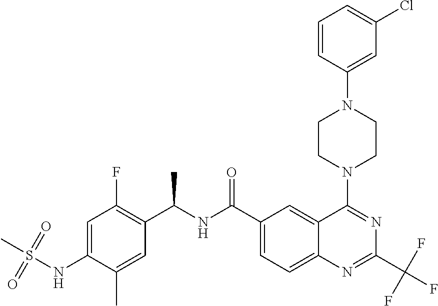 | 665.11 | 665.4 | 60 |
| 86 | 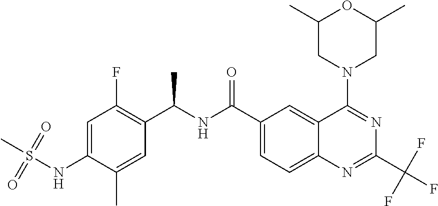 | 583.6 | 584.5 | 7 |
| 87 | 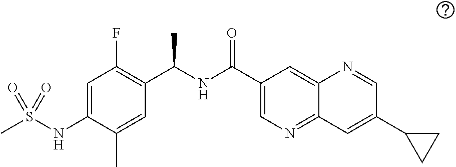 | 442.51 | 443.3 | 111 |
| 88 | 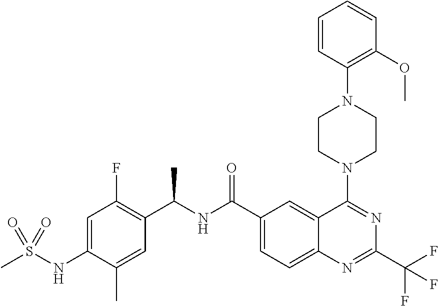 | 660.69 | 661.6 | 50 |
| 90 | 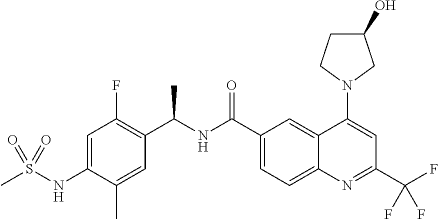 | 554.56 | 555.2 | 112 |
| 91 | 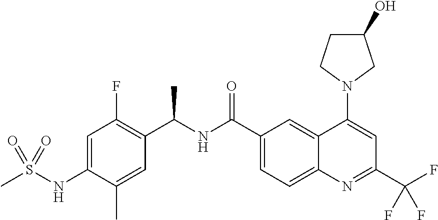 | 554.56 | 555.3 | 366 |
| 92 | 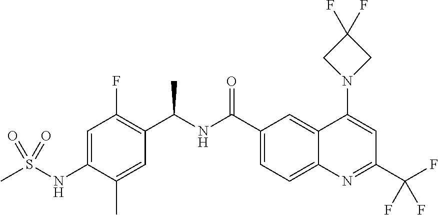 | 560.52 | 561.4 | 24 |
| 93 | 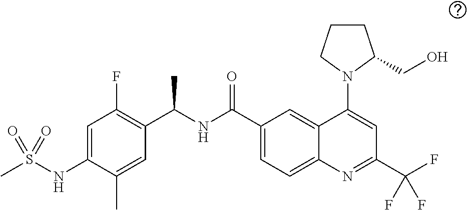 | 568.59 | 569.4 | 370 |
| 94 | 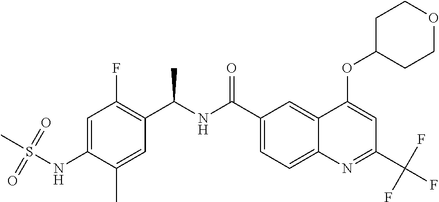 | 569.57 | 570.2 | 35 |
| 95 | 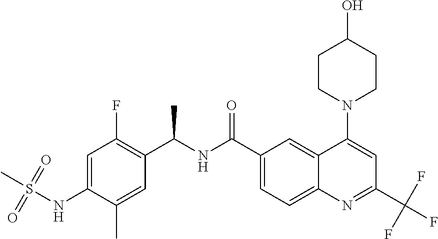 | 568.59 | 568.5 | 1000 |
| 96 | 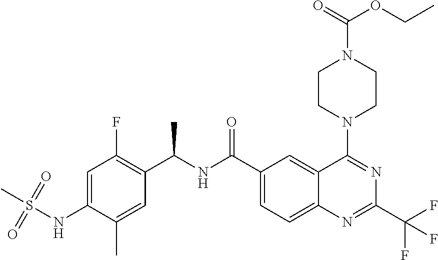 | 626.63 | 627.1 | 41 |
| 97 | 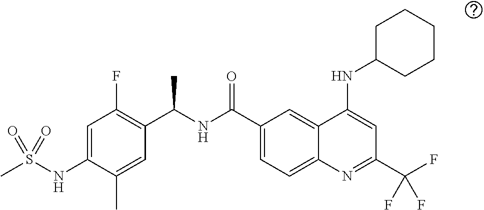 | 566.62 | 567.4 | 41 |
| 98 | 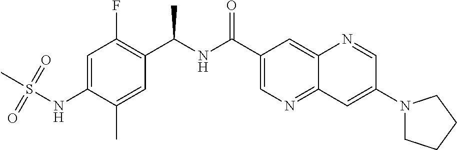 | 471.55 | 472.4 | 1000 |
| 99 | 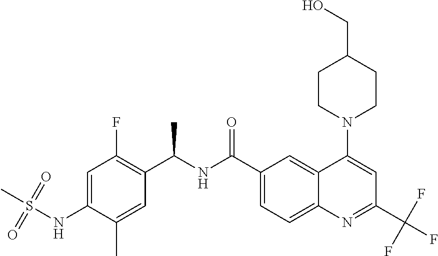 | 582.62 | 583.3 | 630 |
| 100 | 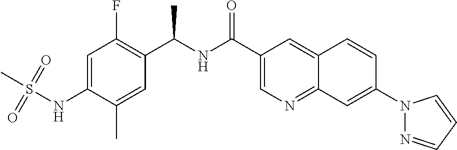 | 468.51 | 469.4 | 1000 |
| 101 | 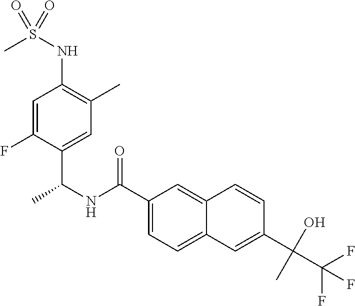 | 512.52 | | 41 |
| 102 | 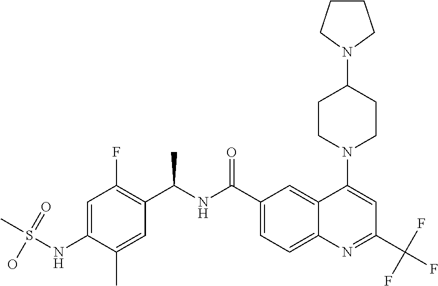 | 621.7 | 622 | 1000 |
| 103 | 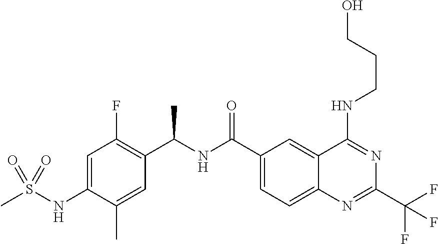 | 543.54 | 544.5 | 1000 |
| 104 | 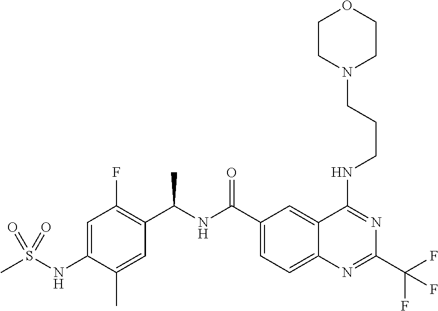 | 612.65 | 613.2 | 1000 |
| 105 | 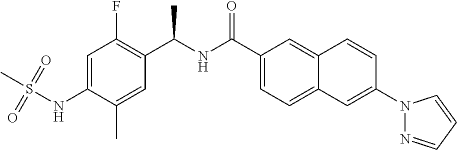 | 466.53 | 467.5 | 147 |
| 106 | 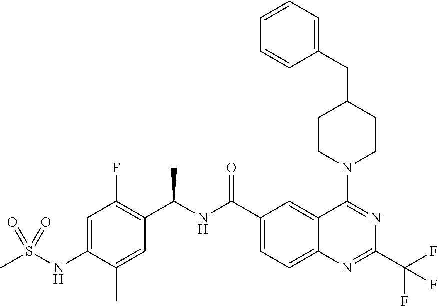 | 643.7 | 644.6 | 6 |
| 107 | 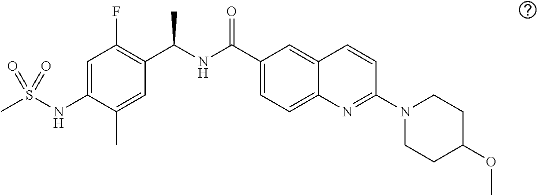 | 514.62 | | 337 |
| 108 | 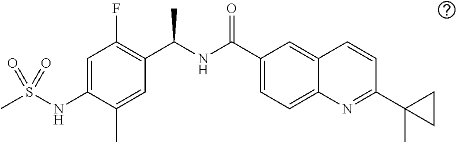 | 455.55 | | 5 |
| 109 | 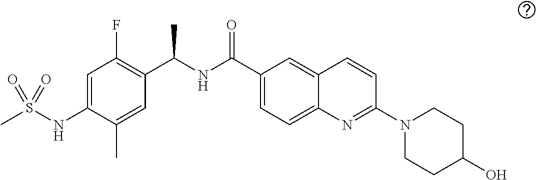 | 500.59 | | 1000 |
| 110 | 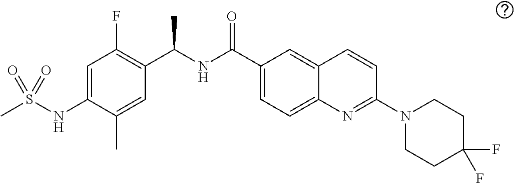 | 520.57 | | 94 |
| 111 | 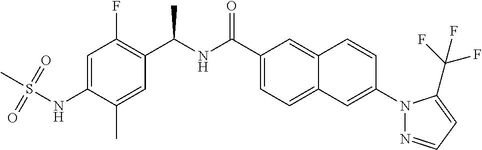 | 534.53 | 535.2 | 1000 |
| 112 | 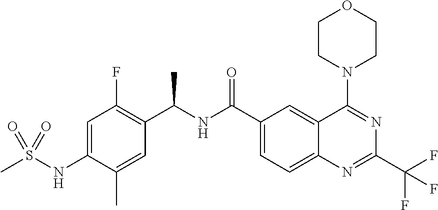 | 555.55 | 556.3 | 33 |
| 113 | 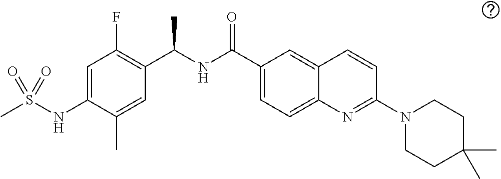 | 512.65 | | 149 |
| 114 | 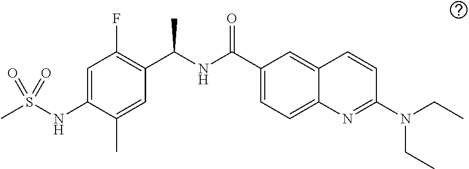 | 472.58 | | |
| 115 | 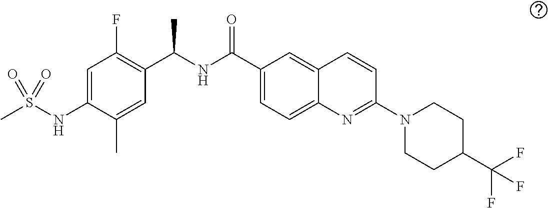 | 552.59 | | |
| 116 | 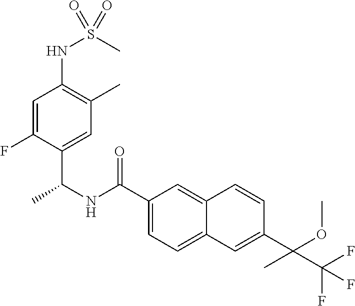 | 526.55 | | |

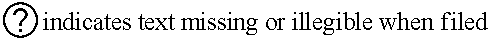

### Half-Life in Human Liver Microsomes (HLM)

Exemplary compounds of the invention were tested (1 μM), and were incubated with 3.3 mM MgCl₂ and 0.78 mg/mL HLM (HL101) in 100 mM potassium phosphate buffer (pH 7A) at 37° C. on the 96-deep well plate. The reaction mixture was split into two groups, a non-P450 and a P450 group. NADPH was only added to the reaction mixture of the P450 group. An aliquot of samples of the P450 group was collected at 0, 10, 30, and 60 minute time points, where the 0 minute time point indicated the time when NADPH was added into the reaction mixture of the P450 group. An aliquot of samples of the non-P450 group was collected at −10 and 65 minute time points. Collected aliquots were extracted with an acetonitrile solution containing an internal standard. The precipitated protein was spun down in a centrifuge (2000 rpm, 15 min). The compound concentration in supernatant was measured by LC/MS/MS system. The half-life value (T_{1/2}) was obtained by plotting the natural logarithm of the peak area ratio of compounds/internal standard versus dine. The slope of the line of best fit through the points yields the rate of metabolism (k). This was converted to a half-life value using following equations:

Half-life=ln 2/k

### Pharmacokinetic Evaluation of Compounds Following Intravenous and Oral Administration in Rats.

Male Sprague-Dawley rats are acclimatized for at least 24 hours prior to experiment initiation. During acclimation period, all animals receive food and water ad libitum. However, food but not water is removed from the animal's cages at least 12 hours before initiation of the experiment. During the first 3 hours of experimentation, the animals receive only water ad libitum. At least three animal each are tested for intravenous and oral dosage. For intravenous formulation, compounds were dissolved (0.25 to 1 mg/mL) in a mixture of 3% dimethyl sulfoxide, 40% PEG 400 and the rest percentage of 40% Captisol in water (w/v). For oral formulation, compounds of this invention are dissolved (2 mg/mL) in a mixture of 5% of 10% Tween 80 in water (v/v) and 95% of 0.5% methyl cellulose in water (w/v). The animals are weighed before dosing. The determined body weight is used to calculate the dose volume for each animal.

Dose volume (mL/kg)=1 mg/kg/formulation concentration (mg/mL)

In instances where the formulation concentrations were less than 0.5 mg/mL, the dosing volume is about 2 mL/kg. PO rats are typically dosed through oral gavage at 2.5 mL/kg to achieve a dose level of 5 mg/kg. For IV dosing, blood samples are collected (using a pre-heparinized syringe) via the jugular vein catheter at 2, 5, 15, 30, 60, 120, 180, 300, 480, and 1440 minutes post dosing. For PO dosing, blood samples are collected (using a pre-heparinized syringe) via the jugular vein catheter before dosing and at 5, 15, 30, 60, 120, 180, 300, 480, and 1440 minutes post dosing. About 250 uL of blood is obtained at each time point from the animal. Equal volumes of 0.9% normal saline are replaced to prevent dehydration. The whole blood samples are maintained on ice until centrifugation. Blood samples are then centrifuged at 14,000 rpm for 10 minutes at 4° C. and the upper plasma layer transferred into a clean vial and stored at −80° C. The resulting plasma samples are then analyzed by liquid chromatography-tandem mass spectrometry. Following the measurement of plasma samples and dosing solutions, plasma concentration-time curve is plotted. Plasma exposure is calculated as the area under the concentration-time curve extrapolated to time infinite (AUC_{inf}). The IV AUC_{inf} is averaged and the oral bioavailability (% F) for individual animal is calculated as:

AUCinf(PO)/AUCinf(IV, average), normalized to their respective dose levels.

The % F is reported as the mean % F of all oral dosed animals.

### Example 1

### Calcium Imaging Assay

VR1 protein is a heat-gated cation channel that exchanges approximately ten calcium ions for every sodium ion resulting in neuronal membrane depolarization and elevated intracellular calcium levels. Therefore the functional activity of compounds at the VR1 receptor may be determined by measuring changes in intracellular calcium levels in neurons such as the dorsal root ganglion.

DRG neurons are grown on PDL coated 96-well black-walled plates, in the presence of DMEM medium containing 5% Penstrep, 5% Glutamax, 200 μg/ml hygromycin, 5 μg/ml blasticide and 10% heat inactivated FBS. Prior to assay, cells are loaded with 5 μg/ml Fura2 in normal saline solution at 37° C. for 40 minutes. Cells are then washed with normal saline to remove dye before commencement of the experiment.

The plated neurons are transferred into a chamber on the stage of a Nikon eclipse TE300 microscope after which neurons are allowed to attain a stable fluorescence for about 10 minutes before beginning the experiment. The assay consists of two stages, a pretreatment phase followed by a treatment phase. First, a solution of the test compound is added from a multivalve perfusion system to the cells for 1 minute (pretreatment). Immediately following, capsaicin (250 nM) is added in the presence of the test compound (treatment) for a specific period between 20 and 60 seconds.

Fura2 is excited at 340 and 380 nM to indicate relative calcium ion concentration. Changes in wavelength measurements are made throughout the course of the experiment. The fluorescence ratio is calculated by dividing fluorescence measured at 340 nM by that at 380 nM. Data are collected using Intelligent Imaging's Slidebook software. All compounds that inhibit capsaicin induced calcium influx greater than 75% are considered positives.

### Example 2

### High Throughput Analysis of VR1 Antagonists for Determination of In Vitro Efficacy Using a Calcium Imaging Assay

Inhibition of the capsaicin response in the presence and absence of the test compound was measured and assessed, using the method for calcium uptake assay, described hereinabove with respect to the data presented in Table 1. No such reduction in response is observed in the absence of the test compound.

### Example 3

### Whole-Cell Patch Clamp Electrophysiology

Dorsal root ganglion (DRG) neurons are recovered from either neonatal or adult rats and plated onto poly-D-lysine coated glass coverslips. The plated neurons are transferred into a chamber to allow drug solutions to be added to the cells using a computer-controlled solenoid-valve based perfusion system. The cells are imaged using standard DIC optics. Cells are patched using finely-pulled glass electrodes. Voltage-clamp electrophysiology experiments are carried out using an Axon Instruments Multiclamp amplified controlled by pCLAMP8 software.

The cells are placed into a whole-cell voltage clamp and held at a voltage of −80 mV while monitoring the membrane current in gap-free recording mode. 500 nM capsaicin is added for 30 seconds as a control. Test compounds at various concentrations are added to the cells for 1 minute prior to a 30 second capsaicin application. Differences between control experiments and drug positive capsaicin experiments are used to determine the efficacy of each test compound. All compounds that inhibit capsaicin induced current greater than 50% are considered positives.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All such modifications coming within the scope of the appended claims are intended to be included therein.

The chemical names of compounds given in this application were generated using Open Eye Software's Lexichem naming tool, Symyx Renaissance Software's Reaction Planner or MDL's ISIS Draw Autonom Software tool and not verified. Preferably, in the event of inconsistency, the depicted structure governs.

1. A compound of a formula: 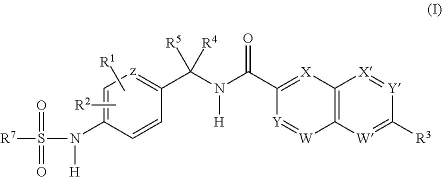
or a pharmaceutically acceptable salt thereof, and isotopic variants thereof, stereoisomers and tautomers thereof, wherein:
W, W′, X, X′, Y, Y′ and Z each independently represents CR⁸ or N;
R¹ and R² each independently represents hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl or (C₁-C₆)alkylsulfonyl;
R³ represents
hydrogen, halogen, hydroxy, (C₁-C₆)alkyl, halo(C₁-C₆)alkyl, hydroxy(C₁-C₆)alkyl, halo hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—, [(C₁-C₆)alkyl]₂N—, [hydroxy(C₁-C₆)alkyl]NH—, 3-6 membered cycloalkyl, [3-6 membered cycloalkyl]oxy, or [3-6 membered heterocycloalkyl]oxy
or
3-6 membered heterocycloalkyl, unsubstituted or substituted with
halo, (C₁-C₆)alkyl, halo(C₁-C₆)alkyl, hydroxy(C₁-C₆)alkyl, (C₁-C₆)alkoxy, [(C₁-C₆)alkyl]₂N—, or hydroxy,
or
3-6 membered heteroaryl, 3-6 membered cycloalkyl (C₁-C₆)alkyl, or 3-6 membered cycloalkyl hydroxy (C₁-C₆)alkyl;
or
1-methylcyclopropyl, 1-hydroxycyclopropyl, or 1-trifluoromethylcyclopropyl;
R⁴ and R⁵ each independently represents hydrogen, (C₁-C₆)alkyl, halogen, halo(C₁-C₆) alkyl, or hydroxy(C₁-C₆)alkyl;
each R⁸ independently represents
hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, halo hydroxy(C₁-C₆)alkyl, (C₁-C₆)alkylthio, (C₁-C₆)alkylsulfinyl, [(C₁-C₆)alkyl]NH—, [(C₁-C₆)cycloalkyl]NH—, [(C₁-C₆)alkyl]₂N—, [hydroxy(C₁-C₆)alkyl]NH—, [3-6 membered cycloalkyl]oxy, [3-6 membered heterocycloalkyl]oxy or
3-6 membered heterocycloalkyl, unsubstituted or substituted with halo, (C₁-C₆)alkyl, (C₁-C₆)alkoxy, halo(C₁-C₆)alkyl, hydroxy(C₁-C₆)alkyl, aryl(C₁-C₆)alkyl, [(C₁-C₆)alkyl]₂N—, (C₁-C₆)carbalkoxy, hydroxy, aryl, (C₁-C₆)alkylaryl, halo(C₁-C₆)alkylaryl, haloaryl, (C₁-C₆)alkoxyaryl, or
3-10 membered heteroaryl, 3-6 membered cycloalkyl (C₁-C₆)alkyl, or 3-6 membered cycloalkyl hydroxy (C₁-C₆)alkyl or (C₁-C₆)alkylsulfonyl; and
R⁷ represents (C₁-C₆)alkyl.

2. (canceled)

3. (canceled)

4. (canceled)

5. A compound of a formula: 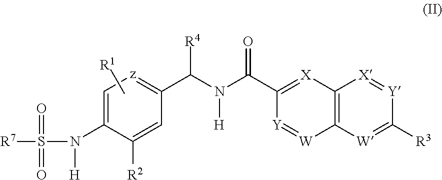
or a pharmaceutically acceptable salt, or thereof, and isotopic variants thereof, stereoisomers and tautomers thereof, wherein W, W′, X, X′, Y, Y′, Z, R¹, R², R³, R⁴, R⁷, and R⁸, are as in claim 1.

6. (canceled)

7. A compound according to claim 5 wherein R⁴ is methyl.

8. A compound according to claim 5 wherein R⁷ is Me, Et, Pr, i-Pr, or t-butyl.

9. (canceled)

10. A compound according to claim 5 wherein R¹ represents hydrogen, halogen or (C₁-C₆)alkyl.

11. A compound according to claim 5 wherein R¹ represents H or F.

12. A compound according to claim 5 wherein R² represents halogen, (C₁-C₆)alkyl, halo(C₁-C₆)alkyl or hydroxy(C₁-C₆)alkyl.

13. A compound according to claim 5 wherein R² represents F or methyl.

14. (canceled)

15. A compound according to claim 5 wherein Z represents N, CH, CF or CCl.

16. (canceled)

17. (canceled)

18. A compound according to claim 5 wherein W, W′, X, X′, Y and Y′ each independently represent CR⁸.

19. A compound according to claim 5 wherein W, W′, X, X′, Y and Y′ each independently represent CH.

20. A compound according to claim 5 wherein one of W, W′, X, X′, Y and Y′ represents N and the rest each independently represents CR⁸.

21. (canceled)

22. (canceled)

23. (canceled)

24. A compound according to claim 5 wherein X is N and each of W, W′, X′, Y and Y′ is independently CH.

25. (canceled)

26. A compound according to claim 5 wherein W′ is N and each of W, X, X′, Y and Y′ is independently CH.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A compound according to claim 5 wherein X is CR⁸ and R⁸ is Me, OH, OMe, Cl or F.

40. A compound according to claim 5 wherein W, W′, X, X′, Y and Y′ each independently represent CH and R³ represents halogen, (C₁-C₆)alkyl, halo(C₁-C₆)alkyl, hydroxy(C₁-C₆)alkyl, (C₁-C₆)alkoxy, hydroxy(C₁-C₆)alkoxy, (C₁-C₆)alkoxy-(C₁-C₆)alkyl, (C₁-C₆)acyl, (C₁-C₆)alkoxy-(C₁-C₆)alkoxy, [(C₁-C₆)alkyl]NH—, [(C₁-C₆)alkyl]₂N—, 3-6 membered cycloalkyl, 3-6 membered heterocycloalkyl, 3-6 membered cycloalkyl (C₁-C₆)alkyl, or 3-6 membered cycloalkyl hydroxy (C₁-C₆)alkyl.

41. (canceled)

42. A compound according to claim 5 wherein W, W′, X, X′, Y and Y′ each independently represent CH; and R³ represents OMe, OEt, COMe, NMe₂, or NEt₂.

43. (canceled)

44. A compound according to claim 5 wherein R³ is F, Cl, CF₃, Me, i-Pr, t-Bu, 1-methyl-1-trifluoromethylethyl, or 1-methyl-1-hydroxyethyl.

45. (canceled)

46. (canceled)

47. A compound according to claim 5 wherein R³ is cyclopropyl, 1-methylcyclopropyl, 1-hydroxycyclopropyl, 1-trifluoromethylcyclopropyl, cyclobutyl or cyclopentyl.

48. (canceled)

49. A compound according to claim 5 wherein R³ is

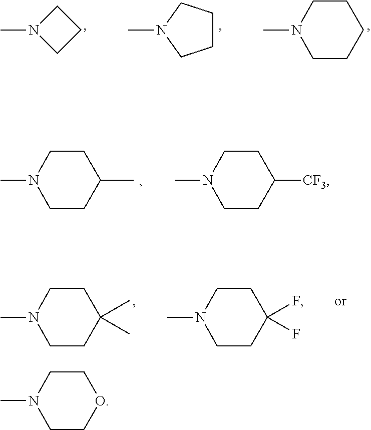

50. (canceled)

51. (canceled)

52. A compound according to claim 1, selected from
6-tert-Butyl-naphthalene-2-carboxylic acid [(R)-1-(4-methanesulfonylamino-3-methyl-phenyl)-ethyl]-amide;
6-tert-Butyl-naphthalene-2-carboxylic acid [(R)-1-(5-methanesulfonylamino-6-methyl-pyridin-2-yl)-ethyl]-amide;
6-Trifluoromethyl-quinoline-2-carboxylic acid [(R)-1-(3,5-difluoro-4-methanesulfonylamino-phenyl)-ethyl]-amide;
7-Trifluoromethyl-quinoline-3-carboxylic acid [(R)-1-(4-methanesulfonylamino-3-methyl-phenyl)-ethyl]-amide;
6-Trifluoromethyl-quinoline-2-carboxylic acid [(R)-1-(4-methanesulfonylamino-3-methyl-phenyl)-ethyl]-amide;
6-tert-Butyl-quinoline-2-carboxylic acid [(R)-1-(4-methanesulfonylamino-3-methyl-phenyl)-ethyl]-amide;
6-tert-Butyl-quinoline-2-carboxylic acid [(R)-1-(3,5-difluoro-4-methanesulfonylamino-phenyl)-ethyl]-amide;
2-tert-Butyl-quinoline-6-carboxylic acid [(R)-1-(4-methanesulfonylamino-3-methyl-phenyl)-ethyl]-amide;
6-Isopropyl-quinoline-2-carboxylic acid [(R)-1-(4-methanesulfonylamino-3-methyl-phenyl)-ethyl]-amide;
2-Trifluoromethyl-quinoline-6-carboxylic acid [(R)-1-(4-methanesulfonylamino-3-methyl-phenyl)-ethyl]-amide;
4-Methyl-7-trifluoromethyl-quinoline-3-carboxylic acid [(R)-1-(4-methanesulfonylamino-3-methyl-phenyl)-ethyl]-amide;
6-Bromo-quinoline-2-carboxylic acid [(R)-1-(4-methanesulfonylamino-3-methyl-phenyl)-ethyl]-amide;
6-tert-Butyl-naphthalene-2-carboxylic acid [(R)-1-(2-fluoro-4-methanesulfonylamino-5-methyl-phenyl)-ethyl]-amide;
6-tert-Butyl-quinoline-2-carboxylic acid [(R)-1-(2-fluoro-4-methanesulfonylamino-5-methyl-phenyl)-ethyl]-amide;
2-tert-Butyl-quinoline-6-carboxylic acid [(R)-1-(3,5-difluoro-4-methanesulfonylamino-phenyl)-ethyl]-amide;
6-(1-Hydroxy-1-methyl-ethyl)-quinoline-2-carboxylic acid [(R)-1-(4-methanesulfonylamino-3-methyl-phenyl)-ethyl]-amide;
6-Bromo-naphthalene-2-carboxylic acid [(R)-1-(4-methanesulfonylamino-3-methyl-phenyl)-ethyl]-amide;
6-Fluoro-naphthalene-2-carboxylic acid [(R)-1-(4-methanesulfonylamino-3-methyl-phenyl)-ethyl]-amide;
Naphthalene-2-carboxylic acid [(R)-1-(4-methanesulfonylamino-3-methyl-phenyl)-ethyl]-amide;
6-Methoxy-naphthalene-2-carboxylic acid [(R)-1-(4-methanesulfonylamino-3-methyl-phenyl)-ethyl]-amide;
6-Pyrrolidin-1-yl-naphthalene-2-carboxylic acid [(R)-1-(4-methanesulfonylamino-3-methyl-phenyl)-ethyl]-amide;
6-Cyclopropyl-naphthalene-2-carboxylic acid [(R)-1-(4-methanesulfonylamino-3-methyl-phenyl)-ethyl]-amide;
7-Chloro-2-methyl-quinoline-3-carboxylic acid [(R)-1-(4-methanesulfonylamino-3-methyl-phenyl)-ethyl]-amide;
6-(1-Methyl-cyclopropyl)-naphthalene-2-carboxylic acid [(R)-1-(5-methanesulfonylamino-4-methyl-pyridin-2-yl)-ethyl]-amide;
7-Trifluoromethyl-quinoline-3-carboxylic acid [(R)-1-(3,5-difluoro-4-methanesulfonylamino-phenyl)-ethyl]-amide;
6-Acetyl-quinoline-2-carboxylic acid [(R)-1-(4-methanesulfonylamino-3-methyl-phenyl)-ethyl]-amide;
6-tert-Butyl-quinoline-3-carboxylic acid [(R)-1-(4-methanesulfonylamino-3-methyl-phenyl)-ethyl]-amide;
6-(1-Cyclopropyl-1-hydroxy-ethyl)-quinoline-2-carboxylic acid [(R)-1-(4-methanesulfonylamino-3-methyl-phenyl)-ethyl]-amide;
2-Isopropyl-quinoline-6-carboxylic acid [(R)-1-(3,5-difluoro-4-methanesulfonylamino-phenyl)-ethyl]-amide;
2-Trifluoromethyl-quinoline-6-carboxylic acid [(R)-1-(2-fluoro-4-methanesulfonylamino-5-methyl-phenyl)-ethyl]-amide;
7-tert-Butyl-quinoline-3-carboxylic acid [(R)-1-(4-methanesulfonylamino-3-methyl-phenyl)-ethyl]-amide;
7-Trifluoromethyl-quinoline-3-carboxylic acid [(R)-1-(3-fluoro-4-methanesulfonylamino-phenyl)-ethyl]-amide;
2-Isopropyl-quinoline-6-carboxylic acid [(R)-1-(4-methanesulfonylamino-3-methyl-phenyl)-ethyl]-amide;
7-tert-Butyl-quinoline-3-carboxylic acid [(R)-1-(3,5-difluoro-4-methanesulfonylamino-phenyl)-ethyl]-amide;
2-Isopropyl-quinoline-6-carboxylic acid [(R)-1-(2-fluoro-4-methanesulfonylamino-5-methyl-phenyl)-ethyl]-amide;
7-Isopropyl-quinoline-3-carboxylic acid [(R)-1-(4-methanesulfonylamino-3-methyl-phenyl)-ethyl]-amide;
7-Isopropyl-quinoline-3-carboxylic acid [(R)-1-(3,5-difluoro-4-methanesulfonylamino-phenyl)-ethyl]-amide;
6-tert-Butyl-naphthalene-2-carboxylic acid [(R)-1-(5-methanesulfonylamino-4-methyl-pyridin-2-yl)-ethyl]-amide;
7-tert-Butyl-quinoline-3-carboxylic acid [(R)-1-(4-methanesulfonylamino-phenyl)-ethyl]amide;
7-tert-Butyl-4-methyl-quinoline-3-carboxylic acid [(R)-1-(4-methanesulfonylamino-3-methyl-phenyl)-ethyl]-amide;
7-Trifluoromethyl-quinoline-3-carboxylic acid [(R)-1-(2-fluoro-4-methanesulfonylamino-5-methyl-phenyl)-ethyl]-amide;
2-Ethoxy-quinoline-6-carboxylic acid [(R)-1-(4-methanesulfonylamino-3-methyl-phenyl)-ethyl]-amide;
6-Cyclopropyl-naphthalene-2-carboxylic acid [(R)-1-(2-fluoro-4-methanesulfonylamino-5-methyl-phenyl)-ethyl]-amide;
2-Cyclopropyl-quinoline-6-carboxylic acid [(R)-1-(3,5-difluoro-4-methanesulfonylamino-phenyl)-ethyl]-amide;
6-Trifluoromethyl-naphthalene-2-carboxylic acid [(R)-1-(2-fluoro-4-methanesulfonylamino-5-methyl-phenyl)-ethyl]-amide;
2-(1-Methyl-cyclopropyl)-quinoline-6-carboxylic acid [(R)-1-(3,5-difluoro-4-methanesulfonylamino-phenyl)-ethyl]-amide;
2-(2,2,2-Trifluoro-1,1-dimethyl-ethyl)-quinoline-6-carboxylic acid [(R)-1-(4-methanesulfonylamino-3-methyl-phenyl)-ethyl]-amide;
6-(1-Methyl-cyclopropyl)-naphthalene-2-carboxylic acid [(R)-1-(2-fluoro-4-methanesulfonylamino-5-methyl-phenyl)-ethyl]-amide;
6-Trifluoromethyl-naphthalene-2-carboxylic acid [(R)-1-(4-methanesulfonylamino-3-methyl-phenyl)-ethyl]-amide;
6-Cyclopropyl-naphthalene-2-carboxylic acid [(R)-1-(2-chloro-4-methanesulfonylamino-5-methyl-phenyl)-ethyl]-amide;
7-Trifluoromethyl-quinoline-3-carboxylic acid [(R)-1-(2-chloro-4-methanesulfonylamino-5-methyl-phenyl)-ethyl]-amide;
6-Trifluoromethyl-naphthalene-2-carboxylic acid [(R)-1-(5-methanesulfonylamino-4-methyl-pyridin-2-yl)-ethyl]-amide;
2-(2,2,2-Trifluoro-1,1-dimethyl-ethyl)-quinoline-6-carboxylic acid [(R)-1-(2-fluoro-4-methanesulfonylamino-5-methyl-phenyl)-ethyl]-amide;
6-Trifluoromethyl-quinoline-2-carboxylic acid [(R)-1-(2-fluoro-4-methanesulfonylamino-5-methyl-phenyl)-ethyl]-amide;
6-Trifluoromethyl-quinoline-2-carboxylic acid [(R)-1-(2-chloro-4-methanesulfonylamino-5-methyl-phenyl)-ethyl]-amide;
4-Hydroxy-7-trifluoromethyl-quinoline-3-carboxylic acid [(R)-1-(4-methanesulfonylamino-3-methyl-phenyl)-ethyl]-amide;
7-Trifluoromethyl-quinoline-2-carboxylic acid [(R)-1-(4-methanesulfonylamino-3-methyl-phenyl)-ethyl]-amide;
Quinoline-3-carboxylic acid [(R)-1-(4-methanesulfonylamino-3-methyl-phenyl)-ethyl]amide;
4-Morpholin-4-yl-2-trifluoromethyl-quinoline-6-carboxylic acid [(R)-1-(2-fluoro-4-methanesulfonylamino-5-methyl-phenyl)-ethyl]-amide;
6-Fluoro-7-trifluoromethyl-quinoline-3-carboxylic acid [(R)-1-(2-fluoro-4-methanesulfonylamino-5-methyl-phenyl)-ethyl]-amide;
6-Chloro-7-trifluoromethyl-quinoline-3-carboxylic acid [(R)-1-(2-fluoro-4-methanesulfonylamino-5-methyl-phenyl)-ethyl]-amide;
2-Pyrrolidin-1-yl-quinoline-6-carboxylic acid [(R)-1-(2-fluoro-4-methanesulfonylamino-5-methyl-phenyl)-ethyl]-amide;
2-Dimethylamino-quinoline-6-carboxylic acid [(R)-1-(2-fluoro-4-methanesulfonylamino-5-methyl-phenyl)-ethyl]-amide;
4-Piperidin-1-yl-2-trifluoromethyl-quinoline-6-carboxylic acid [(R)-1-(2-fluoro-4-methanesulfonylamino-5-methyl-phenyl)-ethyl]-amide;
4-Pyrrolidin-1-yl-2-trifluoromethyl-quinoline-6-carboxylic acid [(R)-1-(2-fluoro-4-methanesulfonylamino-5-methyl-phenyl)-ethyl]-amide;
4-(4,4-Difluoro-piperidin-1-yl)-2-trifluoromethyl-quinoline-6-carboxylic acid [(R)-1-(2-fluoro-4-methanesulfonylamino-5-methyl-phenyl)-ethyl]-amide;
6-Trifluoromethyl-naphthalene-2-carboxylic acid [(R)-1-(3-hydroxymethyl-4-methanesulfonylamino-phenyl)-ethyl]-amide;
2-Piperidin-1-yl-quinoline-6-carboxylic acid [(R)-1-(2-fluoro-4-methanesulfonylamino-5-methyl-phenyl)-ethyl]-amide;
2-Trifluoromethyl-4-(4-trifluoromethyl-piperidin-1-yl)-quinoline-6-carboxylic acid [(R)-1-(2-fluoro-4-methanesulfonylamino-5-methyl-phenyl)-ethyl]-amide;
7-Trifluoromethyl-naphthalene-2-carboxylic acid [(R)-1-(4-methanesulfonylamino-3-methyl-phenyl)-ethyl]-amide;
4-[4-(2-Hydroxy-ethyl)-piperazin-1-yl]-2-trifluoromethyl-quinoline-6-carboxylic acid [(R)-1-(2-fluoro-4-methanesulfonylamino-5-methyl-phenyl)-ethyl]-amide;
2-Morpholin-4-yl-quinoline-6-carboxylic acid [(R)-1-(2-fluoro-4-methanesulfonylamino-5-methyl-phenyl)-ethyl]-amide;
4-Piperidin-1-yl-2-trifluoromethyl-quinazoline-6-carboxylic acid [(R)-1-(2-fluoro-4-methanesulfonylamino-5-methyl-phenyl)-ethyl]-amide;
4-[4-(3-Chloro-phenyl)-piperazin-1-yl]-2-trifluoromethyl-quinazoline-6-carboxylic acid [(R)-1-(2-fluoro-4-methanesulfonylamino-5-methyl-phenyl)-ethyl]-amide;
4-(2,6-Dimethyl-morpholin-4-yl)-2-trifluoromethyl-quinazoline-6-carboxylic acid [(R)-1-(2-fluoro-4-methanesulfonylamino-5-methyl-phenyl)-ethyl]-amide;
7-Cyclopropyl-[1,5]naphthyridine-3-carboxylic acid [(R)-1-(2-fluoro-4-methanesulfonylamino-5-methyl-phenyl)-ethyl]-amide;
4-[4-(2-Methoxy-phenyl)-piperazin-1-yl]-2-trifluoromethyl-quinazoline-6-carboxylic acid [(R)-1-(2-fluoro-4-methanesulfonylamino-5-methyl-phenyl)-ethyl]-amide;
4-((R)-3-Hydroxy-pyrrolidin-1-yl)-2-trifluoromethyl-quinoline-6-carboxylic acid [(R)-1-(2-fluoro-4-methanesulfonylamino-5-methyl-phenyl)-ethyl]-amide;
4-((S)-3-Hydroxy-pyrrolidin-1-yl)-2-trifluoromethyl-quinoline-6-carboxylic acid [(R)-1-(2-fluoro-4-methanesulfonylamino-5-methyl-phenyl)-ethyl]-amide;
4-(3,3-Difluoro-azetidin-1-yl)-2-trifluoromethyl-quinoline-6-carboxylic acid [(R)-1-(2-fluoro-4-methanesulfonylamino-5-methyl-phenyl)-ethyl]-amide;
4-((R)-2-Hydroxymethyl-pyrrolidin-1-yl)-2-trifluoromethyl-quinoline-6-carboxylic acid [(R)-1-(2-fluoro-4-methanesulfonylamino-5-methyl-phenyl)-ethyl]-amide;
4-(Tetrahydro-pyran-4-yloxy)-2-trifluoromethyl-quinoline-6-carboxylic acid [(R)-1-(2-fluoro-4-methanesulfonylamino-5-methyl-phenyl)-ethyl]-amide;
4-(4-Hydroxy-piperidin-1-yl)-2-trifluoromethyl-quinoline-6-carboxylic acid [(R)-1-(2-fluoro-4-methanesulfonylamino-5-methyl-phenyl)-ethyl]-amide;
4-{6-[(R)-1-(2-Fluoro-4-methanesulfonylamino-5-methyl-phenyl)-ethylcarbamoyl]-2-trifluoromethyl-quinazolin-4-yl}-piperazine-1-carboxylic acid ethyl ester
4-Cyclohexylamino-2-trifluoromethyl-quinoline-6-carboxylic acid [(R)-1-(2-fluoro-4-methanesulfonylamino-5-methyl-phenyl)-ethyl]-amide;
7-Pyrrolidin-1-yl-[1,5]naphthyridine-3-carboxylic acid [(R)-1-(2-fluoro-4-methanesulfonylamino-5-methyl-phenyl)-ethyl]-amide;
4-(4-Hydroxymethyl-piperidin-1-yl)-2-trifluoromethyl-quinoline-6-carboxylic acid [(R)-1-(2-fluoro-4-methanesulfonylamino-5-methyl-phenyl)-ethyl]-amide;
6-(2,2,2-Trifluoro-1-hydroxy-1-methyl-ethyl)-naphthalene-2-carboxylic acid [(R)-1-(2-fluoro-4-methanesulfonylamino-5-methyl-phenyl)-ethyl]-amide;
6-Pyrazol-1-yl-naphthalene-2-carboxylic acid [(R)-1-(2-fluoro-4-methanesulfonylamino-5-methyl-phenyl)-ethyl]-amide;
4-(4-Benzyl-piperidin-1-yl)-2-trifluoromethyl-quinazoline-6-carboxylic acid [(R)-1-(2-fluoro-4-methanesulfonylamino-5-methyl-phenyl)-ethyl]-amide;
2-(4-Methoxy-piperidin-1-yl)-quinoline-6-carboxylic acid [(R)-1-(2-fluoro-4-methanesulfonylamino-5-methyl-phenyl)-ethyl]-amide;
2-(1-Methyl-cyclopropyl)-quinoline-6-carboxylic acid [(R)-1-(2-fluoro-4-methanesulfonylamino-5-methyl-phenyl)-ethyl]-amide;
2-(4,4-Difluoro-piperidin-1-yl)-quinoline-6-carboxylic acid [(R)-1-(2-fluoro-4-methanesulfonylamino-5-methyl-phenyl)-ethyl]-amide;
4-Morpholin-4-yl-2-trifluoromethyl-quinazoline-6-carboxylic acid [(R)-1-(2-fluoro-4-methanesulfonylamino-5-methyl-phenyl)-ethyl]-amide;
2-(4,4-Dimethyl-piperidin-1-yl)-quinoline-6-carboxylic acid [(R)-1-(2-fluoro-4-methanesulfonylamino-5-methyl-phenyl)-ethyl]-amide;
2-Diethylamino-quinoline-6-carboxylic acid [(R)-1-(2-fluoro-4-methanesulfonylamino-5-methyl-phenyl)-ethyl]-amide;
2-(4-Trifluoromethyl-piperidin-1-yl)-quinoline-6-carboxylic acid [(R)-1-(2-fluoro-4-methanesulfonylamino-5-methyl-phenyl)-ethyl]-amide; and
6-(2,2,2-Trifluoro-1-methoxy-1-methyl-ethyl)-naphthalene-2-carboxylic acid [(R)-1-(2-fluoro-4-methanesulfonylamino-5-methyl-phenyl)-ethyl]-amide;
or a pharmaceutically acceptable salt thereof, and isotopic variants thereof, stereoisomers and tautomers thereof.

53. A pharmaceutical composition comprising a pharmaceutically acceptable carrier and a pharmaceutically effective amount of a compound of claim 1.

54. The pharmaceutical composition of claim 53 wherein the carrier is a parenteral carrier, oral or topical carrier.

55. A method for treating a disease or condition which comprises administering to a patient in need of a therapeutically effective amount of a compound of claim 1, or a pharmaceutical acceptable salt thereof.

56. (canceled)

57. (canceled)

58. (canceled)

59. (canceled)

60. A method for treating a disease or condition which comprises administering to a patient in need of a therapeutically acceptable amount of a compound of claim 1, or the pharmaceutical composition of claim 53, wherein the disease is: pain including acute, inflammatory and neuropathic pain; chronic pain; dental pain; headache including migraine, cluster headache and tension headache; Parkinson's disease; Alzheimer's disease; multiple sclerosis; diseases and disorders mediated by or result in neuroinflammation, traumatic brain injury, stroke, or encephalitis; centrally-mediated neuropsychiatric diseases and disorders including depression, mania, bipolar disease, anxiety, schizophrenia, eating disorders, sleep disorders and cognition disorders; epilepsy and seizure disorders; prostate, bladder and bowel dysfunction, urinary incontinence, urinary hesitancy, rectal hypersensitivity, fecal incontinence, benign prostatic hypertrophy and inflammatory bowel disease; respiratory and airway disease and disorders including allergic rhinitis, asthma and reactive airway disease and chronic obstructive pulmonary disease; diseases and disorders mediated by or result in inflammation including arthritis, rheumatoid arthritis and osteoarthritis; myocardial infarction; autoimmune diseases and disorders; uveitis and atherosclerosis; itch/pruritus, psoriasis; alopecia (hair loss); obesity; lipid disorders; cancer; high blood pressure; spinal cord injury; irritable bowel syndrome; overactive bladder; or renal disorders.

61. (canceled)

62. (canceled)

63. (canceled)

64. (canceled)

65. (canceled)

66. (canceled)

67. (canceled)

68. A method of treating a mammal suffering from at least one symptom selected from the group consisting of symptoms of exposure to capsaicin, symptoms of burns or irritation due to exposure to heat, symptoms of burns or irritation due to exposure to light, symptoms of burns, bronchoconstriction or irritation due to exposure to tear gas, and symptoms of burns or exposure irritation due to exposure to acid which comprises administering to the mammal an effective disease-treating or condition-treating amount of a compound of claim 1, or the pharmaceutical composition of claim 53.

69. (canceled)

70. (canceled)
